(19) United States
(12) Patent Application Publication
(10) Pub. No.: US 2024/0181081 A1
(43) Pub. Date: Jun. 6, 2024

(54) Compositions and Methods for Treatment of Myotonic Dystrophy Type 1 with CRISPR/SACAS9

(71) Applicant: Vertex Pharmaceuticals Incorporated, Boston, MA (US)

(72) Inventors: Guoxiang Ruan, Boston, MA (US); Jianming Liu, Boston, MA (US); Tudor Fulga, Boston, MA (US); Eric Gunnar Anderson, Cambridge, MA (US); Lingjun Rao, Boston, MA (US); Norzehan Abdul-Manan, Boston, MA (US); Matthias Heidenreich, Boston, MA (US); Gregoriy Aleksandrovich Dokshin, Boston, MA (US); Jesper Gromada, Boston, MA (US)

(73) Assignee: Vertex Pharmaceuticals Incorporated, Boston, MA (US)

(21) Appl. No.: 18/456,269

(22) Filed: Aug. 25, 2023

Related U.S. Application Data
(63) Continuation of application No. PCT/US2022/017850, filed on Feb. 25, 2022.
(60) Provisional application No. 63/306,883, filed on Feb. 4, 2022.
(60) Provisional application No. 63/276,003, filed on Nov. 5, 2021.
(60) Provisional application No. 63/184,462, filed on May 5, 2021.
(60) Provisional application No. 63/159,815, filed on Mar. 11, 2021.
(60) Provisional application No. 63/154,442, filed on Feb. 26, 2021.

(51) Int. Cl. A61K 48/00; C12N 9/22; C12N 15/113; C12N 15/86; C12N 15/90

(52) U.S. Cl. CPC A61K 48/005; C12N 9/22; C12N 15/1137; C12N 15/86; C12N 15/907; C12N 2310/20; C12N 2320/34; C12N 2750/14143; C12N 2800/80

(57) ABSTRACT

Compositions and methods for treating Myotonic Dystrophy Type 1 (DM1) are encompassed.

This application is a bypass continuation of PCT/US2022/017850 filed Feb. 25, 2022, which claims the benefit of priority to U.S. Provisional Application No. 63/154,442, filed Feb. 26, 2021; U.S. Provisional Application No. 63/159,815, filed Mar. 11, 2021; U.S. Provisional Patent Application No. 63/184,462, filed May 5, 2021; U.S. Provisional Application No. 63/276,003, filed Nov. 5, 2021; and U.S. Provisional Patent Application No. 63/306,883, filed Feb. 4, 2022; all of which are incorporated by reference in their entirety.

The instant application contains a Sequence Listing which has been submitted electronically in XML format and is hereby incorporated by reference in its entirety. Said XML copy, created on Aug. 17, 2023, is named 2023-08-17_01245-0025-00US_ST26 and is 387,989 bytes in size.

### INTRODUCTION AND SUMMARY

Myotonic Dystrophy Type 1 (DM1) is an autosomal dominant muscle disorder caused by the expansion of CTG repeats in the 3′ untranslated region (UTR) of human DMPK gene, which leads to RNA foci and mis-splicing of genes important for muscle function. The disorder affects skeletal and smooth muscle as well as the eye, heart, endocrine system, and central nervous system, and causes muscle weakness, wasting, physical disablement, and shortened lifespan.

CRISPR-based genome editing can provide sequence-specific cleavage of genomic DNA using a Cas9 and a guide RNA. For example, a nucleic acid encoding the Cas9 enzyme and a nucleic acid encoding for the appropriate guide RNA can be provided on separate vectors or together on a single vector and administered in vivo or in vitro to knockout or correct a genetic mutation. The approximately 20 nucleotides at the 5′ end of the guide RNA serves as the guide or spacer sequence that can be any sequence complementary to one strand of a genomic target location that has an adjacent protospacer adjacent motif (PAM). The PAM sequence is a short sequence adjacent to the Cas9 nuclease cut site that the Cas9 molecule requires for appropriate binding. The nucleotides 3′ of the guide or spacer sequence of the guide RNA serve as a scaffold sequence for interacting with Cas9. When a guide RNA and a Cas9 are expressed, the guide RNA will bind to Cas9 and direct it to the sequence complementary to the guide sequence, where it will then initiate a double-stranded break (DSB). To repair these breaks, cells typically use an error prone mechanism of non-homologous end joining (NHEJ) which can lead to disruption of function in the target gene through insertions or deletion of codons, shifts in the reading frame, or result in a premature stop codon triggering nonsense-mediated decay. See, e.g., Kumar et al. (2018) Front. Mol. Neurosci. Vol. 11, Article 413.

Adeno-associated virus (AAV) administration of the CRISPR-Cas components in vivo or in vitro is attractive due to the early and ongoing successes of AAV vector design, manufacturing, and clinical stage administration for gene therapy. See, e.g., Wang et al. (2019) Nature Reviews Drug Discovery 18:358-378; Ran et al. (2015a) Nature 520: 186-101. However, the commonly used Streptococcus pyogenes (spCas9) is very large, and when used in AAV-based CRISPR/Cas systems, requires two AAV vectors—one vector carrying the nucleic acid encoding the spCas9, and the other carrying the nucleic acid encoding the guide RNA. One possible way to overcome this technical hurdle is to take advantage of the smaller orthologs of Cas9 derived from different prokaryotic species. Smaller Cas9's may be able to be manufactured on a single AAV vector together with a nucleic acid encoding a guide RNA thereby reducing manufacturing costs and reducing complexity of administration routes and protocols.

Provided herein are compositions and methods for treating DM1 utilizing the smaller Cas9 from Staphylococcus aureus (SaCas9). Compositions comprising i) a single AAV vector comprising a nucleic acid molecule encoding SaCas9, and one or more guide RNAs; and ii) an optional DNA-PK inhibitor are provided. Methods using disclosed compositions to treat DM1 are also provided. Compositions and methods disclosed herein may be used for excising a portion of the CTG repeat region to treat DM1, reduce RNA foci, and/or correct mis-splicing in DM1 patient cells. For example, disclosed herein are guide RNAs and combinations of guide RNAs particularly suitable for use with saCas9 for use in methods of excising a CTG repeat in the 3′ UTR of DMPK, with or without a DNA-PK inhibitor.

Also provided herein are systems comprising more than one vector, whereby one or more guide RNAs are incorporated on a single vector together with a smaller SaCas9 and another vector comprises nucleic acid encoding multiple copies of guide RNAs. Such systems allow extreme design flexibility in situations where more than one guide RNA is desired for optimal performance. For example, one vector may be utilized to express SaCas9 and one or more guide RNAs targeting one or more genomic targets, and a second vector may be utilized to express multiple copies of the same or different guide RNAs targeting the same or different genomic targets. Compositions and methods utilizing these dual vector configurations are provided herein and have the benefit of reducing manufacturing costs, reducing complexity of administration routes and protocols, and allowing maximum flexibility with regard to using multiple copies of the same or different guide RNAs targeting the same or different genomic target sequences. In some instances, providing multiple copies of the same guide RNA improves the efficiency of the guide, improving an already successful system. Accordingly, the following non-limiting embodiments are provided:

[Embodiment 01] A composition comprising a single nucleic acid molecule encoding one or more guide RNAs and a Cas9, wherein the single nucleic acid molecule comprises:
a. a first nucleic acid encoding one or more spacer sequences selected from any one of SEQ ID NOs: 1-8, 10-28, and 101-154, and a second nucleic acid encoding a Staphylococcus aureus Cas9 (SaCas9);
b. a first nucleic acid encoding one or more spacer sequences comprising at least 20, or 21 contiguous nucleotides of a spacer sequence selected from any one of SEQ ID NOs: 1-8, 10-28, and 101-154, and a second nucleic acid encoding a Staphylococcus aureus Cas9 (SaCas9);
c. a first nucleic acid encoding one or more spacer sequences that are at least 90% identical to any one of SEQ ID NOs: 1-8, 10-28, and 101-154, and a second nucleic acid encoding a Staphylococcus aureus Cas9 (SaCas9);
d. a first nucleic acid encoding one or more spacer sequences selected from any one of SEQ ID NOs: 1, 2, 3, 4, 7, 8, 10, 11, 12, 13, 14, 15, 18, 19, 20, 21, 23, 25, 26, 27, and 28, and a second nucleic acid encoding a Staphylococcus aureus Cas9 (SaCas9);
e. a first nucleic acid encoding one or more spacer sequences selected from any one of SEQ ID NOs: 1, 2, 3, 4, 7, 8, 12, and 20, and a second nucleic acid encoding a Staphylococcus aureus Cas9 (SaCas9);
f. a first nucleic acid encoding one or more spacer sequences selected from any one of SEQ ID NOs: 1, 2, 3, 4, 7, 8, and 20, and a second nucleic acid encoding a Staphylococcus aureus Cas9 (SaCas9);
g. a first nucleic acid encoding one or more spacer sequences selected from any one of SEQ ID NOs: 1, 101, and 102, and a second nucleic acid encoding a Staphylococcus aureus Cas9 (SaCas9);
h. a first nucleic acid encoding a pair of guide RNAs comprising a first and second spacer sequence selected from any one of SEQ ID NOs: 1 and 10; 1 and 11; 1 and 12; 1 and 13; 1 and 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28, and a second nucleic acid encoding a Staphylococcus aureus Cas9 (SaCas9);
i. a first nucleic acid encoding a pair of guide RNAs comprising at least 20, or 21 contiguous nucleotides of a first and second spacer sequence selected from any one of SEQ ID NOs: 1 and 10; 1 and 11; 1 and 12; 1 and 13; 1 and 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28, and a second nucleic acid encoding a Staphylococcus aureus Cas9 (SaCas9);
j. a first nucleic acid encoding a pair of guide RNAs that is at least 90% identical to a first and second spacer sequence selected from any one of SEQ ID NOs: 1 and 10; 1 and 11; land 12; land 13; land 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28, and a second nucleic acid encoding a Staphylococcus aureus Cas9 (SaCas9);
k. a first nucleic acid encoding a pair of guide RNAs comprising a first and second spacer sequence selected from any one of SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 8 and 10; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 7 and 10; 1 and 13; 1 and 20; 1 and 28; 4 and 27; 7 and 20; 7 and 23; 7 and 13; 7 and 28; 2 and 27; 8 and 27; 4 and 11; 4 and 25; 4 and 28; 4 and 19; 4 and 15; 8 and 11; 3 and 27; 2 and 25; 2 and 11; 7 and 18; 3 and 25; 8 and 15; 8 and 25; 3 and 11; 3 and 19; 1 and 15; 3 and 15; 1 and 27; 2 and 15; 2 and 19; 1 and 11; 1 and 25; 8 and 19; 4 and 21; 8 and 21; 7 and 27; 7 and 15; 1 and 19; 2 and 21; 7 and 11; 3 and 21; 4 and 14; 7 and 19; 4 and 26; 8 and 26; 7 and 25; 1 and 21; 3 and 26; 2 and 26; 8 and 14; 1 and 14; 2 and 14; 3 and 14; 1 and 26; 7 and 21; 7 and 14; and 7 and 26, and a second nucleic acid encoding a Staphylococcus aureus Cas9 (SaCas9);
l. a first nucleic acid encoding a pair of guide RNAs comprising a first and second spacer sequence selected from any one of SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 8 and 10; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 7 and 10; 1 and 13; 1 and 20; 1 and 28; 4 and 27; 7 and 20; 7 and 23; 7 and 13; 7 and 28; 2 and 27; 8 and 27; 4 and 11; and 4 and 25, and a second nucleic acid encoding a Staphylococcus aureus Cas9 (SaCas9);
m. a first nucleic acid encoding a pair of guide RNAs comprising a first and second spacer sequence selected from any one of SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 1 and 13; 1 and 20; 1 and 28; 4 and 27; 7 and 20; 7 and 23; 7 and 13; 7 and 28; 2 and 27; 4 and 11; 4 and 25; 4 and 28; 4 and 19; 4 and 15; 8 and 11; 3 and 27; 2 and 25; 2 and 11; 7 and 18; 3 and 25; 8 and 15; 3 and 11; 3 and 19; 1 and 15; 3 and 15; 1 and 27; 2 and 15; 2 and 19; 1 and 11; 1 and 25; 4 and 21; 8 and 21; 7 and 27; 7 and 15; land 19; 2 and 21; 7 and 11; 3 and 21; 7 and 19; 7 and 25; 1 and 21; 3 and 26; 3 and 14; 7 and 21; and 7 and 14 and a second nucleic acid encoding a Staphylococcus aureus Cas9 (SaCas9);
n. a first nucleic acid encoding a pair of guide RNAs comprising a first and second spacer sequence selected from any one of SEQ ID NOs: 4 and 12; 2 and 10; 4 and 28; 8 and 12; 4 and 13; 3 and 10; 7 and 12; 7 and 13; 4 and 28; and 7 and 18, and a second nucleic acid encoding a Staphylococcus aureus Cas9 (SaCas9);
o. a first nucleic acid encoding a pair of guide RNAs comprising a first and second spacer sequence selected from any one of SEQ ID NOs: 4 and 12; 2 and 12; 3 and 12; 4 and 20; 4 and 18; 2 and 10; 4 and 10; 1 and 12; 8 and 12; 4 and 13; 7 and 12; 7 and 28; 7 and 18 and a second nucleic acid encoding a Staphylococcus aureus Cas9 (SaCas9);
p. a first nucleic acid encoding a pair of guide RNAs comprising a first and second spacer sequence of SEQ ID NOs: 7 and 12, and a second nucleic acid encoding a Staphylococcus aureus Cas9 (SaCas9);
q. a first nucleic acid encoding a pair of guide RNAs comprising a first and second spacer sequence of SEQ ID NOs: 4 and 12, and a second nucleic acid encoding a Staphylococcus aureus Cas9 (SaCas9); or
r. a first nucleic acid encoding a pair of guide RNAs comprising a first and second spacer sequence of SEQ ID NOs: 4 and 18, and a second nucleic acid encoding a Staphylococcus aureus Cas9 (SaCas9); or
s. a first nucleic acid encoding a pair of guide RNAs comprising a first and second spacer sequence of SEQ ID NOs: 2 and 12, and a second nucleic acid encoding a Staphylococcus aureus Cas9 (SaCas9); or
t. a first nucleic acid encoding a pair of guide RNAs comprising a first and second spacer sequence of SEQ ID NOs: 4 and 13, and a second nucleic acid encoding a Staphylococcus aureus Cas9 (SaCas9); or
u. a first nucleic acid encoding a pair of guide RNAs comprising a first and second spacer sequence of SEQ ID NOs: 8 and 12, and a second nucleic acid encoding a Staphylococcus aureus Cas9 (SaCas9); or
v. a first nucleic acid encoding a pair of guide RNAs comprising a first and second spacer sequence of SEQ ID NOs: 7 and 23, and a second nucleic acid encoding a Staphylococcus aureus Cas9 (SaCas9).

[Embodiment 02] The composition of embodiment 1, further comprising a DNA-PK inhibitor.

[Embodiment 03] The composition of embodiment 1 or 2, further comprising a DNA-PK inhibitor, wherein the DNA-PK inhibitor is Compound 6.

[Embodiment 04] The composition of embodiment 1 or 2, further comprising a DNA-PK inhibitor, wherein the DNA-PK inhibitor is Compound 1.

[Embodiment 05] The composition of embodiment 1 or 2, further comprising a DNA-PK inhibitor, wherein the DNA-PK inhibitor is Compound 2.

[Embodiment 06] The composition of any one of embodiments 1-5, wherein the guide RNA is an sgRNA.

[Embodiment 07] The composition of any one of embodiments 1-6, wherein the guide RNA is modified.

[Embodiment 08] The composition of embodiment 7, wherein the modification alters one or more 2′ positions and/or phosphodiester linkages.

[Embodiment 09] The composition of any one of embodiments 7-9, wherein the modification alters one or more, or all, of the first three nucleotides of the guide RNA.

[Embodiment 10] The composition of any one of embodiments 7-9, wherein the modification alters one or more, or all, of the last three nucleotides of the guide RNA.

[Embodiment 11] The composition of any one of embodiments 7-10, wherein the modification includes one or more of a phosphorothioate modification, a 2′-OMe modification, a 2′-O-MOE modification, a 2′-F modification, a 2′-O-methine-4′ bridge modification, a 3′-thiophosphonoacetate modification, or a 2′-deoxy modification.

[Embodiment 12] The composition of any one of the preceding embodiments, wherein the single nucleic acid molecule is associated with a lipid nanoparticle (LNP).

[Embodiment 13] The composition of any one of embodiments 1-12, wherein the single nucleic acid molecule is a viral vector.

[Embodiment 14] The composition of embodiment 13, wherein the viral vector is an adeno-associated virus vector, a lentiviral vector, an integrase-deficient lentiviral vector, an adenoviral vector, a vaccinia viral vector, an alphaviral vector, or a herpes simplex viral vector.

[Embodiment 15] The composition of embodiment 13, wherein the viral vector is an adeno-associated virus (AAV) vector.

[Embodiment 16] The composition of embodiment 15, wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.

[Embodiment 17] The composition of embodiment 16, wherein the AAV vector is an AAV serotype 9 vector.

[Embodiment 18] The composition of embodiment 16, wherein the AAV vector is an AAVrh10 vector.

[Embodiment 19] The composition of embodiment 16, wherein the AAV vector is an AAVrh74 vector.

[Embodiment 20] The composition of any one of embodiments 11-19, comprising a viral vector, wherein the viral vector comprises a tissue-specific promoter.

[Embodiment 21] The composition of any one of embodiments 11-19, comprising a viral vector, wherein the viral vector comprises a muscle-specific promoter, optionally wherein the muscle-specific promoter is a muscle creatine kinase promoter, a desmin promoter, an MHCK7 promoter, an SPcS-12 promoter, or a CK8e promoter.

[Embodiment 22] The composition of any one of embodiments 11-19, comprising a viral vector, wherein the viral vector comprises a U6, H1, or 7SK promoter.

[Embodiment 23] The composition of any one of embodiments 1-22, comprising a nucleic acid encoding SaCas9, wherein the SaCas9 comprises the amino acid sequence of SEQ ID NO: 711.

[Embodiment 24] The composition of any one of embodiments 1-22, comprising a nucleic acid encoding SaCas9, wherein the SaCas9 is a variant of the amino acid sequence of SEQ ID NO: 711.

[Embodiment 25] The composition of any one of embodiments 1-22, comprising a nucleic acid encoding SaCas9, wherein the SaCas9 comprises an amino acid sequence selected from any one of SEQ ID NOs: 715-717.

[Embodiment 26] The composition of any one of embodiments 1-25 and a pharmaceutically acceptable excipient.

[Embodiment 27] A composition comprising a guide RNA comprising any one of SEQ ID NOs: 1-8, 10-28, and 101-154.

[Embodiment 28] The composition of any one of embodiments 1-27 for use in treating Myotonic Dystrophy Type 1 (DM1).

[Embodiment 29] The composition of any one of embodiments 1-27 for use in making a double strand break in the DMPK gene.

[Embodiment 30] The composition of any one of embodiments 1-27 for use in excising a CTG repeat in the 3′ UTR of the DMPK gene.

[Embodiment 31] A method of treating Myotonic Dystrophy Type 1 (DM1), the method comprising delivering to a cell the composition of any one of embodiments 1-27, and optionally a DNA-PK inhibitor.

[Embodiment 32] A method of treating Myotonic Dystrophy Type 1 (DM1), the method comprising delivering to a cell a single nucleic acid molecule comprising:
a nucleic acid encoding a guide RNA, wherein the guide RNA comprises:
   a. one or more spacer sequences selected from any one of SEQ ID NOs: 1-8, 10-28, and 101-154;
   b. one or more spacer sequences comprising at least 20, or 21 contiguous nucleotides of a spacer sequence selected from any one of SEQ ID NOs: 1-8, 10-28, and 101-154; or
   c. one or more spacer sequences that are at least 90% identical to any one of SEQ ID

NOs: 1-8, 10-28, and 101-154;
a nucleic acid encoding a Staphylococcus aureus Cas9 (SaCas9); and
optionally a DNA-PK inhibitor.

[Embodiment 33] A method of treating Myotonic Dystrophy Type 1 (DM1), the method comprising delivering to a cell a single nucleic acid molecule comprising:
a nucleic acid encoding a pair of guide RNAs comprising:
   a. a first and second spacer sequence selected from any one of SEQ ID NOs: 1 and 10; 1 and 11; 1 and 12; 1 and 13; 1 and 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; band 19; 6 and 20; 6 and 21; 6 and 22; 6 and 23; 6 and 24; 6 and 25; band 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28;
   b. a first and second spacer sequence comprising at least 20, or 21 contiguous nucleotides of any of the first and second spacer sequences of i) a.;
   c. a first and second spacer sequence that is at least 90% identical to any of the first and second spacer sequences of i) a. or i) b.;
a nucleic acid encoding a Staphylococcus aureus Cas9 (SaCas9); and
optionally a DNA-PK inhibitor.

[Embodiment 34] A method of excising a CTG repeat in the 3′ UTR of the DMPK gene, the method comprising delivering to a cell the composition of any one of embodiments 1-27.

[Embodiment 35] A method of excising a CTG repeat in the 3′ UTR of the DMPK gene, the method comprising delivering to a cell a single nucleic acid molecule comprising:
a nucleic acid encoding a guide RNA, wherein the guide RNA comprises:
   a. one or more spacer sequences selected from any one of SEQ ID NOs: 1-8, 10-28, and 101-154;
   b. one or more spacer sequences comprising at least 20, or 21 contiguous nucleotides of a spacer sequence selected from any one of SEQ ID NOs: 1-8, 10-28, and 101-154; or
   c. one or more spacer sequences that are at least 90% identical to any one of SEQ ID NOs: 1-8, 10-28, and 101-154;
a nucleic acid encoding a Staphylococcus aureus Cas9 (SaCas9); and
optionally a DNA-PK inhibitor.

[Embodiment 36] The method of embodiment 35, wherein the one or more spacer sequence is:
selected from any one of SEQ ID NOs: 1, 2, 3, 4, 7, 8, 10, 11, 12, 13, 14, 15, 18, 19, 20, 21, 23, 25, 26, 27, and 28;
selected from any one of SEQ ID NOs: 1, 2, 3, 4, 7, 8, 12, 18, and 20;
selected from any one of SEQ ID NOs: 1, 2, 3, 4, 7, 8, and 20; or
selected from any one of SEQ ID NOs: 4, 12, and 18; or
selected from any one of SEQ ID NOs: 1, 101, and 102.

[Embodiment 37] A method of excising a CTG repeat in the 3′ UTR of the DMPK gene, the method comprising delivering to a cell a single nucleic acid molecule comprising:
a nucleic acid encoding a pair of guide RNAs comprising:
   a. a first and second spacer sequence selected from SEQ ID NOs: 1 and 10; 1 and 11; 1 and 12; 1 and 13; 1 and 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; band 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28;
   b. a first and second spacer sequence comprising at least 20, or 21 contiguous nucleotides of any of the first and second spacer sequences of i) a.;
   c. a first and second spacer sequence that is at least 90% identical to any of the first and second spacer sequences of i) a. or i) b.;
a nucleic acid encoding a Staphylococcus aureus Cas9 (SaCas9); and
optionally a DNA-PK inhibitor.

[Embodiment 38] The method of embodiment 37, wherein the first and second spacer sequences are:
selected from SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 8 and 10; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 7 and 10; 1 and 13; 1 and 20; 1 and 28; 4 and 27; 7 and 20; 7 and 23; 7 and 13; 7 and 28; 2 and 27; 8 and 27; 4 and 11; 4 and 25; 4 and 28; 4 and 19; 4 and 15; 8 and 11; 3 and 27; 2 and 25; 2 and 11; 7 and 18; 3 and 25; 8 and 15; 8 and 25; 3 and 11; 3 and 19; 1 and 15; 3 and 15; 1 and 27; 2 and 15; 2 and 19; land 11; 1 and 25; 8 and 19; 4 and 21; 8 and 21; 7 and 27; 7 and 15; 1 and 19; 2 and 21; 7 and 11; 3 and 21; 4 and 14; 7 and 19; 4 and 26; 8 and 26; 7 and 25; 1 and 21; 3 and 26; 2 and 26; 8 and 14; 1 and 14; 2 and 14; 3 and 14; 1 and 26; 7 and 21; 7 and 14; and 7 and 26; selected from any one of SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 8 and 10; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 7 and 10; 1 and 13; 1 and 20; 1 and 28; 4 and 27; 7 and 20; 7 and 23; 7 and 13; 7 and 28; 2 and 27; 8 and 27; 4 and 11; and 4 and 25;
selected from any one of SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 1 and 13; 1 and 20; 1 and 28; 4 and 27; 7 and 20; 7 and 23; 7 and 13; 7 and 28; 2 and 27; 4 and 11; 4 and 25; 4 and 28; 4 and 19; 4 and 15; 8 and 11; 3 and 27; 2 and 25; 2 and 11; 7 and 18; 3 and 25; 8 and 15; 3 and 11; 3 and 19; 1 and 15; 3 and 15; 1 and 27; 2 and 15; 2 and 19; 1 and 11; 1 and 25; 4 and 21; 8 and 21; 7 and 27; 7 and 15; land 19; 2 and 21; 7 and 11; 3 and 21; 7 and 19; 7 and 25; 1 and 21; 3 and 26; 3 and 14; 7 and 21; and 7 and 14;
selected from any one of SEQ ID NOs: 4 and 12; 4 and 18; 2 and 10; 4 and 28; 8 and 12; 4 and 13; 3 and 10; 7 and 12; 7 and 13; 4 and 28; and 7 and 18;
selected from any one of SEQ ID NOs: 4 and 12; 2 and 12; 3 and 12; 4 and 20; 4 and 18; 2 and 10; 4 and 10; 1 and 12; 8 and 12; 4 and 13; 7 and 12; 7 and 28; 7 and 18; or selected from any one of SEQ ID NOs: 7 and 12; 4 and 12; 4 and 18; 8 and 12; and 7 and 23; or
selected from any one of SEQ ID NOs: 4 and 12; 4 and 18; and 8 and 12.

[Embodiment 39] The method of any one of embodiments 32-38, wherein the single nucleic acid molecule is delivered to the cell on a single vector.

[Embodiment 40] The method of any one of embodiments 32-39, comprising administering a

DNA-PK inhibitor.

[Embodiment 41] The method of embodiment 40, wherein the DNA-PK inhibitor is Compound 6.

[Embodiment 42] The method of embodiment 40, wherein the DNA-PK inhibitor is Compound 1.

[Embodiment 43] The method of embodiment 40, wherein the DNA-PK inhibitor is Compound 2.

[Embodiment 44] The method of any one of embodiments 32-43, wherein the SaCas9 comprises the amino acid sequence of SEQ ID NO: 711.

[Embodiment 45] The method of any one of embodiments 32-43, wherein the SaCas9 is a variant of the amino acid sequence of SEQ ID NO: 711.

[Embodiment 46] The method of any one of embodiments 32-43 or 45, wherein the SaCas9 comprises an amino acid sequence selected from any one of SEQ ID NOs: 715-717.

[Embodiment 47] The composition or method of any one of the preceding embodiments, wherein the single nucleic acid molecule is an AAV vector, and wherein the AAV vector comprises an hU6c promoter.

[Embodiment 48] The composition or method of any one of the preceding embodiments, wherein the single nucleic acid molecule is an AAV vector, and wherein the AAV vector comprises a promoter selected from:
a. a nucleic acid comprising the sequence of SEQ ID NOs: 705, 901, 902, 903, or 904; and
b. a nucleotide sequence that is at least 90%, 91%, 92%, 93%, 94%, 95%, 96%, 97%, 98%, 99% or 100% identical to the sequence of SEQ ID NOs: 705, 901, 902, 903, or 904.

[Embodiment 49] The composition or method of any one of the preceding embodiments, wherein the single nucleic acid molecule is an AAV vector, and wherein the AAV vector comprises a 7SK2 promoter.

[Embodiment 50] The composition or method of any one of the preceding embodiments, wherein the single nucleic acid molecule is an AAV vector, and wherein the AAV vector comprises a promoter selected from:
a. a nucleic acid comprising the sequence of SEQ ID NOs: 706, 906, 907, 908, or 909; and
b. a nucleotide sequence that is at least 90%, 91%, 92%, 93%, 94%, 95%, 96%, 97%, 98%, 99% or 100% identical to the sequence of SEQ ID NOs: 706, 906, 907, 908, or 909.

[Embodiment 51] The composition or method of any one of the preceding embodiments, wherein the single nucleic acid molecule is an AAV vector, and wherein the AAV vector comprises an H1m promoter.

[Embodiment 52] The composition or method of any one of the preceding embodiments, wherein the single nucleic acid molecule is an AAV vector, and wherein the AAV vector comprises a 5′ ITR comprising the sequence of SEQ ID NO: 709.

[Embodiment 53] The composition or method of any one of the preceding embodiments, wherein the single nucleic acid molecule is an AAV vector, and wherein the AAV vector comprises a 3′ ITR comprising the sequence of SEQ ID NO: 710.

[Embodiment 54] The composition or method of any one of the preceding embodiments, wherein the one or more guide RNAs or pair of guide RNAs are sgRNAs comprising a scaffold sequence selected from SEQ ID NOs: 500, 910, 911, 912, 920, or 921.

[Embodiment 55] The composition or method of any one of the preceding embodiments, wherein the one or more guide RNAs or pair of guide RNAs are sgRNAs comprising a scaffold sequence selected from SEQ ID NOs: 910, 911, 912, 920, or 921.

[Embodiment 56] The composition or method of any one of the preceding embodiments, wherein the one or more guide RNAs or pair of guide RNAs are sgRNAs comprising a scaffold sequence is SEQ ID NO: 921.

[Embodiment 57] The composition or method of any one of the preceding embodiments, wherein the nucleic acid molecule encodes at least a first guide RNA and a second guide RNA.

[Embodiment 58] The composition or method of embodiment 57, wherein the nucleic acid molecule encodes a spacer sequence for the first guide RNA, a scaffold sequence for the first guide RNA, a spacer sequence for the second guide RNA, and a scaffold sequence for the second guide RNA.

[Embodiment 59] The composition or method of embodiment 58, wherein the spacer sequence for the first guide RNA and the spacer sequence for the second guide RNA are the same.

[Embodiment 60] The composition or method of embodiment 58, wherein the spacer sequence for the first guide RNA and the spacer sequence for the second guide RNA are different.

[Embodiment 61] The composition or method of embodiment 59 or 60 wherein the scaffold sequence for the first guide RNA and the scaffold sequence for the second guide RNA are the same.

[Embodiment 62] The composition or method of embodiment 59 or 60, wherein the scaffold sequence for the first guide RNA and the scaffold sequence for the second guide RNA are different.

[Embodiment 63] The composition or method of embodiment 61 or embodiment 62, wherein the scaffold sequence for the first guide RNA comprises a sequence selected from the group consisting of SEQ ID NOs: 500, 910, 911, 912, 920, or 921, and wherein the scaffold sequence for the second guide RNA comprises a different sequence selected from the group consisting of SEQ ID NOs: 500, 910, 911, 912, 920, or 921.

[Embodiment 64] The composition or method of any one of embodiments 61-63, wherein the scaffold sequence for the first guide RNA is SEQ ID NO: 921.

[Embodiment 65] The composition or method of any one of embodiments 61-63, wherein the scaffold sequence for the second guide RNA is SEQ ID NO: 921.

[Embodiment 66] The composition or method of embodiment 61, wherein the scaffold sequence for the first guide RNA is SEQ ID NO: 921 and wherein the scaffold sequence for the second guide RNA is SEQ ID NO: 921.

[Embodiment 67] The composition or method of any one of the preceding embodiments, wherein the single nucleic acid molecule is an AAV vector, wherein the vector comprises from 5′ to 3′ with respect to the plus strand: a promoter for expression of a nucleic acid encoding a first sgRNA, a nucleic acid encoding the first sgRNA guide sequence, a first sgRNA scaffold sequence, a promoter for expression of a nucleic acid encoding a SaCas9 (e.g., CK8e), a nucleic acid encoding a SaCas9, a polyadenylation sequence, a promoter for expression of a second sgRNA, the second sgRNA guide sequence, and a second sgRNA scaffold sequence.

[Embodiment 68] The composition or method of any one of the preceding embodiments, wherein the single nucleic acid molecule is an AAV vector, wherein the vector comprises from 5′ to 3′ with respect to the plus strand: the reverse complement of a first sgRNA scaffold sequence, the reverse complement of a nucleic acid encoding a first sgRNA guide sequence, the reverse complement of a promoter for expression of the nucleic acid encoding the first sgRNA, a promoter for expression of a nucleic acid encoding SaCas9 (e.g., CK8e), a nucleic acid encoding a SaCas9, a polyadenylation sequence, a promoter for expression of a second sgRNA in the same direction as the promoter for SaCas9, a second sgRNA guide sequence, and a second sgRNA scaffold sequence.

[Embodiment 69] The composition or method of any one of the preceding embodiments, wherein the single nucleic acid molecule is an AAV vector, wherein the vector comprises from 5′ to 3′ with respect to the plus strand: a promoter for expression of a nucleic acid encoding a first sgRNA, a nucleic acid encoding a first sgRNA guide sequence, a first sgRNA scaffold sequence, a promoter for expression of a second sgRNA, a second sgRNA guide sequence, and a second sgRNA scaffold sequence, a promoter for expression of a nucleic acid encoding SaCas9 (e.g., CK8e), a nucleic acid encoding SaCas9, and a polyadenylation sequence.

[Embodiment 70] The composition or method of any one of the preceding embodiments, wherein the single nucleic acid molecule is an AAV vector, wherein the vector comprises from 5′ to 3′ with respect to the plus strand: a promoter for expression of a nucleic acid encoding SaCas9 (e.g., CK8e), a nucleic acid encoding SaCas9, a polyadenylation sequence, a promoter for expression of a nucleic acid encoding a first guide RNA in the same direction as the promoter for SaCas9, a nucleic acid encoding a first sgRNA guide sequence, a first sgRNA scaffold sequence, a promoter for expression of a second sgRNA in the same direction as the promoter for SaCas9, a second sgRNA guide sequence, and a second sgRNA scaffold sequence.

[Embodiment 71] The composition or method of any one of the preceding embodiments, wherein the single nucleic acid molecule is an AAV vector, wherein the vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence, an 7SK2 promoter for expression of a second sgRNA, a second sgRNA guide sequence, and a second sgRNA scaffold sequence, a promoter for expression of a nucleic acid encoding SaCas9 (e.g., CK8e), a nucleic acid encoding SaCas9, and a polyadenylation sequence.

[Embodiment 72] The composition or method of any one of the preceding embodiments, wherein the single nucleic acid molecule is an AAV vector, wherein the vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence, an 7SK2 promoter for expression of a second sgRNA, a second sgRNA guide sequence, and a second sgRNA scaffold sequence, a promoter for expression of a nucleic acid encoding SaCas9 (e.g., CK8e), a nucleic acid encoding SaCas9, and a polyadenylation sequence.

[Embodiment 73] The composition or method of any one of embodiments 67-72, wherein the first sgRNA comprises SaU4 (SEQ ID NO: 4) and the second sgRNA comprises SaD4 (SEQ ID NO: 12).

[Embodiment 74] The composition or method of any one of embodiments 67-72, wherein the first sgRNA comprises SaU7 (SEQ ID NO: 7) and the second sgRNA comprises SaD10 (SEQ ID NO: 18).

[Embodiment 75] The composition or method of any one of embodiments 67-74, wherein the first sgRNA guide sequence and the second sgRNA guide sequence are the same.

[Embodiment 76] The composition or method of any one of embodiments 67-74, wherein the first sgRNA guide sequence and the second sgRNA guide sequence are different.

[Embodiment 77] The composition or method of any one of embodiments 67-76, wherein the first sgRNA scaffold sequence and the second sgRNA scaffold sequence are the same.

[Embodiment 78] The composition or method of any one of embodiments 67-76, wherein the first sgRNA scaffold sequence and the second sgRNA scaffold sequence are different.

[Embodiment 79] The composition or method of embodiment 77 or embodiment 78, wherein the first sgRNA scaffold sequence comprises a sequence selected from the group consisting of SEQ ID NOs: 500, 910, 911, 912, 920, or 921, and wherein the second sgRNA scaffold sequence comprises a different sequence selected from the group consisting of SEQ ID NOs: 500, 910, 911, 912, 920, or 921.

[Embodiment 80] The composition or method of any one of embodiments 77-79, wherein the first sgRNA scaffold sequence is SEQ ID NO: 921.

[Embodiment 81] The composition or method of any one of embodiments 77-79, wherein the second sgRNA scaffold sequence is SEQ ID NO: 921.

[Embodiment 82] The composition or method of embodiment 77, wherein the first sgRNA scaffold sequence is SEQ ID NO: 921 and wherein the second sgRNA scaffold sequence is SEQ ID NO: 921.

[Embodiment 83] A method of reducing the number of foci-positive cells, the method comprising delivering to a cell one or more acid molecules comprising:
a nucleic acid encoding a guide RNA, wherein the guide RNA comprises:
   a. one or more spacer sequences selected from any one of SEQ ID NOs: 1-8, 10-28, and 101-154;
   b. one or more spacer sequences comprising at least 20, or 21 contiguous nucleotides of a spacer sequence selected from any one of SEQ ID NOs: 1-8, 10-28, and 101-154; or
   c. one or more spacer sequences that are at least 90% identical to any one of SEQ ID NOs: 1-8, 10-28, and 101-154;
a nucleic acid encoding a Staphylococcus aureus Cas9 (SaCas9); and
optionally a DNA-PK inhibitor.

[Embodiment 84] A method of reducing the number of foci-positive cells, the method comprising delivering to a cell one or more nucleic acid molecules comprising:
a nucleic acid encoding a pair of guide RNAs comprising:
   a. a first and second spacer sequence selected from any one of SEQ ID NOs: 1 and 10;
      1 and 11; 1 and 12; 1 and 13; 1 and 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; band 19; 6 and 20; 6 and 21; 6 and 22; 6 and 23; 6 and 24; 6 and 25; band 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28;
   b. a first and second spacer sequence comprising at least 20, or 21 contiguous nucleotides of any of the first and second spacer sequences of i) a.;
   c. a first and second spacer sequence that is at least 90% identical to any of the first and second spacer sequences of i) a. or i) b.;
a nucleic acid encoding a Staphylococcus aureus Cas9 (SaCas9); and
optionally a DNA-PK inhibitor.

[Embodiment 85] The composition or method of any one of the preceding embodiments, comprising a pair of guide RNAs, wherein the pair of guide RNAs function to excise and also function as single guide cutters.

[Embodiment 86] The method of embodiment 83 or 84, wherein the first nucleic acid and the second nucleic acid are in the same nucleic acid molecule.

[Embodiment 87] The method of embodiment 83 or 84, wherein the first nucleic acid and the second nucleic acid are in separate nucleic acid molecules.

[Embodiment 88] The method of embodiment 87, wherein the separate nucleic acid molecules are each in separate vectors.

[Embodiment 89] The method of any one of embodiments 83-88, wherein the nucleic acid encoding the SaCas9 does not encode a guide RNA.

[Embodiment 90] The method of any one of embodiments 84-89, wherein the nucleic acid encoding the SaCas9 encodes one or more guide RNAs comprising:
a. a first and second spacer sequence selected from any one of SEQ ID NOs: 1 and 10; 1 and 11; land 12; land 13; land 14; land 15; land 16; land 17; land 18; land 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28;
b. a first and second spacer sequence comprising at least 20, or 21 contiguous nucleotides of any of the first and second spacer sequences of a.;
c. a first and second spacer sequence that is at least 90% identical to any of the first and second spacer sequences of a. orb.

[Embodiment 91] A composition comprising a first nucleic acid molecule and a second nucleic acid molecule, wherein the nucleic acid molecule encodes a Staphylococcus aureus Cas9 (SaCas9) and the second nucleic acid molecule encodes: one or more guide RNAs comprising:
a. a first and second spacer sequence selected from any one of SEQ ID NOs: 1 and 10; 1 and 11; 1 and 12; 1 and 13; 1 and 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; band 19; 6 and 20; 6 and 21; 6 and 22; 6 and 23; 6 and 24; 6 and 25; band 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28;
b. a first and second spacer sequence comprising at least 20, or 21 contiguous nucleotides of any of the first and second spacer sequences of a.;
c. a first and second spacer sequence that is at least 90% identical to any of the first and second spacer sequences of a. orb.

[Embodiment 92] The composition of embodiment 91, wherein the first nucleic acid molecule does not encode a guide RNA.

[Embodiment 93] The composition of embodiment 91, wherein the first nucleic acid molecule encodes:
a. a first and second spacer sequence selected from any one of SEQ ID NOs: 1 and 10; 1 and 11; land 12; land 13; land 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; band 19; 6 and 20; 6 and 21; 6 and 22; 6 and 23; 6 and 24; 6 and 25; band 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28;
b. a first and second spacer sequence comprising at least 20, or 21 contiguous nucleotides of any of the first and second spacer sequences of a.;
c. a first and second spacer sequence that is at least 90% identical to any of the first and second spacer sequences of a. orb.

[Embodiment 94] The composition of any one of embodiments 91-93, wherein the first nucleic acid molecule is in a first vector, and the second nucleic acid molecule is in a separate second vector.

[Embodiment 95] The composition of embodiment 94, wherein the first and second vectors are AAV vectors.

[Embodiment 96] The composition of embodiment 95, wherein the AAV vectors are AAV9 vectors.

[Embodiment 97] A composition comprising an AAV vector, wherein the vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence, a hU6c promoter for expression of a second sgRNA, a second sgRNA guide sequence, and a second sgRNA scaffold sequence, a promoter for expression of a nucleic acid encoding a Cas9 (e.g., CK8e), a nucleic acid encoding a Cas9, and a polyadenylation sequence.

[Embodiment 98] A composition comprising an AAV vector, wherein the vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence, a 7SK2 promoter for expression of a second sgRNA, a second sgRNA guide sequence, and a second sgRNA scaffold sequence, a promoter for expression of a nucleic acid encoding a Cas9 (e.g., CK8e), a nucleic acid encoding a Cas9, and a polyadenylation sequence.

[Embodiment 99] A composition comprising an AAV vector, wherein the vector comprises from 5′ to 3′ with respect to the plus strand: a promoter for expression of a nucleic acid encoding a Cas9 (e.g., CK8e), a nucleic acid encoding a Cas9, a polyadenylation sequence, an hU6c promoter for expression of a nucleic acid encoding a first sgRNA, a nucleic acid encoding the first sgRNA guide sequence, a first sgRNA scaffold sequence, a hU6c promoter for expression of a second sgRNA, a second sgRNA guide sequence, and a second sgRNA scaffold sequence.

[Embodiment 100] A composition comprising an AAV vector, wherein the vector comprises from 5′ to 3′ with respect to the plus strand: a promoter for expression of a nucleic acid encoding a Cas9 (e.g., CK8e), a nucleic acid encoding a Cas9, a polyadenylation sequence, an hU6c promoter for expression of a nucleic acid encoding a first sgRNA, a nucleic acid encoding the first sgRNA guide sequence, a first sgRNA scaffold sequence, a 7SK2 promoter for expression of a second sgRNA, a second sgRNA guide sequence, and a second sgRNA scaffold sequence.

[Embodiment 101] A composition comprising an AAV vector, wherein the vector comprises from 5′ to 3′ with respect to the plus strand: the reverse complement of a sequence encoding a first sgRNA scaffold sequence, the reverse complement of a sequence encoding a first sgRNA, the reverse complement of an 7SK2 or hU6c promoter for expression of a nucleic acid encoding a first sgRNA, a promoter for expression of a nucleic acid encoding a Cas9 (e.g., CK8e), a nucleic acid encoding a Cas9, a polyadenylation sequence, a hU6c promoter for expression of a second sgRNA, a second sgRNA guide sequence, and a second sgRNA scaffold sequence.

[Embodiment 102] The composition of any one of embodiments 97-101, wherein the first sgRNA guide sequence comprises SEQ ID NO: 7, and the second sgRNA guide sequence comprises SEQ ID NO: 12.

[Embodiment 103] The composition of any one of embodiments 97-101, wherein the first sgRNA guide sequence comprises SEQ ID NO: 4, and the second sgRNA guide sequence comprises SEQ ID NO: 12.

[Embodiment 104] The composition of any one of embodiments 97-101, wherein the first sgRNA guide sequence comprises SEQ ID NO: 4, and the second sgRNA guide sequence comprises SEQ ID NO: 18.

[Embodiment 105] The composition of any one of embodiments 97-101, wherein the first sgRNA guide sequence comprises SEQ ID NO: 2, and the second sgRNA guide sequence comprises SEQ ID NO: 12.

[Embodiment 106] The composition of any one of embodiments 97-101, wherein the first sgRNA guide sequence comprises SEQ ID NO: 4, and the second sgRNA guide sequence comprises SEQ ID NO: 13.

[Embodiment 107] The composition of any one of embodiments 97-101, wherein the first sgRNA guide sequence comprises SEQ ID NO: 8, and the second sgRNA guide sequence comprises SEQ ID NO: 12.

[Embodiment 108] The composition of any one of embodiments 97-101, wherein the first sgRNA guide sequence comprises SEQ ID NO: 7, and the second sgRNA guide sequence comprises SEQ ID NO: 23.

[Embodiment 109] A composition comprising a nucleic acid molecule comprising nucleic acid encoding two different sgRNA guide sequences, wherein the first sgRNA guide sequence comprises SEQ ID NO: 7, and the second sgRNA guide sequence comprises SEQ ID NO: 12.

[Embodiment 110] A composition comprising a nucleic acid molecule comprising nucleic acid encoding two different sgRNA guide sequences, wherein the first sgRNA guide sequence comprises SEQ ID NO: 4, and the second sgRNA guide sequence comprises SEQ ID NO: 12.

[Embodiment 111] A composition comprising a nucleic acid molecule comprising nucleic acid encoding two different sgRNA guide sequences, wherein the first sgRNA guide sequence comprises SEQ ID NO: 4, and the second sgRNA guide sequence comprises SEQ ID NO: 18.

[Embodiment 112] A composition comprising a nucleic acid molecule comprising nucleic acid encoding two different sgRNA guide sequences, wherein the first sgRNA guide sequence comprises SEQ ID NO: 2, and the second sgRNA guide sequence comprises SEQ ID NO: 12.

[Embodiment 113] A composition comprising a nucleic acid molecule comprising nucleic acid encoding two different sgRNA guide sequences, wherein the first sgRNA guide sequence comprises SEQ ID NO: 4, and the second sgRNA guide sequence comprises SEQ ID NO: 13.

[Embodiment 114] A composition comprising a nucleic acid molecule comprising nucleic acid encoding two different sgRNA guide sequences, wherein the first sgRNA guide sequence comprises SEQ ID NO: 8, and the second sgRNA guide sequence comprises SEQ ID NO: 12.

[Embodiment 115] A composition comprising a nucleic acid molecule comprising nucleic acid encoding two different sgRNA guide sequences, wherein the first sgRNA guide sequence comprises SEQ ID NO: 7, and the second sgRNA guide sequence comprises SEQ ID NO: 23.

[Embodiment 116] A method of treating Myotonic Dystrophy Type 1 (DM1), the method comprising delivering to a cell the composition of any one of embodiments 109-115, and optionally a DNA-PK inhibitor.

[Embodiment 117] A method of excising a CTG repeat in the 3′ UTR of the DMPK gene, the method comprising delivering to a cell the composition of any one of embodiments 109-115.

[Embodiment 118] A method of treating Myotonic Dystrophy Type 1 (DM1), the method comprising delivering to a cell a single nucleic acid molecule comprising:
a nucleic acid encoding a pair of guide RNAs comprising:
   a. a first and second spacer sequence, wherein the first spacer sequence comprises SEQ ID NO: 7, and the second spacer sequence comprises SEQ ID NO: 12;
   b. a first and second spacer sequence, wherein the first spacer sequence comprises SEQ ID NO: 4, and the second spacer sequence comprises SEQ ID NO: 12;
   c. a first and second spacer sequence, wherein the first spacer sequence comprises SEQ ID NO: 4, and the second spacer sequence comprises SEQ ID NO: 18;
   d. a first and second spacer sequence, wherein the first spacer sequence comprises SEQ ID NO: 2, and the second spacer sequence comprises SEQ ID NO: 12;
   e. a first and second spacer sequence, wherein the first spacer sequence comprises SEQ ID NO: 4, and the second spacer sequence comprises SEQ ID NO: 13;
   f. a first and second spacer sequence, wherein the first spacer sequence comprises SEQ ID NO: 8, and the second spacer sequence comprises SEQ ID NO: 12;
   g. a first and second spacer sequence, wherein the first spacer sequence comprises SEQ ID NO: 7, and the second spacer sequence comprises SEQ ID NO: 23;
ii) a nucleic acid encoding a Staphylococcus aureus Cas9 (SaCas9); and
iii) optionally a DNA-PK inhibitor.

[Embodiment 119] A method of excising a CTG repeat in the 3′ UTR of the DMPK gene, the method comprising delivering to a cell a single nucleic acid molecule comprising:
i) a nucleic acid encoding a pair of guide RNAs comprising:
   a. a first and second spacer sequence, wherein the first spacer sequence comprises SEQ ID NO: 7, and the second spacer sequence comprises SEQ ID NO: 12;
   b. a first and second spacer sequence, wherein the first spacer sequence comprises SEQ ID NO: 4, and the second spacer sequence comprises SEQ ID NO: 12;
   c. a first and second spacer sequence, wherein the first spacer sequence comprises SEQ ID NO: 4, and the second spacer sequence comprises SEQ ID NO: 18;
   d. a first and second spacer sequence, wherein the first spacer sequence comprises SEQ ID NO: 2, and the second spacer sequence comprises SEQ ID NO: 12;
   e. a first and second spacer sequence, wherein the first spacer sequence comprises SEQ ID NO: 4, and the second spacer sequence comprises SEQ ID NO: 13;
   f. a first and second spacer sequence, wherein the first spacer sequence comprises SEQ ID NO: 8, and the second spacer sequence comprises SEQ ID NO: 12;
   g. a first and second spacer sequence, wherein the first spacer sequence comprises SEQ ID NO: 7, and the second spacer sequence comprises SEQ ID NO: 23;

ii) a nucleic acid encoding a Staphylococcus aureus Cas9 (SaCas9); and

iii) optionally a DNA-PK inhibitor.

[Embodiment 120] The composition of any one of embodiments 109-115, wherein the composition further comprises a Staphylococcus aureus Cas9 (SaCas9) or a nucleic acid encoding an SaCas9.

[Embodiment 121] The composition of any one of embodiments 109-115 or 120, wherein the composition is associated with a lipid nanoparticle.

[Embodiment 122] The composition of any one of embodiments 1-26 or method of any one of claim 31-108 or 116-121, wherein an SV40 nuclear localization signal (NLS) is fused to the N-terminus of the Cas9 and a nucleoplasmin NLS is fused to the C-terminus of the Cas9 protein.

[Embodiment 123] The composition of any one of embodiments 1-26 or method of any one of claim 31-108 or 116-121, wherein a c-myc nuclear localization signal (NLS) is fused to the N-terminus of the Cas9 and an SV40 NLS and/or nucleoplasmin NLS is fused to the C-terminus of the Cas9.

[Embodiment 124] The composition of any one of embodiments 1-26 or method of any one of claim 31-108 or 116-121, wherein a c-myc NLS is fused to the N-terminus of the Cas9 (e.g., by means of a linker such as GSVD (SEQ ID NO: 940)), an SV40 NLS is fused to the C-terminus of the Cas9 (e.g., by means of a linker such as GSGS (SEQ ID NO: 941)), and a nucleoplasmin NLS is fused to the C-terminus of the SV-40 NLS (e.g., by means of a linker such as GSGS (SEQ ID NO: 941)).

### DESCRIPTION OF FIGURES

FIG. 1 depicts a DM1 gene editing therapeutic approach. Single sgRNAs (SINGLE-cut) or paired sgRNAs (DOULBLE-cut) coupled with Cas9 endonuclease are used to excise the CTG repeat expansion in the 3′ UTR of the human DMPK gene.

FIG. 2 shows the location of 27 selected SaCas9 sgRNAs. Eight upstream sgRNAs and 19 downstream sgRNAs were selected for SINGLE-cut screening.

FIG. 3 shows the editing efficiency of 27 selected SaCas9 sgRNAs in primary DM1 patient myoblasts. Editing efficiency was assessed by Sanger sequencing and ICE analysis and shown as mean±standard error (n=4). The sgRNAs are ordered from the highest efficiency to the lowest efficiency. 21 sgRNAs (solid bars) showed greater than 10% indel efficiency and were selected for further DOUBLE-cut screening.

FIGS. 4A-4C show guide RNA SaU1 (SEQ ID NO: 1) induced CTG repeat excision.

FIG. 4a shows the location of SaU1 sgRNA and the deletion of a 370 bp sequence that was induced by SaU1 SINGLE-cut. FIG. 4B shows the chromatogram traces of untreated DM1 myoblasts and SaU1-treated DM1 myoblasts. The wild type allele of the DMPK 3′ UTR region was PCR amplified followed by Sanger sequencing, and chromatogram traces near the cut site were displayed. Based on the chromatogram trace, a 370 bp sequence flanking the CTG repeat was deleted in SaU1-treated myoblasts. FIG. 4C shows TapeStation analysis of the PCR product amplified from the wild type allele of the DMPK 3′ UTR region. The arrow points to the PCR products amplified from non-edited wild type allele or wild type allele with small indels. The circles indicate the PCR products amplified from wild type allele with the 370 bp deletion. DNA-PKi increased the deletion frequency of the 370 bp deletion sequence. DM1 Mock is the DM1 patient myoblasts that only received SaCas9 protein (no sgRNA) nucleofection.

FIG. 5 shows the frequency of CUG foci free myoblast nuclei induced by individual SaCas9 sgRNAs in primary DM1 patient myoblasts. Shown are the percentage of CUG foci free myoblast nuclei (mean±standard deviation (n=2)). The dotted line indicates that CUG foci was abolished in 20% of myoblast nuclei. White bars represent foci reduction efficiency with vehicle treatment, and solid bars represent foci reduction efficiency with DNA-PKi treatment. The sgRNAs are ordered from the highest efficiency to the lowest efficiency in the DNA-PKi-treated group. DM1 Mock served as a negative control, and healthy control served as a positive control.

FIGS. 6A-6B show SaU01 sgRNA reduced CUG foci in primary DM1 patient myoblasts. FIG. 6A shows immunofluorescence images showing CUG foci staining in SaU1-nucleofected DM1 myoblasts treated with vehicle or DNA-Pki, or Mock-nucleofected DM1 myoblasts treated with vehicle. CUG foci are visualized as the brighter spots within DAPI-stained myoblasts. FIG. 6B shows the frequency distribution of myoblast nuclei with different numbers of CUG foci. SaU1 sgRNA not only increased the frequency of CUG foci free myoblast nuclei (Foci number per nucleus=0), but also reduced the frequency of myoblast nuclei that contains more than three CUG foci.

FIG. 7 shows the efficiency of CTG repeat excision induced by SaCas9 sgRNA pairs in primary DM1 patient myoblasts. Shown are the mean efficiency of CTG repeat excision measured by the upstream and downstream ddPCR assays. White bars represent CTG repeat excision efficiency with vehicle treatment, and solid bars represent CTG repeat excision efficiency with DNA-PKi treatment. The sgRNAs are ordered from the highest efficiency to the lowest efficiency in the vehicle-treated group. See Table 4 for the efficiency of CTG repeat excision induced by individual sgRNA pairs.

FIG. 8 shows the frequency of CUG foci free myoblast nuclei induced by SaCas9 sgRNA pairs in primary DM1 patient myoblasts. White bars represent foci reduction efficiency with vehicle treatment, and solid bars represent foci reduction efficiency with DNA-PKi treatment. The sgRNAs are ordered from the highest efficiency to the lowest efficiency in the vehicle-treated group. See Table 4 for the efficiency of CUG foci free myoblast nuclei induced by individual sgRNA pairs.

FIGS. 9A-9B show the pair of SaU4+SaD4 greatly reduced CUG foci in primary DM1 patient myoblasts. FIG. 9A shows immunofluorescence images showing CUG foci staining in healthy or DM1 myoblasts treated with vehicle, or SaU4+SaD4-nucleofected DM1 myoblasts treated with vehicle or DNA-PKi. FIG. 9B shows the frequency distribution of myoblast nuclei with different numbers of CUG foci. The pair of SaU4+SaD4 abolished CUG foci in vast majority of myoblasts treated with either vehicle or DNA-PKi.

FIGS. 10A-B show several representative “all-in-one” vector configurations. FIG. 10A is a schematic showing four representative vector designs. White arrows indicate directionality of expression of the sgRNA(s), while the black arrows indicate directionality of the Cas9 protein. In a particular embodiment, the Cas9 promoter may be CK8e. “Pol III” refers to a representative promoter for the expression of sgRNAs, “g 1” and “g2” each refer to a guide sequence, “scaffold” refers to the scaffold of a guide RNA, and “pa” refers to a polyadenylation sequence. FIG. 10B shows further vector configurations, including representative promoters for the expression of the sgRNAs, and representative sgRNAs indicated as “U7” and “D10.”

FIG. 11 is a schematic showing the AAV size in base pairs (bp) for representative vector configurations referred to as AIO-AAV7, AIO-AA8, AIO-AAV10, AIO-AAV11, and AIO-AAV17.

FIG. 12 shows a schematic of the hU6c promoter.

FIG. 13 shows a schematic of the 7SK2 promoter.

FIGS. 14A-B show further representative “all-in-one” vector configurations modified from AIO-AAV8. FIG. 14A shows a schematic of AIO-AAV8 with the modified region indicated by a bracket. FIG. 14B lists vector configurations with changes compared to AIO-AAV8 as well as vector configurations with changes compared to AIO-AAV31 (modified from AIO-AAV8).

FIGS. 15A-B show editing efficiency of sgRNAs SaU4 (FIG. 15A) and SaD4 (FIG. 15B) with different SaCas9 scaffolds in 293 T cells.

FIG. 16 shows editing efficiency of sgRNAs SaU4 and SaD4 with different SaCas9 scaffolds in primary human myoblasts at three doses (30, 15, and 7.5 pmol).

FIGS. 17A-B show further representative “all-in-one” vector configurations modified from AIO-AAV8. FIG. 17A lists vector configurations with changes compared to AIO-AAV8. FIG. 17B lists vector configurations with changes compared to AIO-AAV51 (modified from AIO-AAV8).

FIGS. 18A-E show DM1 SINGLE-vector ssAAV design optimization. FIG. 18A shows schematics of in vitro AAV vector optimization procedure. FIG. 18B shows the relative expression level of upstream and downstream sgRNA in AAV plasmid transfected C2C12 cells (normalized to mHPRT expression). FIG. 18C shows schematics of myogenic differentiation and AAV transduction in DM1 patient derived myotubes. FIGS. 18D-E shows quantification of CUG foci free myonuclei at different time points in AAV infected myotubes without (D) and with DNA-PKi (E) treatment.

FIGS. 19A-D show AAV transduction in DM1 patient myoblast-differentiated myotubes. FIG. 19A shows the position of the sgRNA pairs used for AAV infection in the DOUBLE-cut screening ranking. FIG. 19B shows schematics of myogenic differentiation and AAV transduction in DM1 patient derived myotubes. FIG. 19C shows the quantification of CUG foci free myonuclei and FIG. 19D shows average CUG foci number per myonucleus (bottom) in AAV infected myotubes.

FIG. 20 shows exemplary SaCas9 sgRNA, which can be used in pairs for in vivo editing evaluation.

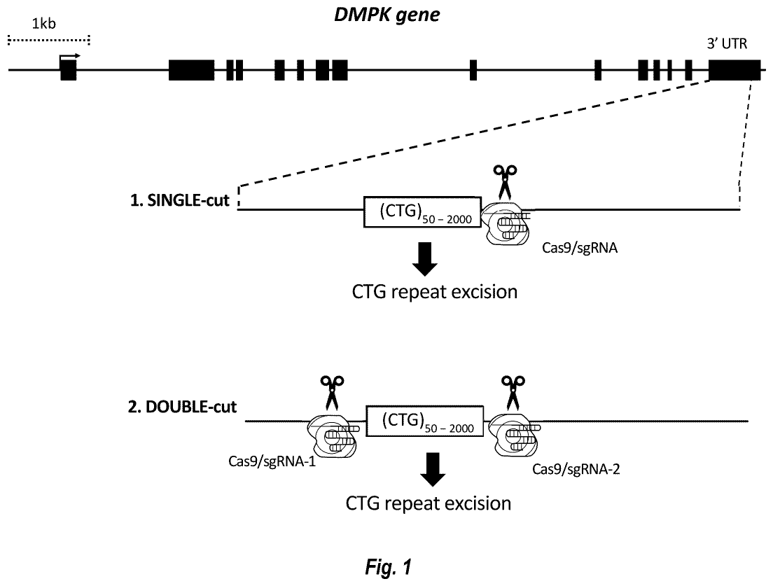

### DETAILED DESCRIPTION

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a guide” includes a plurality of guides and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

“Polynucleotide,” “nucleic acid,” and “nucleic acid molecule,” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and 0 4-alkyl-pyrimidines; U.S. Pat. No. 5,378,825 and PCT No. WO 93/13121). For general discussion see The Biochemistry of the Nucleic Acids 5-36, Adams et al., ed., 11 th ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “guide RNA”, and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “guide RNA” refers to each type. The trRNA may be a naturally-occurring sequence, or a trRNA sequence with modifications or variations compared to naturally-occurring sequences.

As used herein, a “spacer sequence,” sometimes also referred to herein and in the literature as a “spacer,” “protospacer,” “guide sequence,” or “targeting sequence” refers to a sequence within a guide RNA that is complementary to a target sequence and functions to direct a guide RNA to a target sequence for cleavage by a Cas9. A guide sequence can be 24, 23, 22, 21, 20 or fewer base pairs in length, e.g., in the case of Staphylococcus aureus c., SaCas9) and related Cas9 homologs/orthologs. Shorter or longer sequences can also be used as guides, e.g., 15-, 16-, 17-, 18-, 19-, 20-, 21-, 22-, 23-, 24-, or 25-nucleotides in length. In particular embodiments, a guide/spacer sequence in the case of SaCas9 is at least 20 base pairs in length, or more specifically, within 20-25 base pairs in length (see, e.g., Schmidt et al., 2021, Nature Communications, “Improved CRISPR genome editing using small highly active and specific engineered RNA-guided nucleases”). For example, in some embodiments, the guide sequence comprises at least 17, 18, 19, 20, 21, 22, 23, 24, or 25 contiguous nucleotides of a sequence selected from SEQ ID NOs: 1-8, 10-28, or 101-154. In some embodiments, the guide sequence comprises a sequence selected from SEQ ID NOs: 1-8, 10-28, and 101-154. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. For example, in some embodiments, the guide sequence comprises a sequence with about 75%, 80%, 85%, 90%, 95%, 96%, 97%, 98%, 99%, or 100% identity to at least 17, 18, 19, 20, 21, 22, 23, 24, or 25 contiguous nucleotides of a sequence selected from SEQ ID NOs: 1-8, 10-28, and 101-154. In some embodiments, the guide sequence comprises a sequence with about 75%, 80%, 85%, 90%, 95%, 96%, 97%, 98%, 99%, or 100% identity to a sequence selected from SEQ ID NOs: 1-8, 10-28, and 101-154. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides. In some embodiments, the guide sequence and the target region do not contain any mismatches.

In some embodiments, the guide sequence comprises a sequence selected from SEQ ID NOs: 1-8, 10-28, and 101-154, wherein if the 5′ terminal nucleotide is not guanine, one or more guanine (g) is added to the sequence at its 5′ end. The 5′ g or gg may be necessary in some instances for transcription, for example, for expression by the RNA polymerase III-dependent U6 promoter or the T7 promoter. In some embodiments, a 5′ guanine is added to any one of the guide sequences or pairs of guide sequences disclosed herein.

Target sequences for Cas9s include both the positive and negative strands of genomic DNA (i.e., the sequence given and the sequence's reverse compliment), as a nucleic acid substrate for a Cas9 is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, “ribonucleoprotein” (RNP) or “RNP complex” refers to a guide RNA together with a Cas9. In some embodiments, the guide RNA guides the Cas9 such as Cas9 to a target sequence, and the guide RNA hybridizes with and the agent binds to the target sequence, which can be followed by cleaving or nicking (in the context of a modified “nickase” Cas9).

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, NI-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is not DNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof

Guide sequences useful in the guide RNA compositions and methods described herein are shown in Table 1A, and Table 1B and throughout the application.

As used herein, a “target sequence” refers to a sequence of nucleic acid in a target gene that has complementarity to at least a portion of the guide sequence of the guide RNA. The interaction of the target sequence and the guide sequence directs a Cas9 to bind, and potentially nick or cleave (depending on the activity of the agent), within the target sequence.

As used herein, “treatment” refers to any administration or application of a therapeutic for disease or disorder in a subject, and includes inhibiting the disease or development of the disease (which may occur before or after the disease is formally diagnosed, e.g., in cases where a subject has a genotype that has the potential or is likely to result in development of the disease), arresting its development, relieving one or more symptoms of the disease, curing the disease, or preventing reoccurrence of one or more symptoms of the disease. For example, treatment of DM1 may comprise alleviating symptoms of DM1.

As used herein, “ameliorating” refers to any beneficial effect on a phenotype or symptom, such as reducing its severity, slowing or delaying its development, arresting its development, or partially or completely reversing or eliminating it. In the case of quantitative phenotypes such as expression levels, ameliorating encompasses changing the expression level so that it is closer to the expression level seen in healthy or unaffected cells or individuals.

A “pharmaceutically acceptable excipient” refers to an agent that is included in a pharmaceutical formulation that is not the active ingredient. Pharmaceutically acceptable excipients may e.g., aid in drug delivery or support or enhance stability or bioavailability.

The term “about” or “approximately” means an acceptable error for a particular value as determined by one of ordinary skill in the art, which depends in part on how the value is measured or determined.

As used herein, “Staphylococcus aureus Cas9” may also be referred to as SaCas9, and includes wild type SaCas9 (e.g., SEQ ID NO: 711) and variants thereof. A variant of SaCas9 comprises one or more amino acid changes as compared to SEQ ID NO: 711, including insertion, deletion, or substitution of one or more amino acids, or a chemical modification to one or more amino acids.

### II. Compositions

Provided herein are compositions useful for treating Myotonic Dystrophy Type 1 (DM1), e.g., using a single nucleic acid molecule encoding 1) one or more guide RNAs comprising one or more guide sequences of Table 1A and Table 1B; and 2) SaCas9. Such compositions may be administered to subjects having or suspected of having DM1. Any of the guide sequences disclosed herein may be in any of the pair combinations disclosed herein, and may be in a composition comprising any of the Cas9 proteins disclosed herein or a nucleic acid encoding any of the Cas9 proteins disclosed herein. Such compositions may be in any of the vectors disclosed herein (e.g., any of the AAV vectors disclosed herein) or be associated with a lipid nanoparticle.

In some embodiments, the disclosure provides for specific nucleic acid sequence encoding one or more guide RNA components (e.g., any of the spacer and or scaffold sequences disclosed herein). The disclosure contemplates RNA equivalents of any of the DNA sequences provided herein (i.e., in which “T”s are replaced with “U”s), or DNA equivalents of any of the RNA sequences provided herein (e.g., in which “U”s are replaced with “T”s), as well as complements (including reverse complements) of any of the sequences disclosed herein.

In some embodiments, the one or more guide RNAs direct the Cas9 to a site in or near a CTG repeat in the 3′ UTR of the DM1 protein kinase (DMPK) gene. For example, the Cas9 may be directed to cut within 10, 20, 30, 40, or 50 nucleotides of a target sequence.

In some embodiments, a composition comprising a single nucleic acid molecule encoding one or more guide RNAs and a Cas9 is provided, wherein the single nucleic acid molecule comprises:
a. a first nucleic acid encoding one or more spacer sequences selected from any one of SEQ ID NOs: 1-8, 10-28, and 101-154 and a second nucleic acid encoding a Staphylococcus aureus Cas9 (SaCas9);
b. a first nucleic acid encoding one or more spacer sequences comprising at least 17, 18, 19, 20, or 21 contiguous nucleotides of a spacer sequence selected from any one of SEQ ID NOs: 1-8, 10-28, and 101-154 and a second nucleic acid encoding a Staphylococcus aureus Cas9 (SaCas9);
c. a first nucleic acid encoding one or more spacer sequences that are at least 90% identical to any one of 1-8, 10-28, and 101-154 and a second nucleic acid encoding a Staphylococcus aureus Cas9 (SaCas9).
   In some embodiments, the composition further comprises a DNA-PK inhibitor. In some embodiments, the DNA-PK inhibitor is Compound 1. In some embodiments, the DNA-PK inhibitor is Compound 2. In some embodiments, the DNA-PK inhibitor is Compound 6.

In some embodiments, a composition comprising a single nucleic acid molecule encoding one or more guide RNAs and a Cas9 is provided, wherein the single nucleic acid molecule comprises:
a. a first nucleic acid encoding a pair of guide RNAs comprising a first and second spacer sequence selected from any one of SEQ ID NOs: 1 and 10; 1 and 11; land 12; land 13; land 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; Band 17; Band 18; Band 19; 8 and 20; 8 and 21; 8 and 22; 8 and 23; 8 and 24; 8 and 25; 8 and 26; 8 and 27; and 8 and 28; and a second nucleic acid encoding a Staphylococcus aureus Cas9 (SaCas9);
b. a first nucleic acid encoding a pair of guide RNAs comprising at least 17, 18, 19, 20, or 21 contiguous nucleotides of a first and second spacer sequence selected from any one of SEQ ID NOs: 1 and 10; 1 and 11; 1 and 12; 1 and 13; 1 and 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; Band 19; 8 and 20; 8 and 21; 8 and 22; 8 and 23; 8 and 24; 8 and 25; 8 and 26; 8 and 27; and 8 and 28 and a second nucleic acid encoding a Staphylococcus aureus Cas9 (SaCas9);
c. a first nucleic acid encoding a pair of guide RNAs that is at least 90% identical to a first and second spacer sequence selected from any one of SEQ ID NOs: 1 and 10; 1 and 11; 1 and 12; 1 and 13; 1 and 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28 and a second nucleic acid encoding a Staphylococcus aureus Cas9 (SaCas9); or
d. a first nucleic acid encoding a pair of guide RNAs comprising a first and second spacer sequence selected from any one of SEQ ID NOs: 4 and 12; 2 and 12; 3 and 12; 4 and 20; 4 and 18; 2 and 10; 4 and 10; 1 and 12; 8 and 12; 4 and 13; 7 and 12; 7 and 28; 7 and 18 and a second nucleic acid encoding a Staphylococcus aureus Cas9 (SaCas9). In some embodiments, the composition further comprises a DNA-PK inhibitor. In some embodiments, the DNA-PK inhibitor is Compound 1. In some embodiments, the DNA-PK inhibitor is Compound 2. In some embodiments, the DNA-PK inhibitor is Compound 6.

In some embodiments, a nucleic acid encoding a guide RNA and a nucleic acid encoding a Cas9 are provided on a single nucleic acid molecule. In some embodiments, the single nucleic acid molecule comprises a nucleic acid encoding one or more guide RNA and a nucleic acid encoding a SaCas9. In some embodiments, two guide RNAs and a Cas9 are provided on a single nucleic acid molecule. In some embodiments, a nucleic acid encoding three guide RNAs and a nucleic acid encoding a SaCas9 are provided on a single nucleic acid molecule. In some embodiments, single nucleic acid molecule comprises a nucleic acid encoding a Cas9, and a nucleic acid encoding two guide RNAs, wherein the nucleic acid molecule encodes no more than two guide RNAs. In some embodiments, the single nucleic acid molecule comprises a nucleic acid encoding a first guide RNA, a nucleic acid encoding a second guide RNA, and a nucleic acid encoding a SaCas9, where the first and second guide RNA can be the same or different. In some embodiments, the single nucleic acid molecule comprises a nucleic acid encoding a first guide RNA, a nucleic acid encoding a second guide RNA, a nucleic acid encoding a third guide RNA, and a nucleic acid encoding a SaCas9, where the first, second, and third guide RNA can be the same or different. In some embodiments, the spacer sequences of the first and second guide RNAs are identical. In some embodiments, the spacer sequences of the first and second guide RNAs are non-identical (e.g., a pair of guide RNAs). In some embodiments, a system is provided comprising two vectors, wherein the first vector comprises one or more (e.g., 1, 2, 3, 4, 5, or 6) guide RNAs, which can be the same or different, and a second vector comprises one or more guide RNAs (e.g., 1, 2, or 3), which can be the same or different as compared to the other guide RNAs in the second vector or as compared to the other guide RNAs in the first vector, and a nucleic acid encoding a SaCas9. In some embodiments, the first nucleic acid molecule encodes for a Cas9 molecule and also encodes for one or more copies of a first guide RNA and one or more copies of a second guide RNA. In some embodiments, the first nucleic acid molecule encodes for a Cas9 molecule, but does not encode for any guide RNAs. In some embodiments, the second nucleic acid molecule encodes for one or more copies of a first guide RNA and one or more copies of a second guide RNA, wherein the second nucleic acid molecule does not encode for a Cas9 molecule.

In some embodiments, the disclosure provides for a composition comprising two nucleic acid molecules, wherein the first nucleic acid molecule comprises a sequence encoding a SaCas9 protein, and wherein the second nucleic acid molecule encodes for a first guide RNA. In some embodiments, the first nucleic acid molecule also encodes for the first guide RNA. In other embodiments, the first nucleic acid molecule does not encode for any guide RNA. In some embodiments, the second nucleic acid molecule encodes for a second guide RNA. In some embodiments, the first nucleic acid molecule also encodes for the second guide RNA. In particular embodiments, the first guide RNA and the second guide RNA are not identical. In some embodiments, the second nucleic acid molecule encodes for two copies of the first guide RNA. In some embodiments, the second nucleic acid molecule encodes for two copies of the second guide RNA. In some embodiments, the second nucleic acid molecule encodes for three copies of the first guide RNA. In some embodiments, the second nucleic acid molecule encodes for three copies of the second guide RNA. In some embodiments, the second nucleic acid molecule encodes for two copies of the first guide RNA and two copies of the second guide RNA. In some embodiments, the second nucleic acid molecule encodes for two copies of the first guide RNA and one copy of the second guide RNA. In some embodiments, the second nucleic acid molecule encodes for one copy of the first guide RNA and two copies of the second guide RNA. In some embodiments, the second nucleic acid molecule encodes for three copies of the first guide RNA and three copies of the second guide RNA. In particular embodiments, the first guide RNA and the second guide RNA are not identical. In some embodiments, the first nucleic acid is in a first viral vector and the second nucleic acid is in a separate second viral vector. In some embodiments, the first guide RNA comprises a sequence selected from any one of SEQ ID Nos: 1-8, 10-28, and 101-154, and the second guide RNA comprises a sequence selected from any one of SEQ ID Nos: 1-8, 10-28, and 101-154. In some embodiments, the second nucleic acid encodes for one or more copies of a first guide RNA (e.g., a guide RNA comprising a sequence from any one of SEQ ID Nos: 1-8, 10-28, and 101-154), and does not encode for any additional different guide RNAs. In some embodiments, the second nucleic acid encodes for one or more copies of a first guide RNA comprising the nucleotide sequence of SEQ ID NO: 1-8, 10-28, and 101-154, and does not encode for any additional different guide RNAs.

In some embodiments, the single nucleic acid molecule is a single vector. In some embodiments, the single vector expresses the one or two or three guide RNAs and Cas9. In some embodiments, one or more guide RNA and a Cas9 are provided on a single vector. In some embodiments, the single vector comprises a nucleic acid encoding a guide RNA and a nucleic acid encoding a SaCas9. In some embodiments, two guide RNAs and a Cas9 are provided on a single vector. In some embodiments, three guide RNAs and a Cas9 are provided on a single vector. In some embodiments, the single vector comprises a nucleic acid encoding a first guide RNA, a nucleic acid encoding a second guide RNA, and a nucleic acid encoding a SaCas9. In some embodiments, the single vector comprises a nucleic acid encoding a first guide RNA, a nucleic acid encoding a second guide RNA, a nucleic acid encoding a third guide RNA, and a nucleic acid encoding a SaCas9. In some embodiments, the spacer sequences of the first, second, and third guide RNAs, if present, are identical. In some embodiments, the spacer sequences of the first, second, and third guide RNAs, if present are non-identical (e.g., a pair of guide RNAs).

Each of the guide sequences shown in Table 1A and Table 1B may further comprise additional nucleotides to form or encode a crRNA, e.g., using any known sequence appropriate for the Cas9 being used. In some embodiments, the crRNA comprises (5′ to 3′) at least a spacer sequence and a first complementarity domain. The first complementary domain is sufficiently complementary to a second complementarity domain, which may be part of the same molecule in the case of an sgRNA or in a tracrRNA in the case of a dual or modular gRNA, to form a duplex. See, e.g., US 2017/0007679 for detailed discussion of crRNA and gRNA domains, including first and second complementarity domains.

A single-molecule guide RNA (sgRNA) can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and/or an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins. In particular embodiments, the disclosure provides for an sgRNA comprising a spacer sequence and a tracrRNA sequence.

An exemplary scaffold sequence suitable for use with SaCas9 to follow the guide sequence at its 3′ end is:

(SEQ ID NO: 500)
GTTTAAGTACTCTGTGCTGGAAACAGCACAGAATCTACTTAAACA
AGGCAAAATGCCGTGTTTATCTCGTCAACTTGTTGGCGAGA

in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 500, or a sequence that differs from SEQ ID NO: 500 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV1” and is:

(SEQ ID NO: 910)
GTTTTAGTACTCTGGAAACAGAATCTACTAAAACAAGGCAAAATG
CCGTGTTTATCTCGTCAACTTGTTGGCGAGAT

in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 910, or a sequence that differs from SEQ ID NO: 910 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV2” and is:

(SEQ ID NO: 911)
GTTTAAGTACTCTGTGCTGGAAACAGCACAGAATCTACTTAAACA
AGGCAAAATGCCGTGTTTATCTCGTCAACTTGTTGGCGAGAT

in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 911, or a sequence that differs from SEQ ID NO: 911 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV3” and is:

(SEQ ID NO: 912)
GTTTAAGTACTCTGGAAACAGAATCTACTTAAACAAGGCAAAATG
CCGTGTTTATCTCGTCAACTTGTTGGCGAGAT

in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 912, or a sequence that differs from SEQ ID NO: 912 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV4” and is:

(SEQ ID NO: 920)
GTTTCAGTACTCTGTGCTGGAAACAGCACAGAATCTACTGAAACA
AGGCAAAATGCCGTGTTTATCTCGTCAACTTGTTGGCGAGAT

in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 920, or a sequence that differs from SEQ ID NO: 920 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV5” and is:

(SEQ ID NO: 921)
GTTTCAGTACTCTGGAAACAGAATCTACTGAAACAAGGCAAAATG
CCGTGTTTATCTCGTCAACTTGTTGGCGAGAT

in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 921, or a sequence that differs from SEQ ID NO: 921 by no more than 1, 2, 3, 4, 5, 10, 15, 20, or 25 nucleotides.

In some embodiments, the nucleic acid encoding the gRNA or the nucleic acid encoding the pair of gRNAs comprises a sequence comprising SEQ ID NO: 500. In some embodiments, the nucleic acid encoding the gRNA or the nucleic acid encoding the pair of gRNAs comprises a sequence comprising SEQ ID NO: 910. In some embodiments, the nucleic acid encoding the gRNA or the nucleic acid encoding the pair of gRNAs comprises a sequence comprising SEQ ID NO: 911. In some embodiments, the nucleic acid encoding the gRNA or the nucleic acid encoding the pair of gRNAs comprises a sequence comprising SEQ ID NO: 912. In some embodiments, the nucleic acid encoding the gRNA or the nucleic acid encoding the pair of gRNAs comprises a sequence comprising SEQ ID NO: 920. In some embodiments, the nucleic acid encoding the gRNA or the nucleic acid encoding the pair of gRNAs comprises a sequence comprising SEQ ID NO: 921. In some embodiments, in a nucleic acid molecule comprising a pair of gRNAs, one of the gRNAs comprises a sequence selected from any one of SEQ ID NOs: 500, 910, 911, 912, 920, and 921. In some embodiments, in a nucleic acid molecule comprising a pair of gRNAs, both of the gRNAs comprise a sequence selected from any one of SEQ ID NOs: 500, 910, 911, 912, 920, and 921. In some embodiments, in a nucleic acid molecule comprising a pair of gRNAs, the first gRNA in the pair comprises a sequence selected from any one of SEQ ID Nos: 500, 910, 911, 912, 920, and 921, and the second gRNA in the pair comprises a different sequence selected from any one of SEQ ID Nos: 500, 910, 911, 912, 920, and 921. In some embodiments, in a nucleic acid molecule comprising a pair of gRNAs, the nucleotides 3′ of the guide sequence of the gRNAs are the same sequence. In some embodiments, in a nucleic acid molecule comprising a pair of gRNAs, the nucleotides 3′ of the guide sequence of the gRNAs are different sequences. In some embodiments, the scaffold-encoding sequence for the first guide RNA comprises the sequence of SEQ ID NO: 921, and the scaffold-encoding sequence for the second guide RNA comprises the sequence of SEQ ID NO: 921.

In some embodiments, the scaffold sequence comprises one or more alterations in the stem loop 1 as compared to the stem loop 1 of a wildtype SaCas9 scaffold sequence (e.g., a scaffold comprising the sequence of SEQ ID NO: 500) or a reference SaCas9 scaffold sequence (e.g., a scaffold comprising the sequence of SEQ ID NO: 921). In some embodiments, the scaffold sequence comprises one or more alterations in the stem loop 2 as compared to the stem loop 2 of a wildtype SaCas9 scaffold sequence (e.g., a scaffold comprising the sequence of SEQ ID NO: 500) or a reference SaCas9 scaffold sequence (e.g., a scaffold comprising the sequence of SEQ ID NO: 921). In some embodiments, the scaffold sequence comprises one or more alterations in the tetraloop as compared to the tetraloop of a wildtype SaCas9 scaffold sequence (e.g., a scaffold comprising the sequence of SEQ ID NO: 500) or a reference SaCas9 scaffold sequence (e.g., a scaffold comprising the sequence of SEQ ID NO: 921). In some embodiments, the scaffold sequence comprises one or more alterations in the repeat region as compared to the repeat region of a wildtype SaCas9 scaffold sequence (e.g., a scaffold comprising the sequence of SEQ ID NO: 500) or a reference SaCas9 scaffold sequence (e.g., a scaffold comprising the sequence of SEQ ID NO: 921). In some embodiments, the scaffold sequence comprises one or more alterations in the anti-repeat region as compared to the anti-repeat region of a wildtype SaCas9 scaffold sequence (e.g., a scaffold comprising the sequence of SEQ ID NO: 500) or a reference SaCas9 scaffold sequence (e.g., a scaffold comprising the sequence of SEQ ID NO: 921). In some embodiments, the scaffold sequence comprises one or more alterations in the linker region as compared to the linker region of a wildtype SaCas9 scaffold sequence (e.g., a scaffold comprising the sequence of SEQ ID NO: 500) or a reference SaCas9 scaffold sequence (e.g., a scaffold comprising the sequence of SEQ ID NO: 921). See, e.g., Nishimasu et al., 2015, Cell, 162:1113-1126 for description of regions of a scaffold.

Where a tracrRNA is used, in some embodiments, it comprises (5′ to 3′) a second complementary domain and a proximal domain. In the case of a sgRNA, guide sequences together with additional nucleotides (e.g., SEQ ID NOs: 500, 910, 911, 912, 920, or 921) form or encode a sgRNA. In some embodiments, an sgRNA comprises (5′ to 3′) at least a spacer sequence, a first complementary domain, a linking domain, a second complementary domain, and a proximal domain. A sgRNA or tracrRNA may further comprise a tail domain. The linking domain may be hairpin-forming. See, e.g., US 2017/0007679 for detailed discussion and examples of crRNA and gRNA domains, including second complementarity domains, linking domains, proximal domains, and tail domains.

In general, in the case of a DNA nucleic acid construct encoding a guide RNA, the U residues in any of the RNA sequences described herein may be replaced with T residues, and in the case of a guide RNA construct encoded by a DNA, the T residues may be replaced with U residues.

Provided herein are compositions comprising one or more guide RNAs or one or more nucleic acids encoding one or more guide RNAs comprising a guide sequence disclosed herein in Table 1A and Table 1B and throughout the specification.

In some embodiments, a composition is provided comprising a guide RNA, or nucleic acid encoding a guide RNA, wherein the guide RNA comprises 17, 18, 19, 20, or 21 contiguous nucleotides of any one of the guide sequences disclosed herein in Table 1A and Table 1B and throughout the specification.

In some embodiments, a composition is provided comprising a guide RNA, or nucleic acid encoding a guide RNA, wherein the guide RNA comprises a sequence with about 75%, 80%, 85%, 90%, 95%, 96%, 97%, 98%, 99%, or 100% identity to at least 17, 18, 19, 20, or 21 contiguous nucleotides of a guide sequence shown in Table 1A and Table 1B and throughout the specification.

In some embodiments, a composition is provided comprising a guide RNA, or nucleic acid encoding a guide RNA, wherein the guide RNA comprises a sequence with about 75%, 80%, 85%, 90%, 95%, 96%, 97%, 98%, 99%, or 100% identity to a guide sequence shown in Table 1A and Table 1B and throughout the specification.

In some embodiments, a composition is provided comprising at least one guide RNA, or nucleic acid encoding at least one guide RNA, wherein at least one of the guide RNA comprises a spacer sequence selected from any one of SEQ ID NOs: 1-8, 10-28, and 101-154.

In some embodiments, a composition is provided comprising at least one guide RNA, or nucleic acid encoding at least one guide RNA, wherein at least one of the guide RNA comprises a spacer sequence selected from any one of SEQ ID NOs: 1-9, 10-28, and 101-154.

In some embodiments, a composition is provided comprising at least one guide RNA, or nucleic acid encoding at least one guide RNA, wherein at least one of the guide RNA comprises a spacer sequence selected from any one of SEQ ID NOs: 1, 2, 3, 4, 7, 8, 10, 11, 12, 13, 14, 15, 18, 19, 20, 21, 23, 25, 26, 27, and 28. In some embodiments, a composition is provided comprising at least one guide RNA, or nucleic acid encoding at least one guide RNA, wherein the at least one guide RNA comprises a spacer sequence selected from any one of SEQ ID NOs: 1, 2, 3, 4, 7, 8, 12, and 20. In some embodiments, a composition is provided comprising at least one guide RNA, or nucleic acid encoding at least one guide RNA, wherein the at least one guide RNA comprises a spacer sequence selected from any one of SEQ ID NOs: 1, 2, 3, 4, 7, 8, and 20. In some embodiments, the spacer sequence is SEQ ID NO: 1. In some embodiments, the spacer sequence is SEQ ID NO: 2. In some embodiments, the spacer sequence is SEQ ID NO: 3. In some embodiments, the spacer sequence is SEQ ID NO: 4. In some embodiments, the spacer sequence is SEQ ID NO: 7. In some embodiments, the spacer sequence is SEQ ID NO: 8. In some embodiments, the spacer sequence is SEQ ID NO: 10. In some embodiments, the spacer sequence is SEQ ID NO: 11. In some embodiments, the spacer sequence is SEQ ID NO: 12. In some embodiments, the spacer sequence is SEQ ID NO: 13. In some embodiments, the spacer sequence is SEQ ID NO: 14. In some embodiments, the spacer sequence is SEQ ID NO: 15. In some embodiments, the spacer sequence is SEQ ID NO: 18. In some embodiments, the spacer sequence is SEQ ID NO: 19. In some embodiments, the spacer sequence is SEQ ID NO: 20. In some embodiments, the spacer sequence is SEQ ID NO: 21. In some embodiments, the spacer sequence is SEQ ID NO: 23. In some embodiments, the spacer sequence is SEQ ID NO: 25. In some embodiments, the spacer sequence is SEQ ID NO: 26. In some embodiments, the spacer sequence is SEQ ID NO: 27. In some embodiments, the spacer sequence is SEQ ID NO: 28. In some embodiments, the composition further comprises a DNA-PK inhibitor.

In some embodiments, a composition is provided comprising at least one guide RNA, or nucleic acid encoding at least one guide RNA, wherein at least one of the guide RNA comprises a spacer sequence selected from any one of SEQ ID NOs: 101, 102, 103, 104, 105, 106, 107, 113, 114, 115, 116, 117, 118, 119, 120, 121, 122, 123, 124, 125, 126, 127, 128, 133, 134, 135, 136, 137, 138, 139, 140, 143, 144, 147, 148, 149, 150, 151, 152, 153, and 154. In some embodiments, the spacer sequence is SEQ ID NO: 101. In some embodiments, the spacer sequence is SEQ ID NO: 102. In some embodiments, the spacer sequence is SEQ ID NO: 103. In some embodiments, the spacer sequence is SEQ ID NO: 104. In some embodiments, the spacer sequence is SEQ ID NO: 105. In some embodiments, the spacer sequence is SEQ ID NO: 106. In some embodiments, the spacer sequence is SEQ ID NO: 107. In some embodiments, the spacer sequence is SEQ ID NO: 113. In some embodiments, the spacer sequence is SEQ ID NO: 114. In some embodiments, the spacer sequence is SEQ ID NO: 115. In some embodiments, the spacer sequence is SEQ ID NO: 116. In some embodiments, the spacer sequence is SEQ ID NO: 117. In some embodiments, the spacer sequence is SEQ ID NO: 118. In some embodiments, the spacer sequence is SEQ ID NO: 119. In some embodiments, the spacer sequence is SEQ ID NO: 120. In some embodiments, the spacer sequence is SEQ ID NO: 121. In some embodiments, the spacer sequence is SEQ ID NO: 122. In some embodiments, the spacer sequence is SEQ ID NO: 123. In some embodiments, the spacer sequence is SEQ ID NO: 124. In some embodiments, the spacer sequence is SEQ ID NO: 125. In some embodiments, the spacer sequence is SEQ ID NO: 126. In some embodiments, the spacer sequence is SEQ ID NO: 127. In some embodiments, the spacer sequence is SEQ ID NO: 128. In some embodiments, the spacer sequence is SEQ ID NO: 133. In some embodiments, the spacer sequence is SEQ ID NO: 134. In some embodiments, the spacer sequence is SEQ ID NO: 135. In some embodiments, the spacer sequence is SEQ ID NO: 136. In some embodiments, the spacer sequence is SEQ ID NO: 137. In some embodiments, the spacer sequence is SEQ ID NO: 138. In some embodiments, the spacer sequence is SEQ ID NO: 139. In some embodiments, the spacer sequence is SEQ ID NO: 140. In some embodiments, the spacer sequence is SEQ ID NO: 143. In some embodiments, the spacer sequence is SEQ ID NO: 144; In some embodiments, the spacer sequence is SEQ ID NO: 147; In some embodiments, the spacer sequence is SEQ ID NO: 148; In some embodiments, the spacer sequence is SEQ ID NO: 149; In some embodiments, the spacer sequence is SEQ ID NO: 150; In some embodiments, the spacer sequence is SEQ ID NO: 151; In some embodiments, the spacer sequence is SEQ ID NO: 152; In some embodiments, the spacer sequence is SEQ ID NO: 153; In some embodiments, the spacer sequence is SEQ ID NO: 154. In some embodiments, the composition further comprises a DNA-PK inhibitor.

In some embodiments, a composition is provided comprising a pair of guide RNAs, or nucleic acid encoding a pair of guide RNAs, wherein the pair of guide RNAs comprises a first and second spacer sequence selected from any one of SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 8 and 10; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 7 and 10; 1 and 13; 1 and 20; 1 and 28; 4 and 27; 7 and 20; 7 and 23; 7 and 13; 7 and 28; 2 and 27; 8 and 27; 4 and 11; 4 and 25; 4 and 28; 4 and 19; 4 and 15; 8 and 11; 3 and 27; 2 and 25; 2 and 11; 7 and 18; 3 and 25; 8 and 15; 8 and 25; 3 and 11; 3 and 19; 1 and 15; 3 and 15; 1 and 27; 2 and 15; 2 and 19; 1 and 11; 1 and 25; 8 and 19; 4 and 21; 8 and 21; 7 and 27; 7 and 15; land 19; 2 and 21; 7 and 11; 3 and 21; 4 and 14; 7 and 19; 4 and 26; 8 and 26; 7 and 25; 1 and 21; 3 and 26; 2 and 26; 8 and 14; 1 and 14; 2 and 14; 3 and 14; 1 and 26; 7 and 21; 7 and 14; and 7 and 26. In some embodiments, the composition further comprises a DNA-PK inhibitor.

In some embodiments, a composition is provided comprising a pair of guide RNAs, or nucleic acid encoding a pair of guide RNAs, wherein the pair of guide RNAs comprises a first and second spacer sequence selected from any one of SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 8 and 10; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 7 and 10; 1 and 13; 1 and 20; 1 and 28; 4 and 27; 7 and 20; 7 and 23; 7 and 13; 7 and 28; 2 and 27; 8 and 27; 4 and 11; and 4 and 25. In some embodiments, the composition further comprises a DNA-PK inhibitor. In some embodiments, a composition or system comprising more than one vector is provided wherein the first vector comprises a single nucleic acid molecule encoding 1) one or more guide RNA comprising any one or more of the spacer sequences of SEQ ID NOs: 1-8, 10-28, and 101-154; and 2) a SaCas9, and a second vector comprises a nucleic acid encoding multiple copies of guide RNA. In some embodiments, a composition or system comprising more than one vector is provided wherein the first vector comprises a single nucleic acid molecule encoding a SaCas9 and not any guide RNAs, and a second vector comprises a nucleic acid encoding one or more guide RNA comprising any one or more of the spacer sequences of SEQ ID NOs: 1-8, 10-28, and 101-154. In such composition or system encoding for multiple guide RNAs, the guide RNAs can be the same or different.

In some embodiments, a composition is provided comprising a pair of guide RNAs, or nucleic acid encoding a pair of guide RNAs, wherein the pair of guide RNAs comprises a first and second spacer sequence selected from any one of SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 1 and 13; 1 and 20; 1 and 28; 4 and 27; 7 and 20; 7 and 23; 7 and 13; 7 and 28; 2 and 27; 4 and 11; 4 and 25; 4 and 28; 4 and 19; 4 and 15; 8 and 11; 3 and 27; 2 and 25; 2 and 11; 7 and 18; 3 and 25; 8 and 15; 3 and 11; 3 and 19; 1 and 15; 3 and 15; 1 and 27; 2 and 15; 2 and 19; 1 and 11; 1 and 25; 4 and 21; 8 and 21; 7 and 27; 7 and 15; land 19; 2 and 21; 7 and 11; 3 and 21; 7 and 19; 7 and 25; 1 and 21; 3 and 26; 3 and 14; 7 and 21; and 7 and 14. In some embodiments, the composition further comprises a DNA-PK inhibitor.

In some embodiments, a composition is provided comprising a pair of guide RNAs, or nucleic acid encoding a pair of guide RNAs, wherein the pair of guide RNAs comprises a first and second spacer sequence selected from any one of SEQ ID NOs: 4 and 12; 2 and 10; 4 and 28; 8 and 12; 4 and 13; 3 and 10; 7 and 12; 7 and 13; 4 and 28; and 7 and 18. In some embodiments, the composition further comprises a DNA-PK inhibitor.

In some embodiments, a composition is provided comprising a pair of guide RNAs, or nucleic acid encoding a pair of guide RNAs, wherein the pair of guide RNAs comprises a first and second spacer sequence selected from any one of SEQ ID NOs: 4 and 12; 2 and 12; 3 and 12; 4 and 20; 4 and 18; 2 and 10; 4 and 10; 1 and 12; 8 and 12; 4 and 13; 7 and 12; 7 and 28; 7 and 18. In some embodiments, the first and second spacer sequence are SEQ ID NOs: 4 and 12. In some embodiments, the first and second spacer sequence are SEQ ID NOs: 2 and 12. In some embodiments, the first and second spacer sequence are SEQ ID NOs: 3 and 12. In some embodiments, the first and second spacer sequence are SEQ ID NOs: 4 and 20. In some embodiments, the first and second spacer sequence are SEQ ID NOs: 4 and 18. In some embodiments, the first and second spacer sequence are SEQ ID NOs: 2 and 10. In some embodiments, the first and second spacer sequence are SEQ ID NOs: 4 and 10. In some embodiments, the first and second spacer sequence are SEQ ID NOs: 1 and 12. In some embodiments, the first and second spacer sequence are SEQ ID NOs: 8 and 12. In some embodiments, the first and second spacer sequence are SEQ ID NOs: 4 and 13. In some embodiments, the first and second spacer sequence are SEQ ID NOs: 7 and 12. In some embodiments, the first and second spacer sequence are SEQ ID NOs: 7 and 28. In some embodiments, the first and second spacer sequence are SEQ ID NOs: 7 and 18. In some embodiments, the composition further comprises a DNA-PK inhibitor.

In some embodiments, a composition is provided comprising a pair of guide RNAs, or nucleic acid encoding a pair of guide RNAs, wherein the pair of guide RNAs comprises a first and second spacer sequence selected from any one of SEQ ID NOs: 7 and 12; 4 and 12; and 7 and 23. In some embodiments the pair of guide RNAs comprises a first and second spacer sequence of SEQ ID NOs: 7 and 12. In some embodiments the pair of guide RNAs comprises a first and second spacer sequence of SEQ ID NOs: 4 and 12. In some embodiments the pair of guide RNAs comprises a first and second spacer sequence of SEQ ID NOs: 7 and 23. In some embodiments, the composition further comprises a DNA-PK inhibitor.

In some embodiments, a composition is provided comprising a guide RNA, or nucleic acid encoding a guide RNA, wherein the guide RNA further comprises a trRNA. In each composition and method embodiment described herein, the crRNA (comprising the spacer sequence) and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond. In some embodiments, the composition further comprises a DNA-PK inhibitor.

In one aspect, a composition is provided comprising a single nucleic acid molecule encoding 1) one or more guide RNA that comprises a guide sequence selected from any one of SEQ ID NOs: 1-8, 10-28, and 101-154; and 2) a SaCas9. In some embodiments, the composition further comprises a DNA-PK inhibitor.

In one aspect, a composition is provided comprising a single nucleic acid molecule encoding 1) one or more guide RNA that comprises a guide sequence that is at least 99%, 98%, 97%, 96%, 95%, 94%, 93%, 92%, 91%, or 90% identical to any one of SEQ ID NOs: 1-8, 10-28, and 101-154; and 2) a SaCas9. In some embodiments, the composition further comprises a DNA-PK inhibitor.

In another aspect, a composition is provided comprising a single nucleic acid molecule encoding 1) one or more guide RNA that comprises a guide sequence comprising at least 17, 18, 19, 20, or 21 contiguous nucleotides of a spacer sequence selected from any one of SEQ ID NOs: 1-8, 10-28, and 101-154; and 2) a SaCas9. In some embodiments, the composition further comprises a DNA-PK inhibitor.

In one aspect, a composition is provided comprising a single nucleic acid molecule encoding 1) a pair of guide RNAs that comprise a first and second spacer sequence selected from any one of SEQ ID NOs: 1 and 10; 1 and 11; 1 and 12; 1 and 13; 1 and 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; band 14; band 15; band 16; band 17; band 18; band 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28; and 2) a SaCas9. In some embodiments, the composition further comprises a DNA-PK inhibitor.

In one aspect, a composition is provided comprising a single nucleic acid molecule encoding 1) a pair of guide RNAs that comprise a first and second spacer sequence that is at least 99%, 98%, 97%, 96%, 95%, 94%, 93%, 92%, 91%, or 90% identical to any one of SEQ ID NOs: 1 and 10; land 11; land 12; land 13; land 14; land 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; band 16; band 17; band 18; band 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28; and 2) a SaCas9. In some embodiments, the composition further comprises a DNA-PK inhibitor.

In another aspect, a composition is provided comprising a single nucleic acid molecule encoding 1) a pair of guide RNAs that comprise a first and second spacer sequence comprising at least 17, 18, 19, 20, or 21 contiguous nucleotides of a spacer sequence selected from any one of SEQ ID NOs: 1 and 10; 1 and 11; 1 and 12; 1 and 13; 1 and 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; band 16; band 17; band 18; band 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28; and 2) a SaCas9. In some embodiments, the composition further comprises a DNA-PK inhibitor.

In any embodiment comprising a nucleic acid molecule encoding a guide RNA and/or a Cas9, the nucleic acid molecule may be a vector. In some embodiments, a composition is provided comprising a single nucleic acid molecule encoding a guide RNA and Cas9, wherein the nucleic acid molecule is a vector.

Any type of vector, such as any of those described herein, may be used. In some embodiments, the vector is a viral vector. In some embodiments, the viral vector is a non-integrating viral vector (i.e., that does not insert sequence from the vector into a host chromosome). In some embodiments, the viral vector is an adeno-associated virus vector (AAV), a lentiviral vector, an integrase-deficient lentiviral vector, an adenoviral vector, a vaccinia viral vector, an alphaviral vector, or a herpes simplex viral vector. In some embodiments, the vector comprises a muscle-specific promoter. Exemplary muscle-specific promoters include a muscle creatine kinase promoter, a desmin promoter, an MHCK7 promoter, or an SPcS-12 promoter. See US 2004/0175727 A1; Wang et al., Expert Opin Drug Deliv. (2014) 11, 345-364; Wang et al., Gene Therapy (2008) 15, 1489-1499. In some embodiments, the muscle-specific promoter is a CK8 promoter. In some embodiments, the muscle-specific promoter is a CK8e promoter. In any of the foregoing embodiments, the vector may be an adeno-associated virus vector (AAV). In some embodiments, the vector is an AAV9 vector.

In some embodiments, the muscle specific promoter is the CK8 promoter. The CK8 promoter has the following sequence (SEQ ID NO. 700):

| | |
|---|---|
| 1 | CTAGACTAGC ATGCTGCCCA TGTAAGGAGG |
| | CAAGGCCTGG GGACACCCGA GATGCCTGGT |
| 61 | TATAATTAAC CCAGACATGT GGCTGCCCCC |
| | CCCCCCCCAA CACCTGCTGC CTCTAAAAAT |
| 121 | AACCCTGCAT GCCATGTTCC CGGCGAAGGG |
| | CCAGCTGTCC CCCGCCAGCT AGACTCAGCA |
| 181 | CTTAGTTTAG GAACCAGTGA GCAAGTCAGC |
| | CCTTGGGGCA GCCCATACAA GGCCATGGGG |
| 241 | CTGGGCAAGC TGCACGCCTG GGTCCGGGGT |
| | GGGCACGGTG CCCGGGCAAC GAGCTGAAAG |
| 301 | CTCATCTGCT CTCAGGGGCC CCTCCCTGGG |
| | GACAGCCCCT CCTGGCTAGT CACACCCTGT |
| 361 | AGGCTCCTCT ATATAACCCA GGGGCACAGG |
| | GGCTGCCCTC ATTCTACCAC CACCTCCACA |
| 421 | GCACAGACAG ACACTCAGGA GCCAGCCAGC |

In some embodiments, the muscle-cell cell specific promoter is a variant of the CK8 promoter, called CK8e. In some embodiments, the size of the CK8e promoter is 436 bp. The CK8e promoter has the following sequence (SEQ ID NO. 701):

| | |
|---|---|
| 1 | TGCCCATGTA AGGAGGCAAG GCCTGGGGAC |
| | ACCCGAGATG CCTGGTTATA ATTAACCCAG |
| 61 | ACATGTGGCT GCCCCCCCCC CCCCAACACC |
| | TGCTGCCTCT AAAAATAACC CTGCATGCCA |
| 121 | TGTTCCCGGC GAAGGGCCAG CTGTCCCCCG |
| | CCAGCTAGAC TCAGCACTTA GTTTAGGAAC |
| 181 | CAGTGAGCAA GTCAGCCCTT GGGGCAGCCC |
| | ATACAAGGCC ATGGGGCTGG GCAAGCTGCA |
| 241 | CGCCTGGGTC CGGGGTGGGC ACGGTGCCCG |
| | GGCAACGAGC TGAAAGCTCA TCTGCTCTCA |
| 301 | GGGGCCCCTC CCTGGGGACA GCCCCTCCTG |
| | GCTAGTCACA CCCTGTAGGC TCCTCTATAT |
| 361 | AACCCAGGGG CACAGGGGCT GCCCTCATTC |
| | TACCACCACC TCCACAGCAC AGACAGACAC |
| 421 | TCAGGAGCCA GCCAGC |

In some embodiments, the vector comprises one or more of a U6, H1, or 7SK promoter. In some embodiments, the U6 promoter is the human U6 promoter (e.g., the U6L promoter or U6S promoter). In some embodiments, the promoter is the murine U6 promoter. In some embodiments, the 7SK promoter is a human 7SK promoter. In some embodiments, the 7SK promoter is the 7SK1 promoter. In some embodiments, the 7SK promoter is the 7SK2 promoter. In some embodiments, the H1 promoter is a human H1 promoter (e.g., the HIL promoter or the HIS promoter). In some embodiments, the vector comprises multiple guide sequences, wherein each guide sequence is under the control of a separate promoter. In some embodiments, each of the multiple guide sequences comprises a different sequence. In some embodiments, each of the multiple guide sequences comprise the same sequence (e.g., each of the multiple guide sequences comprise the same spacer sequence). In some embodiments, each of the multiple guide sequences comprises the same spacer sequence and the same scaffold sequence. In some embodiments, each of the multiple guide sequences comprises different spacer sequences and different scaffold sequences. In some embodiments, each of the multiple guide sequences comprises the same spacer sequence, but comprises a different scaffold sequence. In some embodiments, each of the multiple guide sequences comprises different spacer sequences and different scaffold sequences. In some embodiments, each of the separate promoters comprises the same nucleotide sequence (e.g., the U6 promoter sequence). In some embodiments, each of the separate promoters comprises a different nucleotide sequence (e.g., the U6, H1, and/or 7SK promoter sequence).

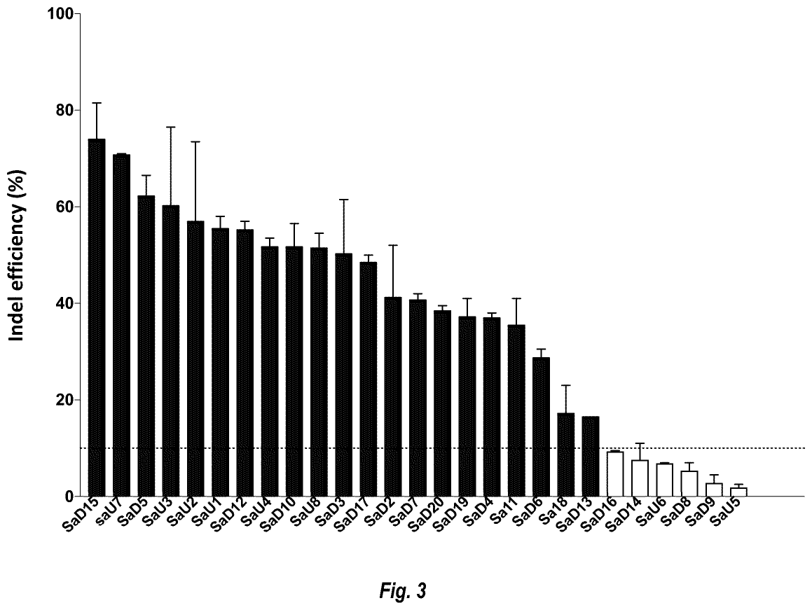

In some embodiments, a single nucleic acid molecule is provided comprising at least two gRNAs, wherein the promoters are selected to allow for about equal editing kinetics of the gRNAs. In some embodiments, a single nucleic acid molecule is provided comprising a pair of gRNAs, wherein the promoters are selected to allow for about equal editing kinetics of the gRNAs. In some embodiments, the pair of guide RNAs comprises a first guide RNA and a second guide RNA, wherein the first guide has higher indel efficiency than the second guide when tested under the same conditions (see, e.g., Example 1 and FIG. 3). In some embodiments, the first guide RNA having higher indel efficiency is operably placed under the control of a first promoter and the second guide RNA having lower indel efficiency is operably placed under the control of a second promoter, wherein the second promoter is stronger (i.e., drives stronger expression) than the first promoter. In some embodiments, the first guide RNA having lower indel efficiency is operably placed under the control of a first promoter and the second guide RNA having higher indel efficiency is operably placed under the control of a second promoter, wherein the second promoter is stronger (i.e., drives stronger expression) than the first promoter. For example, in some embodiments, 7SK2 is a weaker promoter (i.e., drives weaker expression) than a hU6c promoter. In some embodiments, the guide RNA having higher indel efficiency is under the control of a 7SK promoter and the guide having lower indel efficiency is under the control of an hU6 promoter. In some embodiments, the guide RNA having lower indel efficiency is under the control of a 7SK promoter and the guide having higher indel efficiency is under the control of an hU6 promoter. In some embodiments, the guide RNA under control of the weaker promoter (e.g., 7SK2 promoter) comprises the sequence of SEQ ID NO: 4, and the guide RNA under the control of the stronger promoter (e.g., hU6c) comprises the sequence of SEQ ID NO: 12. In some embodiments, the guide RNA under control of the weaker promoter (e.g., 7SK2 promoter) comprises the sequence of SEQ ID NO: 4, and the guide RNA under the control of the stronger promoter (e.g., hU6c) comprises the sequence of SEQ ID NO: 18. In some embodiments, the guide RNA under control of the weaker promoter (e.g., 7SK2 promoter) comprises the sequence of SEQ ID NO: 8, and the guide RNA under the control of the stronger promoter (e.g., hU6c) comprises the sequence of SEQ ID NO: 12. In some embodiments, the guide RNA under control of the weaker promoter (e.g., 7SK2 promoter) comprises the sequence of SEQ ID NO: 2, and the guide RNA under the control of the stronger promoter (e.g., hU6c) comprises the sequence of SEQ ID NO: 12. In some embodiments, the guide RNA under control of the weaker promoter (e.g., 7SK2 promoter) comprises the sequence of SEQ ID NO: 13, and the guide RNA under the control of the stronger promoter (e.g., hU6c) comprises the sequence of SEQ ID NO: 4. In some embodiments, the guide RNA under control of the weaker promoter (e.g., 7SK2 promoter) comprises the sequence of SEQ ID NO: 4, and the guide RNA under the control of the stronger promoter (e.g., hU6c) comprises the sequence of SEQ ID NO: 12. In some embodiments, the guide RNA under control of the weaker promoter (e.g., 7SK2 promoter) comprises the sequence of SEQ ID NO: 7, and the guide RNA under the control of the stronger promoter (e.g., hU6c) comprises the sequence of SEQ ID NO: 18. In some embodiments, the guide RNA having higher indel efficiency is under the control of a 7SK2 promoter and the guide having lower indel efficiency is under the control of an hU6c promoter. In some embodiments, the guide RNA having lower indel efficiency is under the control of a 7SK2 promoter and the guide having higher indel efficiency is under the control of an hU6c promoter.

In some embodiments, the U6 promoter comprises a nucleotide sequence that is at least 90%, 91%, 92%, 93%, 94%, 95%, 96%, 97%, 98%, 99% or 100% identical to the sequence of SEQ ID NO: 702:

cgagtccaac acccgtggga atcccatggg caccatggcc cctcgctcca aaaatgcttt 60
cgcgtcgcgc agacactgct cggtagtttc ggggatcagc gtttgagtaa gagcccgcgt 120
ctgaaccctc cgcgccgccc cggccccagt ggaaagacgc gcaggcaaaa cgcaccacgt 180
gacggagcgt gaccgcgcgc cgagcgcgcg ccaaggtcgg gcaggaagag ggcctatttc 240
ccatgattcc ttcatatttg catatacgat acaaggctgt tagagagata attagaatta 300
atttgactgt aaacacaaag atattagtac aaaatacgtg acgtagaaag taataatttc 360
ttgggtagtt tgcagtttta aaattatgtt ttaaaatgga ctatcatatg cttaccgtaa 420
cttgaaagta tttcgatttc ttggctttat atatcttgtg gaaaggacga aa 472

In some embodiments, the H1 promoter comprises a nucleotide sequence that is at least 90%, 91%, 92%, 93%, 94%, 95%, 96%, 97%, 98%, 99% or 100% identical to the sequence of SEQ ID NO: 703:

gctcggcgcg cccatatttg catgtcgcta tgtgttctgg gaaatcacca taaacgtgaa 60
atgtctttgg atttgggaat cttataagtt ctgtatgaga ccacggta 108

In some embodiments, the 7SK promoter comprises a nucleotide sequence that is at least 90%, 91%, 92%, 93%, 94%, 95%, 96%, 97%, 98%, 99% or 100% identical to the sequence of SEQ ID NO: 704:

tgacggcgcg ccctgcagta tttagcatgc cccacccatc tgcaaggcat tctggatagt 60
gtcaaaacag ccggaaatca agtccgttta tctcaaactt tagcattttg ggaataaatg 120
atatttgcta tgctggttaa attagatttt agttaaattt cctgctgaag ctctagtacg 180
ataagtaact tgacctaagt gtaaagttga gatttccttc aggtttatat agcttgtgcg 240
ccgcctgggt a 251

In some embodiments, the U6 promoter is a hU6c promoter and comprises a nucleotide sequence that is at least 90%, 91%, 92%, 93%, 94%, 95%, 96%, 97%, 98%, 99% or 100% identical to the sequence of SEQ ID NO: 705:

GAGGGCCTATTTCCCATGATTCCTTCATATTTGCATATACGATAC
AAGGCTGTTAGAGAGATAATTGGAATTAATTTGACTGTAAACACA
AAGATATTAGTACAAAATACGTGACGTAGAAAGTAATAATTTCTT
GGGTAGTTTGCAGTTTTAAAATTATGTTTTAAAATGGACTATCAT
ATGCTTACCGTAACTTGAAAGTATTTCGATTTCTTGGCTTTATAT
ATCTTGTGGAAAGGACGAAACACC.

In some embodiments, the U6 promoter is a variant of the hU6c promoter. In some embodiments, the variant of the hU6c promoter comprises alternative nucleotides as compared to the sequence of SEQ ID NO: 705. In some embodiments, the variant of the hU6c promoter comprises fewer nucleotides as compared to the 249 nucleotides of SEQ ID NO: 705. In some embodiments, the variant of the hU6c promoter has fewer nucleotides in the nucleosome binding sequence of the hU6c promoter of SEQ ID NO: 705. In some embodiments, the variant of the hU6c promoter lacks all of or at least a portion of (e.g., at least 5, 10, 15, 20, 25, or 30 nucleotides) the nucleotides corresponding to nucleotides 96-125 of SEQ ID NO: 705. In some embodiments, the variant of the hU6c promoter lacks all of or at least a portion of (e.g., at least 5, 10, 15, 20, 25, 30, 35, 40, 45, 50, 55, or 60 nucleotides) the nucleotides corresponding to nucleotides 81-140 of SEQ ID NO: 705. In some embodiments, the variant of the hU6c promoter lacks all of or at least a portion of (e.g., at least 10, 20, 30, 40, 50, 60, 65, 70, 75, 80, or 85 nucleotides) the nucleotides corresponding to nucleotides 66-150 of SEQ ID NO: 705. In some embodiments, the variant of the hU6c promoter lacks all of or at least a portion of (e.g., at least 10, 20, 30, 40, 50, 60, 70, 80, 90, 100, 110, or 120 nucleotides) the nucleotides corresponding to nucleotides 51-170 of SEQ ID NO: 705. In some embodiments, the variant of the hU6c promoter lacks the nucleotides corresponding to nucleotides 96-125 of SEQ ID NO: 705. In some embodiments, the variant of the hU6c promoter comprises 129-219 nucleotides. In some embodiments, the variant of the hU6c promoter comprises 219 nucleotides. In some embodiments, the variant of the hU6c promoter comprises 189 nucleotides. In some embodiments, the variant of the hU6c promoter comprises 159 nucleotides. In some embodiments, the variant of the hU6c promoter comprises 129 nucleotides.

In some embodiments, the U6 promoter is hU6d30 and comprises a nucleotide sequence that is at least 90%, 91%, 92%, 93%, 94%, 95%, 96%, 97%, 98%, 99% or 100% identical to the sequence of SEQ ID NO: 901:

GAGGGCCTATTTCCCATGATTCCTTCATATTTGCATATACGATAC
AAGGCTGTTAGAGAGATAATTGGAATTAATTTGACTGTAAACACA
AAGATATAATTTCTTGGGTAGTTTGCAGTTTTAAAATTATGTTTT
AAAATGGACTATCATATGCTTACCGTAACTTGAAAGTATTTCGAT
TTCTTGGCTTTATATATCTTGTGGAAAGGACGAAACACC.

In some embodiments, the U6 promoter is hU6d60 and comprises a nucleotide sequence that is at least 90%, 91%, 92%, 93%, 94%, 95%, 96%, 97%, 98%, 99% or 100% identical to the sequence of SEQ ID NO: 902:

GAGGGCCTATTTCCCATGATTCCTTCATATTTGCATATACGATAC
AAGGCTGTTAGAGAGATAATTGGAATTAATTTGACGTTTGCAGTT
TTAAAATTATGTTTTAAAATGGACTATCATATGCTTACCGTAACT
TGAAAGTATTTCGATTTCTTGGCTTTATATATCTTGTGGAAAGGA
CGAAACACC.

In some embodiments, the U6 promoter is hU6d90 and comprises a nucleotide sequence that is at least 90%, 91%, 92%, 93%, 94%, 95%, 96%, 97%, 98%, 99% or 100% identical to the sequence of SEQ ID NO: 903:

GAGGGCCTATTTCCCATGATTCCTTCATATTTGCATATACGATACAA
GGCTGTTAGAGAGATAATATTATGTTTTAAAATGGACTATCATATGC
TTACCGTAACTTGAAAGTATTTCGATTTCTTGGCTTTATATATCTTG
TGGAAAGGACGAAACACC.

In some embodiments, the U6 promoter is hU6d120 and comprises a nucleotide sequence that is at least 90%, 91%, 92%, 93%, 94%, 95%, 96%, 97%, 98%, 99% or 100% identical to the sequence of SEQ ID NO: 904:

GAGGGCCTATTTCCCATGATTCCTTCATATTTGCATATACGATACAA
GGCGGACTATCATATGCTTACCGTAACTTGAAAGTATTTCGATTTCT
TGGCTTTATATATCTTGTGGAAAGGACGAAACACC.

In some embodiments, the 7SK promoter is a 7SK2 promoter and comprises a nucleotide sequence that is at least 90%, 91%, 92%, 93%, 94%, 95%, 96%, 97%, 98%, 99% or 100% identical to the sequence of SEQ ID NO: 706:

CTGCAGTATTTAGCATGCCCCACCCATCTGCAAGGCATTCTGGATAG
TGTCAAAACAGCCGGAAATCAAGTCCGTTTATCTCAAACTTTAGCAT
TTTGGGAATAAATGATATTTGCTATGCTGGTTAAATTAGATTTTAGT
TAAATTTCCTGCTGAAGCTCTAGTACGATAAGCAACTTGACCTAAGT
GTAAAGTTGAGACTTCCTTCAGGTTTATATAGCTTGTGCGCCGCTTG
GGTACCTC.

In some embodiments, the 7SK promoter is a variant of the 7SK2 promoter. In some embodiments, the variant of the 7SK2 promoter comprises alternative nucleotides as compared to the sequence of SEQ ID NO: 706. In some embodiments, the variant of the 7SK2 promoter e.g., comprises fewer nucleotides as compared to the 243 nucleotides of SEQ ID NO: 706. In some embodiments, the variant of the 7SK2 promoter has fewer nucleotides in the nucleosome binding sequence of the 7SK2 promoter of SEQ ID NO: 706. In some embodiments, the variant of the 7SK2 promoter lacks all of or at least a portion of (e.g., at least 5, 10, 15, 20, 25, or 30 nucleotides) the nucleotides corresponding to nucleotides 95-124 of SEQ ID NO: 706. In some embodiments, the variant of the 7SK2 promoter lacks all of or at least a portion of (e.g., at least 5, 10, 15, 20, 25, 30, 35, 40, 45, 50, 55, or 60 nucleotides) the nucleotides corresponding to nucleotides 81-140 of SEQ ID NO: 706. In some embodiments, the variant of the 7SK2 promoter lacks all of or at least a portion of (e.g., at least 10, 20, 30, 40, 50, 60, 65, 70, 75, 80, 85 or 90 nucleotides) the nucleotides corresponding to nucleotides 67-156 of SEQ ID NO: 706. In some embodiments, the variant of the 7SK2 promoter lacks all of or at least a portion of (e.g., at least 10, 20, 30, 40, 50, 60, 70, 80, 90, 100, 110, or 120 nucleotides) the nucleotides corresponding to nucleotides 52-171 of SEQ ID NO: 706. In some embodiments, the variant of the 7SK2 promoter comprises 123-213 nucleotides. In some embodiments, the variant of the 7SK2 promoter comprises 213 nucleotides. In some embodiments, the variant of the 7SK2 promoter comprises 183 nucleotides. In some embodiments, the variant of the 7SK2 promoter comprises 153 nucleotides. In some embodiments, the variant of the 7SK2 promoter comprises 123 nucleotides.

In some embodiments, the 7SK promoter is 7SKd30 and comprises a nucleotide sequence that is at least 90%, 91%, 92%, 93%, 94%, 95%, 96%, 97%, 98%, 99% or 100% identical to the sequence of SEQ ID NO: 906:

CTGCAGTATTTAGCATGCCCCACCCATCTGCAAGGCATTCTGGATAG
TGTCAAAACAGCCGGAAATCAAGTCCGTTTATCTCAAACTTTAGCAT
TTAAATTAGATTTTAGTTAAATTTCCTGCTGAAGCTCTAGTACGATA
AGCAACTTGACCTAAGTGTAAAGTTGAGACTTCCTTCAGGTTTATAT
AGCTTGTGCGCCGCTTGGGTACCTC.

In some embodiments, the 7SK promoter is 7SKd60 and comprises a nucleotide sequence that is at least 90%, 91%, 92%, 93%, 94%, 95%, 96%, 97%, 98%, 99% or 100% identical to the sequence of SEQ ID NO: 907:

CTGCAGTATTTAGCATGCCCCACCCATCTGCAAGGCATTCTGGATAG
TGTCAAAACAGCCGGAAATCAAGTCCGTTTATCTTAAATTTCCTGCT
GAAGCTCTAGTACGATAAGCAACTTGACCTAAGTGTAAAGTTGAGAC
TTCCTTCAGGTTTATATAGCTTGTGCGCCGCTTGGGTACCTC.

In some embodiments, the 7SK promoter is 7SKd90 and comprises a nucleotide sequence that is at least 90%, 91%, 92%, 93%, 94%, 95%, 96%, 97%, 98%, 99% or 100% identical to the sequence of SEQ ID NO: 908:

CTGCAGTATTTAGCATGCCCCACCCATCTGCAAGGCATTCTGGATAG
TGTCAAAACAGCCGGAAATAGCTCTAGTACGATAAGCAACTTGACCT
AAGTGTAAAGTTGAGACTTCCTTCAGGTTTATATAGCTTGTGCGCCG
CTTGGGTACCTC.

In some embodiments, the 7SK promoter is 7SKd120 and comprises a nucleotide sequence that is at least 90%, 91%, 92%, 93%, 94%, 95%, 96%, 97%, 98%, 99% or 100% identical to the sequence of SEQ ID NO: 909:

CTGCAGTATTTAGCATGCCCCACCCATCTGCAAGGCATTCTGGATAG
TGTCAGCAACTTGACCTAAGTGTAAAGTTGAGACTTCCTTCAGGTTT
ATATAGCTTGTGCGCCGCTTGGGTACCTC.

In some embodiments, the H1 promoter is a H1m or mH1 promoter and comprises a nucleotide sequence that is at least 90%, 91%, 92%, 93%, 94%, 95%, 96%, 97%, 98%, 99% or 100% identical to the sequence of SEQ ID NO: 707:

AATATTTGCATGTCGCTATGTGTTCTGGGAAATCACCATAAACGTGA
AATGTCTTTGGATTTGGGAATCTTATAAGTTCTGTATGAGACCACTC
TTTCCC.

In some embodiments, the Ck8e promoter comprises a nucleotide sequence that is at least 90%, 91%, 92%, 93%, 94%, 95%, 96%, 97%, 98%, 99% or 100% identical to the sequence of SEQ ID NO: 701

TGCCCATGTAAGGAGGCAAGGCCTGGGGACACCCGAGATGCCTGGTT
ATAATTAACCCAGACATGTGGCTGCCCCCCCCCCCCCAACACCTGCT
GCCTCTAAAAATAACCCTGCATGCCATGTTCCCGGCGAAGGGCCAGC
TGTCCCCCGCCAGCTAGACTCAGCACTTAGTTTAGGAACCAGTGAGC
AAGTCAGCCCTTGGGGCAGCCCATACAAGGCCATGGGGCTGGGCAAG
CTGCACGCCTGGGTCCGGGGTGGGCACGGTGCCCGGGCAACGAGCTG
AAAGCTCATCTGCTCTCAGGGGCCCCTCCCTGGGGACAGCCCCTCCT
GGCTAGTCACACCCTGTAGGCTCCTCTATATAACCCAGGGGCACAGG
GGCTGCCCTCATTCTACCACCACCTCCACAGCACAGACAGACACTCA
GGAGCCAGCCAGC.

In some embodiments, the vector comprises multiple inverted terminal repeats (ITRs). These ITRs may be of an AAV1, AAV2, AAV3, AAV4, AAV5, AAV6, AAV7, AAV8, or AAV9 serotype. In some embodiments, the ITRs are of an AAV2 serotype. In some embodiments, the 5′

ITR comprises the sequence of SEQ ID NO: 709:

GGCCACTCCCTCTCTGCGCGCTCGCTCGCTCACTGAGGCCGGGCGAC
CAAAGGTCGCCCGACGCCCGGGCTTTGCCCGGGCGGCCTCAGTGAGC
GAGCGAGCGCGCAGAGAGGGAGTGGCCAACTCCATCACTAGGGGTTC
CT.

In some embodiments, the 3′ITR comprises the sequence of SEQ ID NO: 710:

AGGAACCCCTAGTGATGGAGTTGGCCACTCCCTCTCTGCGCGCTCGC
TCGCTCACTGAGGCCGGGCGACCAAAGGTCGCCCGACGCCCGGGCTT
TGCCCGGGCGGCCTCAGTGAGCGAGCGAGCGCGCAGAGAGGGA.

In some embodiments, a vector comprising a single nucleic acid molecule encoding 1) one or more guide RNA comprising any one or more of the spacer sequences of SEQ ID Nos: 1-8, 10-28, and 101-154; and 2) a SaCas9 is provided. In some embodiments, the vector is an AAV vector. In some embodiments, the vector is an AAV9 vector. In some embodiments, the AAV vector is administered to a subject to treat DM1. In some embodiments, only one vector is needed due to the use of a particular guide sequence that is useful in the context of SaCas9. In some embodiments, the composition further comprises a DNA-PK inhibitor.

In some embodiments, a vector comprising a single nucleic acid molecule encoding 1) a pair of guide RNAs comprising a first and second spacer sequence selected from any one of SEQ ID NOs: 1 and 10; 1 and 11; 1 and 12; 1 and 13; 1 and 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; band 16; band 17; band 18; band 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28; and 2) a SaCas9 is provided. In some embodiments, the vector is an AAV vector. In some embodiments, the AAV vector is administered to a subject to treat DM1. In some embodiments, only one vector is needed due to the use of a particular guide sequence that is useful in the context of SaCas9. In some embodiments, the composition further comprises a DNA-PK inhibitor.

In some embodiments, the vector comprises a nucleic acid encoding a Cas9 protein (e.g., an SaCas9 protein) and further comprises a nucleic acid encoding one or more single guide RNA(s). In some embodiments, the nucleic acid encoding the Cas9 protein is under the control of a CK8e promoter. In some embodiments, the nucleic acid encoding the guide RNA sequence is under the control of a hU6c promoter. In some embodiments, the vector is AAV9. In preferred embodiments, the AAV9 vector is less than 5 kb from ITR to ITR in size, inclusive of both ITRs. In particular embodiments, the AAV9 vector is less than 4.9 kb from ITR to ITR in size, inclusive of both ITRs. In further embodiments, the AAV9 vector is less than 4.85 kb from ITR to ITR in size, inclusive of both ITRs. In further embodiments, the AAV9 vector is less than 4.8 kb from ITR to ITR in size, inclusive of both ITRs. In further embodiments, the AAV9 vector is less than 4.75 kb from ITR to ITR in size, inclusive of both ITRs. In further embodiments, the AAV9 vector is less than 4.7 kb from ITR to ITR in size, inclusive of both ITRs. In some embodiments, the AAV9 vector is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb from ITR to ITR in size, inclusive of both ITRs. In some embodiments, the AAV9 vector is between 4.4-4.85 kb from ITR to ITR in size, inclusive of both ITRs.

In some embodiments, the vector comprises multiple nucleic acids encoding more than one guide RNA. In some embodiments, the vector comprises two nucleic acids encoding two guide RNA sequences.

In some embodiments, the vector comprises a nucleic acid encoding a Cas9 protein (e.g., an SaCas9 protein), a nucleic acid encoding a first guide RNA, and a nucleic acid encoding a second guide RNA. In some embodiments, the vector does not comprise a nucleic acid encoding more than two guide RNAs. In some embodiments, the nucleic acid encoding the first guide RNA is the same as the nucleic acid encoding the second guide RNA. In some embodiments, the nucleic acid encoding the first guide RNA is different from the nucleic acid encoding the second guide RNA. In some embodiments, the vector comprises a single nucleic acid molecule, wherein the single nucleic acid molecule comprises a nucleic acid encoding a Cas9 protein, a nucleic acid encoding a first guide RNA, and a nucleic acid that is the reverse complement to the coding sequence for the second guide RNA. In some embodiments, the vector comprises a single nucleic acid molecule, wherein the single nucleic acid molecule comprises a nucleic acid encoding a Cas9 protein, a nucleic acid that is the reverse complement to the coding sequence for the first guide RNA, and a nucleic acid that is the reverse complement to the coding sequence for the second guide RNA. In some embodiments, the nucleic acid encoding a Cas9 protein (e.g., an SaCas9) is under the control of the CK8e promoter. In some embodiments, the first guide is under the control of the 7SK2 promoter, and the second guide is under the control of the H1m promoter. In some embodiments, the first guide is under the control of the H1m promoter, and the second guide is under the control of the 7SK2 promoter. In some embodiments, the first guide is under the control of the hU6c promoter, and the second guide is under the control of the H1m promoter. In some embodiments, the first guide is under the control of the H1m promoter, and the second guide is under the control of the hU6c promoter. In some embodiments, the nucleic acid encoding the Cas9 protein is: a) between the nucleic acids encoding the guide RNAs, b) between the nucleic acids that are the reverse complement to the coding sequences for the guide RNAs, c) between the nucleic acid encoding the first guide RNA and the nucleic acid that is the reverse complement to the coding sequence for the second guide RNA, d) between the nucleic acid encoding the second guide RNA and the nucleic acid that is the reverse complement to the coding sequence for the first guide RNA, e) 5′ to the nucleic acids encoding the guide RNAs, f) 5′ to the nucleic acids that are the reverse complements to the coding sequences for the guide RNAs, g) 5′ to a nucleic acid encoding one of the guide RNAs and 5′ to a nucleic acid that is the reverse complement to the coding sequence for the other guide RNA, h) 3′ to the nucleic acids encoding the guide RNAs, i) 3′ to the nucleic acids that are the reverse complements to the coding sequences for the guide RNAs, or j) 3′ to a nucleic acid encoding one of the guide RNAs and 3′ to a nucleic acid that is the reverse complement to the coding sequence for the other guide RNA. In some embodiments, the AAV vector size is measured in length of nucleotides from ITR to ITR, inclusive of both ITRs. In some embodiments, the AAV vector is less than 5 kb in size from ITR to ITR, inclusive of both ITRs. In particular embodiments, the AAV vector is less than 4.9 kb from ITR to ITR in size, inclusive of both ITRs. In further embodiments, the AAV vector is less than 4.85 kb in size from ITR to ITR, inclusive of both ITRs. In further embodiments, the AAV vector is less than 4.8 kb in size from ITR to ITR, inclusive of both ITRs. In further embodiments, the AAV vector is less than 4.75 kb in size from ITR to ITR, inclusive of both ITRs. In further embodiments, the AAV vector is less than 4.7 kb in size from ITR to ITR, inclusive of both ITRs. In some embodiments, the vector is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb from ITR to ITR in size, inclusive of both ITRs. In some embodiments, the vector is between 4.4-4.85 kb in size from ITR to ITR, inclusive of both ITRs. In some embodiments, the vector is AAV9.

In some embodiments, the disclosure provides for a nucleic acid comprising from 5′ to 3′ with respect to the plus strand: the reverse complement of a first guide RNA scaffold sequence (a scaffold comprising the nucleotide sequence of SEQ ID NOs: 500, 910, 911, 912, 920, and 921), the reverse complement of a nucleotide sequence encoding the first guide RNA sequence, the reverse complement of a promoter for expression of the nucleotide sequence encoding the first guide RNA sequence (e.g., hU6c), a promoter for expression of the second guide RNA in the same direction as the promoter for the endonuclease (e.g., 7SK2), the second guide RNA sequence, and a second guide RNA scaffold sequence (a scaffold comprising the nucleotide sequence of SEQ ID NOs: 500, 910, 911, 912, 920, and 921), a promoter for expression of a nucleotide sequence encoding the endonuclease (e.g., CK8e), a nucleotide sequence encoding an endonuclease (e.g., a SaCas9), a polyadenylation sequence.

In some embodiments, any of the vectors disclosed herein comprises a nucleic acid encoding at least a first guide RNA and a second guide RNA. In some embodiments, the nucleic acid comprises a spacer-encoding sequence for the first guide RNA, a scaffold-encoding sequence for the first guide RNA, a spacer-encoding sequence for the second guide RNA, and a scaffold-encoding sequence of the second guide RNA. In some embodiments, the spacer-encoding sequence (e.g., encoding any of the spacer sequences disclosed herein) for the first guide RNA is identical to the spacer-encoding sequence for the second guide RNA. In some embodiments, the spacer-encoding sequence (e.g., encoding any of the spacer sequences disclosed herein) for the first guide RNA is different from the spacer-encoding sequence for the second guide RNA. In some embodiments, the scaffold-encoding sequence for the first guide RNA is identical to the scaffold-encoding sequence for the second guide RNA. In some embodiments, the scaffold-encoding sequence for the first guide RNA is different from the scaffold-encoding sequence for the nucleic acid encoding the second guide RNA. In some embodiments, the scaffold-encoding sequence for the first guide RNA comprises a sequence selected from the group consisting of SEQ ID Nos: 500, 910, 911, 912, 920, and 921, and the scaffold-encoding sequence for the second guide RNA comprises a different sequence selected from the group consisting of SEQ ID Nos: 500, 910, 911, 912, 920, and 921. In some embodiments, the scaffold-encoding sequence for the first guide RNA comprises the sequence of SEQ ID NO: 921, and the scaffold-encoding sequence for the second guide RNA comprises the sequence of SEQ ID NO: 500. In some embodiments, the scaffold-encoding sequence for the first guide RNA comprises the sequence of SEQ ID NO: 921, and the scaffold-encoding sequence for the second guide RNA comprises the sequence of SEQ ID NO: 910. In some embodiments, the scaffold-encoding sequence for the first guide RNA comprises the sequence of SEQ ID NO: 921, and the scaffold-encoding sequence for the second guide RNA comprises the sequence of SEQ ID NO: 911. In some embodiments, the scaffold-encoding sequence for the first guide RNA comprises the sequence of SEQ ID NO: 921, and the scaffold-encoding sequence for the second guide RNA comprises the sequence of SEQ ID NO: 912. In some embodiments, the scaffold-encoding sequence for the first guide RNA comprises the sequence of SEQ ID NO: 921, and the scaffold-encoding sequence for the second guide RNA comprises the sequence of SEQ ID NO: 920. In some embodiments, the scaffold-encoding sequence for the first guide RNA comprises the sequence of SEQ ID NO: 921, and the scaffold-encoding sequence for the second guide RNA comprises the sequence of SEQ ID NO: 921. In some embodiments, the spacer encoding sequence for the first guide RNA is the same as the spacer-encoding sequence in the second guide RNA, and the scaffold-encoding sequence for the first guide RNA is different from the scaffold-encoding sequence in the nucleic acid encoding the second guide

RNA.

The disclosure provides for novel AAV vector configurations. Some examples of these novel AAV vector configurations are provided herein, and the order of elements in these exemplary vectors are referenced in a 5′ to 3′ manner with respect to the plus strand. For these configurations, it should be understood that the recited elements may not be directly contiguous, and that one or more nucleotides or one or more additional elements may be present between the recited elements. However, in some embodiments, it is possible that no nucleotides or no additional elements are present between the recited elements. Also, unless otherwise stated, “a promoter for expression of element X” means that the promoter is oriented in a manner to facilitate expression of the recited element X. In some embodiments, the disclosure provides for a nucleic acid encoding an SaCas9. In some embodiments, the nucleic acid encodes for a nuclear localization signal (e.g., the SV40 NLS) on the C-terminus of the encoded SaCas9. In some embodiments, the nucleic acid encodes for an NLS (e.g., the SV40 NLS) on the C-terminus of the encoded SaCas9, and the nucleic acid does not encode for an NLS on the N-terminus of the encoded SaCas9. In some embodiments, the nucleic acid encodes for a nuclear localization signal (e.g., the SV40 NLS) on the N-terminus of the encoded SaCas9. In some embodiments, the nucleic acid encodes for an NLS (e.g., the SV40 NLS) on the N-terminus of the encoded SaCas9, and the nucleic acid does not encode for an NLS on the C-terminus of the encoded SaCas9. In some embodiments, the nucleic acid encodes for a nuclear localization signal (e.g., the SV40 NLS) on the C-terminus of the encoded SaCas9 and also encodes for an NLS on the N-terminus of the encoded SaCas9.

In some embodiments, the AAV vector comprises from 5′ to 3′ with respect to the plus strand: a promoter for expression of a nucleic acid encoding a first sgRNA, a nucleic acid encoding the first sgRNA guide sequence, the first sgRNA scaffold sequence, a promoter for expression of SaCas9 (e.g., CK8e), a nucleic acid encoding SaCas9, a polyadenylation sequence, a promoter for expression of a second sgRNA, the second sgRNA guide sequence, and a second sgRNA scaffold sequence. See FIG. 10A at “Design 1”. In some embodiments the promoter for expression of the nucleic acid encoding the first sgRNA is any of the hU6c promoters disclosed herein. In some embodiments the promoter for expression of the nucleic acid encoding the first sgRNA comprises SEQ ID NO: 705. In some embodiments the promoter for expression of the nucleic acid encoding the first sgRNA comprises SEQ ID NO: 901. In some embodiments the promoter for expression of the nucleic acid encoding the first sgRNA comprises SEQ ID NO: 902. In some embodiments the promoter for expression of the nucleic acid encoding the first sgRNA comprises SEQ ID NO: 903. In some embodiments the promoter for expression of the nucleic acid encoding the first sgRNA comprises SEQ ID NO: 904. In some embodiments the promoter for expression of the nucleic acid encoding the first sgRNA is any of the 7SK2 promoters disclosed herein. In some embodiments the promoter for expression of the nucleic acid encoding the first sgRNA comprises SEQ ID NO: 705. In some embodiments the promoter for expression of the nucleic acid encoding the first sgRNA comprises SEQ ID NO: 906. In some embodiments the promoter for expression of the nucleic acid encoding the first sgRNA comprises SEQ ID NO: 907. In some embodiments the promoter for expression of the nucleic acid encoding the first sgRNA comprises SEQ ID NO: 908. In some embodiments the promoter for expression of the nucleic acid encoding the first sgRNA comprises SEQ ID NO: 909. In some embodiments the promoter for expression of the nucleic acid encoding the second sgRNA is any of the hU6c promoters disclosed herein. In some embodiments the promoter for expression of the nucleic acid encoding the second sgRNA comprises SEQ ID NO: 705. In some embodiments the promoter for expression of the nucleic acid encoding the second sgRNA comprises SEQ ID NO: 901. In some embodiments the promoter for expression of the nucleic acid encoding the second sgRNA comprises SEQ ID NO: 902. In some embodiments the promoter for expression of the nucleic acid encoding the second sgRNA comprises SEQ ID NO: 903. In some embodiments the promoter for expression of the nucleic acid encoding the second sgRNA comprises SEQ ID NO: 904. In some embodiments the promoter for expression of the nucleic acid encoding the second sgRNA is any of the 7SK2 promoters disclosed herein. In some embodiments the promoter for expression of the nucleic acid encoding the second sgRNA comprises SEQ ID NO: 706. In some embodiments the promoter for expression of the nucleic acid encoding the second sgRNA comprises SEQ ID NO: 906. In some embodiments the promoter for expression of the nucleic acid encoding the second sgRNA comprises SEQ ID NO: 907. In some embodiments the promoter for expression of the nucleic acid encoding the second sgRNA comprises SEQ ID NO: 908. In some embodiments the promoter for expression of the nucleic acid encoding the second sgRNA comprises SEQ ID NO: 909. In some embodiments, the promoter for SaCas9 is the CK8e promoter. In some embodiments, the first sgRNA comprises SaU7 (SEQ ID NO: 7) and the second sgRNA comprises SaD10 (SEQ ID NO: 18). In some embodiments, the first sgRNA comprises SaU4 (SEQ ID NO: 4) and the second sgRNA comprises SaD4 (SEQ ID NO: 12). In some embodiments, the sgRNA scaffold is SEQ ID NO: 500. In some embodiments, the sgRNA scaffold is SEQ ID NO: 910. In some embodiments, the sgRNA scaffold is SEQ ID NO: 911. In some embodiments, the sgRNA scaffold is SEQ ID NO: 912. In some embodiments, the sgRNA scaffold is SEQ ID NO: 920. In some embodiments, the sgRNA scaffold is SEQ ID NO: 921. In some embodiments, the first sgRNA targets a nucleic acid region upstream of a trinucleotide repeat expansion, and the second sgRNA targets a nucleic acid region downstream of a trinucleotide repeat expansion. In some embodiments, the first sgRNA targets a nucleic acid region downstream of a trinucleotide repeat expansion, and the second sgRNA targets a nucleic acid region upstream of a trinucleotide repeat expansion.

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, the first sgRNA scaffold sequence, a promoter for expression of a nucleic acid encoding SaCas9 (e.g., CK8e), a nucleic acid encoding SaCas9, a polyadenylation sequence, an hU6c promoter for expression of a second sgRNA, a second sgRNA guide sequence, and a second sgRNA scaffold sequence. In some embodiments, the first sgRNA comprises SaU7 (SEQ ID NO: 7) and the second sgRNA comprises SaD10 (SEQ ID NO: 18). In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence, a promoter for expression of a nucleic acid encoding SaCas9 (e.g., CK8e), a nucleic acid encoding SaCas9, a polyadenylation sequence, a 7SK2 promoter for expression of a second sgRNA, the second sgRNA guide sequence, and a second sgRNA scaffold sequence. In some embodiments, the first sgRNA comprises SaU7 (SEQ ID NO: 7) and the second sgRNA comprises SaD10 (SEQ ID NO: 18). In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: an hU6c promoter for expression of the nucleic acid encoding a first sgRNA, a nucleic acid encoding the first sgRNA guide sequence, a first sgRNA scaffold sequence, a promoter for expression of a nucleic acid encoding SaCas9 (e.g., CK8e), a nucleic acid encoding SaCas9, a polyadenylation sequence, an H1m promoter for expression of a second sgRNA, a second sgRNA guide sequence, and a second sgRNA scaffold sequence. In some embodiments, the first sgRNA comprises SaU7 (SEQ ID NO: 7) and the second sgRNA comprises SaD10 (SEQ ID NO: 18). In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of a promoter for expression of the nucleic acid encoding the first sgRNA, a promoter for expression of a nucleic acid encoding SaCas9 (e.g., CK8e), a nucleic acid encoding SaCas9, a polyadenylation sequence, a promoter for expression of a second sgRNA, a second sgRNA guide sequence, and a second sgRNA scaffold sequence. See FIG. 10A at “Design 2”. In some embodiments the promoter for expression of the nucleic acid encoding the first sgRNA is any of the hU6c promoters disclosed herein. In some embodiments the promoter for expression of the nucleic acid encoding the first sgRNA comprises SEQ ID NO: 705. In some embodiments the promoter for expression of the nucleic acid encoding the first sgRNA comprises SEQ ID NO: 901. In some embodiments the promoter for expression of the nucleic acid encoding the first sgRNA comprises SEQ ID NO: 902. In some embodiments the promoter for expression of the nucleic acid encoding the first sgRNA comprises SEQ ID NO: 903. In some embodiments the promoter for expression of the nucleic acid encoding the first sgRNA comprises SEQ ID NO: 904. In some embodiments the promoter for expression of the nucleic acid encoding the first sgRNA is any of the 7SK2 promoters disclosed herein. In some embodiments the promoter for expression of the nucleic acid encoding the first sgRNA comprises SEQ ID NO: 705. In some embodiments the promoter for expression of the nucleic acid encoding the first sgRNA comprises SEQ ID NO: 906. In some embodiments the promoter for expression of the nucleic acid encoding the first sgRNA comprises SEQ ID NO: 907. In some embodiments the promoter for expression of the nucleic acid encoding the first sgRNA comprises SEQ ID NO: 908. In some embodiments the promoter for expression of the nucleic acid encoding the first sgRNA comprises SEQ ID NO: 909. In some embodiments the promoter for expression of the nucleic acid encoding the second sgRNA is any of the hU6c promoters disclosed herein. In some embodiments the promoter for expression of the nucleic acid encoding the second sgRNA comprises SEQ ID NO: 705. In some embodiments the promoter for expression of the nucleic acid encoding the second sgRNA comprises SEQ ID NO: 901. In some embodiments the promoter for expression of the nucleic acid encoding the second sgRNA comprises SEQ ID NO: 902. In some embodiments the promoter for expression of the nucleic acid encoding the second sgRNA comprises SEQ ID NO: 903. In some embodiments the promoter for expression of the nucleic acid encoding the second sgRNA comprises SEQ ID NO: 904. In some embodiments the promoter for expression of the nucleic acid encoding the second sgRNA is any of the 7SK2 promoters disclosed herein. In some embodiments the promoter for expression of the nucleic acid encoding the second sgRNA comprises SEQ ID NO: 706. In some embodiments the promoter for expression of the nucleic acid encoding the second sgRNA comprises SEQ ID NO: 906. In some embodiments the promoter for expression of the nucleic acid encoding the second sgRNA comprises SEQ ID NO: 907. In some embodiments the promoter for expression of the nucleic acid encoding the second sgRNA comprises SEQ ID NO: 908. In some embodiments the promoter for expression of the nucleic acid encoding the second sgRNA comprises SEQ ID NO: 909. In some embodiments, the promoter for SaCas9 is the CK8e promoter. In some embodiments, the first sgRNA comprises SaU7 (SEQ ID NO: 7) and the second sgRNA comprises SaD10 (SEQ ID NO: 18). In some embodiments, the first sgRNA comprises SaU4 (SEQ ID NO: 4) and the second sgRNA comprises SaD4 (SEQ ID NO: 12). In some embodiments, the sgRNA scaffold is SEQ ID NO: 500. In some embodiments, the sgRNA scaffold is SEQ ID NO: 910. In some embodiments, the sgRNA scaffold is SEQ ID NO: 911. In some embodiments, the sgRNA scaffold is SEQ ID NO: 912. In some embodiments, the sgRNA scaffold is SEQ ID NO: 920. In some embodiments, the sgRNA scaffold is SEQ ID NO: 921. In some embodiments, the first sgRNA targets a nucleic acid region upstream of a trinucleotide repeat expansion, and the second sgRNA targets a nucleic acid region downstream of a trinucleotide repeat expansion. In some embodiments, the first sgRNA targets a nucleic acid region downstream of a trinucleotide repeat expansion, and the second sgRNA targets a nucleic acid region upstream of a trinucleotide repeat expansion.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SaCas9 (e.g., CK8e), a nucleic acid encoding SaCas9, a polyadenylation sequence, an hU6c promoter for expression of a second sgRNA, a second sgRNA guide sequence, and a second sgRNA scaffold sequence. In some embodiments, the first sgRNA comprises SaU7 (SEQ ID NO: 7) and the second sgRNA comprises SaD10 (SEQ ID NO: 18). In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SaCas9 (e.g., CK8e), a nucleic acid encoding SaCas9, a polyadenylation sequence, an 7SK2 promoter for expression of a second sgRNA, a second sgRNA guide sequence, and a second sgRNA scaffold sequence. In some embodiments, the first sgRNA comprises SaU7 (SEQ ID NO: 7) and the second sgRNA comprises SaD10 (SEQ ID NO: 18). In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SaCas9 (e.g., CK8e), a nucleic acid encoding SaCas9, a polyadenylation sequence, an H1m promoter for expression of a second sgRNA, a second sgRNA guide sequence, and a second sgRNA scaffold sequence. In some embodiments, the first sgRNA comprises SaU7 (SEQ ID NO: 7) and the second sgRNA comprises SaD10 (SEQ ID NO: 18). In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of a 7SK2 promoter for expression of the nucleic acid encoding the first sgRNA, a promoter for expression of a nucleic acid encoding SaCas9 (e.g., CK8e), a nucleic acid encoding SaCas9, a polyadenylation sequence, an hU6 promoter for expression of a second sgRNA, a second sgRNA guide sequence, and a second sgRNA scaffold sequence. In some embodiments, the first sgRNA comprises SaD10 (SEQ ID NO: 18) and the second sgRNA comprises SaU7 (SEQ ID NO: 7). In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: a promoter for expression of a nucleic acid encoding a first sgRNA, a nucleic acid encoding the first sgRNA guide sequence, a first sgRNA scaffold sequence, a promoter for expression of a second sgRNA, a second sgRNA guide sequence, and a second sgRNA scaffold sequence, a promoter for SaCas9 (e.g., CK8e), a nucleic acid encoding SaCas9, and a polyadenylation sequence. See FIG. 10A at “Design 3”. In some embodiments the promoter for expression of the nucleic acid encoding the first sgRNA is an hU6c promoter. In some embodiments the promoter for expression of the nucleic acid encoding the first sgRNA comprises SEQ ID NO: 705. In some embodiments the promoter for expression of the nucleic acid encoding the first sgRNA comprises SEQ ID NO: 901. In some embodiments the promoter for expression of the nucleic acid encoding the first sgRNA comprises SEQ ID NO: 902. In some embodiments the promoter for expression of the nucleic acid encoding the first sgRNA comprises SEQ ID NO: 903. In some embodiments the promoter for expression of the nucleic acid encoding the first sgRNA comprises SEQ ID NO: 904. In some embodiments the promoter for expression of the nucleic acid encoding the first sgRNA is an 7SK2 promoter. In some embodiments the promoter for expression of the nucleic acid encoding the first sgRNA comprises SEQ ID NO: 705. In some embodiments the promoter for expression of the nucleic acid encoding the first sgRNA comprises SEQ ID NO: 906. In some embodiments the promoter for expression of the nucleic acid encoding the first sgRNA comprises SEQ ID NO: 907. In some embodiments the promoter for expression of the nucleic acid encoding the first sgRNA comprises SEQ ID NO: 908. In some embodiments the promoter for expression of the nucleic acid encoding the first sgRNA comprises SEQ ID NO: 909. In some embodiments the promoter for expression of the nucleic acid encoding the second sgRNA is an hU6c promoter. In some embodiments the promoter for expression of the nucleic acid encoding the second sgRNA comprises SEQ ID NO: 705. In some embodiments the promoter for expression of the nucleic acid encoding the second sgRNA comprises SEQ ID NO: 901. In some embodiments the promoter for expression of the nucleic acid encoding the second sgRNA comprises SEQ ID NO: 902. In some embodiments the promoter for expression of the nucleic acid encoding the second sgRNA comprises SEQ ID NO: 903. In some embodiments the promoter for expression of the nucleic acid encoding the second sgRNA comprises SEQ ID NO: 904. In some embodiments the promoter for expression of the nucleic acid encoding the second sgRNA is an 7SK2 promoter. In some embodiments the promoter for expression of the nucleic acid encoding the second sgRNA comprises SEQ ID NO: 706. In some embodiments the promoter for expression of the nucleic acid encoding the second sgRNA comprises SEQ ID NO: 906. In some embodiments the promoter for expression of the nucleic acid encoding the second sgRNA comprises SEQ ID NO: 907. In some embodiments the promoter for expression of the nucleic acid encoding the second sgRNA comprises SEQ ID NO: 908. In some embodiments the promoter for expression of the nucleic acid encoding the second sgRNA comprises SEQ ID NO: 909. In some embodiments, the promoter for SaCas9 is the CK8e promoter. In some embodiments, the first sgRNA comprises SaU7 (SEQ ID NO: 7) and the second sgRNA comprises SaD10 (SEQ ID NO: 18). In some embodiments, the first sgRNA comprises SaU4 (SEQ ID NO: 4) and the second sgRNA comprises SaD4 (SEQ ID NO: 12). In some embodiments, the sgRNA scaffold is SEQ ID NO: 500. In some embodiments, the sgRNA scaffold is SEQ ID NO: 910. In some embodiments, the sgRNA scaffold is SEQ ID NO: 911. In some embodiments, the sgRNA scaffold is SEQ ID NO: 912. In some embodiments, the sgRNA scaffold is SEQ ID NO: 920. In some embodiments, the sgRNA scaffold is SEQ ID NO: 921. In some embodiments, the first sgRNA targets a nucleic acid region upstream of a trinucleotide repeat expansion, and the second sgRNA targets a nucleic acid region downstream of a trinucleotide repeat expansion. In some embodiments, the first sgRNA targets a nucleic acid region downstream of a trinucleotide repeat expansion, and the second sgRNA targets a nucleic acid region upstream of a trinucleotide repeat expansion.

In some embodiments, the AAV vector comprises from 5′ to 3′ with respect to the plus strand: an hU6c promoter for expression of a nucleic acid encoding a first sgRNAa nucleic acid encoding a first sgRNA guide sequence, a first sgRNA scaffold sequence, an hU6c promoter for expression of a second sgRNA, a second sgRNA guide sequence, and a second sgRNA scaffold sequence, a promoter for expression of a nucleic acid encoding SaCas9 (e.g., CK8e), a nucleic acid encoding SaCas9, and a polyadenylation sequence. In some embodiments, the first sgRNA comprises SaU7 (SEQ ID NO: 7) and the second sgRNA comprises SaD10 (SEQ ID NO: 18). In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence, an 7SK2 promoter for expression of a second sgRNA, a second sgRNA guide sequence, and a second sgRNA scaffold sequence, a promoter for expression of a nucleic acid encoding SaCas9 (e.g., CK8e), a nucleic acid encoding SaCas9, and a polyadenylation sequence. In some embodiments, the first sgRNA comprises SaU7 (SEQ ID NO: 7) and the second sgRNA comprises SaD10 (SEQ ID NO: 18).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence, an 7SK2 promoter for expression of a second sgRNA, a second sgRNA guide sequence, and a second sgRNA scaffold sequence, a promoter for expression of a nucleic acid encoding SaCas9 (e.g., CK8e), a nucleic acid encoding SaCas9, and a polyadenylation sequence. In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence, an 7SK2 promoter for expression of a second sgRNA, a second sgRNA guide sequence, and a second sgRNA scaffold sequence, a promoter for expression of a nucleic acid encoding SaCas9 (e.g., CK8e), a nucleic acid encoding SaCas9, and a polyadenylation sequence. In some embodiments, the first sgRNA comprises SaD4 (SEQ ID NO: 12) and the second sgRNA comprises SaU4 (SEQ ID NO: 4).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 910, an 7SK2 promoter for expression of a second sgRNA, a second sgRNA guide sequence, and a second sgRNA scaffold sequence comprising SEQ ID NO: 910, a promoter for expression of a nucleic acid encoding SaCas9 (e.g., CK8e), a nucleic acid encoding SaCas9, and a polyadenylation sequence. In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 911, an 7SK2 promoter for expression of a second sgRNA, a second sgRNA guide sequence, and a second sgRNA scaffold sequence comprising SEQ ID NO: 911, a promoter for expression of a nucleic acid encoding SaCas9 (e.g., CK8e), a nucleic acid encoding SaCas9, and a polyadenylation sequence. In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 912, an 7SK2 promoter for expression of a second sgRNA, a second sgRNA guide sequence, and a second sgRNA scaffold sequence comprising SEQ ID NO: 912, a promoter for expression of a nucleic acid encoding SaCas9 (e.g., CK8e), a nucleic acid encoding SaCas9, and a polyadenylation sequence. In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: the hU6d30 promoter (SEQ ID NO: 901) for expression of a nucleic acid encoding a first sgRNA, a nucleic acid encoding the first sgRNA guide sequence, a first sgRNA scaffold sequence comprising SEQ ID NO: 911, an 7SK2 promoter for expression of a second sgRNA, a second sgRNA guide sequence, and a second sgRNA scaffold sequence comprising SEQ ID NO: 911, a promoter for expression of a nucleic acid encoding SaCas9 (e.g., CK8e), a nucleic acid encoding SaCas9, and a polyadenylation sequence. In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: the hU6d60 promoter (SEQ ID NO: 902) for expression of a nucleic acid encoding a first sgRNA, a nucleic acid encoding the first sgRNA guide sequence, a first sgRNA scaffold sequence comprising SEQ ID NO: 911, an 7SK2 promoter for expression of a second sgRNA, a second sgRNA guide sequence, and a second sgRNA scaffold sequence comprising SEQ ID NO: 911, a promoter for expression of a nucleic acid encoding SaCas9 (e.g., CK8e), a nucleic acid encoding SaCas9, and a polyadenylation sequence. In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: the hU6d90 promoter (SEQ ID NO: 903) for expression of a nucleic acid encoding a first sgRNAa nucleic acid encoding the first sgRNA guide sequence, a first sgRNA scaffold sequence comprising SEQ ID NO: 911, an 7SK2 promoter for expression of a second sgRNA, a second sgRNA guide sequence, and a second sgRNA scaffold sequence comprising SEQ ID NO: 911, a promoter for expression of a nucleic acid encoding SaCas9 (e.g., CK8e), a nucleic acid encoding SaCas9, and a polyadenylation sequence. In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: the hU6d120 promoter (SEQ ID NO: 904) for expression of a nucleic acid encoding a first sgRNA, a nucleic acid encoding the first sgRNA guide sequence, a first sgRNA scaffold sequence comprising SEQ ID NO: 911, an 7SK2 promoter for expression of a second sgRNA, a second sgRNA guide sequence, and a second sgRNA scaffold sequence comprising SEQ ID NO: 911, a promoter for expression of a nucleic acid encoding SaCas9 (e.g., CK8e), a nucleic acid encoding SaCas9, and a polyadenylation sequence. In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 911, the 7SKd30 promoter (SEQ ID NO: 906) for expression of a second sgRNA, a second sgRNA guide sequence, and a second sgRNA scaffold sequence comprising SEQ ID NO: 911, a promoter for expression of a nucleic acid encoding SaCas9 (e.g., CK8e), a nucleic acid encoding SaCas9, and a polyadenylation sequence. In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 911, the 7SKd60 promoter (SEQ ID NO: 907) for expression of a second sgRNA, a second sgRNA guide sequence, and a second sgRNA scaffold sequence comprising SEQ ID NO: 911, a promoter for expression of a nucleic acid encoding SaCas9 (e.g., CK8e), a nucleic acid encoding SaCas9, and a polyadenylation sequence. In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: an hU6c promoter for expression of the nucleic acid encoding a first sgRNA, a nucleic acid encoding the first sgRNA guide sequence, a first sgRNA scaffold sequence comprising SEQ ID NO: 911, the 7SKd90 promoter (SEQ ID NO: 908) for expression of a second sgRNA, a second sgRNA guide sequence, and a second sgRNA scaffold sequence comprising SEQ ID NO: 911, a promoter for expression of a nucleic acid encoding SaCas9 (e.g., CK8e), a nucleic acid encoding SaCas9, and a polyadenylation sequence. In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 911, the 7SKd120 promoter (SEQ ID NO: 909) for expression of a second sgRNA, a second sgRNA guide sequence, and a second sgRNA scaffold sequence comprising SEQ ID NO: 911, a promoter for expression of a nucleic acid encoding SaCas9 (e.g., CK8e), a nucleic acid encoding SaCas9, and a polyadenylation sequence. In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 911, an 7SK2 promoter for expression of a second sgRNA, a second sgRNA guide sequence, and a second sgRNA scaffold sequence comprising SEQ ID NO: 911, a promoter for expression of a nucleic acid encoding SaCas9 (e.g., CK8e), an SV40 nuclear localization sequence (NLS), a nucleic acid encoding SaCas9, and a polyadenylation sequence. In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 911, an 7SK2 promoter for expression of a second sgRNA, a second sgRNA guide sequence, and a second sgRNA scaffold sequence comprising SEQ ID NO: 911, a promoter for expression of a nucleic acid encoding SaCas9 (e.g., CK8e), a nucleic acid encoding SaCas9, an SV40 nuclear localization sequence (NLS), and a polyadenylation sequence. In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence, an H1m promoter for expression of a second sgRNA, a second sgRNA guide sequence, and a second sgRNA scaffold sequence, a promoter for expression of a nucleic acid encoding SaCas9 (e.g., CK8e), a nucleic acid encoding SaCas9, and a polyadenylation sequence. In some embodiments, the first sgRNA comprises SaU7 (SEQ ID NO: 7) and the second sgRNA comprises SaD10 (SEQ ID NO: 18). In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 911, an 7SK2 promoter for expression of a second sgRNA, a second sgRNA guide sequence, and a second sgRNA scaffold sequence comprising SEQ ID NO: 911, a promoter for expression of a nucleic acid encoding SaCas9 (e.g., CK8e), a nucleic acid encoding SaCas9, and a polyadenylation sequence. In some embodiments, the first sgRNA comprises SaU7 (SEQ ID NO: 7) and the second sgRNA comprises SaD10 (SEQ ID NO: 18).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 921, an 7SK2 promoter for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaD4 (SEQ ID NO: 12) and the second sgRNA comprises SaU4 (SEQ ID NO: 4).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 921, an 7SK2 promoter for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaD10 (SEQ ID NO: 18) and the second sgRNA comprises SaU4 (SEQ ID NO: 4).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 921, an 7SK2 promoter for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaD4 (SEQ ID NO: 12) and the second sgRNA comprises SaU8 (SEQ ID NO: 8).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 921, an 7SK2 promoter for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaD4 (SEQ ID NO: 12) and the second sgRNA comprises SaU2 (SEQ ID NO: 2).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO:

921, an 7SK2 promoter for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaU4 (SEQ ID NO: 4) and the second sgRNA comprises SaD5 (SEQ ID NO: 13).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 921, an 7SK2 promoter for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 921, an hU6c promoter for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaD4 (SEQ ID NO: 12) and the second sgRNA comprises SaU4 (SEQ ID NO: 4).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 921, an 7SK2 promoter for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaU7 (SEQ ID NO: 7) and the second sgRNA comprises SaD10 (SEQ ID NO: 18).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 921, an 7SK2 promoter for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaU1 (SEQ ID NO: 1) and the second sgRNA comprises SaU1 (SEQ ID NO: 1).

In some embodiments, the AAV vector comprises from 5′ to 3′ with respect to the plus strand: the hU6d30 promoter (SEQ ID NO: 901) for expression of a nucleic acid encoding a first sgRNA, a nucleic acid encoding the first sgRNA guide sequence, a first sgRNA scaffold sequence comprising SEQ ID NO: 921, an 7SK2 promoter for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaD4 (SEQ ID NO: 12) and the second sgRNA comprises SaU4 (SEQ ID NO: 4).

In some embodiments, the AAV vector comprises from 5′ to 3′ with respect to the plus strand: the hU6d60 promoter (SEQ ID NO: 902) for expression of a nucleic acid encoding a first sgRNA, a nucleic acid encoding the first sgRNA guide sequence, a first sgRNA scaffold sequence comprising SEQ ID NO: 921, an 7SK2 promoter for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaD4 (SEQ ID NO: 12) and the second sgRNA comprises SaU4 (SEQ ID NO: 4).

In some embodiments, the AAV vector comprises from 5′ to 3′ with respect to the plus strand: the hU6d90 promoter (SEQ ID NO: 903) for expression of a nucleic acid encoding a first sgRNAa nucleic acid encoding the first sgRNA guide sequence, a first sgRNA scaffold sequence comprising SEQ ID NO: 921, an 7SK2 promoter for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaD4 (SEQ ID NO: 12) and the second sgRNA comprises SaU4 (SEQ ID NO: 4).

In some embodiments, the AAV vector comprises from 5′ to 3′ with respect to the plus strand: the hU6d120 promoter (SEQ ID NO: 904) for expression of a nucleic acid encoding a first sgRNA, a nucleic acid encoding the first sgRNA guide sequence, a first sgRNA scaffold sequence comprising SEQ ID NO: 921, an 7SK2 promoter for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaD4 (SEQ ID NO: 12) and the second sgRNA comprises SaU4 (SEQ ID NO: 4).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 921, the 7SKd30 promoter (SEQ ID NO: 906) for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaD4 (SEQ ID NO: 12) and the second sgRNA comprises SaU4 (SEQ ID NO: 4).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 921, the 7SKd60 promoter (SEQ ID NO: 907) for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaD4 (SEQ ID NO: 12) and the second sgRNA comprises SaU4 (SEQ ID NO: 4).

In some embodiments, the AAV vector comprises from 5′ to 3′ with respect to the plus strand: an hU6c promoter for expression of the nucleic acid encoding a first sgRNA, a nucleic acid encoding the first sgRNA guide sequence, a first sgRNA scaffold sequence comprising SEQ ID NO: 921, the 7SKd90 promoter (SEQ ID NO: 908) for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaD4 (SEQ ID NO: 12) and the second sgRNA comprises SaU4 (SEQ ID NO: 4).

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 921, the 7SKd120 promoter (SEQ ID NO: 909) for expression of a second sgRNA, a second sgRNA guide sequence, and a second sgRNA scaffold sequence comprising SEQ ID NO: 921, a promoter for expression of a nucleic acid encoding SaCas9 (e.g., CK8e), a nucleic acid encoding SaCas9, and a polyadenylation sequence. In some embodiments, the first sgRNA comprises SaD4 (SEQ ID NO: 12) and the second sgRNA comprises SaU4 (SEQ ID NO: 4).

In some embodiments, the AAV vector comprises from 5′ to 3′ with respect to the plus strand: a promoter for expression of a nucleic acid encoding SaCas9 (e.g., CK8e), a nucleic acid encoding SaCas9, a polyadenylation sequence, a promoter for expression of the nucleic acid encoding a first guide RNA, a nucleic acid encoding the first sgRNA guide sequence, a first sgRNA scaffold sequence, a promoter for expression of the second sgRNA, a second sgRNA guide sequence, and a second sgRNA scaffold sequence. See FIG. 10A at “Design 4”. In some embodiments the promoter for expression of the nucleic acid encoding the first sgRNA is an hU6c promoter. In some embodiments the promoter for expression of the nucleic acid encoding the first sgRNA comprises SEQ ID NO: 705. In some embodiments the promoter for expression of the nucleic acid encoding the first sgRNA comprises SEQ ID NO: 901. In some embodiments the promoter for expression of the nucleic acid encoding the first sgRNA comprises SEQ ID NO: 902. In some embodiments the promoter for expression of the nucleic acid encoding the first sgRNA comprises SEQ ID NO: 903. In some embodiments the promoter for expression of the nucleic acid encoding the first sgRNA comprises SEQ ID NO: 904. In some embodiments the promoter for expression of the nucleic acid encoding the first sgRNA is an 7SK2 promoter. In some embodiments the promoter for expression of the nucleic acid encoding the first sgRNA comprises SEQ ID NO: 706. In some embodiments the promoter for expression of the nucleic acid encoding the first sgRNA comprises SEQ ID NO: 906. In some embodiments the promoter for expression of the nucleic acid encoding the first sgRNA comprises SEQ ID NO: 907. In some embodiments the promoter for expression of the nucleic acid encoding the first sgRNA comprises SEQ ID NO: 908. In some embodiments the promoter for expression of the nucleic acid encoding the first sgRNA comprises SEQ ID NO: 909. In some embodiments the promoter for expression of the nucleic acid encoding the second sgRNA is an hU6c promoter. In some embodiments the promoter for expression of the nucleic acid encoding the second sgRNA comprises SEQ ID NO: 705. In some embodiments the promoter for expression of the nucleic acid encoding the second sgRNA comprises SEQ ID NO: 901. In some embodiments the promoter for expression of the nucleic acid encoding the second sgRNA comprises SEQ ID NO: 902. In some embodiments the promoter for expression of the nucleic acid encoding the second sgRNA comprises SEQ ID NO: 903. In some embodiments the promoter for expression of the nucleic acid encoding the second sgRNA comprises SEQ ID NO: 904. In some embodiments the promoter for expression of the nucleic acid encoding the second sgRNA is an 7SK2 promoter. In some embodiments the promoter for expression of the nucleic acid encoding the second sgRNA comprises SEQ ID NO: 705. In some embodiments the promoter for expression of the nucleic acid encoding the second sgRNA comprises SEQ ID NO: 906. In some embodiments the promoter for expression of the nucleic acid encoding the second sgRNA comprises SEQ ID NO: 907. In some embodiments the promoter for expression of the nucleic acid encoding the second sgRNA comprises SEQ ID NO: 908. In some embodiments the promoter for expression of the nucleic acid encoding the second sgRNA comprises SEQ ID NO: 909. In some embodiments, the promoter for SaCas9 is the CK8e promoter. In some embodiments, the first sgRNA comprises SaU7 (SEQ ID NO: 7) and the second sgRNA comprises SaD10 (SEQ ID NO: 18).

In some embodiments, the first sgRNA comprises SaU4 (SEQ ID NO: 4) and the second sgRNA comprises SaD4 (SEQ ID NO: 12). In some embodiments, the sgRNA scaffold is SEQ ID NO: 500. In some embodiments, the sgRNA scaffold is SEQ ID NO: 910. In some embodiments, the sgRNA scaffold is SEQ ID NO: 911. In some embodiments, the sgRNA scaffold is SEQ ID NO: 912. In some embodiments, the sgRNA scaffold is SEQ ID NO: 920. In some embodiments, the sgRNA scaffold is SEQ ID NO: 921. In some embodiments, the first sgRNA targets a nucleic acid region upstream of a trinucleotide repeat expansion, and the second sgRNA targets a nucleic acid region downstream of a trinucleotide repeat expansion. In some embodiments, the first sgRNA targets a nucleic acid region downstream of a trinucleotide repeat expansion, and the second sgRNA targets a nucleic acid region upstream of a trinucleotide repeat expansion.

In some embodiments, the AAV vector comprises from 5′ to 3′ with respect to the plus strand: a promoter for expression of a nucleic acid encoding SaCas9 (e.g., CK8e), a nucleic acid encoding SaCas9, a polyadenylation sequence, an hU6c promoter for expression of a nucleic acid encoding a first guide RNA, a nucleic acid encoding the first sgRNA guide sequence, a first sgRNA scaffold sequence, an hU6c promoter for expression of a second sgRNA, a second sgRNA guide sequence, and a second sgRNA scaffold sequence. In some embodiments, the first sgRNA comprises SaU7 (SEQ ID NO: 7) and the second sgRNA comprises SaD10 (SEQ ID NO: 18). In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: a promoter for expression of a nucleic acid encoding SaCas9 (e.g., CK8e), a nucleic acid encoding SaCas9, a polyadenylation sequence, an hU6c promoter for expression of a nucleic acid encoding a first guide RNA, a nucleic acid encoding the first sgRNA guide sequence, a first sgRNA scaffold sequence, a 7SK2 promoter for expression of a second sgRNA, a second sgRNA guide sequence, and a second sgRNA scaffold sequence. In some embodiments, the first sgRNA comprises SaU7 (SEQ ID NO: 7) and the second sgRNA comprises SaD10 (SEQ ID NO: 18). In some embodiments, the first sgRNA comprises SaU4 (SEQ ID NO: 4) and the second sgRNA comprises SaD4 (SEQ ID NO: 12).

In some embodiments, the AAV vector comprises from 5′ to 3′ with respect to the plus strand: a promoter for expression of a nucleic acid encoding SaCas9 (e.g., CK8e), a nucleic acid encoding SaCas9, a polyadenylation sequence, an hU6c promoter for expression of a nucleic acid encoding a first guide RNA, a nucleic acid encoding a first sgRNA guide sequence, a first sgRNA scaffold sequence, a H1m promoter for expression of a second sgRNA, a second sgRNA guide sequence, and a second sgRNA scaffold sequence. In some embodiments, the first sgRNA comprises

SaU7 (SEQ ID NO: 7) and the second sgRNA comprises SaD10 (SEQ ID NO: 18). In some embodiments, the first sgRNA comprises SaU4 (SEQ ID NO: 4) and the second sgRNA comprises SaD4 (SEQ ID NO: 12).

In particular embodiments, the AAV vector comprises from 5′ to 3′ with respect to the plus strand: an hU6c promoter for expression of the nucleic acid encoding a first sgRNA, a nucleic acid encoding the first sgRNA guide sequence, a first sgRNA scaffold sequence, the hU6c promoter for expression of a second sgRNA, a second sgRNA guide sequence, a second sgRNA scaffold sequence, a promoter for expression of a nucleic acid encoding SaCas9 (e.g., CK8e), a nucleic acid encoding SaCas9, and a polyadenylation sequence.

In particular embodiments, the AAV vector comprises from 5′ to 3′ with respect to the plus strand: an hU6c promoter for expression of the nucleic acid encoding a first sgRNA, a nucleic acid encoding the first sgRNA guide sequence, a first sgRNA scaffold sequence, a 7SK2 promoter for expression of a second sgRNA, a second sgRNA guide sequence, and a second sgRNA scaffold sequence, a promoter for expression of a nucleic acid encoding SaCas9 (e.g., CK8e), a nucleic acid encoding SaCas9, and a polyadenylation sequence.

In particular embodiments, the AAV vector comprises from 5′ to 3′ with respect to the plus strand: a promoter for expression of a nucleic acid encoding SaCas9 (e.g., CK8e), a nucleic acid encoding SaCas9, a polyadenylation sequence, an hU6c promoter for expression of a nucleic acid encoding a first guide RNA, a nucleic acid encoding a first sgRNA guide sequence, a first sgRNA scaffold sequence, a hU6c promoter for expression of a second sgRNA, a second sgRNA guide sequence, and a second sgRNA scaffold sequence.

In particular embodiments, the AAV vector comprises from 5′ to 3′ with respect to the plus strand: a promoter for expression of a nucleic acid encoding SaCas9 (e.g., CK8e), a nucleic acid encoding SaCas9, a polyadenylation sequence, an hU6c promoter for expression of a nucleic acid encoding a first guide RNA, a nucleic acid encoding a first sgRNA guide sequence, a first sgRNA scaffold sequence, a 7SK2 promoter for expression of a second sgRNA, a second sgRNA guide sequence, and a second sgRNA scaffold sequence.

In some embodiments, the nucleic acid encoding SaCas9 encodes an SaCas9 comprising an amino acid sequence that is at least 80%, 85%, 90%, 91%, 92%, 93%, 94%, 95%, 96%, 97%, 98%, 99% or 100% identical to the sequence of SEQ ID NO: 711:

KRNYILGLDIGITSVGYGIIDYETRDVIDAGVRLFKEANVENNEGRR
SKRGARRLKRRRRHRIQRVKKLLFDYNLLTDHSELSGINPYEARVKG
LSQKLSEEEFSAALLHLAKRRGVHNVNEVEEDTGNELSTKEQISRNS
KALEEKYVAELQLERLKKDGEVRGSINRFKTSDYVKEAKQLLKVQKA
YHQLDQSFIDTYIDLLETRRTYYEGPGEGSPFGWKDIKEWYEMLMGH
CTYFPEELRSVKYAYNADLYNALNDLNNLVITRDENEKLEYYEKFQI
IENVFKQKKKPTLKQIAKEILVNEEDIKGYRVTSTGKPEFTNLKVYH
DIKDITARKEIIENAELLDQIAKILTIYQSSEDIQEELTNLNSELTQ
EEIEQISNLKGYTGTHNLSLKAINLILDELWHTNDNQIAIFNRLKLV
PKKVDLSQQKEIPTTLVDDFILSPVVKRSFIQSIKVINAIIKKYGLP
NDIIIELAREKNSKDAQKMINEMQKRNRQTNERIEEIIRTTGKENAK
YLIEKIKLHDMQEGKCLYSLEAIPLEDLLNNPFNYEVDHIIPRSVSF
DNSFNNKVLVKQEENSKKGNRTPFQYLSSSDSKISYETFKKHILNLA
KGKGRISKTKKEYLLEERDINRFSVQKDFINRNLVDTRYATRGLMNL
LRSYFRVNNLDVKVKSINGGFTSFLRRKWKFKKERNKGYKHHAEDAL
IIANADFIFKEWKKLDKAKKVMENQMFEEKQAESMPEIETEQEYKEI
FITPHQIKHIKDFKDYKYSHRVDKKPNRELINDTLYSTRKDDKGNTL
IVNNLNGLYDKDNDKLKKLINKSPEKLLMYHHDPQTYQKLKLIMEQY
GDEKNPLYKYYEETGNYLTKYSKKDNGPVIKKIKYYGNKLNAHLDIT
DDYPNSRNKVVKLSLKPYRFDVYLDNGVYKFVTVKNLDVIKKENYYE
VNSKCYEEAKKLKKISNQAEFIASFYNNDLIKINGELYRVIGVNNDL
LNRIEVNMIDITYREYLENMNDKRPPRIIKTIASKTQSIKKYSTDIL
GNLYEVKSKKHPQIIKKG.

In some embodiments, the nucleic acid encoding SaCas9 comprises the nucleic acid of

SEQ ID NO: 914:

AAGCGCAATTACATCCTGGGCCTGGATATCGGCATCACCTCCGTGGG
CTACGGCATCATCGACTATGAGACACGGGATGTGATCGACGCCGGCG
TGAGACTGTTCAAGGAGGCCAACGTGGAGAACAATGAGGGCCGGCGG
AGCAAGAGGGGAGCAAGGCGCCTGAAGCGGAGAAGGCGCCACAGAAT
CCAGAGAGTGAAGAAGCTGCTGTTCGATTACAACCTGCTGACCGACC
ACTCCGAGCTGTCTGGCATCAATCCTTATGAGGCCCGGGTGAAGGGC
CTGTCCCAGAAGCTGTCTGAGGAGGAGTTTTCTGCCGCCCTGCTGCA
CCTGGCAAAGAGGAGAGGCGTGCACAACGTGAATGAGGTGGAGGAGG
ACACCGGCAACGAGCTGAGCACAAAGGAGCAGATCAGCCGCAATTCC
AAGGCCCTGGAGGAGAAGTATGTGGCCGAGCTGCAGCTGGAGCGGCT
GAAGAAGGATGGCGAGGTGAGGGGCTCCATCAATCGCTTCAAGACCT
CTGACTACGTGAAGGAGGCCAAGCAGCTGCTGAAGGTGCAGAAGGCC
TACCACCAGCTGGATCAGAGCTTTATCGATACATATATCGACCTGCT
GGAGACCAGGCGCACATACTATGAGGGACCAGGAGAGGGCTCCCCCT
TCGGCTGGAAGGACATCAAGGAGTGGTACGAGATGCTGATGGGCCAC
TGCACCTATTTTCCAGAGGAGCTGAGATCCGTGAAGTACGCCTATAA
CGCCGATCTGTACAACGCCCTGAATGACCTGAACAACCTGGTCATCA
CCAGGGATGAGAACGAGAAGCTGGAGTACTATGAGAAGTTCCAGATC
ATCGAGAACGTGTTCAAGCAGAAGAAGAAGCCTACACTGAAGCAGAT
CGCCAAGGAGATCCTGGTGAACGAGGAGGACATCAAGGGCTACCGCG
TGACCAGCACAGGCAAGCCAGAGTTCACCAATCTGAAGGTGTATCAC
GATATCAAGGACATCACAGCCCGGAAGGAGATCATCGAGAACGCCGA
GCTGCTGGATCAGATCGCCAAGATCCTGACCATCTATCAGAGCTCCG
AGGACATCCAGGAGGAGCTGACCAACCTGAATAGCGAGCTGACACAG
GAGGAGATCGAGCAGATCAGCAATCTGAAGGGCTACACCGGCACACA
CAACCTGTCCCTGAAGGCCATCAATCTGATCCTGGATGAGCTGTGGC
ACACAAACGACAATCAGATCGCCATCTTTAACAGGCTGAAGCTGGTG
CCAAAGAAGGTGGACCTGAGCCAGCAGAAGGAGATCCCAACCACACT
GGTGGACGATTTCATCCTGTCCCCCGTGGTGAAGCGGAGCTTCATCC
AGAGCATCAAAGTGATCAACGCCATCATCAAGAAGTACGGCCTGCCC
AATGATATCATCATCGAGCTGGCCAGGGAGAAGAACTCTAAGGACGC
CCAGAAGATGATCAATGAGATGCAGAAGAGGAACCGCCAGACCAATG
AGCGGATCGAGGAGATCATCAGAACCACAGGCAAGGAGAACGCCAAG
TACCTGATCGAGAAGATCAAGCTGCACGATATGCAGGAGGGCAAGTG
TCTGTATAGCCTGGAGGCCATCCCTCTGGAGGACCTGCTGAACAATC
CATTCAACTACGAGGTGGATCACATCATCCCCCGGAGCGTGAGCTTC
GACAATTCCTTTAACAATAAGGTGCTGGTGAAGCAGGAGGAGAACTC
TAAGAAGGGCAATAGGACCCCTTTCCAGTACCTGTCTAGCTCCGATT
CTAAGATCAGCTACGAGACCTTCAAGAAGCACATCCTGAATCTGGCC
AAGGGCAAGGGCCGCATCTCTAAGACCAAGAAGGAGTACCTGCTGGA
GGAGCGGGACATCAACAGATTCAGCGTGCAGAAGGACTTCATCAACC
GGAATCTGGTGGACACCAGATACGCCACACGCGGCCTGATGAATCTG
CTGCGGTCCTATTTCAGAGTGAACAATCTGGATGTGAAGGTGAAGAG
CATCAACGGCGGCTTCACCTCCTTTCTGCGGAGAAAGTGGAAGTTTA
AGAAGGAGAGAAACAAGGGCTATAAGCACCACGCCGAGGATGCCCTG
ATCATCGCCAATGCCGACTTCATCTTTAAGGAGTGGAAGAAGCTGGA
CAAGGCCAAGAAAGTGATGGAGAACCAGATGTTCGAGGAGAAGCAGG
CCGAGAGCATGCCCGAGATCGAGACCGAGCAGGAGTACAAGGAGATT
TTCATCACACCTCACCAGATCAAGCACATCAAGGACTTCAAGGACTA
CAAGTATTCCCACAGGGTGGATAAGAAGCCCAACCGCGAGCTGATCA
ATGACACCCTGTATTCTACAAGGAAGGACGATAAGGGCAATACCCTG
ATCGTGAACAATCTGAACGGCCTGTACGACAAGGATAATGACAAGCT
GAAGAAGCTGATCAACAAGAGCCCCGAGAAGCTGCTGATGTACCACC
ACGATCCTCAGACATATCAGAAGCTGAAGCTGATCATGGAGCAGTAC
GGCGACGAGAAGAACCCACTGTATAAGTACTATGAGGAGACCGGCAA
CTACCTGACAAAGTATTCCAAGAAGGATAATGGCCCCGTGATCAAGA
AGATCAAGTACTATGGCAACAAGCTGAATGCCCACCTGGACATCACC
GACGATTACCCCAACAGCCGGAATAAGGTGGTGAAGCTGAGCCTGAA
GCCATACAGGTTCGACGTGTACCTGGACAACGGCGTGTATAAGTTTG
TGACAGTGAAGAATCTGGATGTGATCAAGAAGGAGAACTACTATGAA
GTGAATAGCAAGTGCTACGAGGAGGCCAAGAAGCTGAAGAAGATCAG
CAACCAGGCCGAGTTCATCGCCTCTTTTTACAACAATGACCTGATCA
AGATCAATGGCGAGCTGTATAGAGTGATCGGCGTGAACAATGATCTG
CTGAACCGCATCGAAGTGAATATGATCGACATCACCTACCGGGAGTA
TCTGGAGAACATGAATGATAAGAGGCCCCCTCGCATCATCAAGACCA
TCGCCTCTAAGACACAGAGCATCAAGAAGTACTCTACAGACATCCTG
GGCAACCTGTATGAGGTGAAGAGCAAGAAGCACCCTCAGATCATCAA
GAAGGGC.

In some embodiments, the composition comprises a nucleic acid encoding SaCas9, the SaCas9 comprises an amino acid sequence of SEQ ID NO: 711.

In some embodiments, the SaCas9 is a variant of the amino acid sequence of SEQ ID NO: 711. In some embodiments, the SaCas9 comprises an amino acid other than an E at the position corresponding to position 781 of SEQ ID NO: 711. In some embodiments, the SaCas9 comprises an amino acid other than an N at the position corresponding to position 967 of SEQ ID NO: 711. In some embodiments, the SaCas9 comprises an amino acid other than an R at the position corresponding to position 1014 of SEQ ID NO: 711. In some embodiments, the SaCas9 comprises a K at the position corresponding to position 781 of SEQ ID NO: 711. In some embodiments, the SaCas9 comprises a K at the position corresponding to position 967 of SEQ ID NO: 711. In some embodiments, the SaCas9 comprises an H at the position corresponding to position 1014 of SEQ ID NO: 711. In some embodiments, the SaCas9 comprises an amino acid other than an Eat the position corresponding to position 781 of SEQ ID NO: 711; an amino acid other than an N at the position corresponding to position 967 of SEQ ID NO: 711; and an amino acid other than an Rat the position corresponding to position 1014 of SEQ ID NO: 711. In some embodiments, the SaCas9 comprises a K at the position corresponding to position 781 of SEQ ID NO: 711; a K at the position corresponding to position 967 of SEQ ID NO: 711; and an H at the position corresponding to position 1014 of SEQ ID NO: 711.

In some embodiments, the SaCas9 comprises an amino acid other than an R at the position corresponding to position 244 of SEQ ID NO: 711. In some embodiments, the SaCas9 comprises an amino acid other than an N at the position corresponding to position 412 of SEQ ID NO: 711. In some embodiments, the SaCas9 comprises an amino acid other than an N at the position corresponding to position 418 of SEQ ID NO: 711. In some embodiments, the SaCas9 comprises an amino acid other than an Rat the position corresponding to position 653 of SEQ ID NO: 711. In some embodiments, the SaCas9 comprises an amino acid other than an R at the position corresponding to position 244 of SEQ ID NO: 711; an amino acid other than an N at the position corresponding to position 412 of SEQ ID NO: 711; an amino acid other than an N at the position corresponding to position 418 of SEQ ID NO: 711; and an amino acid other than an Rat the position corresponding to position 653 of SEQ ID NO: 711. In some embodiments, the SaCas9 comprises an A at the position corresponding to position 244 of SEQ ID NO: 711. In some embodiments, the SaCas9 comprises an A at the position corresponding to position 412 of SEQ ID NO: 711. In some embodiments, the SaCas9 comprises an A at the position corresponding to position 418 of SEQ ID NO: 711. In some embodiments, the SaCas9 comprises an A at the position corresponding to position 653 of SEQ ID NO: 711. In some embodiments, the SaCas9 comprises an A at the position corresponding to position 244 of SEQ ID NO: 711; an A at the position corresponding to position 412 of SEQ ID NO: 711; an A at the position corresponding to position 418 of SEQ ID NO: 711; and an A at the position corresponding to position 653 of SEQ ID NO: 711.

In some embodiments, the SaCas9 comprises an amino acid other than an R at the position corresponding to position 244 of SEQ ID NO: 711; an amino acid other than an N at the position corresponding to position 412 of SEQ ID NO: 711; an amino acid other than an N at the position corresponding to position 418 of SEQ ID NO: 711; an amino acid other than an R at the position corresponding to position 653 of SEQ ID NO: 711; an amino acid other than an E at the position corresponding to position 781 of SEQ ID NO: 711; an amino acid other than an N at the position corresponding to position 967 of SEQ ID NO: 711; and an amino acid other than an Rat the position corresponding to position 1014 of SEQ ID NO: 711. In some embodiments, the SaCas9 comprises an A at the position corresponding to position 244 of SEQ ID NO: 711; an A at the position corresponding to position 412 of SEQ ID NO: 711; an A at the position corresponding to position 418 of SEQ ID NO: 711; an A at the position corresponding to position 653 of SEQ ID NO: 711; a K at the position corresponding to position 781 of SEQ ID NO: 711; a K at the position corresponding to position 967 of SEQ ID NO: 711; and an H at the position corresponding to position 1014 of SEQ ID NO: 711.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 715 (designated herein as SaCas9-KKH or SACAS9KKH):

KRNYILGLDIGITSVGYGIIDYETRDVIDAGVRLFKEANVENNEGRR
SKRGARRLKRRRRHRIQRVKKLLFDYNLLTDHSELSGINPYEARVKG
LSQKLSEEEFSAALLHLAKRRGVHNVNEVEEDTGNELSTKEQISRNS
KALEEKYVAELQLERLKKDGEVRGSINRFKTSDYVKEAKQLLKVQKA
YHQLDQSFIDTYIDLLETRRTYYEGPGEGSPFGWKDIKEWYEMLMGH
CTYFPEELRSVKYAYNADLYNALNDLNNLVITRDENEKLEYYEKFQI
IENVFKQKKKPTLKQIAKEILVNEEDIKGYRVTSTGKPEFTNLKVYH
DIKDITARKEIIENAELLDQIAKILTIYQSSEDIQEELTNLNSELTQ
EEIEQISNLKGYTGTHNLSLKAINLILDELWHTNDNQIAIFNRLKLV
PKKVDLSQQKEIPTTLVDDFILSPVVKRSFIQSIKVINAIIKKYGLP
NDIIIELAREKNSKDAQKMINEMQKRNRQTNERIEEIIRTTGKENAK
YLIEKIKLHDMQEGKCLYSLEAIPLEDLLNNPFNYEVDHIIPRSVSF
DNSFNNKVLVKQEENSKKGNRTPFQYLSSSDSKISYETFKKHILNLA
KGKGRISKTKKEYLLEERDINRFSVQKDFINRNLVDTRYATRGLMNL
LRSYFRVNNLDVKVKSINGGFTSFLRRKWKFKKERNKGYKHHAEDAL
IIANADFIFKEWKKLDKAKKVMENQMFEEKQAESMPEIETEQEYKEI
FITPHQIKHIKDFKDYKYSHRVDKKPNRKLINDTLYSTRKDDKGNTL
IVNNLNGLYDKDNDKLKKLINKSPEKLLMYHHDPQTYQKLKLIMEQY
GDEKNPLYKYYEETGNYLTKYSKKDNGPVIKKIKYYGNKLNAHLDIT
DDYPNSRNKVVKLSLKPYRFDVYLDNGVYKFVTVKNLDVIKKENYYE
VNSKCYEEAKKLKKISNQAEFIASFYKNDLIKINGELYRVIGVNNDL
LNRIEVNMIDITYREYLENMNDKRPPHIIKTIASKTQSIKKYSTDIL
GNLYEVKSKKHPQIIKKG.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 716 (designated herein as SaCas9-HF):

KRNYILGLDIGITSVGYGIIDYETRDVIDAGVRLFKEANVENNEGRR
SKRGARRLKRRRRHRIQRVKKLLFDYNLLTDHSELSGINPYEARVKG
LSQKLSEEEFSAALLHLAKRRGVHNVNEVEEDTGNELSTKEQISRNS
KALEEKYVAELQLERLKKDGEVRGSINRFKTSDYVKEAKQLLKVQKA
YHQLDQSFIDTYIDLLETRRTYYEGPGEGSPFGWKDIKEWYEMLMGH
CTYFPEELASVKYAYNADLYNALNDLNNLVITRDENEKLEYYEKFQI
IENVFKQKKKPTLKQIAKEILVNEEDIKGYRVTSTGKPEFTNLKVYH
DIKDITARKEIIENAELLDQIAKILTIYQSSEDIQEELTNLNSELTQ
EEIEQISNLKGYTGTHNLSLKAINLILDELWHTNDAQIAIFARLKLV
PKKVDLSQQKEIPTTLVDDFILSPVVKRSFIQSIKVINAIIKKYGLP
NDIIIELAREKNSKDAQKMINEMQKRNRQTNERIEEIIRTTGKENAK
YLIEKIKLHDMQEGKCLYSLEAIPLEDLLNNPFNYEVDHIIPRSVSF
DNSFNNKVLVKQEENSKKGNRTPFQYLSSSDSKISYETFKKHILNLA
KGKGRISKTKKEYLLEERDINRFSVQKDFINRNLVDTRYATAGLMNL
LRSYFRVNNLDVKVKSINGGFTSFLRRKWKFKKERNKGYKHHAEDAL
IIANADFIFKEWKKLDKAKKVMENQMFEEKQAESMPEIETEQEYKEI
FITPHQIKHIKDFKDYKYSHRVDKKPNRELINDTLYSTRKDDKGNTL
IVNNLNGLYDKDNDKLKKLINKSPEKLLMYHHDPQTYQKLKLIMEQY
GDEKNPLYKYYEETGNYLTKYSKKDNGPVIKKIKYYGNKLNAHLDIT
DDYPNSRNKVVKLSLKPYRFDVYLDNGVYKFVTVKNLDVIKKENYYE
VNSKCYEEAKKLKKISNQAEFIASFYNNDLIKINGELYRVIGVNNDL
LNRIEVNMIDITYREYLENMNDKRPPRIIKTIASKTQSIKKYSTDIL
GNLYEVKSKKHPQIIKKG.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 717 (designated herein as SaCas9-KKH-HF):

KRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINRFKTSDYVKEAKQLLKVQKAYHQLDQSFIDTY
IDLLETRRTYYEGPGEGSPFGWKDIKEWYEMLMGHCTYFPEELASVKYAY
NADLYNALNDLNNLVITRDENEKLEYYEKFQIIENVFKQKKKPTLKQIAK
EILVNEEDIKGYRVTSTGKPEFTNLKVYHDIKDITARKEIIENAELLDQI
AKILTIYQSSEDIQEELTNLNSELTQEEIEQISNLKGYTGTHNLSLKAIN
LILDELWHTNDAQIAIFARLKLVPKKVDLSQQKEIPTTLVDDFILSPVVK
RSFIQSIKVINAIIKKYGLPNDIIIELAREKNSKDAQKMINEMQKRNRQT
NERIEEIIRTTGKENAKYLIEKIKLHDMQEGKCLYSLEAIPLEDLLNNPF
NYEVDHIIPRSVSFDNSFNNKVLVKQEENSKKGNRTPFQYLSSSDSKISY
ETFKKHILNLAKGKGRISKTKKEYLLEERDINRFSVQKDFINRNLVDTRY
ATAGLMNLLRSYFRVNNLDVKVKSINGGFTSFLRRKWKFKKERNKGYKHH
AEDALIIANADFIFKEWKKLDKAKKVMENQMFEEKQAESMPEIETEQEYK
EIFITPHQIKHIKDFKDYKYSHRVDKKPNRKLINDTLYSTRKDDKGNTLI
VNNLNGLYDKDNDKLKKLINKSPEKLLMYHHDPQTYQKLKLIMEQYGDEK
NPLYKYYEETGNYLTKYSKKDNGPVIKKIKYYGNKLNAHLDITDDYPNSR
NKVVKLSLKPYRFDVYLDNGVYKFVTVKNLDVIKKENYYEVNSKCYEEAK
KLKKISNQAEFIASFYKNDLIKINGELYRVIGVNNDLLNRIEVNMIDITY
REYLENMNDKRPPHIIKTIASKTQSIKKYSTDILGNLYEVKSKKHPQIIK
KG.

In some embodiments, the Cas protein is any of the engineered Cas proteins disclosed in Schmidt et al., 2021, Nature Communications, “Improved CRISPR genome editing using small highly active and specific engineered RNA-guided nucleases.”

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 708 (designated herein as sRGN1):

MNQKFILGLDIGITSVGYGLIDYETKNIIDAGVRLFPEANVENNEGRRSK
RGSRRLKRRRIHRLDRVKHLLAEYDLLDLTNIPKSTNPYQTRVKGLNEKL
SKDELVIALLHIAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGKGSPFGWEGNIKKWFEQMMGHCTYFPEELR
SVKYSYSAELFNALNDLNNLVITRDEDAKLNYGEKFQIIENVFKQKKTPN
LKQIAIEIGVHETEIKGYRVNKSGTPEFTEFKLYHDLKSIVFDKSILENE
AILDQIAEILTIYQDEQSIKEELNKLPEILNEQDKAEIAKLIGYNGTHRL
SLKCIHLINEELWQTSRNQMEIFNYLNIKPNKVDLSEQNKIPKDMVNDFI
LSPVVKRTFIQSINVINKVIEKYGIPEDIIIELARENNSDDRKKFINNLQ
KKNEATRKRINEIIGQTGNQNAKRIVEKIRLHDQQEGKCLYSLKDIPLED
LLRNPNNYDIDHIIPRSVSFDDSMHNKVLVRREQNAKKNNQTPYQYLTSG
YADIKYSVFKQHVLNLAENKDRMTKKKREYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKYSHRVDKKPNRQLINDTLYSTRKKDNS
TYIVQTIKDIYAKDNTTLKKQFDKSPEKFLMYQHDPRTFEKLEVIMKQYA
NEKNPLAKYHEETGEYLTKYSKKNNGPIVKSLKYIGNKLGSHLDVTHQFK
SSTKKLVKLSIKPYRFDVYLTDKGYKFITISYLDVLKKDNYYYIPEQKYD
KLKLGKAIDKNAKFIASFYKNDLIKLDGEIYKIIGVNSDTRNMIELDLPD
IRYKEYCELNNIKGEPRIKKTIGKKVNSIEKLTTDVLGNVFTNTQYTKPQ
LLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 712 (designated herein as sRGN2):

MNQKFILGLDIGITSVGYGLIDYETKNIIDAGVRLFPEANVENNEGRRSK
RGSRRLKRRRIHRLERVKSLLSEYKIISGLAPTNNQPYNIRVKGLTEQLT
KDELAVALLHIAKRRGIHKIDVIDSNDDVGNELSTKEQLNKNSKLLKDKF
VCQIQLERMNEGQVRGEKNRFKTADIIKEIIQLLNVQKNFHQLDENFINK
YIELVEMRREYFEGPGQGSPFGWNGDLKKWYEMLMGHCTYFPQELRSVKY
AYSADLFNALNDLNNLIIQRDNSEKLEYHEKYHIIENVFKQKKKPTLKQI
AKEIGVNPEDIKGYRITKSGTPEFTEFKLYHDLKSVLFDQSILENEDVLD
QIAEILTIYQDKDSIKSKLTELDILLNEEDKENIAQLTGYNGTHRLSLKC
IRLVLEEQWYSSRNQMEIFTHLNIKPKKINLTAANKIPKAMIDEFILSPV
VKRTFIQSINVINKVIEKYGIPEDIIIELARENNSDDRKKFINNLQKKNE
ATRKRINEIIGQTGNQNAKRIVEKIRLHDQQEGKCLYSLESIALMDLLNN
PQNYEVDHIIPRSVAFDNSIHNKVLVKQIENSKKGNRTPYQYLNSSDAKL
SYNQFKQHILNLSKSKDRISKKKKDYLLEERDINKFEVQKEFINRNLVDT
RYATRELTSYLKAYFSANNMDVKVKTINGSFTNHLRKVWRFDKYRNHGYK
HHAEDALIIANADFLFKENKKLKAVNSVLEKPEIETKQLDIQVDSEDNYS
EMFIIPKQVQDIKDFRNFKYSHRVDKKPNRQLINDTLYSTRKKDNSTYIV
QTIKDIYAKDNTTLKKQFDKSPEKFLMYQHDPRTFEKLEVIMKQYANEKN
PLAKYHEETGEYLTKYSKKNNGPIVKSLKYIGNKLGSHLDVTHQFKSSTK
KLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYK
EYCELNNIKGEPRIKKTIGKKVNSIEKLTTDVLGNVFTNTQYTKPQLLFK
RGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 718 (designated herein as sRGN3):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKYSHRVDKKPNRQLINDTLYSTRKKDNS
TYIVQTIKDIYAKDNTTLKKQFDKSPEKFLMYQHDPRTFEKLEVIMKQYA
NEKNPLAKYHEETGEYLTKYSKKNNGPIVKSLKYIGNKLGSHLDVTHQFK
SSTKKLVKLSIKPYRFDVYLTDKGYKFITISYLDVLKKDNYYYIPEQKYD
KLKLGKAIDKNAKFIASFYKNDLIKLDGEIYKIIGVNSDTRNMIELDLPD
IRYKEYCELNNIKGEPRIKKTIGKKVNSIEKLTTDVLGNVFTNTQYTKPQ
LLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 719 (designated herein as sRGN3.1):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKYSHRVDKKPNRQLINDTLYSTRKKDNS
TYIVQTIKDIYAKDNTTLKKQFDKSPEKFLMYQHDPRTFEKLEVIMKQYA
NEKNPLAKYHEETGEYLTKYSKKNNGPIVKSLKYIGNKLGSHLDVTHQFK
SSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 720 (designated herein as sRGN3.2):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKFSHRVDKKPNRQLINDTLYSTRMKDEH
DYIVQTITDIYGKDNTNLKKQFNKNPEKFLMYQNDPKTFEKLSIIMKQYS
DEKNPLAKYYEETGEYLTKYSKKNNGPIVKKIKLLGNKVGNHLDVTNKYE
NSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 721 (designated herein as sRGN3.3):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTSYLKAYFSANNMDVKVKTINGSFTNHLRKVWRFDKYRN
HGYKHHAEDALIIANADFLFKENKKLQNTNKILEKPTIENNTKKVTVEKE
EDYNNVFETPKLVEDIKQYRDYKFSHRVDKKPNRQLINDTLYSTRMKDEH
DYIVQTITDIYGKDNTNLKKQFNKNPEKFLMYQNDPKTFEKLSIIMKQYS
DEKNPLAKYYEETGEYLTKYSKKNNGPIVKKIKLLGNKVGNHLDVTNKYE
NSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 722 (designated herein as sRGN4):

MNQKFILGLDIGITSVGYGLIDYETKNIIDAGVRLFPEANVENNEGRRSK
RGSRRLKRRRIHRLERVKKLLEDYNLLDQSQIPQSTNPYAIRVKGLSEAL
SKDELVIALLHIAKRRGIHNINVSSEDEDASNELSTKEQINRNNKLLKDK
YVCEVQLQRLKEGQIRGEKNRFKTTDILKEIDQLLKVQKDYHNLDIDFIN
VETQYKEIRREYFEGPGKGSPYGWEGDPKAWYETLMGHCTYFPDELRSVK
YAYSADLFNALNDLNNLVIQRDGLSKLEYHEKYHIIENVFKQKKKPTLKQ
IANEINVNPEDIKGYRITKSGKPEFTSFKLFHDLKKVVKDHAILDDIDLL
NQIAEILTIYQDKDSIVAELGQLEYLMSEADKQSISELTGYTGTHSLSLK
CMNMIIDELWHSSMNQMEVFTYLNMRPKKYELKGYQRIPTDMIDDAILSP
VVKRTFIQSINVINKVIEKYGIPEDIIIELARENNSDDRKKFINNLQKKN
EATRKRINEIIGQTGNQNAKRIVEKIRLHDQQEGKCLYSLESIPLEDLLN
NPNHYEVDHIIPRSVSFDNSYHNKVLVKQSENSKKSNLTPYQYFNSGKSK
LSYNQFKQHILNLSKSQDRISKKKKEYLLEERDINKFEVQKEFINRNLVD
TRYATRELTNYLKAYFSANNMNVKVKTINGSFTDYLRKVWKFKKERNHGY
KHHAEDALIIANADFLFKENKKLKAVNSVLEKPEIETKQLDIQVDSEDNY
SEMFIIPKQVQDIKDFRNFKYSHRVDKKPNRQLINDTLYSTRKKDNSTYI
VQTIKDIYAKDNTTLKKQFDKSPEKFLMYQHDPRTFEKLEVIMKQYANEK
NPLAKYHEETGEYLTKYSKKNNGPIVKSLKYIGNKLGSHLDVTHQFKSST
KKLVKLSIKPYRFDVYLTDKGYKFITISYLDVLKKDNYYYIPEQKYDKLK
LGKAIDKNAKFIASFYKNDLIKLDGEIYKIIGVNSDTRNMIELDLPDIRY
KEYCELNNIKGEPRIKKTIGKKVNSIEKLTTDVLGNVFTNTQYTKPQLLF
KRGN

Modified guide RNAs

In some embodiments, the guide RNA is chemically modified. A guide RNA comprising one or more modified nucleosides or nucleotides is called a “modified” guide RNA or “chemically modified” guide RNA, to describe the presence of one or more non-naturally and/or naturally occurring components or configurations that are used instead of or in addition to the canonical A, G, C, and U residues. In some embodiments, a modified guide RNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified guide RNAs comprising nucleosides and nucleotides (collectively “residues”) that can have two, three, four, or more modifications. For example, a modified residue can have a modified sugar and a modified nucleobase, or a modified sugar and a modified phosphodiester. In some embodiments, every base of a guide RNA is modified, e.g., all bases have a modified phosphate group, such as a phosphorothioate group. In certain embodiments, all, or substantially all, of the phosphate groups of an guide RNA molecule are replaced with phosphorothioate groups. In some embodiments, modified guide RNAs comprise at least one modified residue at or near the 5′ end of the RNA. In some embodiments, modified guide RNAs comprise at least one modified residue at or near the 3′ end of the RNA.

In some embodiments, the guide RNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified guide RNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the guide RNAs described herein can contain one or more modified nucleosides or nucleotides, e.g., to introduce stability toward intracellular or serum-based nucleases. In some embodiments, the modified guide RNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C1-6 alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising sgRNA, one or more residues at one or both ends of the sgRNA may be chemically modified, and/or internal nucleosides may be modified, and/or the entire sgRNA may be chemically modified. Certain embodiments comprise a 5′ end modification. Certain embodiments comprise a 3′ end modification.

Modifications of 2′-O-methyl are encompassed.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Modifications of 2′-fluoro (2′-F) are encompassed.

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

Abasic nucleotides refer to those which lack nitrogenous bases.

Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide.

### Ribonucleoprotein Complex

In some embodiments, a composition is encompassed comprising: a) one or more guide RNAs comprising one or more guide sequences from Table 1A and Table 1B and b) saCas9, or any of the variant Cas9 proteins disclosed herein. In some embodiments, the guide RNA together with a Cas9 is called a ribonucleoprotein complex (RNP).

In some embodiments, the disclosure provides for an RNP complex, wherein the guide RNA (e.g., any of the guide RNAs disclosed herein) binds to or is capable of binding to a target sequence in the DMPK gene, or a target sequence bound by any of the sequences disclosed in Table 1A and Table 1B, wherein the DMPK gene comprises a PAM recognition sequence position upstream of the target sequence, and wherein the RNP cuts at a position that is 3 nucleotides upstream (−3) of the PAM in the DMPK gene. In some embodiments, the RNP also cuts at a position that is 2 nucleotides upstream (−2), 4 nucleotides upstream (−4), 5 nucleotides upstream (−5), or 6 nucleotides upstream (−6) of the PAM in the DMPK gene. In some embodiments, the RNP cuts at a position that is 3 nucleotides upstream (−3) and 4 nucleotides upstream (−4) of the PAM in the DMPK gene.

In some embodiments, chimeric Cas9 (SaCas9) nucleases are used, where one domain or region of the protein is replaced by a portion of a different protein. In some embodiments, a Cas9 nuclease domain may be replaced with a domain from a different nuclease such as Fok1. In some embodiments, a Cas9 nuclease may be a modified nuclease.

In some embodiments, the Cas9 is modified to contain only one functional nuclease domain. For example, the agent protein may be modified such that one of the nuclease domains is mutated or fully or partially deleted to reduce its nucleic acid cleavage activity.

In some embodiments, a conserved amino acid within a Cas9 protein nuclease domain is substituted to reduce or alter nuclease activity. In some embodiments, a Cas9 nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas9 nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB-A0Q7Q2 (CPF1_FRATN)). Further exemplary amino acid substitutions include D10A and N580A (based on the S. aureus Cas9 protein). See, e.g., Friedland et al., 2015, Genome Biol., 16:257.

In some embodiments, the Cas9 lacks cleavase activity. In some embodiments, the Cas9 comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the Cas9 lacking cleavase activity or the dCas DNA-binding polypeptide is a version of a Cas nuclease (e.g., a Cas9 nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the Cas9 comprises one or more heterologous functional domains (e.g., is or comprises a fusion polypeptide).

In some embodiments, the heterologous functional domain may facilitate transport of the Cas9 into the nucleus of a cell. For example, the heterologous functional domain may be a nuclear localization signal (NLS). In some embodiments, the Cas9 may be fused with 1-10 NLS(s). In some embodiments, the Cas9 may be fused with 1-5 NLS(s). In some embodiments, the Cas9 may be fused with one NLS. Where one NLS is used, the NLS may be attached at the N-terminus or the C-terminus of the Cas9 sequence, and may be directly attached. In some embodiments, where more than one NLS is used, one or more NLS may be attached at the N-terminus and/or one or more NLS may be attached at the C-terminus. In some embodiments, one or more NLSs are directly attached to the Cas9. In some embodiments, one or more NLSs are attached to the Cas9 by means of a linker. In some embodiments, the linker is between 3-25 amino acids in length. In some embodiments, the linker is between 3-6 amino acids in length. In some embodiments, the linker comprises glycine and serine. In some embodiments, the linker comprises the sequence of GSVD (SEQ ID NO: 940) or GSGS (SEQ ID NO: 941). It may also be inserted within the Cas9 sequence. In other embodiments, the Cas9 may be fused with more than one NLS. In some embodiments, the Cas9 may be fused with 2, 3, 4, or 5 NLSs. In some embodiments, the Cas9 may be fused with two NLSs. In certain circumstances, the two NLSs may be the same (e.g., two SV40 NLSs) or different. In some embodiments, the Cas9 protein is fused with an SV40 NLS. In some embodiments, the SV40 NLS comprises the amino acid sequence of SEQ ID NO: 713 (PKKKRKV). In some embodiments, the Cas9 protein (e.g., the SaCas9) is fused to a nucleoplasmin NLS. In some embodiments, the nucleoplasmin NLS comprises the amino acid sequence of SEQ ID NO: 714 (KRPAATKKAGQAKKKK). In some embodiments, the Cas9 protein is fused with a c-Myc NLS. In some embodiments, the c-Myc NLS is SEQ ID NO: 942 (PAAKKKKLD) and/or is encoded by the nucleic acid sequence of SEQ ID NO: 943 (CCGGCAGCTAAGAAAAAGAAACTGGAT). In some embodiments, the Cas9 is fused to two SV40 NLS sequences linked at the carboxy terminus. In some embodiments, the Cas9 may be fused with two NLSs, one linked at the N-terminus and one at the C-terminus. In some embodiments, the Cas9 may be fused with 3 NLSs. In some embodiments, the Cas9 may be fused with no NLS. In some embodiments, the Cas9 may be fused with one NLS. In some embodiments, the Cas9 may be fused with an NLS on the C-terminus and does not comprise an NLS fused on the N-terminus. In some embodiments, the Cas9 may be fused with an NLS on the N-terminus and does not comprise an NLS fused on the C-terminus. In some embodiments, the Cas9 protein is fused to an SV40 NLS and to a nucleoplasmin NLS. In some embodiments, the SV40 NLS is fused to the C-terminus of the Cas9, while the nucleoplasmin NLS is fused to the N-terminus of the Cas9 protein. In some embodiments, the SV40 NLS is fused to the N-terminus of the Cas9, while the nucleoplasmin NLS is fused to the C-terminus of the Cas9 protein. In some embodiments, a c-myc NLS is fused to the N-terminus of the Cas9 and an SV40 NLS and/or nucleoplasmin NLS is fused to the C-terminus of the Cas9. In some embodiments, a c-myc NLS is fused to the N-terminus of the Cas9 (e.g., by means of a linker such as GSVD (SEQ ID NO: 940)), an SV40 NLS is fused to the C-terminus of the Cas9 (e.g., by means of a linker such as GSGS (SEQ ID NO: 941)), and a nucleoplasmin NLS is fused to the C-terminus of the SV-40 NLS (e.g., by means of a linker such as GSGS (SEQ ID NO: 941)). In some embodiments, the SV40 NLS is fused to the Cas9 protein by means of a linker. In some embodiments, the nucleoplasmin NLS is fused to the Cas9 protein by means of a linker.

In some embodiments, the heterologous functional domain may be capable of modifying the intracellular half-life of the Cas9. In some embodiments, the half-life of the Cas9 may be increased. In some embodiments, the half-life of the Cas9 may be reduced. In some embodiments, the heterologous functional domain may be capable of increasing the stability of the Cas9. In some embodiments, the heterologous functional domain may be capable of reducing the stability of the Cas9. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the Cas9 may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rubl in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AUS, E, ECS, E2, FLAG, HA, nus, Softag 1, Softag 3, Strep, SBP, Glu-Glu, HSV, KT3, S, 51, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the Cas9 to a specific organelle, cell type, tissue, or organ. In some embodiments, the heterologous functional domain may target the Cas9 to muscle.

In further embodiments, the heterologous functional domain may be an effector domain. When the Cas9 is directed to its target sequence, e.g., when a Cas9 is directed to a target sequence by a guide RNA, the effector domain may modify or affect the target sequence. In some embodiments, the effector domain may be chosen from a nucleic acid binding domain or a nuclease domain (e.g., a non-Cas nuclease domain). In some embodiments, the heterologous functional domain is a nuclease, such as a FokI nuclease. See, e.g., U.S. Pat. No. 9,023,649.

### Determination of Efficacy of Guide RNAs

In some embodiments, the efficacy of a guide RNA is determined when delivered or expressed together with other components forming an RNP. In some embodiments, the guide RNA is expressed together with a SaCas9. In some embodiments, the guide RNA is delivered to or expressed in a cell line that already stably expresses an SaCas9. In some embodiments the guide RNA is delivered to a cell as part of an RNP. In some embodiments, the guide RNA is delivered to a cell along with a nucleic acid (e.g., mRNA) encoding SaCas9.

In some embodiments, the efficacy of particular guide RNAs is determined based on in vitro models. In some embodiments, the in vitro model is a cell line.

In some embodiments, the efficacy of particular guide RNAs is determined across multiple in vitro cell models for a guide RNA selection process. In some embodiments, a cell line comparison of data with selected guide RNAs is performed. In some embodiments, cross screening in multiple cell models is performed.

In some embodiments, the efficacy of particular guide RNAs is determined based on in vivo models. In some embodiments, the in vivo model is a rodent model. In some embodiments, the rodent model is a mouse which expresses a gene comprising an expanded trinucleotide repeat or a self-complementary region. The gene may be the human version or a rodent (e.g., murine) homolog of any of the genes listed in Table 1. In some embodiments, the gene is human DMPK. In some embodiments, the gene is a rodent (e.g., murine) homolog of DMPK. In some embodiments, the in vivo model is a non-human primate, for example cynomolgus monkey. See, e.g., the mouse model described in Huguet et al., 2012, PLoS Genet, 8(11):e1003043. In some embodiments, the in vivo model is a non-human primate, for example cynomolgus monkey.

### III. Methods of Gene Editing, CTG Repeat Excision, and Treating DM1

This disclosure provides methods and uses for treating Myotonic Dystrophy Type 1 (DM1). In some embodiments, any of the compositions or systems described herein may be administered to a subject in need thereof for use in making a double strand break in the DMPK gene. In some embodiments, any of the compositions or systems described herein may be administered to a subject in need thereof for use in excising a CTG repeat in the 3′ untranslated region (UTR) of the DMPK gene. In some embodiments, any of the compositions or systems described herein may be administered to a subject in need thereof for use in treating DM1. In some embodiments, a nucleic acid molecule comprising a first nucleic acid encoding one or more guide RNAs of Table 1A and Table 1B and a second nucleic acid encoding SaCas9 is administered to a subject to treat DM1. In some embodiments, a single nucleic acid molecule (which may be a vector, including an AAV vector) comprising a first nucleic acid encoding one or more guide RNAs of Table 1A and Table 1B and a second nucleic acid encoding SaCas9 is administered to a subject to treat DM1.

In some embodiments, any of the compositions described herein is administered to a subject in need thereof to treat Myotonic Dystrophy Type 1 (DM1).

For treatment of a subject (e.g., a human), any of the compositions disclosed herein may be administered in a manner compatible with the dosage formulation and in such amount as is therapeutically effective. The compositions may be readily administered in a variety of dosage forms, such as injectable solutions. For parenteral administration in an aqueous solution, for example, the solution will generally be suitably buffered and the liquid diluent first rendered isotonic with, for example, sufficient saline or glucose. Such aqueous solutions may be used, for example, for intravenous, intramuscular, subcutaneous, and/or intraperitoneal administration.

In some embodiments, any of the compositions described herein is administered to a subject in need thereof to induce a double strand break in the DMPK gene.

In some embodiments, any of the compositions described herein is administered to a subject in need thereof to excise a CTG repeat in the 3′ UTR of the DMPK gene.

In some embodiments, any of the compositions described herein is administered to a subject in need thereof to treat DM1, e.g., in a subject having a CTG repeat in the 3′ UTR of the DMPK gene.

In some embodiments, a method of treating Myotonic Dystrophy Type 1 (DM1) is provided, the method comprising delivering to a cell any one of the compositions described herein. In some embodiments, the method further comprises administering a DNA-PK inhibitor. In some embodiments, the DNA-PK inhibitor is Compound 1. In some embodiments, the DNA-PK inhibitor is Compound 2. In some embodiments, the DNA-PK inhibitor is Compound 6.

In particular, in some embodiments, a method of treating Myotonic Dystrophy Type 1 (DM1) is provided, the method comprising delivering to a cell: 1) a nucleic acid molecule comprising: a nucleic acid encoding one or more spacer sequences selected from SEQ ID NOs: 1-8, 10-28, or 101-154; a nucleic acid encoding one or more spacer sequences comprising at least 17, 18, 19, 20, or 21 contiguous nucleotides of one or more spacer sequences selected from SEQ ID NOs: 1-8, 10-28, or 101-154; or a nucleic acid encoding a spacer sequence that is at least 90% identical to any one of SEQ ID NOs: 1-8, 10-28, or 101-154; and 2) a Staphylococcus aureus Cas9 (SaCas9) or a nucleic acid encoding SaCas9. In some embodiments, the cell comprises a CTG repeat in the 3′ UTR of the DMPK gene. In some embodiments, the method further comprises administering a DNA-PK inhibitor.

In particular, in some embodiments, a method of treating Myotonic Dystrophy Type 1 (DM1) is provided, the method comprising delivering to a cell: 1) a nucleic acid molecule comprising: a nucleic acid encoding one or more spacer sequences selected from SEQ ID NOs: 1-9, 10-28, or 101-154; a nucleic acid encoding one or more spacer sequences comprising at least 17, 18, 19, 20, or 21 contiguous nucleotides of a spacer sequence selected from SEQ ID NOs: 1-9, 10-28, or 101-154; or a nucleic acid encoding one or more spacer sequences that are at least 90% identical to any one of SEQ ID NOs: 1-9, 10-28, or 101-154; and 2) a Staphylococcus aureus Cas9 (SaCas9) or a nucleic acid encoding SaCas9. In some embodiments, the cell comprises a CTG repeat in the 3′ UTR of the DMPK gene. In some embodiments, the method further comprises administering a DNA-PK inhibitor.

In some embodiments, a method of treating Myotonic Dystrophy Type 1 (DM1) is provided, the method comprising delivering to a cell a single nucleic acid molecule comprising: 1) a nucleic acid encoding one or more spacer sequences selected from any one of SEQ ID NOs: 1, 2, 3, 4, 7, 8, 10, 11, 12, 13, 14, 15, 18, 19, 20, 21, 23, 25, 26, 27, and 28; a nucleic acid encoding one or more spacer sequence comprising at least 17, 18, 19, 20, or 21 contiguous nucleotides of a spacer sequence selected from any one of SEQ ID NOs: 1, 2, 3, 4, 7, 8, 10, 11, 12, 13, 14, 15, 18, 19, 20, 21, 23, 25, 26, 27, and 28; or a nucleic acid encoding one or more spacer sequence that is at least 90% identical to any one of SEQ ID NOs: 1, 2, 3, 4, 7, 8, 10, 11, 12, 13, 14, 15, 18, 19, 20, 21, 23, 25, 26, 27, and 28; and 2) a nucleic acid encoding a Staphylococcus aureus Cas9 (SaCas9). In some embodiments, a method of treating Myotonic Dystrophy Type 1 (DM1) is provided, the method comprising delivering to a cell: a single nucleic acid molecule comprising: 1) a nucleic acid encoding one or more spacer sequences selected from any one of SEQ ID NOs: 1, 2, 3, 4, 7, 8, 10, 11, 12, 13, 14, 15, 18, 19, 20, 21, 23, 25, 26, 27, and 28; a nucleic acid encoding one or more spacer sequences comprising at least 17, 18, 19, 20, or 21 contiguous nucleotides of one or more spacer sequences selected from any one of SEQ ID NOs: 1, 2, 3, 4, 7, 8, 10, 11, 12, 13, 14, 15, 18, 19, 20, 21, 23, 25, 26, 27, and 28; or a nucleic acid encoding one or more spacer sequences that is at least 90% identical to any one of SEQ ID NOs: 1, 2, 3, 4, 7, 8, 10, 11, 12, 13, 14, 15, 18, 19, 20, 21, 23, 25, 26, 27, and 28; and 2) a nucleic acid encoding SaCas9. In some embodiments, the spacer sequence is SEQ ID NO: 1. In some embodiments, the spacer sequence is SEQ ID NO: 2. In some embodiments, the spacer sequence is SEQ ID NO: 3. In some embodiments, the spacer sequence is SEQ ID NO: 4. In some embodiments, the spacer sequence is SEQ ID NO: 7. In some embodiments, the spacer sequence is SEQ ID NO: 8. In some embodiments, the spacer sequence is SEQ ID NO: 10. In some embodiments, the spacer sequence is SEQ ID NO: 11. In some embodiments, the spacer sequence is SEQ ID NO: 12. In some embodiments, the spacer sequence is SEQ ID NO: 13. In some embodiments, the spacer sequence is SEQ ID NO: 14. In some embodiments, the spacer sequence is SEQ ID NO: 15. In some embodiments, the spacer sequence is SEQ ID NO: 18. In some embodiments, the spacer sequence is SEQ ID NO: 19. In some embodiments, the spacer sequence is SEQ ID NO: 21. In some embodiments, the spacer sequence is SEQ ID NO: 23. In some embodiments, the spacer sequence is SEQ ID NO: 25. In some embodiments, the spacer sequence is SEQ ID NO: 26. In some embodiments, the spacer sequence is SEQ ID NO: 27. In some embodiments, the spacer sequence is SEQ ID NO: 28. In some embodiments, the method further comprises administering a DNA-PK inhibitor.

In some embodiments, a method of treating Myotonic Dystrophy Type 1 (DM1) is provided, the method comprising delivering to a cell a single nucleic acid molecule comprising: 1) a nucleic acid encoding one or more spacer sequences selected from any one of SEQ ID NOs: 101, 102, 103, 104, 105, 106, 107, 113, 114, 115, 116, 117, 118, 119, 120, 121, 122, 123, 124, 125, 126, 127, 128, 133, 134, 135, 136, 137, 138, 139, 140, 143, 144, 147, 148, 149, 150, 151, 152, 153, and 154; a nucleic acid encoding one or more spacer sequence comprising at least 17, 18, 19, 20, or 21 contiguous nucleotides of a spacer sequence selected from any one of SEQ ID NOs: 101, 102, 103, 104, 105, 106, 107, 113, 114, 115, 116, 117, 118, 119, 120, 121, 122, 123, 124, 125, 126, 127, 128, 133, 134, 135, 136, 137, 138, 139, 140, 143, 144, 147, 148, 149, 150, 151, 152, 153, and 154; or a nucleic acid encoding one or more spacer sequence that is at least 90% identical to any one of SEQ ID NOs: 101, 102, 103, 104, 105, 106, 107, 113, 114, 115, 116, 117, 118, 119, 120, 121, 122, 123, 124, 125, 126, 127, 128, 133, 134, 135, 136, 137, 138, 139, 140, 143, 144, 147, 148, 149, 150, 151, 152, 153, and 154; and 2) a nucleic acid encoding a Staphylococcus aureus Cas9 (SaCas9). In some embodiments, a method of treating Myotonic Dystrophy Type 1 (DM1) is provided, the method comprising delivering to a cell: a single nucleic acid molecule comprising: 1) a nucleic acid encoding one or more spacer sequences selected from any one of SEQ ID NOs: 101, 102, 103, 104, 105, 106, 107, 113, 114, 115, 116, 117, 118, 119, 120, 121, 122, 123, 124, 125, 126, 127, 128, 133, 134, 135, 136, 137, 138, 139, 140, 143, 144, 147, 148, 149, 150, 151, 152, 153, and 154; a nucleic acid encoding one or more spacer sequences comprising at least 17, 18, 19, 20, or 21 contiguous nucleotides of one or more spacer sequences selected from any one of SEQ ID NOs: 101, 102, 103, 104, 105, 106, 107, 113, 114, 115, 116, 117, 118, 119, 120, 121, 122, 123, 124, 125, 126, 127, 128, 133, 134, 135, 136, 137, 138, 139, 140, 143, 144, 147, 148, 149, 150, 151, 152, 153, and 154; or a nucleic acid encoding one or more spacer sequences that is at least 90% identical to any one of SEQ ID NOs: 101, 102, 103, 104, 105, 106, 107, 113, 114, 115, 116, 117, 118, 119, 120, 121, 122, 123, 124, 125, 126, 127, 128, 133, 134, 135, 136, 137, 138, 139, 140, 143, 144, 147, 148, 149, 150, 151, 152, 153, and 154; and 2) a nucleic acid encoding SaCas9. In some embodiments, the spacer sequence is SEQ ID NO: 101. In some embodiments, the spacer sequence is SEQ ID NO: 102. In some embodiments, the spacer sequence is SEQ ID NO: 103. In some embodiments, the spacer sequence is SEQ ID NO: 104. In some embodiments, the spacer sequence is SEQ ID NO: 105. In some embodiments, the spacer sequence is SEQ ID NO: 106. In some embodiments, the spacer sequence is SEQ ID NO: 107. In some embodiments, the spacer sequence is SEQ ID NO: 113. In some embodiments, the spacer sequence is SEQ ID NO: 114. In some embodiments, the spacer sequence is SEQ ID NO: 115. In some embodiments, the spacer sequence is SEQ ID NO: 116. In some embodiments, the spacer sequence is SEQ ID NO: 117. In some embodiments, the spacer sequence is SEQ ID NO: 118. In some embodiments, the spacer sequence is SEQ ID NO: 119. In some embodiments, the spacer sequence is SEQ ID NO: 120. In some embodiments, the spacer sequence is SEQ ID NO: 121. In some embodiments, the spacer sequence is SEQ ID NO: 122. In some embodiments, the spacer sequence is SEQ ID NO: 123. In some embodiments, the spacer sequence is SEQ ID NO: 124. In some embodiments, the spacer sequence is SEQ ID NO: 125. In some embodiments, the spacer sequence is SEQ ID NO: 126. In some embodiments, the spacer sequence is SEQ ID NO: 127. In some embodiments, the spacer sequence is SEQ ID NO: 128. In some embodiments, the spacer sequence is SEQ ID NO: 133. In some embodiments, the spacer sequence is SEQ ID NO: 134. In some embodiments, the spacer sequence is SEQ ID NO: 135. In some embodiments, the spacer sequence is SEQ ID NO: 136. In some embodiments, the spacer sequence is SEQ ID NO: 137. In some embodiments, the spacer sequence is SEQ ID NO: 138. In some embodiments, the spacer sequence is SEQ ID NO: 139. In some embodiments, the spacer sequence is SEQ ID NO: 140. In some embodiments, the spacer sequence is SEQ ID NO: 143. In some embodiments, the spacer sequence is SEQ ID NO: 144; In some embodiments, the spacer sequence is SEQ ID NO: 147; In some embodiments, the spacer sequence is SEQ ID NO: 148; In some embodiments, the spacer sequence is SEQ ID NO: 149; In some embodiments, the spacer sequence is SEQ ID NO: 150; In some embodiments, the spacer sequence is SEQ ID NO: 151; In some embodiments, the spacer sequence is SEQ ID NO: 152; In some embodiments, the spacer sequence is SEQ ID NO: 153; In some embodiments, the spacer sequence is SEQ ID NO: 154. In some embodiments, the method further comprises administering a DNA-PK inhibitor.

In some embodiments, a method of treating Myotonic Dystrophy Type 1 (DM1) is provided, the method comprising delivering to a cell a single nucleic acid molecule comprising: i) a nucleic acid encoding a pair of guide RNAs comprising: a) a first and second spacer sequence selected from any one of SEQ ID NOs: 1 and 10; 1 and 11; 1 and 12; 1 and 13; 1 and 14; 1 and 15; 1 and 16; 1 and 17; 1 and 18; 1 and 19; 1 and 20; 1 and 21; 1 and 22; 1 and 23; 1 and 24; 1 and 25; 1 and 26; land 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28; b) a first and second spacer sequence comprising at least 17, 18, 19, 20, or 21 contiguous nucleotides of any of the first and second spacer sequences of i) a); or c) a first and second spacer sequence that is at least 90% identical to any of the first and second spacer sequences of i) a) or i) b); and ii) a nucleic acid encoding a Staphylococcus aureus Cas9 (SaCas9). In some embodiments, the method further comprises administering a DNA-PK inhibitor.

In some embodiments, a method of treating Myotonic Dystrophy Type 1 (DM1) is provided, the method comprising delivering to a cell a single nucleic acid molecule comprising: i) a nucleic acid encoding a pair of guide RNAs comprising: a) a first and second spacer sequence selected from any one of SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 8 and 10; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 7 and 10; 1 and 13; 1 and 20; 1 and 28; 4 and 27; 7 and 20; 7 and 23; 7 and 13; 7 and 28; 2 and 27; 8 and 27; 4 and 11; 4 and 25; 4 and 28; 4 and 19; 4 and 15; 8 and 11; 3 and 27; 2 and 25; 2 and 11; 7 and 18; 3 and 25; 8 and 15; 8 and 25; 3 and 11; 3 and 19; 1 and 15; 3 and 15; 1 and 27; 2 and 15; 2 and 19; 1 and 11; 1 and 25; 8 and 19; 4 and 21; 8 and 21; 7 and 27; 7 and 15; 1 and 19; 2 and 21; 7 and 11; 3 and 21; 4 and 14; 7 and 19; 4 and 26; 8 and 26; 7 and 25; 1 and 21; 3 and 26; 2 and 26; 8 and 14; 1 and 14; 2 and 14; 3 and 14; 1 and 26; 7 and 21; 7 and 14; and 7 and 26; b) a first and second spacer sequence comprising at least 17, 18, 19, 20, or 21 contiguous nucleotides of any of the first and second spacer sequences of i) a); or c) a first and second spacer sequence that is at least 90% identical to any of the first and second spacer sequences of i) a) or i) b); and ii) a nucleic acid encoding a Staphylococcus aureus Cas9 (SaCas9). In some embodiments, the method further comprises administering a DNA-PK inhibitor.

In some embodiments, a method of treating Myotonic Dystrophy Type 1 (DM1) is provided, the method comprising delivering to a cell a single nucleic acid molecule comprising: i) a nucleic acid encoding a pair of guide RNAs comprising: a) a first and second spacer sequence selected from any one of SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 8 and 10; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 7 and 10; 1 and 13; 1 and 20; 1 and 28; 4 and 27; 7 and 20; 7 and 23; 7 and 13; 7 and 28; 2 and 27; 8 and 27; 4 and 11; and 4 and 25; b) a first and second spacer sequence comprising at least 17, 18, 19, 20, or 21 contiguous nucleotides of any of the first and second spacer sequences of i) a); or c) a first and second spacer sequence that is at least 90% identical to any of the first and second spacer sequences of i) a) or i) b); and ii) a nucleic acid encoding a Staphylococcus aureus Cas9 (SaCas9). In some embodiments, the method further comprises administering a DNA-PK inhibitor.

In some embodiments, a method of treating Myotonic Dystrophy Type 1 (DM1) is provided, the method comprising delivering to a cell a single nucleic acid molecule comprising: i) a nucleic acid encoding a pair of guide RNAs comprising: a) a first and second spacer sequence selected from any one of SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 1 and 13; 1 and 20; 1 and 28; 4 and 27; 7 and 20; 7 and 23; 7 and 13; 7 and 28; 2 and 27; 4 and 11; 4 and 25; 4 and 28; 4 and 19; 4 and 15; 8 and 11; 3 and 27; 2 and 25; 2 and 11; 7 and 18; 3 and 25; 8 and 15; 3 and 11; 3 and 19; 1 and 15; 3 and 15; 1 and 27; 2 and 15; 2 and 19; 1 and 11; 1 and 25; 4 and 21; 8 and 21; 7 and 27; 7 and 15; 1 and 19; 2 and 21; 7 and 11; 3 and 21; 7 and 19; 7 and 25; land 21; 3 and 26; 3 and 14; 7 and 21; and 7 and 14; b) a first and second spacer sequence comprising at least 17, 18, 19, 20, or 21 contiguous nucleotides of any of the first and second spacer sequences of i) a); or c) a first and second spacer sequence that is at least 90% identical to any of the first and second spacer sequences of i) a. or i) b); and ii) a nucleic acid encoding a Staphylococcus aureus Cas9 (SaCas9). In some embodiments, the method further comprises administering a DNA-PK inhibitor.

In some embodiments, a method of treating Myotonic Dystrophy Type 1 (DM1) is provided, the method comprising delivering to a cell a single nucleic acid molecule comprising: i) a nucleic acid encoding a pair of guide RNAs comprising: a) a first and second spacer sequence selected from any one of SEQ ID NOs: 4 and 12; 2 and 10; 4 and 28; 8 and 12; 4 and 13; 3 and 10; 7 and 12; 7 and 13; 4 and 28; and 7 and 18; b) a first and second spacer sequence comprising at least 17, 18, 19, 20, or 21 contiguous nucleotides of any of the first and second spacer sequences of i) a); or c) a first and second spacer sequence that is at least 90% identical to any of the first and second spacer sequences of i) a) or i) b); and ii) a nucleic acid encoding a Staphylococcus aureus Cas9 (SaCas9). In some embodiments, the method further comprises administering a DNA-PK inhibitor.

In some embodiments, a method of treating Myotonic Dystrophy Type 1 (DM1) is provided, the method comprising delivering to a cell a single nucleic acid molecule comprising: i) a nucleic acid encoding a pair of guide RNAs comprising: a) a first and second spacer sequence selected from any one of SEQ ID NOs: 4 and 12; 2 and 12; 3 and 12; 4 and 20; 4 and 18; 2 and 10; 4 and 10; 1 and 12; 8 and 12; 4 and 13; 7 and 12; 7 and 28; 7 and 18; b) a first and second spacer sequence comprising at least 17, 18, 19, 20, or 21 contiguous nucleotides of any of the first and second spacer sequences of i) a); or c) a first and second spacer sequence that is at least 90% identical to any of the first and second spacer sequences of i) a) or i) b); and ii) a nucleic acid encoding a Staphylococcus aureus Cas9 (SaCas9). In some embodiments, the first and second spacer sequence are SEQ ID NOs: 4 and 12. In some embodiments, the first and second spacer sequence are SEQ ID NOs: 2 and 12. In some embodiments, the first and second spacer sequence are SEQ ID NOs: 3 and 12. In some embodiments, the first and second spacer sequence are SEQ ID NOs: 4 and 20. In some embodiments, the first and second spacer sequence are SEQ ID NOs: 4 and 18. In some embodiments, the first and second spacer sequence are SEQ ID NOs: 2 and 10. In some embodiments, the first and second spacer sequence are SEQ ID NOs: 4 and 10. In some embodiments, the first and second spacer sequence are SEQ ID NOs: 1 and 12. In some embodiments, the first and second spacer sequence are SEQ ID NOs: 8 and 12. In some embodiments, the first and second spacer sequence are SEQ ID NOs: 4 and 13. In some embodiments, the first and second spacer sequence are SEQ ID NOs: 7 and 12. In some embodiments, the first and second spacer sequence are SEQ ID NOs: 7 and 28. In some embodiments, the first and second spacer sequence are SEQ ID NOs: 7 and 18. In some embodiments, the method further comprises administering a DNA-PK inhibitor.

In some embodiments, a method of treating Myotonic Dystrophy Type 1 (DM1) is provided, the method comprising delivering to a cell a single nucleic acid molecule comprising: i) a nucleic acid encoding a pair of guide RNAs comprising: a) a first and second spacer sequence selected from any one of SEQ ID NOs: 7 and 12; 4 and 12; or 7 and 23; b) a first and second spacer sequence comprising at least 17, 18, 19, 20, or 21 contiguous nucleotides of any of the first and second spacer sequences of i) a); or c) a first and second spacer sequence that is at least 90% identical to any of the first and second spacer sequences of i) a) or i) b); and ii) a nucleic acid encoding a Staphylococcus aureus Cas9 (SaCas9). In some embodiments, the first and second spacer sequence are SEQ ID NOs: 7 and 12. In some embodiments, the first and second spacer sequence are SEQ ID NOs: 4 and 12. In some embodiments, the first and second spacer sequence are SEQ ID NOs: 7 and 23. In some embodiments, the method further comprises administering a DNA-PK inhibitor.

In some embodiments, a method of excising a CTG repeat in the 3′ UTR of the DMPK gene is provided, the method comprising delivering to a cell a single nucleic acid molecule comprising: 1) a nucleic acid molecule comprising: a nucleic acid encoding one or more spacer sequences selected from SEQ ID NOs: 1-8, 10-28, or 101-154; a nucleic acid encoding one or more spacer sequences comprising at least 17, 18, 19, 20, or 21 contiguous nucleotides of a spacer sequence selected from SEQ ID NOs: 1-8, 10-28, or 101-154; or a nucleic acid encoding one or more spacer sequences that are at least 90% identical to any one of SEQ ID NOs: 1-8, 10-28, or 101-154; and 2) a Staphylococcus aureus Cas9 (SaCas9) or a nucleic acid encoding SaCas9. In some embodiments, the method further comprises administering a DNA-PK inhibitor. In some embodiments, the DNA-PK inhibitor is Compound 1. In some embodiments, the DNA-PK inhibitor is Compound 2. In some embodiments, the DNA-PK inhibitor is Compound 6.

In some embodiments, a method of excising a CTG repeat in the 3′ UTR of the DMPK gene is provided, the method comprising delivering to a cell a single nucleic acid molecule comprising: 1) a nucleic acid molecule comprising: a nucleic acid encoding one or more spacer sequences selected from SEQ ID NOs: 1, 2, 3, 4, 7, 8, 12, or 20; a nucleic acid encoding one or more spacer sequences comprising at least 17, 18, 19, 20, or 21 contiguous nucleotides of a spacer sequence selected from SEQ ID NOs: 1, 2, 3, 4, 7, 8, 12, or 20; or a nucleic acid encoding one or more spacer sequences that are at least 90% identical to any one of SEQ ID NOs: 1, 2, 3, 4, 7, 8, 12, or 20; and 2) a Staphylococcus aureus Cas9 (SaCas9) or a nucleic acid encoding SaCas9. In some embodiments, the spacer sequence is SEQ ID NO: 1. In some embodiments, the spacer sequence is SEQ ID NO: 2. In some embodiments, the spacer sequence is SEQ ID NO: 3. In some embodiments, the spacer sequence is SEQ ID NO: 4. In some embodiments, the spacer sequence is SEQ ID NO: 7. In some embodiments, the spacer sequence is SEQ ID NO: 8. In some embodiments, the spacer sequence is SEQ ID NO: 12. In some embodiments, the spacer sequence is SEQ ID NO: 20. In some embodiments, the method further comprises administering a DNA-PK inhibitor.

In some embodiments, a method of excising a CTG repeat in the 3′ UTR of the DMPK gene is provided, the method comprising delivering to a cell a single nucleic acid molecule comprising: 1) a nucleic acid molecule comprising: a nucleic acid encoding one or more spacer sequences selected from SEQ ID NOs: 1, 101, and 102; a nucleic acid encoding one or more spacer sequences comprising at least 17, 18, 19, 20, or 21 contiguous nucleotides of a spacer sequence selected from SEQ ID NOs: 1, 101, and 102; or a nucleic acid encoding one or more spacer sequences that are at least 90% identical to any one of SEQ ID NOs: 1, 101, and 102; and 2) a Staphylococcus aureus Cas9 (SaCas9) or a nucleic acid encoding SaCas9. In some embodiments, the spacer sequence is SEQ ID NO: 1. In some embodiments, the spacer sequence is SEQ ID NO: 101. In some embodiments, the spacer sequence is SEQ ID NO: 102. In some embodiments, the method further comprises administering a DNA-PK inhibitor.

In some embodiments, only one guide RNA is administered and a CTG repeat in the 3′ UTR is excised. In some embodiments, a pair of guide RNAs is administered and a CTG repeat in the 3′ UTR is excised.

In some embodiments, a method of excising a CTG repeat in the 3′ UTR of the DMPK gene is provided, the method comprising delivering to a cell a single nucleic acid molecule comprising: 1) a nucleic acid molecule encoding a pair of guide RNAs comprising: a) a first and second spacer sequence selected from any one of SEQ ID NOs: 1 and 10; 1 and 11; 1 and 12; 1 and 13; 1 and 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28; b) a first and second spacer sequence comprising at least 17, 18, 19, 20, or 21 contiguous nucleotides of a spacer sequence selected from any one of 1) a); or c) a first and second spacer sequence that is at least 90% identical to any one of 1) a) or 1) b); and 2) a Staphylococcus aureus Cas9 (SaCas9) or a nucleic acid encoding SaCas9. In some embodiments, the method further comprises administering a DNA-PK inhibitor. In some embodiments, the DNA-PK inhibitor is Compound 1. In some embodiments, the DNA-PK inhibitor is Compound 2. In some embodiments, the DNA-PK inhibitor is Compound 6.

In some embodiments, a method of excising a CTG repeat in the 3′ UTR of the DMPK gene is provided, the method comprising delivering to a cell a single nucleic acid molecule comprising: 1) a nucleic acid molecule encoding a pair of guide RNAs comprising: a) a first and second spacer sequence selected from any one of SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 8 and 10; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 7 and 10; 1 and 13; 1 and 20; 1 and 28; 4 and 27; 7 and 20; 7 and 23; 7 and 13; 7 and 28; 2 and 27; 8 and 27; 4 and 11; 4 and 25; 4 and 28; 4 and 19; 4 and 15; 8 and 11; 3 and 27; 2 and 25; 2 and 11; 7 and 18; 3 and 25; 8 and 15; 8 and 25; 3 and 11; 3 and 19; 1 and 15; 3 and 15; 1 and 27; 2 and 15; 2 and 19; 1 and 11; 1 and 25; 8 and 19; 4 and 21; 8 and 21; 7 and 27; 7 and 15; land 19; 2 and 21; 7 and 11; 3 and 21; 4 and 14; 7 and 19; 4 and 26; 8 and 26; 7 and 25; 1 and 21; 3 and 26; 2 and 26; 8 and 14; 1 and 14; 2 and 14; 3 and 14; 1 and 26; 7 and 21; 7 and 14; and 7 and 26; b) a first and second spacer sequence comprising at least 17, 18, 19, 20, or 21 contiguous nucleotides of a spacer sequence selected from any one of 1) a); or c) a first and second spacer sequence that is at least 90% identical to any one of 1) a) or 1) b); and 2) a Staphylococcus aureus Cas9 (SaCas9) or a nucleic acid encoding SaCas9. In some embodiments, the method further comprises administering a DNA-PK inhibitor.

In some embodiments, a method of excising a CTG repeat in the 3′ UTR of the DMPK gene is provided, the method comprising delivering to a cell a single nucleic acid molecule comprising: 1) a nucleic acid molecule encoding a pair of guide RNAs comprising: a) a first and second spacer sequence selected from any one of SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 8 and 10; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 7 and 10; 1 and 13; 1 and 20; 1 and 28; 4 and 27; 7 and 20; 7 and 23; 7 and 13; 7 and 28; 2 and 27; 8 and 27; and 4 and 11; b) a first and second spacer sequence comprising at least 17, 18, 19, 20, or 21 contiguous nucleotides of a spacer sequence selected from any one of 1) a); or c) a first and second spacer sequence that is at least 90% identical to any one of 1) a) or 1) b); and 2) a Staphylococcus aureus Cas9 (SaCas9) or a nucleic acid encoding SaCas9. In some embodiments, the method further comprises administering a DNA-PK inhibitor.

In some embodiments, a method of excising a CTG repeat in the 3′ UTR of the DMPK gene is provided, the method comprising delivering to a cell a single nucleic acid molecule comprising: 1) a nucleic acid molecule encoding a pair of guide RNAs comprising: a) a first and second spacer sequence selected from any one of SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 1 and 13; 1 and 20; 1 and 28; 4 and 27; 7 and 20; 7 and 23; 7 and 13; 7 and 28; 2 and 27; 4 and 11; 4 and 25; 4 and 28; 4 and 19; 4 and 15; 8 and 11; 3 and 27; 2 and 25; 2 and 11; 7 and 18; 3 and 25; 8 and 15; 3 and 11; 3 and 19; 1 and 15; 3 and 15; 1 and 27; 2 and 15; 2 and 19; 1 and 11; 1 and 25; 4 and 21; 8 and 21; 7 and 27; 7 and 15; land 19; 2 and 21; 7 and 11; 3 and 21; 7 and 19; 7 and 25; 1 and 21; 3 and 26; 3 and 14; 7 and 21; and 7 and 14; b) a first and second spacer sequence comprising at least 17, 18, 19, 20, or 21 contiguous nucleotides of a spacer sequence selected from any one of 1) a); or c) a first and second spacer sequence that is at least 90% identical to any one of 1) a) or 1) b); and 2) a Staphylococcus aureus Cas9 (SaCas9) or a nucleic acid encoding SaCas9. In some embodiments, the method further comprises administering a DNA-PK inhibitor.

In some embodiments, a method of excising a CTG repeat in the 3′ UTR of the DMPK gene is provided, the method comprising delivering to a cell a single nucleic acid molecule comprising: 1) a nucleic acid molecule encoding a pair of guide RNAs comprising: a) a first and second spacer sequence selected from any one of SEQ ID NOs: 4 and 12; 2 and 10; 4 and 28; 8 and 12; 4 and 13; 3 and 10; 7 and 12; 7 and 13; 4 and 28; and 7 and 18; b) a first and second spacer sequence comprising at least 17, 18, 19, 20, or 21 contiguous nucleotides of a spacer sequence selected from any one of 1) a); or c) a first and second spacer sequence that is at least 90% identical to any one of 1) a) or 1) b); and 2) a Staphylococcus aureus Cas9 (SaCas9) or a nucleic acid encoding SaCas9. In some embodiments, the method further comprises administering a DNA-PK inhibitor.

In some embodiments, a method of excising a CTG repeat in the 3′ UTR of the DMPK gene is provided, the method comprising delivering to a cell a single nucleic acid molecule comprising: i) a nucleic acid encoding a pair of guide RNAs comprising: a) a first and second spacer sequence selected from any one of SEQ ID NOs: 4 and 12; 2 and 12; 3 and 12; 4 and 20; 4 and 18; 2 and 10; 4 and 10; 1 and 12; 8 and 12; 4 and 13; 7 and 12; 7 and 28; 7 and 18; b) a first and second spacer sequence comprising at least 17, 18, 19, 20, or 21 contiguous nucleotides of any of the first and second spacer sequences of i) a); or c) a first and second spacer sequence that is at least 90% identical to any of the first and second spacer sequences of i) a) or i) b); and ii) a nucleic acid encoding a Staphylococcus aureus Cas9 (SaCas9). In some embodiments, the first and second spacer sequence are SEQ ID NOs: 4 and 12. In some embodiments, the first and second spacer sequence are SEQ ID NOs: 2 and 12. In some embodiments, the first and second spacer sequence are SEQ ID NOs: 3 and 12. In some embodiments, the first and second spacer sequence are SEQ ID NOs: 4 and 20. In some embodiments, the first and second spacer sequence are SEQ ID NOs: 4 and 18. In some embodiments, the first and second spacer sequence are SEQ ID NOs: 2 and 10. In some embodiments, the first and second spacer sequence are SEQ ID NOs: 4 and 10. In some embodiments, the first and second spacer sequence are SEQ ID NOs: 1 and 12. In some embodiments, the first and second spacer sequence are SEQ ID NOs: 8 and 12. In some embodiments, the first and second spacer sequence are SEQ ID NOs: 4 and 13. In some embodiments, the first and second spacer sequence are SEQ ID NOs: 7 and 12. In some embodiments, the first and second spacer sequence are SEQ ID NOs: 7 and 28. In some embodiments, the first and second spacer sequence are SEQ ID NOs: 7 and 18. In some embodiments, the method further comprises administering a DNA-PK inhibitor.

In some embodiments, a method of excising a CTG repeat in the 3′ UTR of the DMPK gene is provided, the method comprising delivering to a cell a single nucleic acid molecule comprising: 1) a nucleic acid molecule encoding a pair of guide RNAs comprising: a) a first and second spacer sequence selected from any one of SEQ ID NOs: 7 and 12, 4 and 12, and 7 and 23; b) a first and second spacer sequence comprising at least 17, 18, 19, 20, or 21 contiguous nucleotides of a spacer sequence selected from any one of 1) a); or c) a first and second spacer sequence that is at least 90% identical to any one of 1) a) or 1) b); and 2) a Staphylococcus aureus Cas9 (SaCas9) or a nucleic acid encoding SaCas9. In some embodiments, the first and second spacer sequence are SEQ ID NOs: 7 and 12. In some embodiments, the first and second spacer sequence are SEQ ID NOs: 4 and 12. In some embodiments, the first and second spacer sequence are SEQ ID NOs: 7 and 23. In some embodiments, the method further comprises administering a DNA-PK inhibitor.

In some embodiments, the methods comprise delivering to a cell a nucleic acid molecule encoding SaCas9, wherein the SaCas9 comprises the amino acid sequence of SEQ ID NO: 711. In some embodiments, the methods comprise delivering to a cell a nucleic acid molecule encoding SaCas9, wherein the SaCas9 is a variant of the amino acid sequence of SEQ ID NO: 711. In some embodiments, the methods comprise delivering to a cell a nucleic acid molecule encoding SaCas9, wherein the SaCas9 comprises an amino acid sequence selected from any one of SEQ ID NOs: 715-717.

In some embodiments, the subject is a mammal. In some embodiments, the subject is human.

IV. DNA-PK Inhibitor

Where a DNA-PK inhibitor is used in a composition or method disclosed herein, it may be any DNA-PK inhibitor known in the art. DNA-PK inhibitors are discussed in detail, for example, in WO2014/159690; WO2013/163190; WO2018/013840; WO2019/143675; WO 2019/143677; WO 2019/143678; US2014275059; US2013281431; US2020361877; US2020353101 and Robert et al., Genome Medicine (2015) 7:93, each of which are incorporated by reference herein. In some embodiments, the DNA-PK inhibitor is NU7441, KU-0060648, or any one of Compounds 1, 2, 3, 4, 5, or 6 (structures shown below), each of which is also described in at least one of the foregoing citations. In some embodiments, the DNA-PK inhibitor is Compound 1. In some embodiments, the DNA-PK inhibitor is Compound 2. In some embodiments, the DNA-PK inhibitor is Compound 6. In some embodiments, the DNA-PK inhibitor is Compound 3. Structures for exemplary DNA-PK inhibitors are as follows. Unless otherwise indicated, reference to a DNA-PK inhibitor by name or structure encompasses pharmaceutically acceptable salts thereof

| DNA-PK Inhibitor | Structure |
|---|---|
| NU7441 | 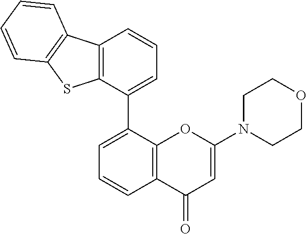 [SMILES: O=c1cc(N2CCOCC2)oc2c(-c3cccc4c3sc3ccccc34)cccc12] |
| KU-0060648 | 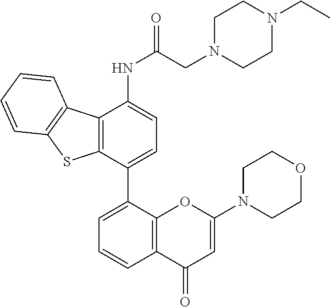 [SMILES: CCN1CCN(CC(=O)Nc2ccc(-c3cccc4c(=O)cc(N5CCOCC5)oc34)c3sc4ccccc4c23)CC1] |
| Compound 1 | 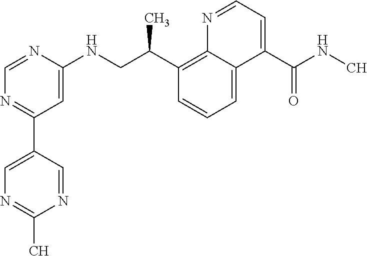 [SMILES: CNC(=O)c1ccnc2c([C@H](C)CNc3cc(-c4cnc(C)nc4)ncn3)cccc12] |
| Compound 2 | 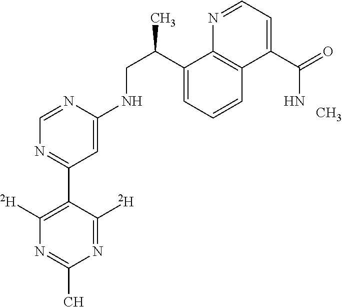 |
| Compound 3 | 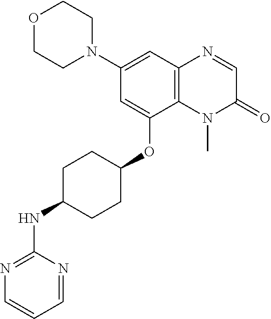 [SMILES: Cn1c(=O)cnc2cc(N3CCOCC3)cc(O[C@H]3CC[C@@H](Nc4ncccn4)CC3)c21] |
| Compound 4 | 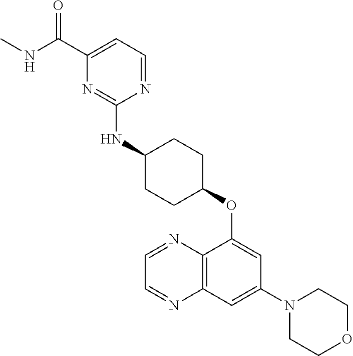 [SMILES: CNC(=O)c1ccnc(N[C@H]2CC[C@@H](Oc3cc(N4CCOCC4)cc4nccnc34)CC2)n1] |
| Compound 5 | 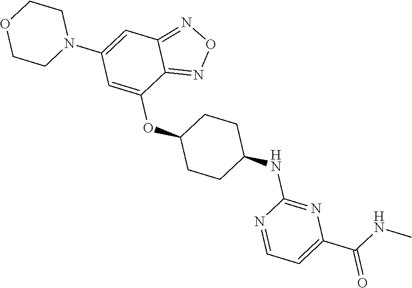 [SMILES: CNC(=O)c1ccnc(N[C@H]2CC[C@@H](Oc3cc(N4CCOCC4)cc4nonc34)CC2)n1] |
| Compound 6 | 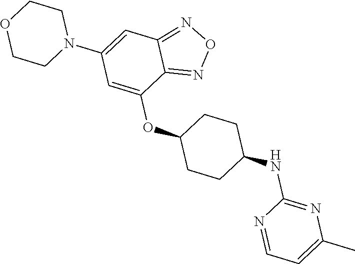 [SMILES: Cc1ccnc(N[C@H]2CC[C@@H](Oc3cc(N4CCOCC4)cc4nonc34)CC2)n1] |

In any of the foregoing embodiments where a DNA-PK inhibitor is used, it may be used in combination with only one gRNA or vector encoding only one gRNA to promote excision, i.e., the method does not always involve providing two or more guides that promote cleavage near a CTG repeat.

In some embodiments where a DNA-PK inhibitor is used, it may be used in combination with a pair of gRNAs or vector encoding a pair of guide RNAs to promote excision. In some embodiments, the pair of gRNAs comprise gRNAs that are not the same. In particular embodiments, the pair of gRNAs together target sequences that flank a CTG repeat region in the genome of a cell.

V. Combination Therapy

In some embodiments, the invention comprises combination therapies comprising any of the methods or uses described herein together with an additional therapy suitable for ameliorating DM1.

VI. Delivery of Guide RNA Compositions

The methods and uses disclosed herein may use any suitable approach for delivering the guide RNAs and compositions described herein. Exemplary delivery approaches include vectors, such as viral vectors; lipid nanoparticles; transfection; and electroporation. In some embodiments, vectors or LNPs associated with the single-vector guide RNAs/Cas9's disclosed herein are for use in preparing a medicament for treating DM1.

Where a vector is used, it may be a viral vector, such as a non-integrating viral vector. In some embodiments, the viral vector is an adeno-associated virus vector, a lentiviral vector, an integrase-deficient lentiviral vector, an adenoviral vector, a vaccinia viral vector, an alphaviral vector, or a herpes simplex viral vector. In some embodiments, the viral vector is an adeno-associated virus (AAV) vector. In some embodiments, the AAV vector is an AAV1, AAV2, AAV3, AAV4, AAVS, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of US 2015/0111955, which is incorporated by reference herein in its entirety), or AAV9 vector, wherein the number following AAV indicates the AAV serotype. Any variant of an AAV vector or serotype thereof, such as a self-complementary AAV (scAAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors.

In some embodiments, the vector (e.g., viral vector, such as an adeno-associated viral vector) comprises a tissue-specific (e.g., muscle-specific) promoter, e.g., which is operatively linked to a sequence encoding the guide RNA. In some embodiments, the muscle-specific promoter is a muscle creatine kinase promoter, a desmin promoter, an MHCK7 promoter, or an SPc5-12 promoter. In some embodiments, the muscle-specific promoter is a CK8 promoter. In some embodiments, the muscle-specific promoter is a CK8e promoter. Muscle-specific promoters are described in detail, e.g., in US2004/0175727 A1; Wang et al., Expert Opin Drug Deliv. (2014) 11, 345-364; Wang et al., Gene Therapy (2008) 15, 1489-1499. In some embodiments, the tissue-specific promoter is a neuron-specific promoter, such as an enolase promoter. See, e.g., Naso et al., BioDrugs 2017; 31:317-334; Dashkoff et al., Mol Ther Methods Clin Dev. 2016; 3:16081, and references cited therein for detailed discussion of tissue-specific promoters including neuron-specific promoters.

In some embodiments, in addition to guide RNA and Cas9 sequences, the vectors further comprise nucleic acids that do not encode guide RNAs. Nucleic acids that do not encode guide RNA and Cas9 include, but are not limited to, promoters, enhancers, and regulatory sequences. In some embodiments, the vector comprises one or more nucleotide sequence(s) encoding a crRNA, a trRNA, or a crRNA and trRNA.

Lipid nanoparticles (LNPs) are a known means for delivery of nucleotide and protein cargo, and may be used for delivery of the guide RNAs, compositions, or pharmaceutical formulations disclosed herein. In some embodiments, the LNPs deliver nucleic acid, protein, or nucleic acid together with protein.

Electroporation is a well-known means for delivery of cargo, and any electroporation methodology may be used for delivering the single vectors disclosed herein.

In some embodiments, the invention comprises a method for delivering any one of the single vectors disclosed herein to an ex vivo cell, wherein the guide RNA is encoded by a vector, associated with an LNP, or in aqueous solution. In some embodiments, the guide RNA/LNP or guide RNA is also associated with a Cas9 or sequence encoding Cas9 (e.g., in the same vector, LNP, or solution).

### EXAMPLES

The following examples are provided to illustrate certain disclosed embodiments and are not to be construed as limiting the scope of this disclosure in any way.

### Example 1: Evaluation of DM1 sgRNAs

A. Materials and Methods

1. sgRNA Selection

The 3′ UTR of the human DMPK gene was scanned for the canonical NNGRRT PAM sequence on either the sense or antisense strand, and 28 sgRNA protospacer sequences (22-nucleotide in length) adjacent to the canonical PAMs were identified (Table 1A). 27 sgRNAs were selected for evaluation in primary DM1 patient myoblasts based on in silico off-target assessment. Further exemplary guide sequences are shown in Table 1B.

TABLE 1A
SaCas9 sgRNAs with the canonical NNGRRT PAM sequences in the 3′ UTR region of
human DMPK gene

| | | | | | | | | | Pre- |
|---|---|---|---|---|---|---|---|---|---|
| | | | | | | | | | dicted |
| | | | | | | | | | off- |
| SaCas9 | SEQ | | | Proto- | Proto- | PAM | | | target |
| sgRNA | ID | | Protospacer sequence | spacer | spacer | se- | PAM | PAM | site |
| name | NO | Strand | (22bp) | start | end | quence | start | end | number |
| SaU1 | 1 | + | GCCCCGGAGTCGAAGACAGTTC | 45770460 | 45770481 | TAGGGT | 45770482 | 45770487 | 3 |
| SaU2 | 2 | + | ACTCAGTCTTCCAACGGGGCCC | 45770442 | 45770463 | CGGAGT | 45770464 | 45770469 | 2 |
| SaU3 | 3 | − | ACTCCGGGGCCCCGTTGGAAGA | 45770448 | 45770469 | CTGAGT | 45770442 | 45770447 | 3 |
| SaU4 | 4 | − | CCAGTTCACAACCGCTCCGAGC | 45770383 | 45770404 | GTGGGT | 45770377 | 45770382 | 0 |
| SaU5 | 5 | + | CCCCGGCCGCTAGGGGGGGGC | 45770326 | 45770347 | CCGGAT | 45770348 | 45770353 | 8 |
| SaU6 | 6 | − | CTAGCGGCCGGGGAGGGAGGGG | 45770317 | 45770338 | CCGGGT | 45770311 | 45770316 | 28 |
| SaU7 | 7 | − | CGCGGCCGGCGAACGGGGCTCG | 45770288 | 45770309 | AAGGGT | 45770282 | 45770287 | 9 |
| SaU8 | 8 | − | GGCTCGAAGGGTCCTTGTAGCC | 45770272 | 45770293 | GGGAAT | 45770266 | 45770271 | 2 |
| SaD1* | 9 | − | ctgctgctgctgctgctgctgG | 45770204 | 45770225 | GGGGAT | 45770198 | 45770203 | 1123 |
| SaD2 | 10 | − | TCGGCCAGGCTGAGGCCCTGAC | 45770157 | 45770178 | GTGGAT | 45770151 | 45770156 | 6 |
| SaD3 | 11 | + | CAACGATAGGTGGGGGTGCGTG | 45770074 | 45770095 | GAGGAT | 45770096 | 45770101 | 1 |
| SaD4 | 12 | + | ACTTTGCGAACCAACGATAGGT | 45770063 | 45770084 | GGGGGT | 45770085 | 45770090 | 2 |
| SaD5 | 13 | + | GGGTTTGGCAAAAGCAAATTTC | 45769972 | 45769993 | CCGAGT | 45769994 | 45769999 | 5 |
| SaD6 | 14 | − | CTTTTGCCAAACCCGCTTTTTC | 45769964 | 45769985 | GGGGAT | 45769958 | 45769963 | 5 |
| SaD7 | 15 | + | GGGGCGCGGGATCCCCGAAAAA | 45769948 | 45769969 | GCGGGT | 45769970 | 45769975 | 0 |
| SaD8 | 16 | + | AGCGCAAGTGAGGAGGGGGGCG | 45769932 | 45769953 | CGGGAT | 45769954 | 45769959 | 10 |
| SaD9 | 17 | − | CGGCTCCGCCCGCTTCGGCGGT | 45769893 | 45769914 | TTGGAT | 45769887 | 45769892 | 4 |
| SaD10 | 18 | + | TTGGGGGTCCTGTAGCCTGTCA | 45769838 | 45769859 | GCGAGT | 45769860 | 45769865 | 4 |
| SaD11 | 19 | + | CAAAACGTGGATTGGGGTTGTT | 45769818 | 45769839 | GGGGGT | 45769840 | 45769845 | 2 |
| SaD12 | 20 | + | TCAGTGCATCCAAAACGTGGAT | 45769808 | 45769829 | TGGGGT | 45769830 | 45769835 | 1 |
| SaD13 | 21 | − | CCCAACAACCCCAATCCACGTT | 45769821 | 45769842 | TTGGAT | 45769815 | 45769820 | 7 |
| SaD14 | 22 | + | GGGGTCTCAGTGCATCCAAAAC | 45769802 | 45769823 | GTGGAT | 45769824 | 45769829 | 0 |
| SaD15 | 23 | + | GACAATAAATACCGAGGAATGT | 45769779 | 45769800 | CGGGGT | 45769801 | 45769806 | 4 |
| SaD16 | 24 | + | TGGGGACAGACAATAAATACCG | 45769771 | 45769792 | AGGAAT | 45769793 | 45769798 | 8 |
| SaD17 | 25 | + | TTTATTCGCGAGGGTCGGGGGT | 45769737 | 45769758 | GGGGGT | 45769759 | 45769764 | 2 |
| SaD18 | 26 | + | GGGCCTTTTATTCGCGAGGGTC | 45769731 | 45769752 | GGGGGT | 45769753 | 45769758 | 13 |
| SaD19 | 27 | − | AGGACCCCCACCCCCGACCCTC | 45769746 | 45769767 | GCGAAT | 45769740 | 45769745 | 2 |
| SaD20 | 28 | + | AGATGGAGGGCCTTTTATTCGC | 45769724 | 45769745 | GAGGGT | 45769746 | 45769751 | 3 |

*SaD1 was not evaluated in primary DM1 patient myoblasts due to its high number of predicted OFF-target sites.

TABLE 1B
Exemplary SaCas9 sgRNAs with the
canonical NNGRRT PAM sequences
in the 3′ UTR region of human
DMPK gene

| SEQ | | |
|---|---|---|
| ID | | |
| NO | Strand | Guide Sequence |
| 1 | + | GCCCCGGAGTCGAAGACAGTTC |
| 101 | + | CCCCGGAGTCGAAGACAGTTC |
| 102 | + | CCCGGAGTCGAAGACAGTTC |
| 2 | + | ACTCAGTCTTCCAACGGGGCCC |
| 103 | + | CTCAGTCTTCCAACGGGGCCC |
| 104 | + | TCAGTCTTCCAACGGGGCCC |
| 3 | − | ACTCCGGGGCCCCGTTGGAAGA |
| 105 | − | ACTCCGGGGCCCCGTTGGAAG |
| 106 | − | ACTCCGGGGCCCCGTTGGAA |
| 4 | − | CCAGTTCACAACCGCTCCGAGC |
| 107 | − | CCAGTTCACAACCGCTCCGAG |
| 108 | − | CCAGTTCACAACCGCTCCGA |
| 5 | + | CCCCGGCCGCTAGGGGGCGGGC |
| 109 | + | CCCGGCCGCTAGGGGGCGGGC |
| 110 | + | CCGGCCGCTAGGGGGCGGGC |
| 6 | − | CTAGCGGCCGGGGAGGGAGGGG |
| 111 | − | CTAGCGGCCGGGGAGGGAGGG |
| 112 | − | CTAGCGGCCGGGGAGGGAGG |
| 7 | − | CGCGGCCGGCGAACGGGGCTCG |
| 113 | − | CGCGGCCGGCGAACGGGGCTC |
| 114 | − | CGCGGCCGGCGAACGGGGCT |
| 8 | − | GGCTCGAAGGGTCCTTGTAGCC |
| 115 | − | GGCTCGAAGGGTCCTTGTAGC |
| 116 | − | GGCTCGAAGGGTCCTTGTAG |
| 10 | − | TCGGCCAGGCTGAGGCCCTGAC |
| 117 | − | TCGGCCAGGCTGAGGCCCTGA |
| 118 | − | TCGGCCAGGCTGAGGCCCTG |
| 11 | + | CAACGATAGGTGGGGGTGCGTG |
| 119 | + | AACGATAGGTGGGGGTGCGTG |
| 120 | + | ACGATAGGTGGGGGTGCGTG |
| 12 | + | ACTTTGCGAACCAACGATAGGT |
| 121 | + | CTTTGCGAACCAACGATAGGT |
| 122 | + | TTTGCGAACCAACGATAGGT |
| 13 | + | GGGTTTGGCAAAAGCAAATTTC |
| 123 | + | GGTTTGGCAAAAGCAAATTTC |
| 124 | + | GTTTGGCAAAAGCAAATTTC |
| 14 | − | CTTTTGCCAAACCCGCTTTTTC |
| 125 | − | CTTTTGCCAAACCCGCTTTTT |
| 126 | − | CTTTTGCCAAACCCGCTTTT |
| 15 | + | GGGGCGCGGGATCCCCGAAAAA |
| 127 | + | GGGCGCGGGATCCCCGAAAAA |
| 128 | + | GGCGCGGGATCCCCGAAAAA |
| 16 | + | AGCGCAAGTGAGGAGGGGGGCG |
| 129 | + | GCGCAAGTGAGGAGGGGGGCG |
| 130 | + | CGCAAGTGAGGAGGGGGGCG |
| 17 | − | CGGCTCCGCCCGCTTCGGCGGT |
| 131 | − | CGGCTCCGCCCGCTTCGGCGG |
| 132 | − | CGGCTCCGCCCGCTTCGGCG |
| 18 | + | TTGGGGGTCCTGTAGCCTGTCA |
| 133 | + | TGGGGGTCCTGTAGCCTGTCA |
| 134 | + | GGGGGTCCTGTAGCCTGTCA |
| 19 | + | CAAAACGTGGATTGGGGTTGTT |
| 135 | + | AAAACGTGGATTGGGGTTGTT |
| 136 | + | AAACGTGGATTGGGGTTGTT |
| 20 | + | TCAGTGCATCCAAAACGTGGAT |
| 137 | + | CAGTGCATCCAAAACGTGGAT |
| 138 | + | AGTGCATCCAAAACGTGGAT |
| 21 | − | CCCAACAACCCCAATCCACGTT |
| 139 | − | CCCAACAACCCCAATCCACGT |
| 140 | − | CCCAACAACCCCAATCCACG |
| 22 | + | GGGGTCTCAGTGCATCCAAAAC |
| 141 | + | GGGTCTCAGTGCATCCAAAAC |
| 142 | + | GGTCTCAGTGCATCCAAAAC |
| 23 | + | GACAATAAATACCGAGGAATGT |
| 143 | + | ACAATAAATACCGAGGAATGT |
| 144 | + | CAATAAATACCGAGGAATGT |
| 24 | + | TGGGGACAGACAATAAATACCG |
| 145 | + | GGGGACAGACAATAAATACCG |
| 146 | + | GGGACAGACAATAAATACCG |
| 25 | + | TTTATTCGCGAGGGTCGGGGGT |
| 147 | + | TTATTCGCGAGGGTCGGGGGT |
| 148 | + | TATTCGCGAGGGTCGGGGGT |
| 26 | + | GGGCCTTTTATTCGCGAGGGTC |
| 149 | + | GGCCTTTTATTCGCGAGGGTC |
| 150 | + | GCCTTTTATTCGCGAGGGTC |
| 27 | − | AGGACCCCCACCCCCGACCCTC |
| 151 | − | AGGACCCCCACCCCCGACCCT |
| 152 | − | AGGACCCCCACCCCCGACCC |
| 28 | + | AGATGGAGGGCCTTTTATTCGC |
| 153 | + | GATGGAGGGCCTTTTATTCGC |
| 154 | + | ATGGAGGGCCTTTTATTCGC |

2. In Silico Off-Target Assessment

Off-target sites were computationally predicted for each sgRNA based on sequence similarity to the hg38 human reference genome, specifically, any site that was identified to have a PAM sequence and have up to 3 mismatches, or up to 2 mismatches and 1 DNA/RNA bulge, relative to the protospacer sequence.

3. Genomic DNA Extraction, PCR Amplification and TapeStation

Genomic DNA of DM1 myoblasts was isolated with the Kingfisher Flex purification system (Thermal Fisher) in 96-well format following the manufacturer's instruction. The DMPK 3′ UTR region was amplified using GoTaq Green Master Mix (Promega) and PCR primers flanking the 3′ UTR region. The forward primer sequence was CGCTAGGAAGCAGCCAATGA (SEQ ID NO: 723), and the reverse primer sequence was TAGCTCCTCCCAGACCTTCG (SEQ ID NO: 724). Amplification was conducted using the following cycling parameters: 1 cycle at 95° C. for 2 min; 40 cycles of 95° C. for 30 sec, 63° C. for 30 sec, and 72° C. for 90 sec; 1 cycle at 72° C. for 5 min. Only the wild type allele was amplified by the PCR reaction. The PCR products were analyzed on the Tape Station system with High Sensitivity D5000 ScreenTape (Agilent Technologies).

4. Sanger Sequencing and ICE Analysis

PCR products were sent to GeneWiz for purification and Sanger sequencing. Sequencing primer UTRsF3 (AATGACGAGTTCGGACGG) (SEQ ID NO: 725) was used for sequencing upstream sgRNAs, and the reverse PCR primer (TAGCTCCTCCCAGACCTTCG) (SEQ ID NO: 724) was used for sequencing downstream sgRNAs. Indel values were estimated using the ICE analysis algorithm (Synthego) with the chromatogram files obtained from Sanger sequencing.

5. Primary Myoblast Culture

Primary healthy myoblasts (P01431-18F) and DM1 patient myoblasts (03001-32F) were obtained from Cook MyoSite. Myoblasts were cultured in myoblast growth medium consisting of Myotonic Basal Medium (Cook MyoSite, MB-2222) and MyoTonic Growth Supplement (Cook MyoSite, MS-3333). Three days before nucleofection, primary human myoblasts were further purified with EasySep Human CD56 Positive Selection Kit II (StemCell Tech, 17855) following the manufacturer's instruction, and then maintained in myoblast growth medium until nucleofection.

6. Preparation of RNPs

RNPs were assembled with recombinant SaCas9 protein (Aldevron) and chemically modified sgRNAs (Synthego) at a ratio of 1:3 (protein:sgRNA). For SINGLE-cut screening, RNP complexes were assembled with 30 pmol of SaCas9 and 90 pmol of sgRNA in P5 Primary Cell Nucleofector Solution (Lonza). After incubation at room temperature for 20 minutes, 10 μL of RNP complex was mixed with two hundred thousand primary myoblasts resuspended in 10 μL of P5 Nucleofector Solution. For DOUBLE-cut screening, RNP complexes were first assembled for individual sgRNAs with 20 pmol of SaCas9 protein and 60 pmol of sgRNAs in 5 μL of P5 Nucleofector Solution. After incubation at room temperature for 20 minutes, the two RNP complexes (one for upstream sgRNA and one for downstream sgRNA) were mixed at 1:1 ratio and then further mixed with two hundred thousand primary myoblasts resuspended in 10 μL of P5 Nucleofector Solution.

7. Nucleofection of RNPs into Primary DM1 Myoblasts

The Nucleofector 96-well Shuttle System (Lonza) was used to deliver the SaCa9/sgRNA RNPs into primary DM1 patient myoblasts using the nucleofection program CM138. Following nucleofection, myoblasts from each well of nucleofection shuttle were split into six wells of the 96-well cell culture plate (Greiner, 655090) coated with matrigel. The first three wells were treated with DMSO for 48 hrs before changing to fresh myoblast growth medium, and the other three wells were treated with 3 μM of DNA-PKi Compound 6 for 48 hrs before changing to fresh myoblast growth medium. 72 hrs post nucleofection, two wells of DMSO-treated myoblasts and two wells of DNA-PKi-treated myoblasts from each nucleofection were harvested for genomic DNA extraction using the Kingfisher Flex purification system (Thermal Fisher), whereas one well of DMSO-treated myoblasts and one well of DNA-PKi-treated myoblasts were stained for RNA foci by FISH staining.

8. ddPCR

The primers and probes of ddPCR were designed using the online primer design software Primer3Plus (http://www.bioinformatics.nl/cgi-bin/primer3plus/primer3plus.cgi). The two Target primers/probe sets were used to detect CTG repeat excision, and the Reference primers/probe set was used to amplify a region located in Exon 1 of human DMPK gene and it served as a reference control for the Target sets. The ddPCR primer and probe sequences are listed in Table 2. The 24 μL of ddPCR reaction consisted of 12 μL of Supermix for Probes (no dUTP) (Bio-Rad Laboratories), 1 μL of Reference primers mix (21.6 μM), 1 μL of Reference probe (6 μM), 1 μL of Target primers mix (21.6 μM), 1 μL of Target probe (6 μM), and 8 μL of sample genomic DNA. Droplets were generated using probe oil with the QX200 Droplet Generator (Bio-Rad Laboratories). Droplets were transferred to a 96-well PCR plate, sealed and cycled in a C1000 deep well Thermocycler (Bio-Rad Laboratories) under the following cycling protocol: 1 cycle at 95° C. for 10 min; 40 cycles of 94° C. for 30 sec, and 58° C. for 1 min; 1 cycle at 98° C. for 10 min (for enzyme inactivation). The cycled plate was then transferred and read in the FAM and HEX channels using the Bio-Rad QX200 Droplet Reader (Bio-Rad Laboratories). ddPCR analysis was performed with the Bio-Rad QuantaSoft Pro Software.

TABLE 2
Primer and probe sequences for loss-of-
signal ddPCR assays

| | Oligo | | Sequence |
|---|---|---|---|
| ddPCR set | type | Name | (5′ á 3′) |
| Target_ | Forward | UTRF1 | GGGGATCACAGA |
| Downstream | Primer | | CCATTTCT |
| | Reverse | UTRR14 | TGGAGGATGGAA |
| | Primer | | CACGGAC |
| | Probe | UTRP2-FAM | TTCTTTCGGCCA |
| | | | GGCTGAGGCCCT |
| Target_ | Forward | UpExcisionF | CTAGCGGCCGGG |
| Upstream | Primer | | GAG |
| | Reverse | UpExcisionR | AGCAGCATTCCC |
| | Primer | | GGCTA |
| | Probe | UpExcisionP- | CGAACGGGGCTC |
| | | FAM | GAAGGGTCCTTG |
| Reference | Forward | DMPKF8 | GGATATGTGACC |
| | Primer | | ATGCTACC |
| | Reverse | DMPKR7 | GGGTTGTATCCA |
| | Primer | | GTACCTCT |
| | Probe | DMPKP6- | TGTCCTGTTCCT |
| | | HEX | TCCCCCAGCCCCA |

9. FISH Staining of RNA Foci

Primary myoblasts were fixed for 15 min with 4% paraformaldehyde (PFA) and washed five times with 1×PBS for 10 min each at room temperature. Before staining, cells were permeabilized with 0.5% triton X-100 in 1×PBS for 5 min at room temperature, and then washed with 30% formamide and 2× saline-sodium citrate (SSC) mixture for 10 min at room temperature. Cells were then stained with 1 ng/μL of Cy3-PNA(CAG) 5 probe (PNA Bio, F5001) diluted in 30% formamide, 2×SSC, 2 μg/mL BSA, 66 μg/mL yeast tRNA, and 2 mM vanadyl complex for 15 min at 80° C. Following probe staining, cells were then washed in 30% formamide and 2×SSC mixture for 30 min at 42° C., then washed in 30% formamide and 2×SSC mixture for 30 min at 37° C., and then washed in 1×SSC solution for 10 min at room temperature, and finally washed in 1×PBS for 10 min at room temperature. Cells were next stained with anti-MBNL1 antibody (Santa Cruz, 3A4) diluted in 1% bovine serum albumin (BSA) for overnight at 4° C., and washed twice with 1×PBS for 10 min each at room temperature. Cells were then incubated with the secondary antibody goat anti-rabbit Alexa 647 (Thermo Fisher, A32728) diluted in 1% BSA for 1 hr at room temperature, and washed twice with 1×PBS for 10 min each at room temperature. Next, cells were stained with Hoechst solution (Thermo Fisher, H3569) at 0.1 mg/ml for 5 min, and washed once with 1×PBS for 5 min. PBS was aspirated and fresh 100 μl of fresh PBS was added to each well. High-throughput acquisition of images was completed with the ImageXpress Micro Confocal High-Content Imaging System (Molecular Devices). RNA foci quantifications were accomplished with a customized analysis module of the MetaXpress program (Molecular Devices).

B. Results

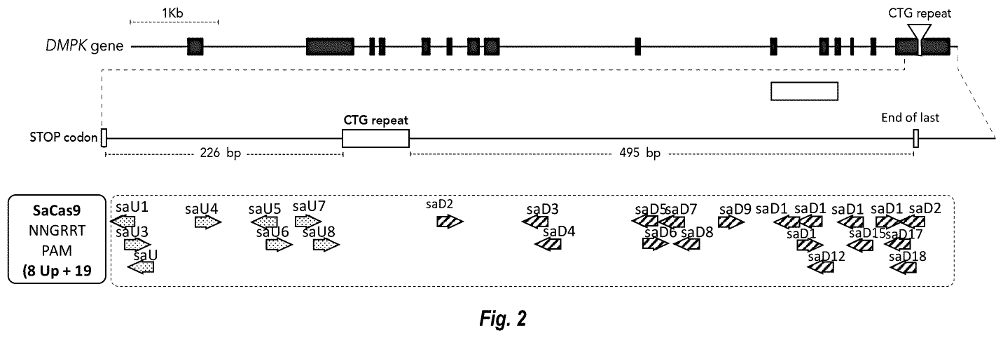

Twenty eight SaCas9 sgRNAs with the canonical NNGRRT Protospacer Adjacent Motif (PAM) sequences were selected for editing the CTG repeat expansion in the human DMPK gene (Table 1A and FIG. 2). To avoid interference with the DMPK coding sequence and mRNA maturation, all selected SaCas9 sgRNAs were located within the 3′ UTR of the DMPK gene between the stop codon and the end of the last exon. Among these 28 sgRNAs, 8 sgRNAs (SaU1-SaU8) (SEQ ID NOs: 1-8) are located upstream of the CTG repeat expansion (between the stop codon and the CTG repeat expansion); 20 sgRNAs (SaD1-SaD20) (SEQ ID NOs: 9-28) are located downstream of the CTG repeat expansion (between the CTG repeat expansion and the end of the last exon of DMPK). One sgRNA (SaD1) (SEQ ID NO: 9) was excluded from further evaluation due to its high number of predicted OFF-target sites (Table 1).

To assess the efficiency of INDEL editing and CTG repeat excision, SINGLE-cut screening was performed in which individual SaCas9 sgRNAs were assembled with recombinant SaCas9 protein into ribonucleoprotein (RNP) and delivered into primary DM1 patient myoblasts with Amaxa 4D-Nucleofector. Nucleofected myoblasts were treated with either DMSO (vehicle) or 3 μM of DNA-dependent Protein Kinase Inhibitor (DNA-PKi) Compound 6 for 48 hrs. 72 hrs post nucleofection, myoblasts were subjected to either genomic DNA isolation or RNA foci staining by fluorescence in situ hybridization (FISH).

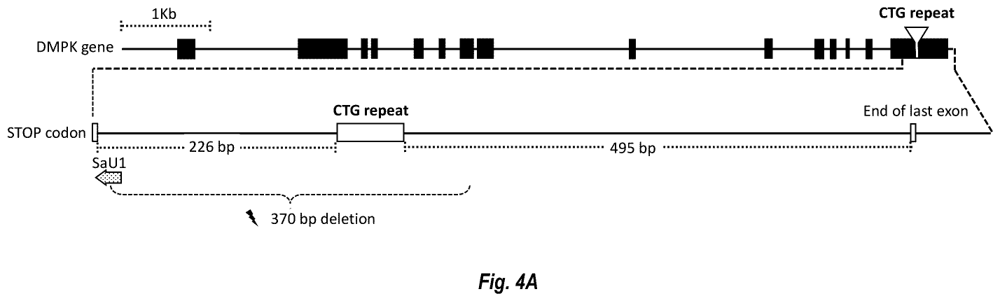

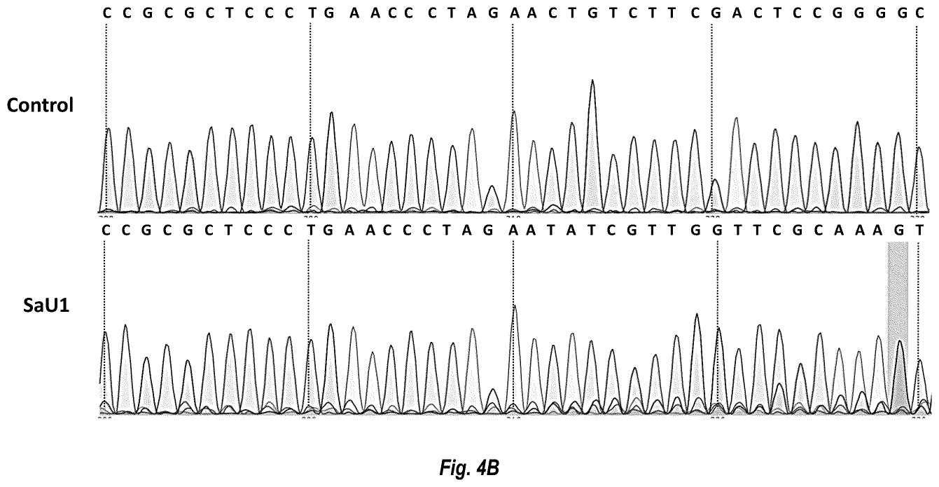

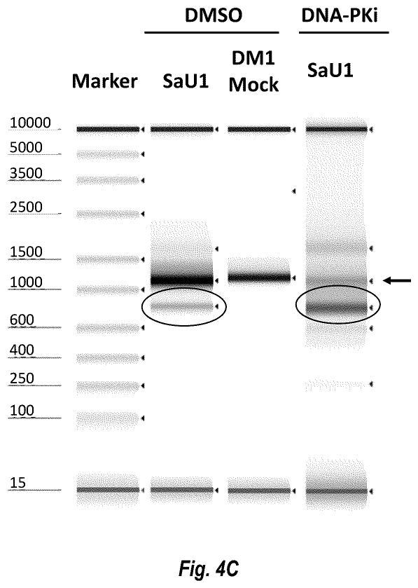

A 1174 bp sequence covering the CTG repeat expansion and the sgRNAs target region was amplified by PCR from the extracted genomic DNA. Sanger sequencing and ICE analysis were then used to quantify the frequency of indels induced by each sgRNA. Among the 27 sgRNA evaluated, 21 sgRNAs (6 upstream sgRNAs and 15 downstream sgRNAs) induced INDELs greater than 10% and were selected for further DOUBLE-cut screening (FIG. 3 and Table 3) (SEQ ID NOs: 1, 2, 3, 4, 7, 8, 10, 11, 12, 13, 14, 15, 18, 19, 20, 21, 23, 25, 26, 27, 28). It is of note that SINGLE-cut with SaU1 (SEQ ID NO: 1) induced most efficient large indel that resulted in CTG repeat excision (FIG. 4A, FIG. 4B). The DNA-PKi treatment further enhanced the efficiency of CTG repeat excision induced by SaU1 (SEQ ID NO: 1) (FIG. 4C).

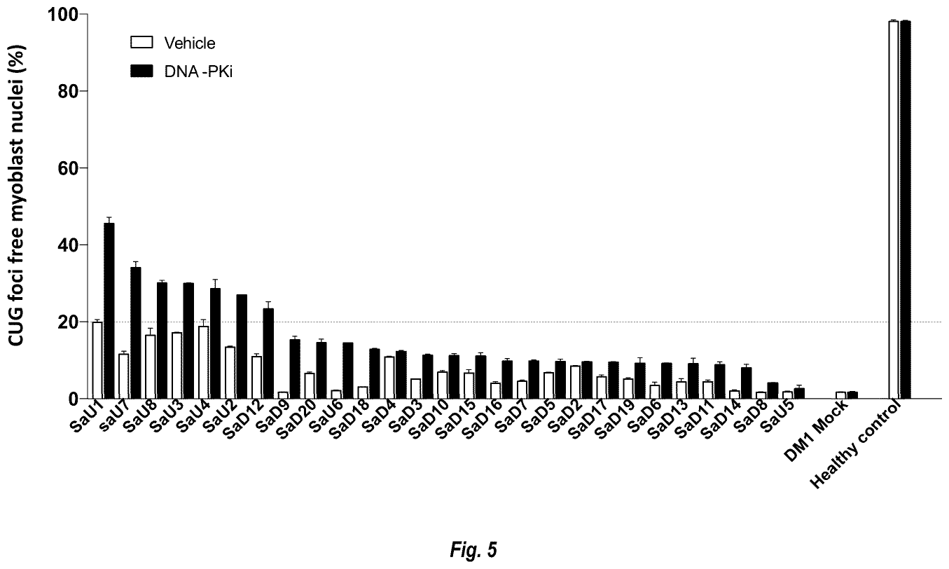

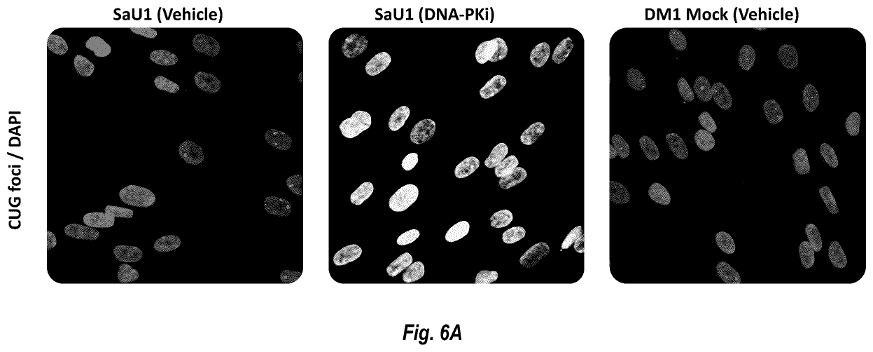

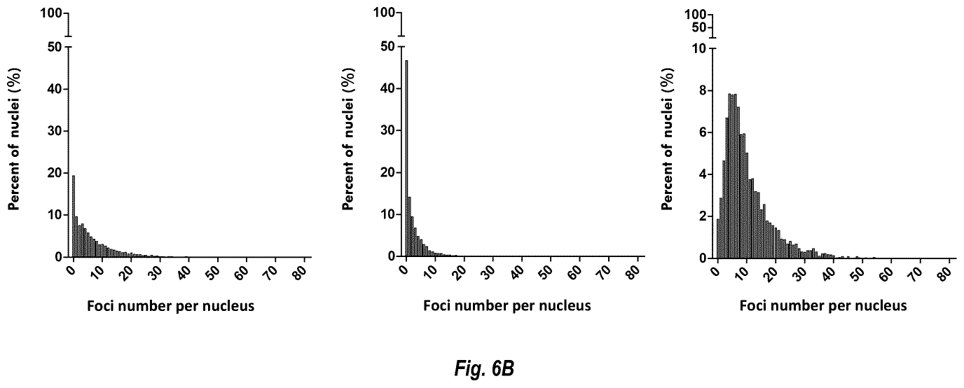

FISH staining of RNA foci showed reduction of CUG foci (formed by CUG repeat expansion in the DMPK mRNA) in DM1 patient myoblasts by individual sgRNAs (FIG. 5 and Table 3). Among the 27 sgRNAs evaluated, 8 sgRNAs completely abolished CUG RNA foci in more than 10% of myoblast nuclei with vehicle treatment (SEQ ID NOs: 1, 2, 3, 4, 7, 8, 12, 20), and 7 sgRNAs completely abolished CUG RNA foci in more than 20% of myoblast nuclei with DNA-PKi treatment (SEQ ID NOs: 1, 2, 3, 4, 7, 8, 20), in which SaU1 (SEQ ID NO: 1) sgRNA with DNA-PKi treatment abolished CUG RNA foci in 45.59% of myoblast nuclei. RNA foci distribution analysis showed that SaU1 (SEQ ID NO: 1) not only eliminated the CUG foci in a large fraction of myoblast nuclei, but also reduced the frequency of myoblast nuclei that contains more than 3 CUG foci (FIG. 6A, FIG. 6B).

TABLE 3
Efficiency of indel editing (by ICE analysis in vehicle-treated
DM1 myoblasts) and RNA foci reduction (by FISH analysis) induced
by 27 selected SaCas9 sgRNAs in primary DM1 patient myoblasts

| SaCas9 | | | | % of CUG foci | % of CUG foci |
|---|---|---|---|---|---|
| sgRNA | SEQ | Indel | Indel | free nuclei | free nuclei |
| name | ID NO | score_Mean | score_SEM | (Vehicle) | (DNA-PKi) |
| SaU1 | 1 | 55.50 | 2.50 | 19.90 | 45.59 |
| SaU2 | 2 | 57.00 | 16.50 | 13.46 | 27.06 |
| SaU3 | 3 | 60.25 | 16.25 | 17.21 | 30.03 |
| SaU4 | 4 | 51.75 | 1.75 | 18.87 | 28.65 |
| SaU5 | 5 | 1.75 | 0.75 | 1.81 | 2.63 |
| SaU6 | 6 | 6.75 | 0.25 | 2.15 | 14.53 |
| SaU7 | 7 | 70.75 | 0.25 | 11.62 | 34.12 |
| SaU8 | 8 | 51.50 | 3.00 | 16.60 | 30.17 |
| SaD2 | 10 | 41.25 | 10.75 | 8.50 | 9.58 |
| SaD3 | 11 | 50.25 | 11.25 | 5.18 | 11.36 |
| SaD4 | 12 | 37.00 | 1.00 | 10.93 | 12.28 |
| SaD5 | 13 | 62.25 | 4.25 | 6.87 | 9.74 |
| SaD6 | 14 | 28.75 | 1.75 | 3.55 | 9.20 |
| SaD7 | 15 | 40.75 | 1.25 | 4.59 | 9.80 |
| SaD8 | 16 | 5.25 | 1.75 | 1.70 | 4.10 |
| SaD9 | 17 | 2.75 | 1.75 | 1.73 | 15.45 |
| SaD10 | 18 | 51.75 | 4.75 | 6.97 | 11.26 |
| SaD11 | 19 | 35.50 | 5.50 | 4.48 | 8.87 |
| SaD12 | 20 | 55.25 | 1.75 | 11.04 | 23.40 |
| SaD13 | 21 | 16.50 | 0.00 | 4.47 | 9.12 |
| SaD14 | 22 | 7.50 | 3.50 | 2.08 | 7.99 |
| SaD15 | 23 | 74.00 | 7.50 | 6.73 | 11.08 |
| SaD16 | 24 | 9.25 | 0.25 | 4.06 | 9.84 |
| SaD17 | 25 | 48.50 | 1.50 | 5.79 | 9.50 |
| SaD18 | 26 | 17.25 | 5.75 | 3.11 | 12.89 |
| SaD19 | 27 | 37.25 | 3.75 | 5.14 | 9.27 |
| SaD20 | 28 | 38.50 | 1.00 | 6.59 | 14.65 |

Next, DOUBLE-cut screening was performed to assess the efficiency of paired sgRNAs-induced CTG repeat excision and RNA foci reduction. 90 SaCas9 sgRNA pairs formed by one upstream sgRNA and one downstream sgRNA were assembled with recombinant SaCas9 protein into RNP complex and nucleofected into primary DM1 patient myoblasts. Nucleofected myoblasts were treated with either DMSO (vehicle) or 3 μM of Compound 6 (DNA-PKi) for 48 hrs. 72 hrs post nucleofection, myoblasts were subjected to either genomic DNA isolation or RNA foci staining.

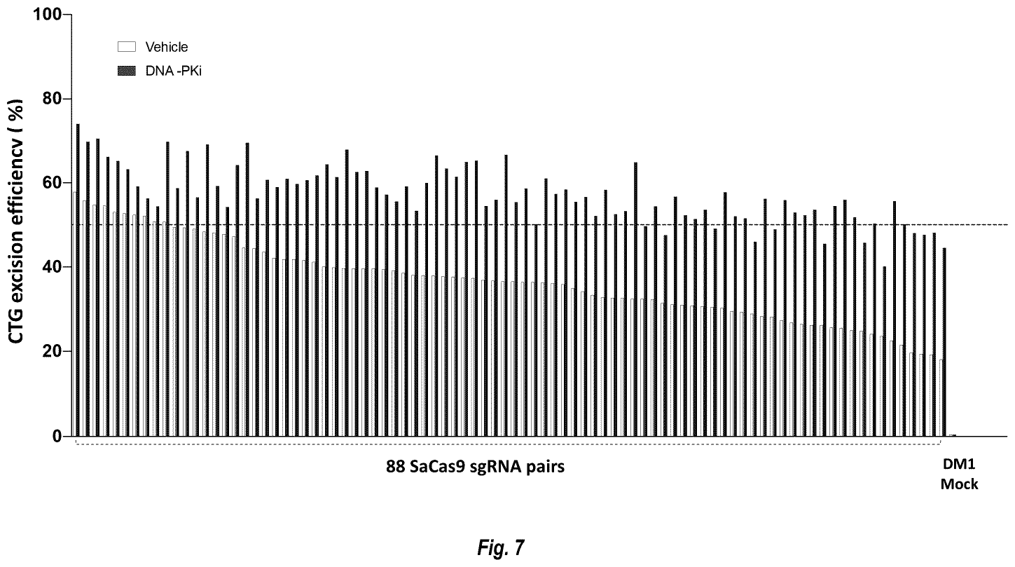

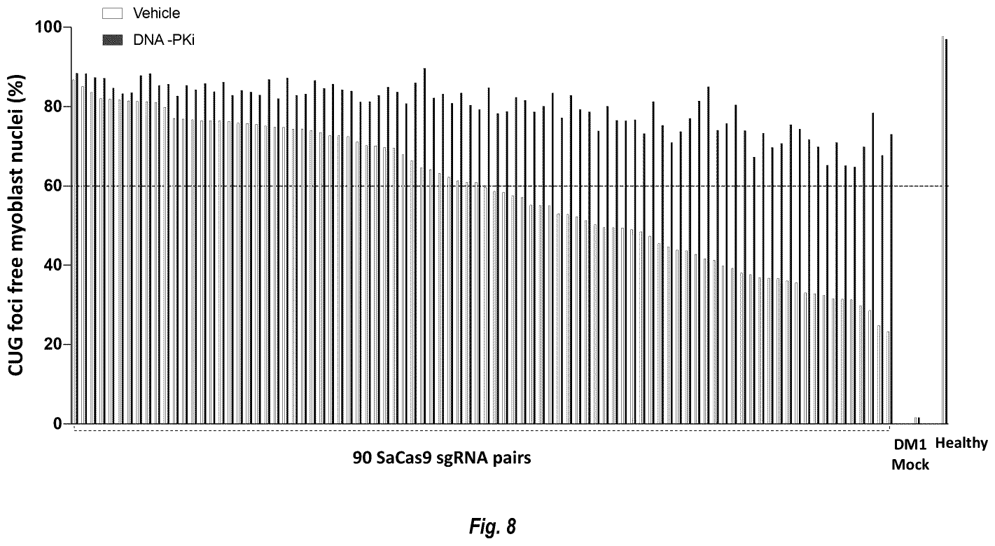

One upstream and one downstream loss-of-signal droplet digital PCR (ddPCR) assays were used to determine the efficiency of CTG repeat excision. ddPCR signals would be abolished upon excision of the CTG repeat expansion in these loss-of-signal ddPCR assays. The mean excision efficacy obtained from the two ddPCR assays were used to rank the sgRNA pairs (FIG. 7 and Table 4). It is of note that the ddPCR assays were not able to measure the CTG repeat excision efficiency of two sgRNA pairs (SaU7+SaD2 (SEQ ID NOs: 7 and 10) and SaU8+SaD2 (SEQ ID NOs: 8 and 10)) because the indels induced by these individual sgRNAs would interfere with the binding of the ddPCR primers and/or probes to the PCR template. Among the remaining 88 sgRNA pairs, 10 sgRNA pairs induced greater than 50% of CTG repeat excision efficiency with vehicle treatment, with the pair of SaU7+SaD4 (SEQ ID NOs: 7 and 12) inducing the highest CTG repeat excision efficiency (57.96%). With DNA-PKi treatment of DM1 myoblasts, 76 sgRNA pairs induced greater than 50% of CTG repeat excision efficiency, with the pair of SaU7+SaD4 (SEQ ID NOs: 7 and 12) inducing the highest CTG repeat excision efficiency (73.99%) (Table 4). Overall, the DNA-PKi treatment had a higher impact on the less efficient sgRNA pairs for the efficiency of CTG repeat excision compared to its impact on those more efficient sgRNA pairs (FIG. 7).

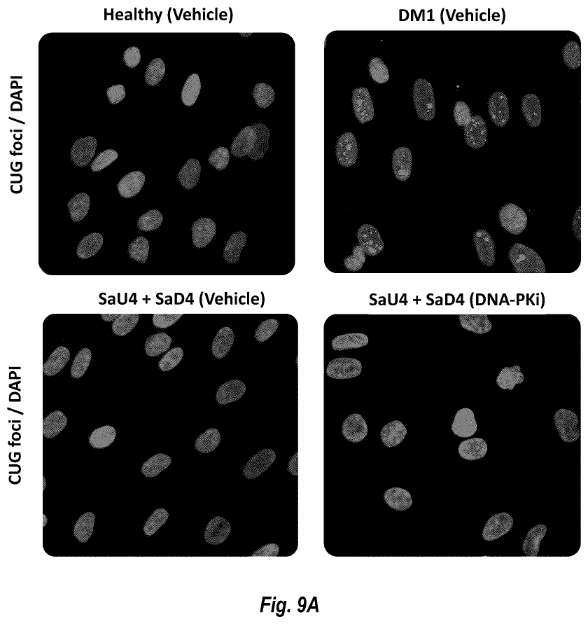

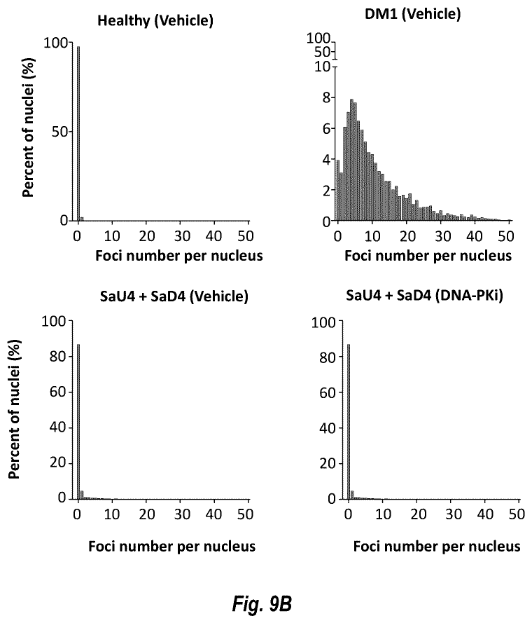

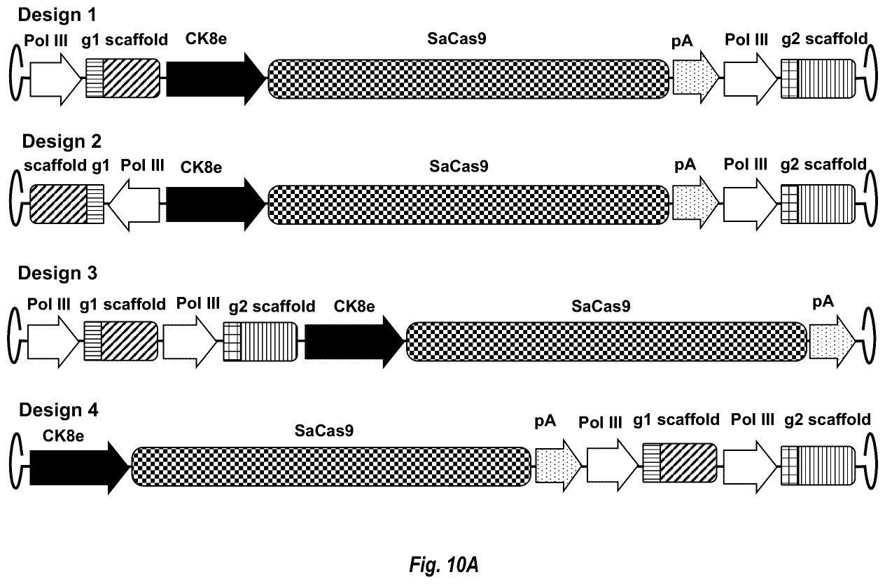

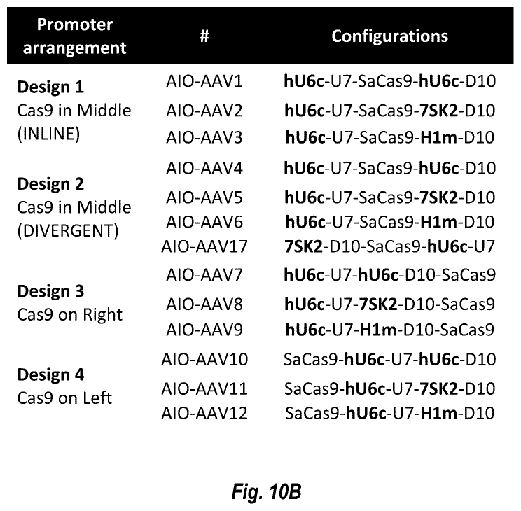

FISH staining of RNA foci showed robust reduction of CUG foci in primary DM1 patient myoblasts by both vehicle treatment and DNA-PKi treatment (FIG. 8). With vehicle treatment of DM1 myoblasts, 45 sgRNA pairs abolished CUG RNA foci in more than 60% of myoblast nuclei, with the pair of SaU4+SaD4 (SEQ ID NOs: 4 and 12) induced the highest percentage of CUG foci free myoblast nuclei (86.81%). With DNA-PKi treatment of DM1 myoblasts, all 90 sgRNA pairs abolished CUG RNA foci in more than 60% of myoblast nuclei, with the pair of SaU7+SaD15 (SEQ ID NOs: 7 and 23) induced the highest percentage of CUG foci free myoblast nuclei (89.69%). Similar to CTG repeat excision, the DNA-PKi treatment had a higher impact on the less efficient sgRNA pairs for the efficiency of RNA foci reduction compared to its impact on those more efficient sgRNA pairs. RNA foci distribution analysis showed that the pair of SaU4+SaD4 (SEQ ID NOs: 4 and 12) abolished CUG foci in vast majority of myoblasts treated with either vehicle or DNA-PKi, with small number of nuclei showed 1 remaining foci (FIG. 9A and FIG. 9B).

TABLE 4
Efficiency of CTG repeat excision (by ddPCR) and RNA foci reduction (by FISH
analysis) induced by 90 SaCas9 sgRNA pairs in primary DM1 patient myoblasts

| | | % of CTG | % of CTG | | |
|---|---|---|---|---|---|
| | | repeat | repeat | | |
| | | excision | excision | % of CUG | % of CUG |
| SaCas9 | | efficiency | efficiency | foci free | foci free |
| sgRNA | | by ddPCR | by ddPCR | nuclei | nuclei |
| pairs | SEQ ID NOS | (Vehicle) | (DNA-PKi) | (Vehicle) | (DNA-PKi) |
| SaU4 + SaD4 | SEQ ID NOs: | 54.65 | 66.15 | 86.81 | 88.43 |
| | 4 and 12 | | | | |
| SaU2 + SaD4 | SEQ ID NOs: | 41.89 | 59.76 | 85.13 | 88.39 |
| | 2 and 12 | | | | |
| SaU3 + SaD4 | SEQ ID NOs: | 47.22 | 64.28 | 83.67 | 87.42 |
| | 3 and 12 | | | | |
| SaU4 + SaD12 | SEQ ID NOS: | 42.13 | 59.06 | 82.10 | 87.22 |
| | 4 and 20 | | | | |
| SaU4 + SaD10 | SEQ ID NOs: | 49.44 | 67.61 | 82.01 | 84.75 |
| | 4 and 18 | | | | |
| SaU2 + SaD2 | SEQ ID NOs: | 52.26 | 56.39 | 81.76 | 83.29 |
| | 2 and 10 | | | | |
| SaU4 + SaD20 | SEQ ID NOs: | 52.49 | 59.19 | 81.47 | 83.56 |
| | 4 and 28 | | | | |
| SaU1 + SaD4 | SEQ ID NOS: | 41.92 | 61.00 | 81.45 | 87.86 |
| | 1 and 12 | | | | |
| SaU8 + SaD4 | SEQ ID NOs: | 55.89 | 69.71 | 81.32 | 88.35 |
| | 8 and 12 | | | | |
| SaU4 + SaD5 | SEQ ID NOs: | 52.75 | 63.28 | 81.08 | 85.34 |
| | 4 and 13 | | | | |
| SaU4 + SaD15 | SEQ ID NOs: | 39.70 | 58.99 | 79.84 | 85.73 |
| | 4 and 23 | | | | |
| SaU3 + SaD2 | SEQ ID NOs: | 50.85 | 54.49 | 77.08 | 82.73 |
| | 3 and 10 | | | | |
| SaU8 + SaD12 | SEQ ID NOs: | 36.64 | 55.46 | 76.92 | 85.37 |
| | 8 and 20 | | | | |
| SaU1 + SaD2 | SEQ ID NOS: | 47.82 | 54.31 | 76.74 | 84.27 |
| | 1 and 10 | | | | |
| SaU2 + SaD15 | SEQ ID NOs: | 41.70 | 60.64 | 76.57 | 85.85 |
| | 2 and 23 | | | | |
| SaU2 + SaD12 | SEQ ID NOs: | 36.47 | 58.68 | 76.56 | 83.77 |
| | 2 and 20 | | | | |
| SaU8 + SaD15 | SEQ ID NOS: | 39.47 | 57.24 | 76.50 | 86.21 |
| | 8 and 23 | | | | |
| SaU8 + SaD2 | SEQ ID NOS: | NA | NA | 76.39 | 82.91 |
| | 8 and 10 | | | | |
| SaU1 + SaD10 | SEQ ID NOs: | 37.72 | 61.44 | 75.91 | 84.12 |
| | 1 and 18 | | | | |
| SaU2 + SaD5 | SEQ ID NOS: | 38.64 | 59.19 | 75.82 | 83.71 |
| | 2 and 13 | | | | |
| SaU2 + SaD10 | SEQ ID NOs: | 38.02 | 66.52 | 75.56 | 82.97 |
| | 2 and 18 | | | | |
| SaU3 + SaD10 | SEQ ID NOs: | 44.56 | 69.50 | 75.19 | 86.88 |
| | 3 and 18 | | | | |
| SaU2 + SaD20 | SEQ ID NOs: | 36.35 | 61.08 | 74.86 | 82.08 |
| | 2 and 28 | | | | |
| SaU7 + SaD4 | SEQ ID NOs: | 57.96 | 73.99 | 74.84 | 87.34 |
| | 7 and 12 | | | | |
| SaU8 +SaD10 | SEQ ID NOs: | 48.45 | 69.07 | 74.46 | 82.92 |
| | 8 and 18 | | | | |
| SaU3 + SaD12 | SEQ ID NOs: | 38.05 | 59.96 | 74.46 | 83.21 |
| | 3 and 20 | | | | |
| SaU3 + SaD15 | SEQ ID NOs: | 37.42 | 65.28 | 74.00 | 86.64 |
| | 3 and 23 | | | | |
| SaU3 + SaD5 | SEQ ID NOS: | 43.61 | 60.74 | 73.56 | 84.63 |
| | 2 and 13 | | | | |
| SaU1 + SaD15 | SEQ ID NOs: | 37.54 | 64.96 | 72.80 | 85.70 |
| | 1 and 23 | | | | |
| SaU8 + SaD5 | SEQ ID NOs: | 49.47 | 58.77 | 72.77 | 84.28 |
| | 8 and 13 | | | | |
| SaU3 + SaD20 | SEQ ID NOS: | 40.12 | 64.44 | 72.56 | 83.99 |
| | 3 and 28 | | | | |
| SaU8 +SaD20 | SEQ ID NOS: | 39.72 | 62.59 | 71.20 | 81.21 |
| | 8 and 28 | | | | |
| SaU7 + SaD2 | SEQ ID NOs: | NA | NA | 70.29 | 81.28 |
| | 7 and 10 | | | | |
| SaU1 + SaD5 | SEQ ID NOS: | 30.65 | 53.67 | 70.23 | 82.90 |
| | 1 and 13 | | | | |
| SaU1 + SaD12 | SEQ ID NOS: | 37.77 | 63.43 | 69.78 | 84.94 |
| | 1 and 20 | | | | |
| SaU1 + SaD20 | SEQ ID NOS: | 39.94 | 61.37 | 69.61 | 83.73 |
| | 1 and 28 | | | | |
| SaU4 + SaD19 | SEQ ID NOS: | 36.41 | 50.21 | 67.92 | 80.80 |
| | 4 and 27 | | | | |
| SaU7 + SaD12 | SEQ ID NOs: | 39.71 | 62.84 | 66.41 | 86.06 |
| | 7 and 20 | | | | |
| SaU7 + SaD15 | SEQ ID NOS: | 36.65 | 66.66 | 64.62 | 89.69 |
| | 7 and 23 | | | | |
| SaU7 + SaD5 | SEQ ID NOS: | 50.76 | 69.71 | 64.24 | 82.27 |
| | 7 and 13 | | | | |
| SaU7 +SaD20 | SEQ ID NOs: | 39.72 | 67.93 | 63.33 | 83.24 |
| | 7 and 28 | | | | |
| SaU2 + SaD19 | SEQ ID NOs: | 49.08 | 56.62 | 62.27 | 80.94 |
| | 2 and 27 | | | | |
| SaU8 + SaD19 | SEQ ID NOs: | 28.95 | 46.04 | 61.41 | 83.45 |
| | 8 and 27 | | | | |
| SaU4 + SaD3 | SEQ ID NOs: | 44.46 | 56.38 | 60.98 | 80.39 |
| | 4 and 11 | | | | |
| SaU4 + SaD17 | SEQ ID NOs: | 35.01 | 55.51 | 60.96 | 79.31 |
| | 4 and 25 | | | | |
| SaU4 + SaD20 | SEQ ID NOs: | 53.12 | 65.25 | 59.76 | 84.77 |
| | 4 and 28 | | | | |
| SaU4 + SaD11 | SEQ ID NOs: | 36.98 | 54.55 | 58.71 | 78.29 |
| | 4 and 19 | | | | |
| SaU4 + SaD7 | SEQ ID NOs: | 39.17 | 55.59 | 58.50 | 78.79 |
| | 4 and 15 | | | | |
| SaU8 +SaD3 | SEQ ID NOs: | 48.19 | 59.28 | 57.67 | 82.41 |
| | 8 and 11 | | | | |
| SaU3 + SaD19 | SEQ ID NOs: | 32.67 | 53.36 | 57.16 | 81.67 |
| | 3 and 27 | | | | |
| SaU2 + SaD17 | SEQ ID NOs: | 31.11 | 52.34 | 55.20 | 78.68 |
| | 2 and 25 | | | | |
| SaU2 + SaD3 | SEQ ID NOS: | 36.75 | 56.04 | 55.11 | 80.14 |
| | 2 and 11 | | | | |
| SaU7 +SaD10 | SEQ ID NOS: | 54.86 | 70.47 | 55.03 | 83.49 |
| | 7 and 18 | | | | |
| SaU3 + SaD17 | SEQ ID NOS: | 31.18 | 56.80 | 53.10 | 77.22 |
| | 3 and 25 | | | | |
| SaU8 + SaD7 | SEQ ID NOS: | 38.19 | 53.42 | 52.92 | 82.90 |
| | 8 and 15 | | | | |
| SaU8 + SaD17 | SEQ ID NOs: | 30.50 | 49.09 | 52.34 | 79.35 |
| | 8 and 25 | | | | |
| SaU3 + SaD3 | SEQ ID NOs: | 36.25 | 57.46 | 51.28 | 78.72 |
| | 3 and 11 | | | | |
| SaU3 + SaD11 | SEQ ID NOS: | 32.84 | 58.33 | 50.28 | 73.87 |
| | 3 and 19 | | | | |
| SaU1 + SaD7 | SEQ ID NOS: | 34.21 | 56.69 | 49.51 | 80.16 |
| | 1 and 15 | | | | |
| SaU3 + SaD7 | SEQ ID NOs: | 35.98 | 58.50 | 49.49 | 76.54 |
| | 3 and 15 | | | | |
| SaU1 + SaD19 | SEQ ID NOS: | 32.74 | 52.62 | 49.44 | 76.49 |
| | 1 and 27 | | | | |
| SaU2 + SaD7 | SEQ ID NOS: | 29.62 | 52.12 | 49.05 | 76.74 |
| | 2 and 15 | | | | |
| SaU2 + SaD11 | SEQ ID NOs: | 26.90 | 52.97 | 48.38 | 73.23 |
| | 2 and 19 | | | | |
| SaU1 + SaD3 | SEQ ID NOs: | 33.47 | 52.18 | 47.32 | 81.32 |
| | 1 and 11 | | | | |
| SaU1 + SaD17 | SEQ ID NOS: | 26.29 | 53.66 | 45.51 | 75.32 |
| | 1 and 25 | | | | |
| SaU8 +SaD11 | SEQ ID NOS: | 31.52 | 47.57 | 44.65 | 70.97 |
| | 8 and 19 | | | | |
| SaU4 + SaD13 | SEQ ID NOS: | 32.38 | 54.46 | 43.83 | 73.71 |
| | 4 and 21 | | | | |
| SaU8 +SaD13 | SEQ ID NOS: | 29.46 | 51.64 | 43.66 | 77.05 |
| | 8 and 21 | | | | |
| SaU7 + SaD19 | SEQ ID NOs: | 28.45 | 56.22 | 42.75 | 81.47 |
| | 7 and 27 | | | | |
| SaU7 + SaD7 | SEQ ID NOs: | 32.55 | 64.88 | 41.68 | 85.03 |
| | 7 and 15 | | | | |
| SaU1 + SaD11 | SEQ ID NOS: | 26.52 | 52.38 | 41.32 | 74.03 |
| | 1 and 19 | | | | |
| SaU2 + SaD13 | SEQ ID NOS: | 30.92 | 51.47 | 39.86 | 75.83 |
| | 2 and 21 | | | | |
| SaU7 +SaD3 | SEQ ID NOs: | 41.26 | 61.82 | 39.24 | 80.45 |
| | 7 and 11 | | | | |
| SaU3 + SaD13 | SEQ ID NOs: | 30.34 | 57.82 | 38.07 | 74.02 |
| | 3 and 21 | | | | |
| SaU4 + SaD6 | SEQ ID NOs: | 28.23 | 48.99 | 37.76 | 67.36 |
| | 4 and 14 | | | | |
| SaU7 +SaD11 | SEQ ID NOs: | 25.10 | 51.90 | 36.96 | 73.38 |
| | 7 and 19 | | | | |
| SaU4 + SaD18 | SEQ ID NOS: | 26.25 | 45.55 | 36.81 | 69.76 |
| | 4 and 26 | | | | |
| SaU8 + SaD18 | SEQ ID NOs: | 18.17 | 44.51 | 36.66 | 70.76 |
| | 8 and 26 | | | | |
| SaU7 + SaD17 | SEQ ID NOs: | 27.41 | 55.97 | 36.12 | 75.53 |
| | 7 and 25 | | | | |
| SaU1 + SaD13 | SEQ ID NOS: | 25.67 | 54.59 | 35.75 | 74.44 |
| | 1 and 21 | | | | |
| SaU3 + SaD18 | SEQ ID NOS: | 21.51 | 50.13 | 33.15 | 71.73 |
| | 3 and 26 | | | | |
| SaU2 + SaD18 | SEQ ID NOs: | 19.28 | 48.16 | 32.87 | 69.93 |
| | 2 and 26 | | | | |
| SaU8 + SaD6 | SEQ ID NOs: | 23.57 | 40.19 | 32.43 | 65.28 |
| | 8 and 14 | | | | |
| SaU1 + SaD6 | SEQ ID NOs: | 32.51 | 49.69 | 31.66 | 70.98 |
| | 1 and 14 | | | | |
| SaU2 + SaD6 | SEQ ID NOs: | 24.88 | 45.77 | 31.53 | 65.23 |
| | 2 and 14 | | | | |
| SaU3 + SaD6 | SEQ ID NOs: | 24.10 | 50.32 | 31.37 | 64.84 |
| | 3 and 14 | | | | |
| SaU1 + SaD18 | SEQ ID NOS: | 19.40 | 47.64 | 29.85 | 69.94 |
| | 1 and 26 | | | | |
| SaU7 +SaD13 | SEQ ID NOS: | 25.62 | 55.97 | 28.69 | 78.52 |
| | 7 and 21 | | | | |
| SaU7 + SaD6 | SEQ ID NOS: | 22.54 | 55.71 | 24.89 | 67.73 |
| | 7 and 14 | | | | |
| SaU7 + SaD18 | SEQ ID NOs: | 19.71 | 48.08 | 23.22 | 73.07 |
| | 7 and 26 | | | | |
| DM383 Mock | | 0.53 | 0.49 | 1.69 | 1.71 |
| Control | | | | | |
| Healthy | | NA | NA | 97.94 | 97.82 |
| Control | | | | | |

### Example 2: AAV Vector Configurations

A series of “all-in-one” vector configurations were designed as depicted in FIG. 10A and synthesized as depicted in FIG. 10B. Four representative vector designs were constructed (FIG. 10A).

Design 1 (“Cas9 in Middle Inline”) included in order from 5′ to 3′ with respect to the plus strand: a promoter for expression of the nucleic acid encoding the first sgRNA in the same direction as the promoter for SaCas9, the first sgRNA guide sequence and scaffold sequence, a promoter for expression of a nucleic acid encoding SaCas9 (e.g., CK8e), a nucleic acid encoding SaCas9, polyadenylation sequence, a promoter for expression of the nucleic acid encoding the second sgRNA in the same direction as the promoter for SaCas9, and the second sgRNA guide sequence and scaffold sequence. Specific variations of the Design 1 configuration are shown in FIG. 10B, including e.g., AIO-AA1, AIO-AAV2, and AIO-AAV3.

Design 2 (“Cas9 in Middle Divergent”) included in order from 5′ to 3′ with respect to the plus strand: the reverse complement of a first sgRNA scaffold sequence, the reverse complement of a nucleic acid encoding a first sgRNA guide sequence, the reverse complement of a promoter for expression of the nucleic acid encoding the first sgRNA, a promoter for expression of a nucleic acid encoding SaCas9 (e.g., CK8e), a nucleic acid encoding SaCas9, a polyadenylation sequence, a promoter for expression of the nucleic acid encoding the second sgRNA in the same direction as the promoter for SaCas9, and the second sgRNA guide sequence and scaffold sequence. Variations of the Design 2 configuration are shown in FIG. 10B, including e.g., AIO-AAV4, AIO-AAVS, AIO-AAV6, and AIO-AAV17.

Design 3 (“Cas9 on Right”) included in order from 5′ to 3′ with respect to the plus strand: a promoter for expression of the nucleic acid encoding the first sgRNA in the same direction as the promoter for SaCas9, the first sgRNA guide sequence and scaffold sequence, a promoter for expression of the nucleic acid encoding the second sgRNA in the same direction as the promoter for SaCas9, the second sgRNA guide sequence and scaffold sequence, a promoter for expression of a nucleic acid encoding SaCas9 (e.g., CK8e), a nucleic acid encoding SaCas9, and a polyadenylation sequence. Variations of the Design 3 configuration are shown in FIG. 10B, including e.g., AIO-AAV7, AIO-AAV8, and AIO-AAV9.

Design 4 (“Cas9 on Left”) included in order from 5′ to 3′ with respect to the plus strand: a promoter for expression of a nucleic acid encoding SaCas9 (e.g., CK8e), a nucleic acid encoding SaCas9, a polyadenylation sequence, a promoter for expression of the nucleic acid encoding the first sgRNA in the same direction as the promoter for SaCas9, the first sgRNA guide sequence and scaffold sequence, a promoter for expression of the nucleic acid encoding the second sgRNA in the same direction as the promoter for SaCas9, and the second sgRNA guide sequence and scaffold sequence. Variations of the Design 4 configuration are shown in FIG. 10B, including e.g., AIO-AAV10, AIO-AAV11, and AIO-AAV12.

The sequences for components of the vector configurations are shown in Table 5.

TABLE 5
Sequences for AAV vector components

| | SEQ | | |
|---|---|---|---|
| | ID | Size | |
| Component | NO | (bp) | Sequence |
| hU6c | 705 | 249 | GAGGGCCTATTTCCCATGATTCCTTCA |
| | | | TATTTGCATATACGATACAAGGCTGTT |
| | | | AGAGAGATAATTGGAATTAATTTGACT |
| | | | GTAAACACAAAGATATTAGTACAAAAT |
| | | | ACGTGACGTAGAAAGTAATAATTTCTT |
| | | | GGGTAGTTTGCAGTTTTAAAATTATGT |
| | | | TTTAAAATGGACTATCATATGCTTACC |
| | | | GTAACTTGAAAGTATTTCGATTTCTTG |
| | | | GCTTTATATATCTTGTGGAAAGGACGA |
| | | | AACACC |
| 7SK2 | 706 | 243 | CTGCAGTATTTAGCATGCCCCACCCAT |
| | | | CTGCAAGGCATTCTGGATAGTGTCAAA |
| | | | ACAGCCGGAAATCAAGTCCGTTTATCT |
| | | | CAAACTTTAGCATTTTGGGAATAAATG |
| | | | ATATTTGCTATGCTGGTTAAATTAGAT |
| | | | TTTAGTTAAATTTCCTGCTGAAGCTCT |
| | | | AGTACGATAAGCAACTTGACCTAAGTG |
| | | | TAAAGTTGAGACTTCCTTCAGGTTTAT |
| | | | ATAGCTTGTGCGCCGCTTGGGTACCTC |
| H1m | 707 | 100 | AATATTTGCATGTCGCTATGTGTTCTG |
| | | | GGAAATCACCATAAACGTGAAATGTCT |
| | | | TTGGATTTGGGAATCTTATAAGTTCTG |
| | | | TATGAGACCACTCTTTCCC |
| SaU7 | 7 | 22 | CGCGGCCGGCGAACGGGGCTCG |
| sgRNA | | | |
| SaD10 | 18 | 22 | TTGGGGGTCCTGTAGCCTGTCA |
| sgRNA | | | |
| SaCas9 | 911 | 87 | GTTTAAGTACTCTGTGCTGGAAACAGC |
| Scaffold | | | ACAGAATCTACTTAAACAAGGCAAAAT |
| V2 | | | GCCGTGTTTATCTCGTCAACTTGTTGG |
| | | | CGAGAT |
| SV40 NLS | 915 | 21 | CCAAAGAAGAAGCGGAAGGTC |
| (N- | | | (SEQ ID NO: 915) |
| terminus) | | | (amino acid sequence is: |
| | | | PKKKRKV; |
| | | | SEQ ID NO: 916) |
| Nucleoplas | 917 | 48 | AAGCGACCTGCCGCCACAAAGAAGGCT |
| min NLS | | | GGACAGGCTAAGAAGAAGAAA |
| (C- | | | (SEQ ID NO: 917) |
| terminus) | | | (amino acid sequence is: |
| | | | KRPAATKKAGQAKKKK; |
| | | | SEQ ID NO: 918) |
| Poly(A) | 919 | 49 | AATAAAATATCTTTATTTTCATTACAT |
| sequence | | | CTGTGTGTTGGTTTTTTGTGTG |
| 5′ ITR | 709 | 143 | GGCCACTCCCTCTCTGCGCGCTCGCTC |
| | | | GCTCACTGAGGCCGGGCGACCAAAGGT |
| | | | CGCCCGACGCCCGGGCTTTGCCCGGGC |
| | | | GGCCTCAGTGAGCGAGCGAGCGCGCAG |
| | | | AGAGGGAGTGGCCAACTCCATCACTAG |
| | | | GGGTTCCT |
| 3′ ITR | 710 | 137 | AGGAACCCCTAGTGATGGAGTTGGCCA |
| | | | CTCCCTCTCTGCGCGCTCGCTCGCTCA |
| | | | CTGAGGCCGGGCGACCAAAGGTCGCCC |
| | | | GACGCCCGGGCTTTGCCCGGGCGGCCT |
| | | | CAGTGAGCGAGCGAGCGCGCAGAGAGG |
| | | | GA |

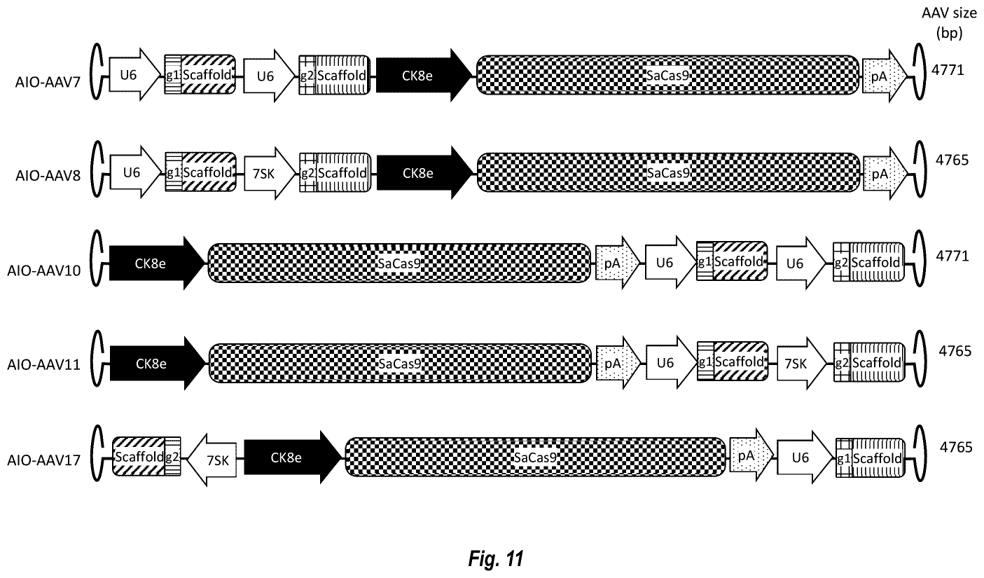

The sizes of representative vector configurations were determined. The sizes of AIO-AAV7, AIO-AAV8, AIO-AA10, AIO-AAV11, and AIO-AAV17 were 4771, 4765, 4771, 4765, and 4765 base pairs (bp), respectively (FIG. 11).

Variants of the SaCas9 scaffold were designed as show in Table 6 below.

TABLE 6
SaCas9 scaffold variants

| | SEQ | | |
|---|---|---|---|
| Designa- | ID | | |
| tion | NO | Sequence | Size |
| SaScaffold | 910 | GTTTTAGTACTCTGGAAACAGAA | 77 |
| V1 | | TCTACTAAAACAAGGCAAAATGC | |
| | | CGTGTTTATCTCGTCAACTTGTT | |
| | | GGCGAGAT | |
| SaScaffold | 911 | GTTTAAGTACTCTGTGCTGGAAA | 87 |
| V2 | | CAGCACAGAATCTACTTAAACAA | |
| | | GGCAAAATGCCGTGTTTATCTCG | |
| | | TCAACTTGTTGGCGAGAT | |
| SaScaffold | 912 | GTTTAAGTACTCTGGAAACAGAA | 77 |
| V3 | | TCTACTTAAACAAGGCAAAATGC | |
| | | CGTGTTTATCTCGTCAACTTGTT | |
| | | GGCGAGAT | |

* Bold, underlined nucleotides show variation among the three variants; all other sequences are identical in the three variants

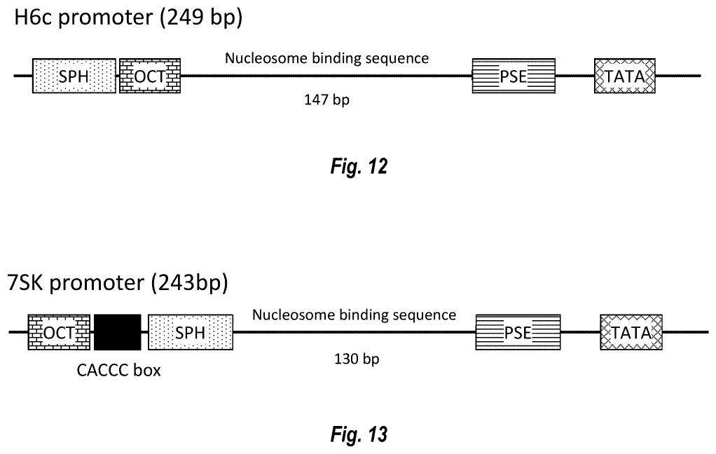

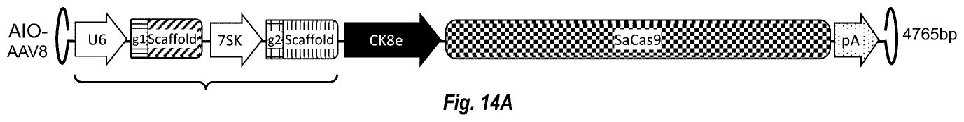

To further reduce the size of the AAV vectors, variants of the hU6c promoter (Table 7; SEQ ID NOs: 901-904) and 7SKs promoter (Table 8; SEQ ID NOs: 906-909) were designed by shortening the sequence within the nucleosome binding sequence. FIG. 12 and FIG. 13 show schematics of the hU6c and 7SK promoters, respectively.

TABLE 7
hU6c promoter variants

| | SEQ ID | | |
|---|---|---|---|
| Designation | NO | Sequence | Size |
| hU6c | 705 | 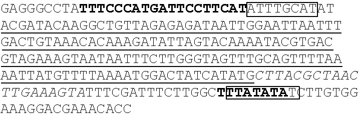 | 249 |
| hU6d30 | 901 | 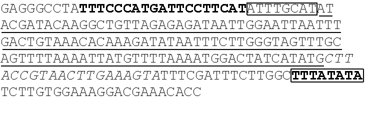 | 219 |
| hU6d60 | 902 | 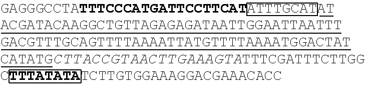 | 189 |
| hU6d90 | 903 | 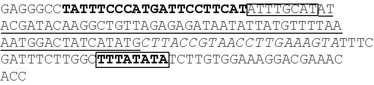 | 159 |
| hU6d120 | 904 | 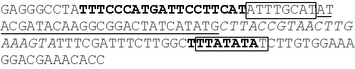 | 129 |

*Bold indicates nucleotides in the SPH region of the promoter; boxed
nucleotides indicate nucleotides in the OCT region of the promoter;
underlined nucleotides indicate nucleotides in the nucleosome binding
sequence of the promoter; bold italics indicate nucleotides in the PSE
region of the promoter; and bold boxed nucleotides indicate nucleotides
in the TATA region of the promoter.

TABLE 8
7Sk2 promoter variants

| | SEQ ID | | |
|---|---|---|---|
| Designation | NO | Sequence | Size |
| 7SK2 | 706 | 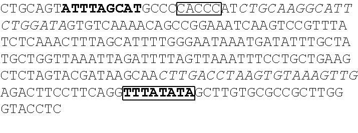 | 243 |
| 7SKd30 | 906 | 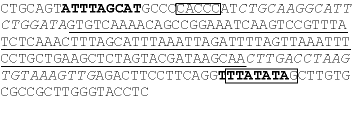 | 213 |
| 7SKd60 | 907 | 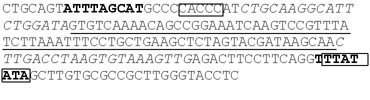 | 183 |
| 7SKd90 | 908 | 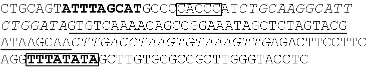 | 153 |
| 7SKd120 | 909 | 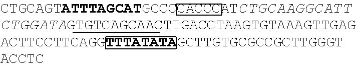 | 123 |

*Bold indicates nucleotides in the OCT region of the promoter; boxed
nucleotides indicate nucleotides in the CACCC box of the promoter;
italics indicate nucleotides in the SPH region of the promoter;
underlined nucleotides indicate nucleotides in the nucleosome binding
sequence of the promoter; bold italics indicate nucleotides in the PSE
region of the promoter; and bold boxed nucleotides indicate nucleotides
in the TATA region of the promoter.

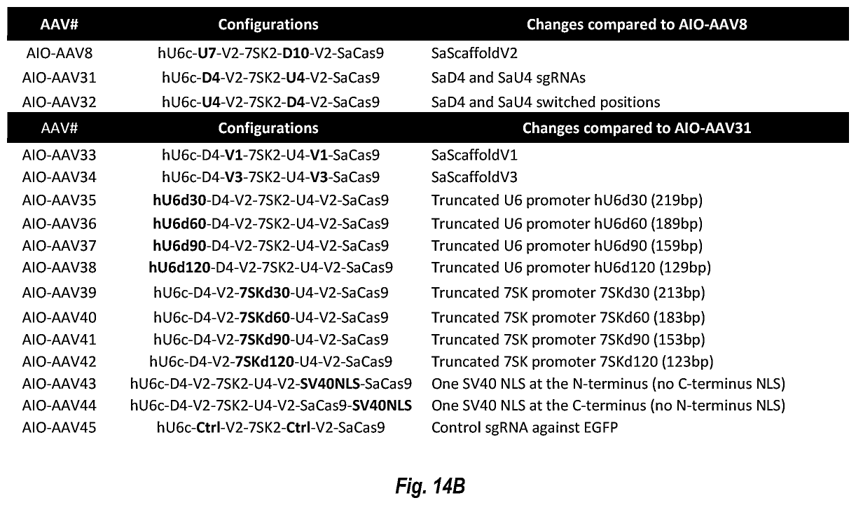

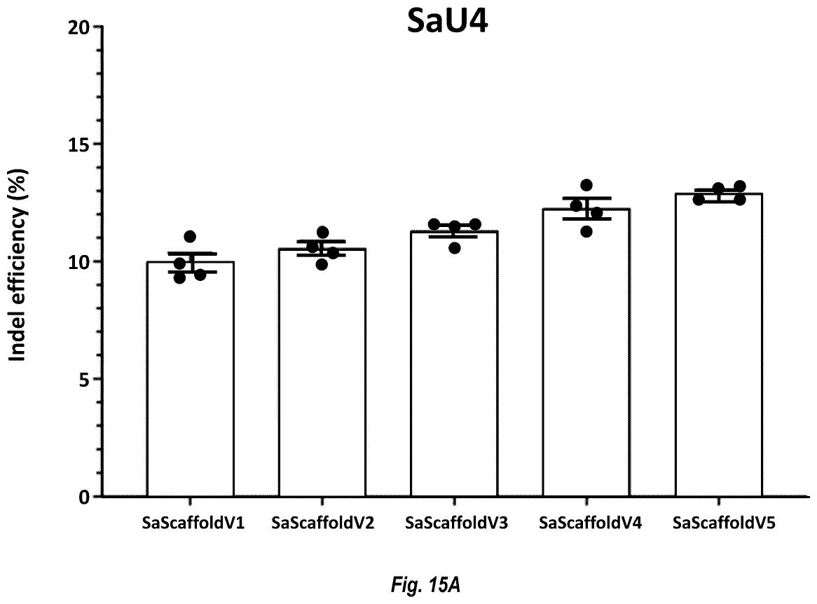

Utilizing variants of the hU6c promoter (Table 7), variants of the 7SK2 promoter (Table 8), and SaCas9 scaffold variants (Table 6), further vector configurations were designed (FIG. 14B). For example, the vector configurations AIO-AA31 and AIO-AAV32 were designed based off of AIO-AAV8 (schematic of AIO-AAV8 is shown in FIG. 14A). Additional vector configurations were designed based off of AIO-AA31, including AIO-AAV33, AIO-AAV34, AIO-AAV-35, AIO-AAV36, AIO-AAV-37, AIO-AAV-38, AIO-AAV39, AIO-AAV40, AIO-AAV41, AIO-AAV42, AIO-AAV43, AIO-AAV44, AIO-AAV45.

Certain sgRNA pairs were selected for testing in the vector configurations including certain pairings of sgRNAs selected from SaU1 (SEQ ID NO: 1), SaU2 (SEQ ID NO: 2), SaU3 (SEQ ID NO: 3), SaU4 (SEQ ID NO: 4), SaU7 (SEQ ID NO: 7), SaU8 (SEQ ID NO: 8), SaD2 (SEQ ID NO: 10), SaD4 (SEQ ID NO: 12), SaD5 (SEQ ID NO: 13), SaD10 (SEQ ID NO: 18), SaD12 (SEQ ID NO: 20), and SaD20 (SEQ ID NO: 28). For example, SaU7 (SEQ ID NO: 7) and SaD10 (SEQ ID NO: 18) were incorporated in AIO-AA8, and SaU4 (SEQ ID NO: 4) and SaD4 (SEQ ID NO: 12) were incorporated in AIO-AAV31.

### Example 3: Editing Efficiency With Different Scaffolds

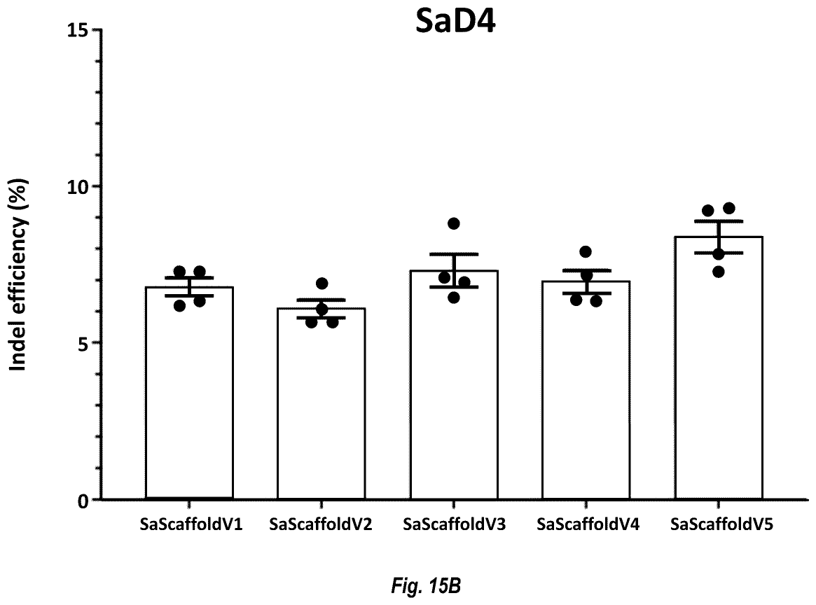

To assess editing efficiencies with different scaffolds, plasmids carrying SaCas9 and SaU4 (FIG. 15A) or SaD4 (FIG. 15B) were transfected into HEK293T cells with Lipofectamine 2000. Scaffold sequences are shown in Table 6 and Table 9. Genomic DNA was extracted at 48 h post transfection, and a 1174 bp sequence covering the CTG repeat expansion and the sgRNA target sites was amplified by PCR. Sanger sequencing and TIDE analysis were then used to quantify the frequency of indels generated by each sgRNA. Results are shown as mean±standard error (n=4).

TABLE 9
Additional SaCas9 scaffold variants

| | SEQ | | |
|---|---|---|---|
| Designa- | ID | | |
| tion | NO | Sequence | Size |
| SaScaffold | 920 | GTTTCAGTACTCTGTGCTGGAAACAG | 87 |
| V4 | | CACAGAATCTACTGAAACAAGGCAAA | |
| | | ATGCCGTGTTTATCTCGTCAACTTGT | |
| | | TGGCGAGAT | |
| SaScaffold | 921 | GTTTCAGTACTCTGGAAACAGAATCT | 77 |
| V5 | | ACTGAAACAAGGCAAAATGCCGTGTT | |
| | | TATCTCGTCAACTTGTTGGCGAGAT | |

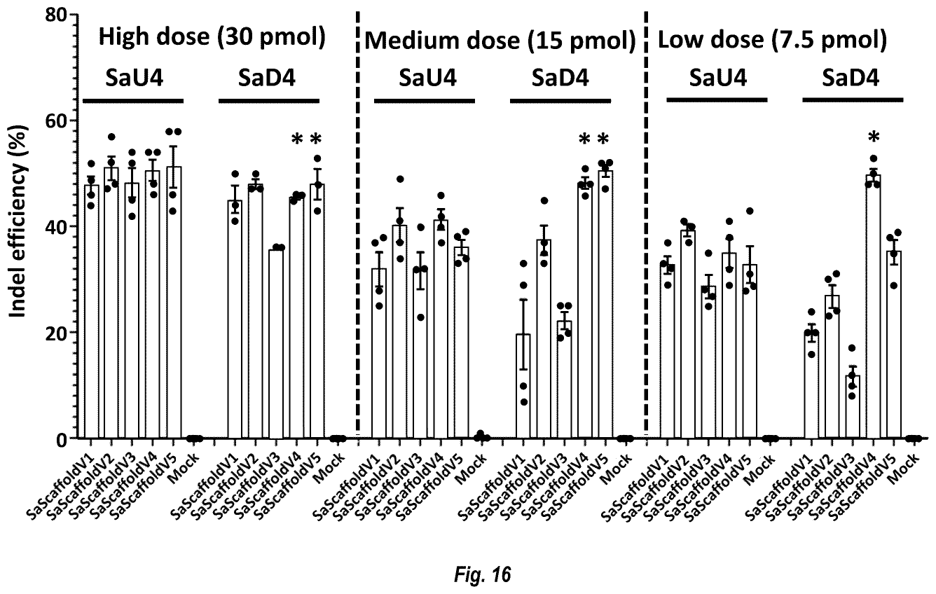

Editing efficiencies were further assessed in primary human myoblasts. SaCas9 protein and synthetic SaU4 or SaD4 sgRNAs with different scaffolds (see Table 6 and Table 9 for scaffold sequences) were nucleofected into primary human myoblasts at a ratio of 1:3. Three doses were evaluated: high dose, 30 pmol SaCas9 protein; medium dose, 15 pmol SaCas9 protein; and low dose, 7.5 pmol SaCas9 protein. Genomic DNA was extracted at 72 h post nucleofection, and a 1195 bp sequence covering the CTG repeat expansion and the sgRNA target sites was amplified by PCR. Sanger sequencing and ICE analysis were then used to quantify the frequency of indels generated by each sgRNA. Results are shown in FIG. 16 as mean±standard error (n=4). Bars with “*” above contain some data points with lower R².

### Example 4: Additional AAV Vector Configurations

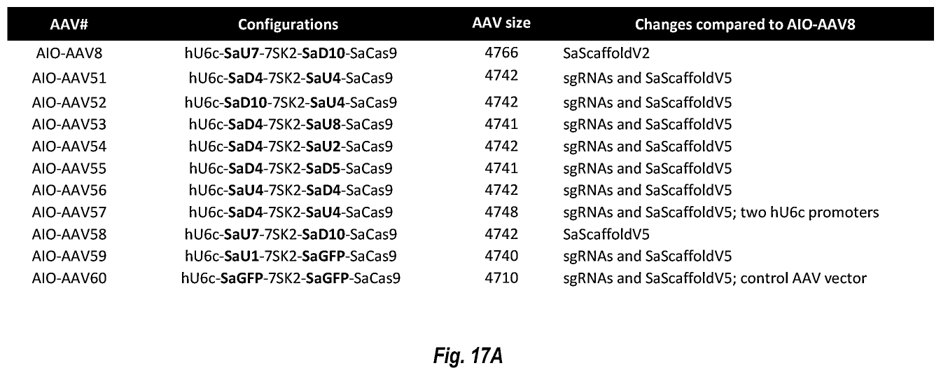

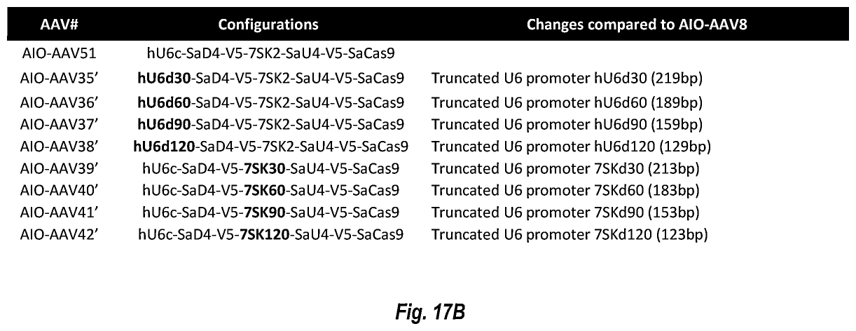

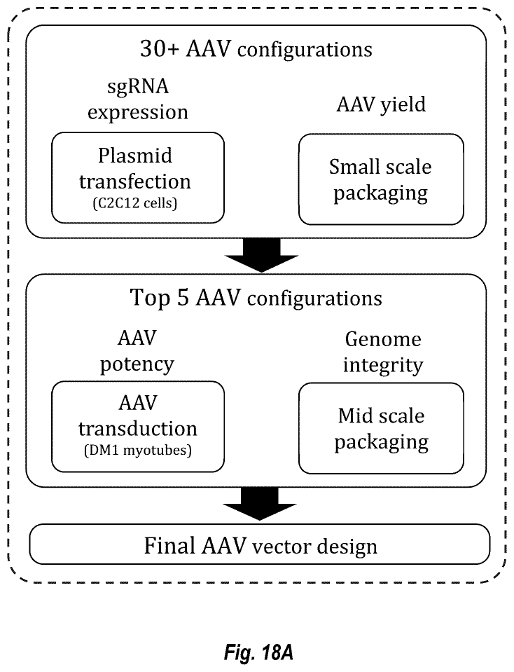

Utilizing SaScaffoldV5 (Table 9), further vector configurations were designed (FIG. 17A). For example, the vector configurations AIO-AA51, AIO-AAV52, AIO-AAV53, AIO-AAV-54, AIO-AAV55, AIO-AAV-56, AIO-AAV-57, AIO-AAV58, AIO-AAV59, and AIO-AAV60 were designed based off of AIO-AAV8 (schematic of AIO-AAV8 is shown in FIG. 17A). Sequences of vectors are shown in Table 10 below.

Additional vector configurations were designed based off of AIO-AA51 utilizing variants of the hU6c promoter (Table 7), variants of the 7SK2 promoter (Table 8), including AIO-AIO-AAV-35′, AIO-AAV36′, AIO-AAV-37′, AIO-AAV-38′, AIO-AAV39′, AIO-AAV40′, AIO-AAV41′, and AIO-AAV42′ as shown in FIG. 17B. Sequences of vectors are shown in Table 10 below.

TABLE 10
Exemplary Vector Sequences

| | SEQ | |
|---|---|---|
| | ID | |
| AAV # | NO | Sequence |
| AIO-AAV51 | 922 | TGTACTAGTGAGGGCCTATTTCCCATGATTCCTTCATATTTG |
| | | CATATACGATACAAGGCTGTTAGAGAGATAATTGGAATTAA |
| | | TTTGACTGTAAACACAAAGATATTAGTACAAAATACGTGAC |
| | | GTAGAAAGTAATAATTTCTTGGGTAGTTTGCAGTTTTAAAA |
| | | TTATGTTTTAAAATGGACTATCATATGCTTACCGTAACTTGA |
| | | AAGTATTTCGATTTCTTGGCTTTATATATCTTGTGGAAAGGA |
| | | CGAAACACCGACTTTGCGAACCAACGATAGGTGTTTCAGTA |
| | | CTCTGGAAACAGAATCTACTGAAACAAGGCAAAATGCCGT |
| | | GTTTATCTCGTCAACTTGTTGGCGAGATTTTTTTCTGCAGTA |
| | | TTTAGCATGCCCCACCCATCTGCAAGGCATTCTGGATAGTG |
| | | TCAAAACAGCCGGAAATCAAGTCCGTTTATCTCAAACTTTA |
| | | GCATTTTGGGAATAAATGATATTTGCTATGCTGGTTAAATT |
| | | AGATTTTAGTTAAATTTCCTGCTGAAGCTCTAGTACGATAA |
| | | GCAACTTGACCTAAGTGTAAAGTTGAGACTTCCTTCAGGTT |
| | | TATATAGCTTGTGCGCCGCTTGGGTACCTCGCCAGTTCACA |
| | | ACCGCTCCGAGCGTTTCAGTACTCTGGAAACAGAATCTACT |
| | | GAAACAAGGCAAAATGCCGTGTTTATCTCGTCAACTTGTTG |
| | | GCGAGATTTTTTTAGATCTGCCCATGTAAGGAGGCAAGGCC |
| | | TGGGGACACCCGAGATGCCTGGTTATAATTAA |
| AIO-AAV52 | 923 | TGTACTAGTGAGGGCCTATTTCCCATGATTCCTTCATATTTGCATA |
| | | TACGATACAAGGCTGTTAGAGAGATAATTGGAATTAATTTGACTG |
| | | TAAACACAAAGATATTAGTACAAAATACGTGACGTAGAAAGTAA |
| | | TAATTTCTTGGGTAGTTTGCAGTTTTAAAATTATGTTTTAAAATGG |
| | | ACTATCATATGCTTACCGTAACTTGAAAGTATTTCGATTTCTTGGC |
| | | TTTATATATCTTGTGGAAAGGACGAAACACCGTTGGGGGTCCTGT |
| | | AGCCTGTCAGTTTCAGTACTCTGGAAACAGAATCTACTGAAACAA |
| | | GGCAAAATGCCGTGTTTATCTCGTCAACTTGTTGGCGAGATTTTTT |
| | | TCTGCAGTATTTAGCATGCCCCACCCATCTGCAAGGCATTCTGGA |
| | | TAGTGTCAAAACAGCCGGAAATCAAGTCCGTTTATCTCAAACTTT |
| | | AGCATTTTGGGAATAAATGATATTTGCTATGCTGGTTAAATTAGA |
| | | TTTTAGTTAAATTTCCTGCTGAAGCTCTAGTACGATAAGCAACTTG |
| | | ACCTAAGTGTAAAGTTGAGACTTCCTTCAGGTTTATATAGCTTGTG |
| | | CGCCGCTTGGGTACCTCGCCAGTTCACAACCGCTCCGAGCGTTTC |
| | | AGTACTCTGGAAACAGAATCTACTGAAACAAGGCAAAATGCCGT |
| | | GTTTATCTCGTCAACTTGTTGGCGAGATTTTTTTAGATCTGCCCAT |
| | | GTAAGGAGGCAAGGCCTGGGGACACCCGAGATGCCTGGTTATAA |
| | | TTAA |
| AIO-AAV53 | 924 | TGTACTAGTGAGGGCCTATTTCCCATGATTCCTTCATATTTGCATA |
| | | TACGATACAAGGCTGTTAGAGAGATAATTGGAATTAATTTGACTG |
| | | TAAACACAAAGATATTAGTACAAAATACGTGACGTAGAAAGTAA |
| | | TAATTTCTTGGGTAGTTTGCAGTTTTAAAATTATGTTTTAAAATGG |
| | | ACTATCATATGCTTACCGTAACTTGAAAGTATTTCGATTTCTTGGC |
| | | TTTATATATCTTGTGGAAAGGACGAAACACCGACTTTGCGAACCA |
| | | ACGATAGGTGTTTCAGTACTCTGGAAACAGAATCTACTGAAACAA |
| | | GGCAAAATGCCGTGTTTATCTCGTCAACTTGTTGGCGAGATTTTTT |
| | | TCTGCAGTATTTAGCATGCCCCACCCATCTGCAAGGCATTCTGGA |
| | | TAGTGTCAAAACAGCCGGAAATCAAGTCCGTTTATCTCAAACTTT |
| | | AGCATTTTGGGAATAAATGATATTTGCTATGCTGGTTAAATTAGA |
| | | TTTTAGTTAAATTTCCTGCTGAAGCTCTAGTACGATAAGCAACTTG |
| | | ACCTAAGTGTAAAGTTGAGACTTCCTTCAGGTTTATATAGCTTGTG |
| | | CGCCGCTTGGGTACCTCGGCTCGAAGGGTCCTTGTAGCCGTTTCA |
| | | GTACTCTGGAAACAGAATCTACTGAAACAAGGCAAAATGCCGTG |
| | | TTTATCTCGTCAACTTGTTGGCGAGATTTTTTTAGATCTGCCCATG |
| | | TAAGGAGGCAAGGCCTGGGGACACCCGAGATGCCTGGTTATAAT |
| | | TAA |
| AIO-AAV54 | 925 | TGTACTAGTGAGGGCCTATTTCCCATGATTCCTTCATATTTGCATA |
| | | TACGATACAAGGCTGTTAGAGAGATAATTGGAATTAATTTGACTG |
| | | TAAACACAAAGATATTAGTACAAAATACGTGACGTAGAAAGTAA |
| | | TAATTTCTTGGGTAGTTTGCAGTTTTAAAATTATGTTTTAAAATGG |
| | | ACTATCATATGCTTACCGTAACTTGAAAGTATTTCGATTTCTTGGC |
| | | TTTATATATCTTGTGGAAAGGACGAAACACCGACTTTGCGAACCA |
| | | ACGATAGGTGTTTCAGTACTCTGGAAACAGAATCTACTGAAACAA |
| | | GGCAAAATGCCGTGTTTATCTCGTCAACTTGTTGGCGAGATTTTTT |
| | | TCTGCAGTATTTAGCATGCCCCACCCATCTGCAAGGCATTCTGGA |
| | | TAGTGTCAAAACAGCCGGAAATCAAGTCCGTTTATCTCAAACTTT |
| | | AGCATTTTGGGAATAAATGATATTTGCTATGCTGGTTAAATTAGA |
| | | TTTTAGTTAAATTTCCTGCTGAAGCTCTAGTACGATAAGCAACTTG |
| | | ACCTAAGTGTAAAGTTGAGACTTCCTTCAGGTTTATATAGCTTGTG |
| | | CGCCGCTTGGGTACCTCGACTCAGTCTTCCAACGGGGCCCGTTTC |
| | | AGTACTCTGGAAACAGAATCTACTGAAACAAGGCAAAATGCCGT |
| | | GTTTATCTCGTCAACTTGTTGGCGAGATTTTTTTAGATCTGCCCAT |
| | | GTAAGGAGGCAAGGCCTGGGGACACCCGAGATGCCTGGTTATAA |
| | | TTAA |
| AIO-AAV55 | 926 | TGTACTAGTGAGGGCCTATTTCCCATGATTCCTTCATATTTGCATA |
| | | TACGATACAAGGCTGTTAGAGAGATAATTGGAATTAATTTGACTG |
| | | TAAACACAAAGATATTAGTACAAAATACGTGACGTAGAAAGTAA |
| | | TAATTTCTTGGGTAGTTTGCAGTTTTAAAATTATGTTTTAAAATGG |
| | | ACTATCATATGCTTACCGTAACTTGAAAGTATTTCGATTTCTTGGC |
| | | TTTATATATCTTGTGGAAAGGACGAAACACCGCCAGTTCACAACC |
| | | GCTCCGAGCGTTTCAGTACTCTGGAAACAGAATCTACTGAAACAA |
| | | GGCAAAATGCCGTGTTTATCTCGTCAACTTGTTGGCGAGATTTTTT |
| | | TCTGCAGTATTTAGCATGCCCCACCCATCTGCAAGGCATTCTGGA |
| | | TAGTGTCAAAACAGCCGGAAATCAAGTCCGTTTATCTCAAACTTT |
| | | AGCATTTTGGGAATAAATGATATTTGCTATGCTGGTTAAATTAGA |
| | | TTTTAGTTAAATTTCCTGCTGAAGCTCTAGTACGATAAGCAACTTG |
| | | ACCTAAGTGTAAAGTTGAGACTTCCTTCAGGTTTATATAGCTTGTG |
| | | CGCCGCTTGGGTACCTCGGGTTTGGCAAAAGCAAATTTCGTTTCA |
| | | GTACTCTGGAAACAGAATCTACTGAAACAAGGCAAAATGCCGTG |
| | | TTTATCTCGTCAACTTGTTGGCGAGATTTTTTTAGATCTGCCCATG |
| | | TAAGGAGGCAAGGCCTGGGGACACCCGAGATGCCTGGTTATAAT |
| | | TAA |
| AIO-AAV56 | 927 | TGTACTAGTGAGGGCCTATTTCCCATGATTCCTTCATATTTGCATA |
| | | TACGATACAAGGCTGTTAGAGAGATAATTGGAATTAATTTGACTG |
| | | TAAACACAAAGATATTAGTACAAAATACGTGACGTAGAAAGTAA |
| | | TAATTTCTTGGGTAGTTTGCAGTTTTAAAATTATGTTTTAAAATGG |
| | | ACTATCATATGCTTACCGTAACTTGAAAGTATTTCGATTTCTTGGC |
| | | TTTATATATCTTGTGGAAAGGACGAAACACCGCCAGTTCACAACC |
| | | GCTCCGAGCGTTTCAGTACTCTGGAAACAGAATCTACTGAAACAA |
| | | GGCAAAATGCCGTGTTTATCTCGTCAACTTGTTGGCGAGATTTTTT |
| | | TCTGCAGTATTTAGCATGCCCCACCCATCTGCAAGGCATTCTGGA |
| | | TAGTGTCAAAACAGCCGGAAATCAAGTCCGTTTATCTCAAACTTT |
| | | AGCATTTTGGGAATAAATGATATTTGCTATGCTGGTTAAATTAGA |
| | | TTTTAGTTAAATTTCCTGCTGAAGCTCTAGTACGATAAGCAACTTG |
| | | ACCTAAGTGTAAAGTTGAGACTTCCTTCAGGTTTATATAGCTTGTG |
| | | CGCCGCTTGGGTACCTCGACTTTGCGAACCAACGATAGGTGTTTC |
| | | AGTACTCTGGAAACAGAATCTACTGAAACAAGGCAAAATGCCGT |
| | | GTTTATCTCGTCAACTTGTTGGCGAGATTTTTTTAGATCTGCCCAT |
| | | GTAAGGAGGCAAGGCCTGGGGACACCCGAGATGCCTGGTTATAA |
| | | TTAA |
| AIO-AAV57 | 928 | TGTACTAGTGAGGGCCTATTTCCCATGATTCCTTCATATTTGCATA |
| | | TACGATACAAGGCTGTTAGAGAGATAATTGGAATTAATTTGACTG |
| | | TAAACACAAAGATATTAGTACAAAATACGTGACGTAGAAAGTAA |
| | | TAATTTCTTGGGTAGTTTGCAGTTTTAAAATTATGTTTTAAAATGG |
| | | ACTATCATATGCTTACCGTAACTTGAAAGTATTTCGATTTCTTGGC |
| | | TTTATATATCTTGTGGAAAGGACGAAACACCGACTTTGCGAACCA |
| | | ACGATAGGTGTTTCAGTACTCTGGAAACAGAATCTACTGAAACAA |
| | | GGCAAAATGCCGTGTTTATCTCGTCAACTTGTTGGCGAGATTTTTT |
| | | TGAGGGCCTATTTCCCATGATTCCTTCATATTTGCATATACGATAC |
| | | AAGGCTGTTAGAGAGATAATTGGAATTAATTTGACTGTAAACACA |
| | | AAGATATTAGTACAAAATACGTGACGTAGAAAGTAATAATTTCTT |
| | | GGGTAGTTTGCAGTTTTAAAATTATGTTTTAAAATGGACTATCATA |
| | | TGCTTACCGTAACTTGAAAGTATTTCGATTTCTTGGCTTTATATAT |
| | | CTTGTGGAAAGGACGAAACACCGCCAGTTCACAACCGCTCCGAG |
| | | CGTTTCAGTACTCTGGAAACAGAATCTACTGAAACAAGGCAAAAT |
| | | GCCGTGTTTATCTCGTCAACTTGTTGGCGAGATTTTTTTAGATCTG |
| | | CCCATGTAAGGAGGCAAGGCCTGGGGACACCCGAGATGCCTGGT |
| | | TATAATTAA |
| AIO-AAV58 | 929 | TGTACTAGTGAGGGCCTATTTCCCATGATTCCTTCATATTTGCATA |
| | | TACGATACAAGGCTGTTAGAGAGATAATTGGAATTAATTTGACTG |
| | | TAAACACAAAGATATTAGTACAAAATACGTGACGTAGAAAGTAA |
| | | TAATTTCTTGGGTAGTTTGCAGTTTTAAAATTATGTTTTAAAATGG |
| | | ACTATCATATGCTTACCGTAACTTGAAAGTATTTCGATTTCTTGGC |
| | | TTTATATATCTTGTGGAAAGGACGAAACACCGCGCGGCCGGCGAA |
| | | CGGGGCTCGGTTTCAGTACTCTGGAAACAGAATCTACTGAAACAA |
| | | GGCAAAATGCCGTGTTTATCTCGTCAACTTGTTGGCGAGATTTTTT |
| | | TCTGCAGTATTTAGCATGCCCCACCCATCTGCAAGGCATTCTGGA |
| | | TAGTGTCAAAACAGCCGGAAATCAAGTCCGTTTATCTCAAACTTT |
| | | AGCATTTTGGGAATAAATGATATTTGCTATGCTGGTTAAATTAGA |
| | | TTTTAGTTAAATTTCCTGCTGAAGCTCTAGTACGATAAGCAACTTG |
| | | ACCTAAGTGTAAAGTTGAGACTTCCTTCAGGTTTATATAGCTTGTG |
| | | CGCCGCTTGGGTACCTCGTTGGGGGTCCTGTAGCCTGTCAGTTTCA |
| | | GTACTCTGGAAACAGAATCTACTGAAACAAGGCAAAATGCCGTG |
| | | TTTATCTCGTCAACTTGTTGGCGAGATTTTTTTAGATCTGCCCATG |
| | | TAAGGAGGCAAGGCCTGGGGACACCCGAGATGCCTGGTTATAAT |
| | | TAA |
| AIO-AAV59 | 930 | TGTACTAGTGAGGGCCTATTTCCCATGATTCCTTCATATTTGCATA |
| | | TACGATACAAGGCTGTTAGAGAGATAATTGGAATTAATTTGACTG |
| | | TAAACACAAAGATATTAGTACAAAATACGTGACGTAGAAAGTAA |
| | | TAATTTCTTGGGTAGTTTGCAGTTTTAAAATTATGTTTTAAAATGG |
| | | ACTATCATATGCTTACCGTAACTTGAAAGTATTTCGATTTCTTGGC |
| | | TTTATATATCTTGTGGAAAGGACGAAACACCGCCCCGGAGTCGAA |
| | | GACAGTTCGTTTCAGTACTCTGGAAACAGAATCTACTGAAACAAG |
| | | GCAAAATGCCGTGTTTATCTCGTCAACTTGTTGGCGAGATTTTTTT |
| | | CTGCAGTATTTAGCATGCCCCACCCATCTGCAAGGCATTCTGGAT |
| | | AGTGTCAAAACAGCCGGAAATCAAGTCCGTTTATCTCAAACTTTA |
| | | GCATTTTGGGAATAAATGATATTTGCTATGCTGGTTAAATTAGATT |
| | | TTAGTTAAATTTCCTGCTGAAGCTCTAGTACGATAAGCAACTTGA |
| | | CCTAAGTGTAAAGTTGAGACTTCCTTCAGGTTTATATAGCTTGTGC |
| | | GCCGCTTGGGTACCTCGCCCCGGAGTCGAAGACAGTTCGTTTCAG |
| | | TACTCTGGAAACAGAATCTACTGAAACAAGGCAAAATGCCGTGTT |
| | | TATCTCGTCAACTTGTTGGCGAGATTTTTTTAGATCTGCCCATGTA |
| | | AGGAGGCAAGGCCTGGGGACACCCGAGATGCCTGGTTATAATTA |
| | | A |
| AIO-AAV60 | 931 | TGTACTAGTGAGGGCCTATTTCCCATGATTCCTTCATATTTGCATA |
| | | TACGATACAAGGCTGTTAGAGAGATAATTGGAATTAATTTGACTG |
| | | TAAACACAAAGATATTAGTACAAAATACGTGACGTAGAAAGTAA |
| | | TAATTTCTTGGGTAGTTTGCAGTTTTAAAATTATGTTTTAAAATGG |
| | | ACTATCATATGCTTACCGTAACTTGAAAGTATTTCGATTTCTTGGC |
| | | TTTATATATCTTGTGGAAAGGACGAAACACCGGCAACATCCTGGG |
| | | GCACAAGCGTTTCAGTACTCTGGAAACAGAATCTACTGAAACAAG |
| | | GCAAAATGCCGTGTTTATCTCGTCAACTTGTTGGCGAGATTTTTTT |
| | | CTGCAGTATTTAGCATGCCCCACCCATCTGCAAGGCATTCTGGAT |
| | | AGTGTCAAAACAGCCGGAAATCAAGTCCGTTTATCTCAAACTTTA |
| | | GCATTTTGGGAATAAATGATATTTGCTATGCTGGTTAAATTAGATT |
| | | TTAGTTAAATTTCCTGCTGAAGCTCTAGTACGATAAGCAACTTGA |
| | | CCTAAGTGTAAAGTTGAGACTTCCTTCAGGTTTATATAGCTTGTGC |
| | | GCCGCTTGGGTACCTCGGCAACATCCTGGGGCACAAGCGTTTCAG |
| | | TACTCTGGAAACAGAATCTACTGAAACAAGGCAAAATGCCGTGTT |
| | | TATCTCGTCAACTTGTTGGCGAGATTTTTTTAGATCTGCCCATGTA |
| | | AGGAGGCAAGGCCTGGGGACACCCGAGATGCCTGGTTATAATTA |
| | | A |
| AIO-AAV35′ | 932 | TGTACTAGTGAGGGCCTATTTCCCATGATTCCTTCATATTTGCATA |
| | | TACGATACAAGGCTGTTAGAGAGATAATTGGAATTAATTTGACTG |
| | | TAAACACAAAGATATAATTTCTTGGGTAGTTTGCAGTTTTAAAAT |
| | | TATGTTTTAAAATGGACTATCATATGCTTACCGTAACTTGAAAGT |
| | | ATTTCGATTTCTTGGCTTTATATATCTTGTGGAAAGGACGAAACAC |
| | | CGACTTTGCGAACCAACGATAGGTGTTTCAGTACTCTGGAAACAG |
| | | AATCTACTGAAACAAGGCAAAATGCCGTGTTTATCTCGTCAACTT |
| | | GTTGGCGAGATTTTTTTCTGCAGTATTTAGCATGCCCCACCCATCT |
| | | GCAAGGCATTCTGGATAGTGTCAAAACAGCCGGAAATCAAGTCC |
| | | GTTTATCTCAAACTTTAGCATTTTGGGAATAAATGATATTTGCTAT |
| | | GCTGGTTAAATTAGATTTTAGTTAAATTTCCTGCTGAAGCTCTAGT |
| | | ACGATAAGCAACTTGACCTAAGTGTAAAGTTGAGACTTCCTTCAG |
| | | GTTTATATAGCTTGTGCGCCGCTTGGGTACCTCGCCAGTTCACAAC |
| | | CGCTCCGAGCGTTTCAGTACTCTGGAAACAGAATCTACTGAAACA |
| | | AGGCAAAATGCCGTGTTTATCTCGTCAACTTGTTGGCGAGATTTTT |
| | | TTAGATCTGCCCATGTAAGGAGGCAAGGCCTGGGGACACCCGAG |
| | | ATGCCTGGTTATAATTAA |
| AIO-AAV36′ | 933 | TGTACTAGTGAGGGCCTATTTCCCATGATTCCTTCATATTTGCATA |
| | | TACGATACAAGGCTGTTAGAGAGATAATTGGAATTAATTTGACGT |
| | | TTGCAGTTTTAAAATTATGTTTTAAAATGGACTATCATATGCTTAC |
| | | CGTAACTTGAAAGTATTTCGATTTCTTGGCTTTATATATCTTGTGG |
| | | AAAGGACGAAACACCGACTTTGCGAACCAACGATAGGTGTTTCA |
| | | GTACTCTGGAAACAGAATCTACTGAAACAAGGCAAAATGCCGTG |
| | | TTTATCTCGTCAACTTGTTGGCGAGATTTTTTTCTGCAGTATTTAG |
| | | CATGCCCCACCCATCTGCAAGGCATTCTGGATAGTGTCAAAACAG |
| | | CCGGAAATCAAGTCCGTTTATCTCAAACTTTAGCATTTTGGGAAT |
| | | AAATGATATTTGCTATGCTGGTTAAATTAGATTTTAGTTAAATTTC |
| | | CTGCTGAAGCTCTAGTACGATAAGCAACTTGACCTAAGTGTAAAG |
| | | TTGAGACTTCCTTCAGGTTTATATAGCTTGTGCGCCGCTTGGGTAC |
| | | CTCGCCAGTTCACAACCGCTCCGAGCGTTTCAGTACTCTGGAAAC |
| | | AGAATCTACTGAAACAAGGCAAAATGCCGTGTTTATCTCGTCAAC |
| | | TTGTTGGCGAGATTTTTTTAGATCTGCCCATGTAAGGAGGCAAGG |
| | | CCTGGGGACACCCGAGATGCCTGGTTATAATTAA |
| AIO-AAV37′ | 934 | TGTACTAGTGAGGGCCTATTTCCCATGATTCCTTCATATTTGCATA |
| | | TACGATACAAGGCTGTTAGAGAGATAATATTATGTTTTAAAATGG |
| | | ACTATCATATGCTTACCGTAACTTGAAAGTATTTCGATTTCTTGGC |
| | | TTTATATATCTTGTGGAAAGGACGAAACACCGACTTTGCGAACCA |
| | | ACGATAGGTGTTTCAGTACTCTGGAAACAGAATCTACTGAAACAA |
| | | GGCAAAATGCCGTGTTTATCTCGTCAACTTGTTGGCGAGATTTTTT |
| | | TCTGCAGTATTTAGCATGCCCCACCCATCTGCAAGGCATTCTGGA |
| | | TAGTGTCAAAACAGCCGGAAATCAAGTCCGTTTATCTCAAACTTT |
| | | AGCATTTTGGGAATAAATGATATTTGCTATGCTGGTTAAATTAGA |
| | | TTTTAGTTAAATTTCCTGCTGAAGCTCTAGTACGATAAGCAACTTG |
| | | ACCTAAGTGTAAAGTTGAGACTTCCTTCAGGTTTATATAGCTTGTG |
| | | CGCCGCTTGGGTACCTCGCCAGTTCACAACCGCTCCGAGCGTTTC |
| | | AGTACTCTGGAAACAGAATCTACTGAAACAAGGCAAAATGCCGT |
| | | GTTTATCTCGTCAACTTGTTGGCGAGATTTTTTTAGATCTGCCCAT |
| | | GTAAGGAGGCAAGGCCTGGGGACACCCGAGATGCCTGGTTATAA |
| | | TTAA |
| AIO-AAV38′ | 935 | TGTACTAGTGAGGGCCTATTTCCCATGATTCCTTCATATTTGCATA |
| | | TACGATACAAGGCGGACTATCATATGCTTACCGTAACTTGAAAGT |
| | | ATTTCGATTTCTTGGCTTTATATATCTTGTGGAAAGGACGAAACAC |
| | | CGACTTTGCGAACCAACGATAGGTGTTTCAGTACTCTGGAAACAG |
| | | AATCTACTGAAACAAGGCAAAATGCCGTGTTTATCTCGTCAACTT |
| | | GTTGGCGAGATTTTTTTCTGCAGTATTTAGCATGCCCCACCCATCT |
| | | GCAAGGCATTCTGGATAGTGTCAAAACAGCCGGAAATCAAGTCC |
| | | GTTTATCTCAAACTTTAGCATTTTGGGAATAAATGATATTTGCTAT |
| | | GCTGGTTAAATTAGATTTTAGTTAAATTTCCTGCTGAAGCTCTAGT |
| | | ACGATAAGCAACTTGACCTAAGTGTAAAGTTGAGACTTCCTTCAG |
| | | GTTTATATAGCTTGTGCGCCGCTTGGGTACCTCGCCAGTTCACAAC |
| | | CGCTCCGAGCGTTTCAGTACTCTGGAAACAGAATCTACTGAAACA |
| | | AGGCAAAATGCCGTGTTTATCTCGTCAACTTGTTGGCGAGATTTTT |
| | | TTAGATCTGCCCATGTAAGGAGGCAAGGCCTGGGGACACCCGAG |
| | | ATGCCTGGTTATAATTAA |
| AIO-AAV39′ | 936 | TGTACTAGTGAGGGCCTATTTCCCATGATTCCTTCATATTTGCATA |
| | | TACGATACAAGGCTGTTAGAGAGATAATTGGAATTAATTTGACTG |
| | | TAAACACAAAGATATTAGTACAAAATACGTGACGTAGAAAGTAA |
| | | TAATTTCTTGGGTAGTTTGCAGTTTTAAAATTATGTTTTAAAATGG |
| | | ACTATCATATGCTTACCGTAACTTGAAAGTATTTCGATTTCTTGGC |
| | | TTTATATATCTTGTGGAAAGGACGAAACACCGACTTTGCGAACCA |
| | | ACGATAGGTGTTTCAGTACTCTGGAAACAGAATCTACTGAAACAA |
| | | GGCAAAATGCCGTGTTTATCTCGTCAACTTGTTGGCGAGATTTTTT |
| | | TCTGCAGTATTTAGCATGCCCCACCCATCTGCAAGGCATTCTGGA |
| | | TAGTGTCAAAACAGCCGGAAATCAAGTCCGTTTATCTCAAACTTT |
| | | AGCATTTAAATTAGATTTTAGTTAAATTTCCTGCTGAAGCTCTAGT |
| | | ACGATAAGCAACTTGACCTAAGTGTAAAGTTGAGACTTCCTTCAG |
| | | GTTTATATAGCTTGTGCGCCGCTTGGGTACCTCGCCAGTTCACAAC |
| | | CGCTCCGAGCGTTTCAGTACTCTGGAAACAGAATCTACTGAAACA |
| | | AGGCAAAATGCCGTGTTTATCTCGTCAACTTGTTGGCGAGATTTTT |
| | | TTAGATCTGCCCATGTAAGGAGGCAAGGCCTGGGGACACCCGAG |
| | | ATGCCTGGTTATAATTAA |
| AIO-AAV40′ | 937 | TGTACTAGTGAGGGCCTATTTCCCATGATTCCTTCATATTTGCATA |
| | | TACGATACAAGGCTGTTAGAGAGATAATTGGAATTAATTTGACTG |
| | | TAAACACAAAGATATTAGTACAAAATACGTGACGTAGAAAGTAA |
| | | TAATTTCTTGGGTAGTTTGCAGTTTTAAAATTATGTTTTAAAATGG |
| | | ACTATCATATGCTTACCGTAACTTGAAAGTATTTCGATTTCTTGGC |
| | | TTTATATATCTTGTGGAAAGGACGAAACACCGACTTTGCGAACCA |
| | | ACGATAGGTGTTTCAGTACTCTGGAAACAGAATCTACTGAAACAA |
| | | GGCAAAATGCCGTGTTTATCTCGTCAACTTGTTGGCGAGATTTTTT |
| | | TCTGCAGTATTTAGCATGCCCCACCCATCTGCAAGGCATTCTGGA |
| | | TAGTGTCAAAACAGCCGGAAATCAAGTCCGTTTATCTTAAATTTC |
| | | CTGCTGAAGCTCTAGTACGATAAGCAACTTGACCTAAGTGTAAAG |
| | | TTGAGACTTCCTTCAGGTTTATATAGCTTGTGCGCCGCTTGGGTAC |
| | | CTCGCCAGTTCACAACCGCTCCGAGCGTTTCAGTACTCTGGAAAC |
| | | AGAATCTACTGAAACAAGGCAAAATGCCGTGTTTATCTCGTCAAC |
| | | TTGTTGGCGAGATTTTTTTAGATCTGCCCATGTAAGGAGGCAAGG |
| | | CCTGGGGACACCCGAGATGCCTGGTTATAATTAA |
| AIO-AAV41′ | 938 | TGTACTAGTGAGGGCCTATTTCCCATGATTCCTTCATATTTGCATA |
| | | TACGATACAAGGCTGTTAGAGAGATAATTGGAATTAATTTGACTG |
| | | TAAACACAAAGATATTAGTACAAAATACGTGACGTAGAAAGTAA |
| | | TAATTTCTTGGGTAGTTTGCAGTTTTAAAATTATGTTTTAAAATGG |
| | | ACTATCATATGCTTACCGTAACTTGAAAGTATTTCGATTTCTTGGC |
| | | TTTATATATCTTGTGGAAAGGACGAAACACCGACTTTGCGAACCA |
| | | ACGATAGGTGTTTCAGTACTCTGGAAACAGAATCTACTGAAACAA |
| | | GGCAAAATGCCGTGTTTATCTCGTCAACTTGTTGGCGAGATTTTTT |
| | | TCTGCAGTATTTAGCATGCCCCACCCATCTGCAAGGCATTCTGGA |
| | | TAGTGTCAAAACAGCCGGAAATAGCTCTAGTACGATAAGCAACTT |
| | | GACCTAAGTGTAAAGTTGAGACTTCCTTCAGGTTTATATAGCTTGT |
| | | GCGCCGCTTGGGTACCTCGCCAGTTCACAACCGCTCCGAGCGTTT |
| | | CAGTACTCTGGAAACAGAATCTACTGAAACAAGGCAAAATGCCG |
| | | TGTTTATCTCGTCAACTTGTTGGCGAGATTTTTTTAGATCTGCCCA |
| | | TGTAAGGAGGCAAGGCCTGGGGACACCCGAGATGCCTGGTTATA |
| | | ATTAA |
| AIO-AAV42′ | 939 | TGTACTAGTGAGGGCCTATTTCCCATGATTCCTTCATATTTGCATA |
| | | TACGATACAAGGCTGTTAGAGAGATAATTGGAATTAATTTGACTG |
| | | TAAACACAAAGATATTAGTACAAAATACGTGACGTAGAAAGTAA |
| | | TAATTTCTTGGGTAGTTTGCAGTTTTAAAATTATGTTTTAAAATGG |
| | | ACTATCATATGCTTACCGTAACTTGAAAGTATTTCGATTTCTTGGC |
| | | TTTATATATCTTGTGGAAAGGACGAAACACCGACTTTGCGAACCA |
| | | ACGATAGGTGTTTCAGTACTCTGGAAACAGAATCTACTGAAACAA |
| | | GGCAAAATGCCGTGTTTATCTCGTCAACTTGTTGGCGAGATTTTTT |
| | | TCTGCAGTATTTAGCATGCCCCACCCATCTGCAAGGCATTCTGGA |
| | | TAGTGTCAGCAACTTGACCTAAGTGTAAAGTTGAGACTTCCTTCA |
| | | GGTTTATATAGCTTGTGCGCCGCTTGGGTACCTCGCCAGTTCACA |
| | | ACCGCTCCGAGCGTTTCAGTACTCTGGAAACAGAATCTACTGAAA |
| | | CAAGGCAAAATGCCGTGTTTATCTCGTCAACTTGTTGGCGAGATT |
| | | TTTTTAGATCTGCCCATGTAAGGAGGCAAGGCCTGGGGACACCCG |
| | | AGATGCCTGGTTATAATTAA |

### Example 4: All-in-One AAV Vector Development

Many parameters can contribute to a more potent AAV vector, including high level of transgene expression, high efficiency of viral packaging, low level of viral genome truncation and recombination, etc. These features can be affected by AAV vector size, the specificity and robustness of the promoter used, the sequence of the sgRNA scaffold, and the arrangement of transgene expression cassettes. Thirty-five (35) different AAV vector configurations were designed (Table 11) with the same sgRNA pairs, SaU7 (SEQ ID NO: 7)+SaD10 (SEQ ID NO: 18) to evaluate these parameters. In general, two rounds of evaluation were implemented into the optimization procedure

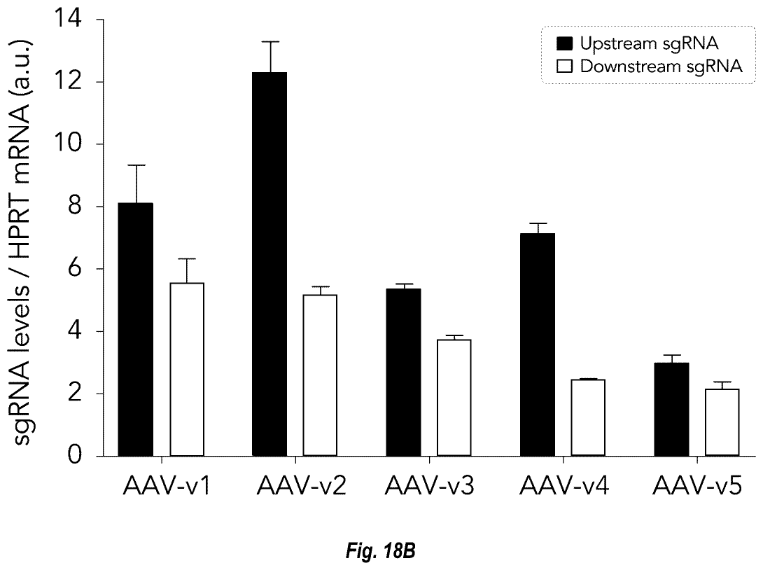

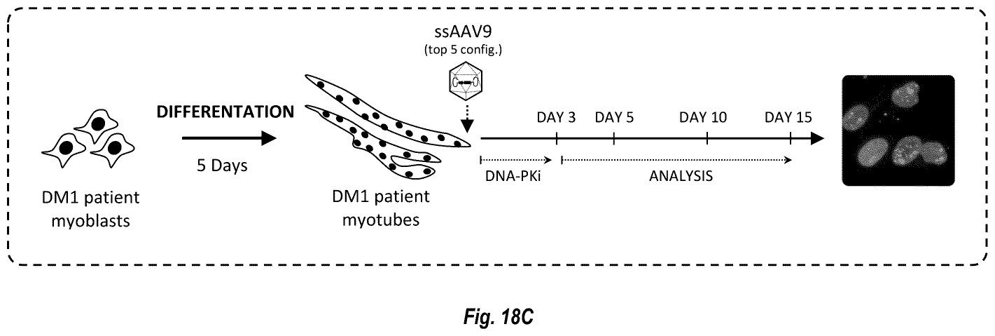

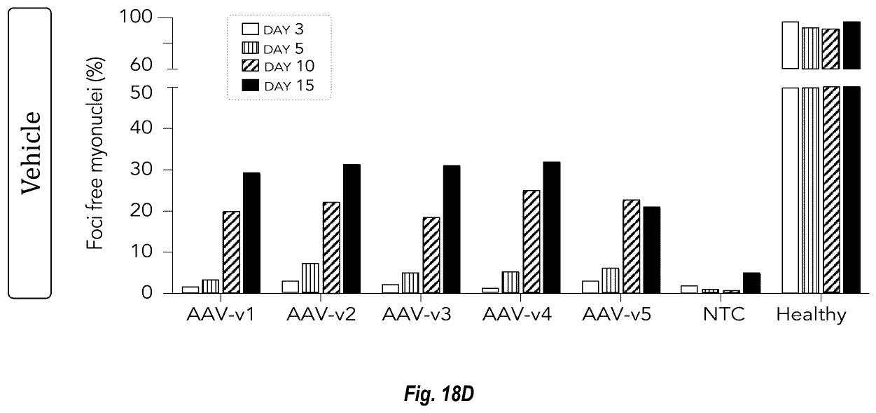

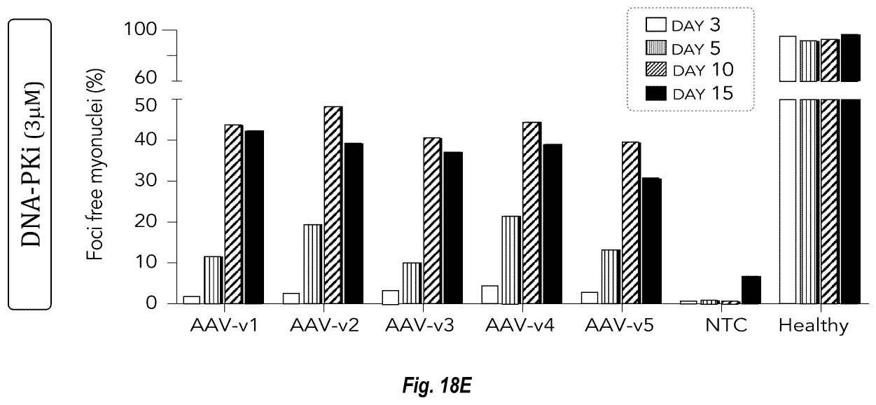

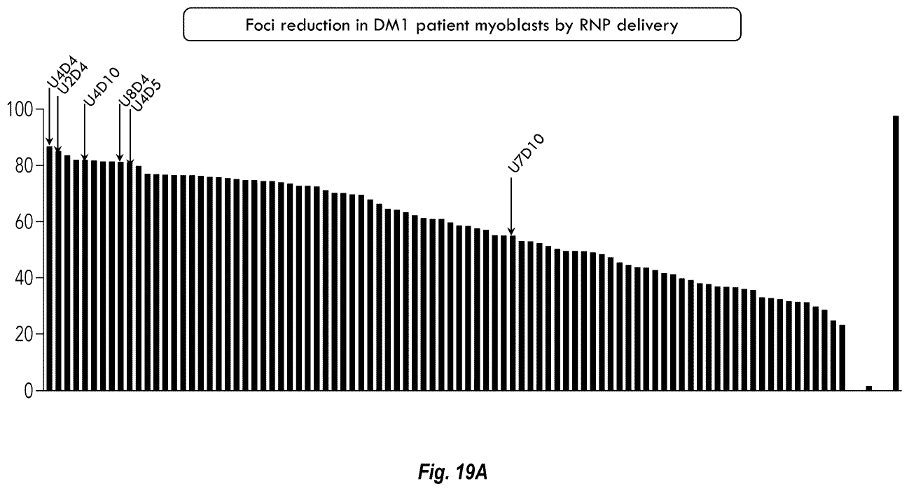

(FIG. 18A). In the first round, sgRNA expression was assessed through plasmid transfection in C2C12 cells and AAV packaging efficiency through small scale packaging in AAV 293 cells. The top 5 AAV configurations were selected from the first round of selection and are named AAV-v1-5 (Table 11). “AAV-v1” has a “Design 3 Cas9 on Right” geometry as shown in FIG. 10B, specifically hU6c-U7-hU6c-D10-Cas9. “AAV-v2” has a “Design 3 Cas9 on Right” geometry as shown in FIG. 10B, specifically hU6c-U7-7SK2-D10-Cas9. “AAV-v3” has a “Design 4 Cas9 on Left” geometry as shown in FIG. 10B, specifically Cas9-hU6c-U7-hU6c-D10. “AAV-v4” has a “Design 4 Cas9 on Left” geometry as shown in FIG. 10B, specifically Cas9-hU6c-U7-7SK2-D10. “AAV-v5” has a “Design 2 Cas9 in Middle” geometry as shown in FIG. 10B, specifically 7SK2-D10-Cas9-hU6c-U7 (but where the 7SK2 and D10 are in the reverse orientation, see FIG. 10A). Among the 5 configurations, AAV-v1 and AAV-v2 showed relative higher sgRNA expression (FIG. 18B), although all configurations showed good expression.

In the second round of evaluation, ssAAV virus were produced at relative large scale for ally configurations, AAV titer was assessed by ddPCR, and AAV genome integrity was assessed by Alkaline gel, then all 5 ssAAVs were used to transduce in vitro differentiated DM1 patient myotubes (FIG. 18C). In general, primary DM1 patient myoblasts were differentiated for 5 days to form postmitotic, multinucleated myotubes, and AAV transduction was performed on Day 5 with the treatment of neuraminidase (3.33 Units/mL). Infected myotubes were then treated with DMSO (vehicle) or 3 μM of Compound 6 (DNA-PKi) for 72 hours. Myotube samples were kept in differentiation media until fixation on day 3, 5, 10 and 15 post infection, and subjected to RNA foci staining (FIG. 18D (vehicle) and 18E (DNA-PKi)).

In the vehicle group, RNA foci staining showed increased foci-free myonuclei with extended culture time post infection (FIG. 18D). By day 15 post-infection, all AAV configurations achieved −30% foci-free myonuclei except for AAV-v5 (21%). Consistent with the previous DOUBLE-cut screening in myoblasts, the DNA-PKi treated group showed more robust foci reduction (with highest efficiency of 48% foci-free myonuclei from AAV-v2 at day 10) compared to the vehicle group, which can be observed at a relatively early time point, around day 5 post-infection. Different foci reduction kinetics were also observed between the DNA-PKi and vehicle group, and in the DNA-PKi group, ally configurations appeared to reach a plateau at day 10, while most configurations from the vehicle group continued to increase until day 15 except for AAV-v5.

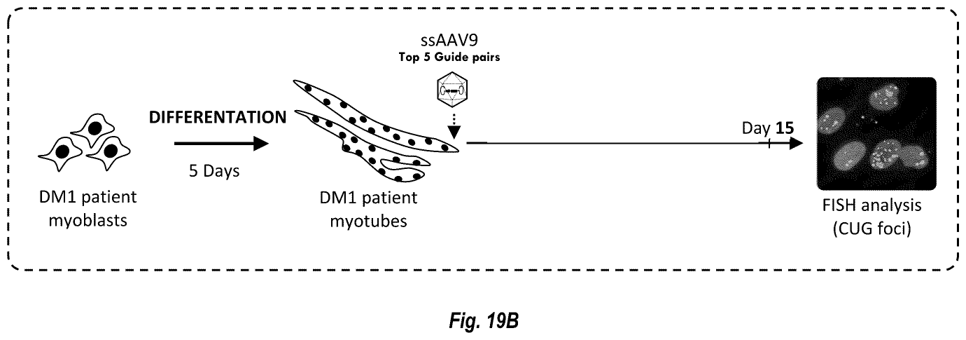

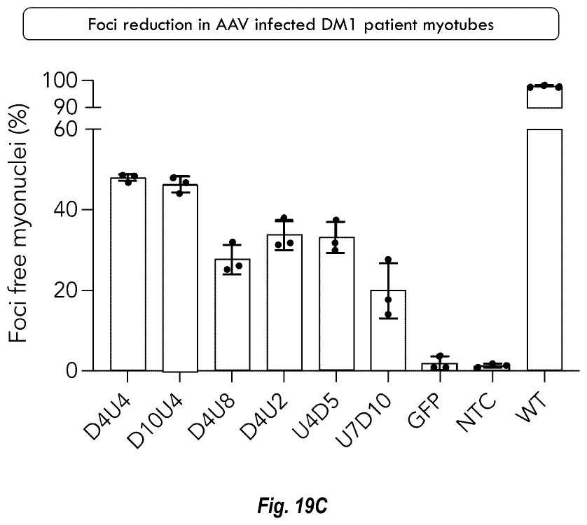

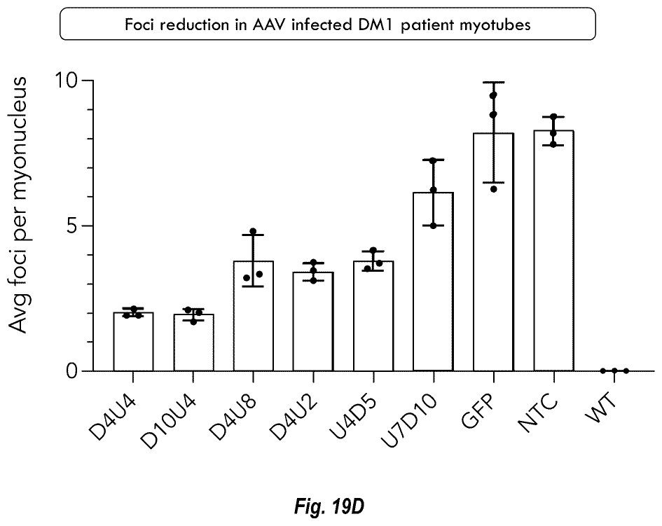

To assess the foci reduction efficiency of the top 5 sgRNA pairs from DOUBLE-cut screening, ssAAV viruses were produced for ally pairs (SaD4+SaU4; SaD10+SaU4; SaD4+SaU8; SaD4+SaU2; and SaU4+SaDS) together with a sixth pair: SaU7 (SEQ ID NO: 7)+SaD10 (SEQ ID NO: 18), which has been used for previous optimization processes (FIG. 19A). AAV infection was performed as described herein, and for this analysis, DNA-PKi treatment was not included. For the control group, in addition to non-treated ctrl (NTC), a GFP control group (GFP) was included with the same AAV-v2 configuration as CK8e-SaCas9, but with guides targeting a GFP sequence which should not bind to any human sequence, and where the myotubes are fixed only at day 15 post transduction for Foci staining (FIG. 19B). Consistent with DOUBLE-cut sgRNA screening ranking, SaU7 (SEQ ID NO: 7)+SaD10 (SEQ ID NO: 18) showed lower efficiency of foci reduction (19.9% foci-free myonuclei at day 15) compared to each of the 5 top guide pairs in AAV transduction platformed in postmitotic myotubes (FIG. 19C). Among the 5 top sgRNA pairs, SaD4+SaU4 (SEQ ID NOs: 12+4, respectively) and SaD10+SaU4 (SEQ ID NOs: 18+4, respectively) showed the highest foci-reduction efficiency to achieve close to 50% of foci free myonuclei (48.0% for SaD4+SaU4 [SEQ ID NOS: 12+4], 46.3% for SaD10+SaU4 [SEQ ID NOs: 18+4]) by day 15, followed by SaD4+SaU2 (33.7%) (SEQ ID NOs: 12+2, respectively), SaU4+SaD5 (33.0%) (SEQ ID NOs: 4+13, respectively), and SaD4+SaU8 (27.6%) (SEQ ID NOs: 12+8, respectively). Similar trends were observed from the quantification of average foci number per myonuclei (FIG. 19D).

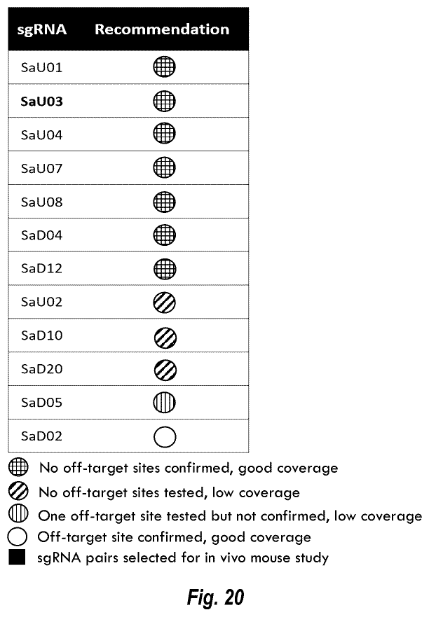

Several exemplary SaCas9 sgRNA pairs, all of which computationally predicted off-target sites associated with the 12 SaCas9 sgRNAs, were evaluated for safety using the hybrid capture method coupled with ultra-deep sequencing: 6 upstream sgRNA: SaU1, SaU2, SaU3, SaU4, SaU7, SaU8 (SEQ ID NOs: 1, 2, 3, 4, 7, 8, respectively), and 6 downstream sgRNAs: SaD2, SaD4, SaD5, SaD10, SaD12, SaD20 (SEQ ID NOs: 10, 12, 13, 18, 20, 28, respectively). HSMM cells from three healthy donors were nucleofected with the SaCas9/sgRNA pairs RNP complex at the dose used in the guide screening that showed therapeutic relevant efficacy of CTG excision and RNA Foci reduction. To ensure analysis in the relevant cell type, the nucleofected myoblast cells were purified and enriched using a myoblast specific cell surface marker prior to genomic DNA isolation. Edited genomic DNA samples were hybridized with the oligonucleotide probes to allow capture and enrichment of the regions around the off-target sites. Editing efficiency at the computationally predicted off-target sites were determined by ultra-deep-sequencing. The analysis results confirm one off-target site for SaD2 (SEQ ID NO: 10) at region chr4, 52837941-52837969 with p=0.032. Another off-target site for SaD5 (SEQ ID NO: 13) was also detected by the analysis with a non-significant p value of 0.21. A summary of the off-target analysis is illustrated in FIG. 20.

TABLE 11

| | | Vector size (bp)* | Top 5 |
|---|---|---|---|
| Promoter arrangement | Guide promoter combinations | Including ITR | candidates |
| Cas9 in Middle | hU6c-U7-Cas9-hU6c-D10 | 4771 | |
| (in line) | | | |
| (“Design 1” geometries | hU6c-U7-Cas9-7SK2-D10 | 4765 | |
| from FIG. 10B) | hU6c-U7-Cas9-H1m-D10 | 4623 | |
| Cas9 in Middle | hU6c-U7-Cas9-hU6c-D10 | 4771 | |
| (divergent) | hU6c-U7-Cas9-7SK2-D10 | 4765 | |
| (“Design 2” geometries | hU6c-U7-Cas9-H1m-D10 | 4623 | |
| from FIG. 10B) | 7SK2-D10-Cas9-hU6c-U7 | 4765 | AAV-v5 |
| | M11-D10-Cas9-hU6c-U7 | 4622 | |
| | M11-U7-Cas9-hU6c-D10 | 4622 | |
| | M11-U7-Cas9-7SK-D10 | 4616 | |
| | M11-U7-Cas9-H1m-D10 | 4473 | |
| Cas9 on Right | hU6c-U7-hU6c-D10-Cas9 | 4771 | AAV-v1 |
| (in line) | hU6c-U7-7SK2-D10-Cas9 | 4765 | AAV-v2 |
| (“Design 3” geometries | hU6c-U7-H1m-D10-Cas9 | 4623 | |
| from FIG. 10B) | hU6c-U7(v5)-7SK2-D10(v5)-Cas9 | 4766 | |
| | hU6c-U7(v2)-7SK2-D10(v2)-Cas9 | 4790 | |
| | hU6c(d30)-7SK2-Cas9 | 4736 | |
| | hU6c(d60)-7SK2-Cas9 | 4706 | |
| | hU6c(d90)-7SK2-Cas9 | 4676 | |
| | hU6c(d120)-7SK2-Cas9 | 4646 | |
| | hU6c-7SK2(d30)-Cas9 | 4736 | |
| | hU6c-7SK2(d60)-Cas9 | 4706 | |
| | hU6c-7SK2(d90)-Cas9 | 4676 | |
| | hU6c-7SK2(d120)-Cas9 | 4646 | |
| Cas9 on Left | Cas9-hU6c-U7-hU6c-D10 | 4771 | AAV-v3 |
| (in line) | Cas9-hU6c-U7-7SK2-D10 | 4765 | AAV-v4 |
| (“Design 4” geometries | Cas9-hU6c-U7-H1m-D10 | 4623 | |
| from FIG. 10B) | Cas9-hU6m-U7-hU6c-D10 | 4633 | |
| | Cas9-hU6m-U7-7SK2-D10 | 4627 | |
| | Cas9-hU6m-U7-H1m-D10 | 4485 | |
| | Cas9-hU6c-U7-M11-D10 | 4622 | |
| | Cas9-M11-D10-hU6c-U7 | 4619 | |
| | Cas9-7SK2-D10-hU6c-U7 | 4762 | |
| | Cas9-7SK2-U7-M11-D10 | 4613 | |
| | Cas9-M11-D10-7SK2-U7 | 4613 | |

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

1. A composition comprising a single nucleic acid molecule encoding one or more guide RNAs and a Cas9, wherein the single nucleic acid molecule comprises:
a. a first nucleic acid encoding one or more spacer sequences selected from any one of SEQ ID NOs: 1-8, 10-28, and 101-154, and a second nucleic acid encoding a Staphylococcus aureus Cas9 (SaCas9);
b. a first nucleic acid encoding one or more spacer sequences comprising at least 20, or 21 contiguous nucleotides of a spacer sequence selected from any one of SEQ ID NOs: 1-8, 10-28, and 101-154, and a second nucleic acid encoding a Staphylococcus aureus Cas9 (SaCas9);
c. a first nucleic acid encoding one or more spacer sequences that are at least 90% identical to any one of SEQ ID NOs: 1-8, 10-28, and 101-154, and a second nucleic acid encoding a Staphylococcus aureus Cas9 (SaCas9);
d. a first nucleic acid encoding one or more spacer sequences selected from any one of SEQ ID NOs: 1, 2, 3, 4, 7, 8, 10, 11, 12, 13, 14, 15, 18, 19, 20, 21, 23, 25, 26, 27, and 28, and a second nucleic acid encoding a Staphylococcus aureus Cas9 (SaCas9);
e. a first nucleic acid encoding one or more spacer sequences selected from any one of SEQ ID NOs: 1, 2, 3, 4, 7, 8, 12, and 20, and a second nucleic acid encoding a Staphylococcus aureus Cas9 (SaCas9);
f. a first nucleic acid encoding one or more spacer sequences selected from any one of SEQ ID NOs: 1, 2, 3, 4, 7, 8, and 20, and a second nucleic acid encoding a Staphylococcus aureus Cas9 (SaCas9);
g. a first nucleic acid encoding one or more spacer sequences selected from any one of SEQ ID NOs: 1, 101, and 102, and a second nucleic acid encoding a Staphylococcus aureus Cas9 (SaCas9);
h. a first nucleic acid encoding a pair of guide RNAs comprising a first and second spacer sequence selected from any one of SEQ ID NOs: 1 and 10; 1 and 11; 1 and 12; land 13; land 14; land 15; land 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28, and a second nucleic acid encoding a Staphylococcus aureus Cas9 (SaCas9);
i. a first nucleic acid encoding a pair of guide RNAs comprising at least 20, or 21 contiguous nucleotides of a first and second spacer sequence selected from any one of SEQ ID NOs: 1 and 10; 1 and 11; 1 and 12; 1 and 13; 1 and 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28, and a second nucleic acid encoding a Staphylococcus aureus Cas9 (SaCas9);
j. a first nucleic acid encoding a pair of guide RNAs that is at least 90% identical to a first and second spacer sequence selected from any one of SEQ ID NOs: 1 and 10; land 11; land 12; land 13; land 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28, and a second nucleic acid encoding a Staphylococcus aureus Cas9 (SaCas9);
k. a first nucleic acid encoding a pair of guide RNAs comprising a first and second spacer sequence selected from any one of SEQ ID NOs: 4 and 12; 2 and 12; 3 and 12; 4 and 20; 4 and 18; 2 and 10; 4 and 28; I and 12; 8 and 12; 4 and 13; 4 and 23; 3 and 10; 8 and 20; 1 and 10; 2 and 23; 2 and 20; 8 and 23; 8 and 10; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 7 and 10; 1 and 13; 1 and 20; 1 and 28; 4 and 27; 7 and 20; 7 and 23; 7 and 13; 7 and 28; 2 and 27; 8 and 27; 4 and 11; 4 and 25; 4 and 28; 4 and 19; 4 and 15; 8 and 11; 3 and 27; 2 and 25; 2 and 11; 7 and 18; 3 and 25; 8 and 15; 8 and 25; 3 and 11; 3 and 19; 1 and 15; 3 and 15; 1 and 27; 2 and 15; 2 and 19; 1 and 11; 1 and 25; 8 and 19; 4 and 21; 8 and 21; 7 and 27; 7 and 15; 1 and 19; 2 and 21; 7 and 11; 3 and 21; 4 and 14; 7 and 19; 4 and 26; 8 and 26; 7 and 25; 1 and 21; 3 and 26; 2 and 26; 8 and 14; 1 and 14; 2 and 14; 3 and 14; 1 and 26; 7 and 21; 7 and 14; and 7 and 26, and a second nucleic acid encoding a Staphylococcus aureus Cas9 (SaCas9);
l. a first nucleic acid encoding a pair of guide RNAs comprising a first and second spacer sequence selected from any one of SEQ ID NOs: 4 and 12; 2 and 12; 3 and 12; 4 and 20; 4 and 18; 2 and 10; 4 and 28; I and 12; 8 and 12; 4 and 13; 4 and 23; 3 and 10; 8 and 20; 1 and 10; 2 and 23; 2 and 20; 8 and 23; 8 and 10; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 7 and 10; 1 and 13; 1 and 20; 1 and 28; 4 and 27; 7 and 20; 7 and 23; 7 and 13; 7 and 28; 2 and 27; 8 and 12; 8 and 27; 4 and 11; and 4 and 25, and a second nucleic acid encoding a Staphylococcus aureus Cas9 (SaCas9);
m. a first nucleic acid encoding a pair of guide RNAs comprising a first and second spacer sequence selected from any one of SEQ ID NOs: 4 and 12; 2 and 12; 3 and 12; 4 and 20; 4 and 18; 2 and 10; 4 and 28; I and 12; 8 and 12; 4 and 13; 4 and 23; 3 and 10; 8 and 20; 1 and 10; 2 and 23; 2 and 20; 8 and 23; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 1 and 13; 1 and 20; 1 and 28; 4 and 27; 7 and 20; 7 and 23; 7 and 13; 7 and 28; 2 and 27; 4 and 11; 4 and 25; 4 and 28; 4 and 19; 4 and 15; 8 and 11; 3 and 27; 2 and 25; 2 and 11; 7 and 18; 3 and 25; 8 and 15; 3 and 11; 3 and 19; 1 and 15; 3 and 15; 1 and 27; 2 and 15; 2 and 19; 1 and 11; 1 and 25; 4 and 21; 8 and 21; 7 and 27; 7 and 15; 1 and 19; 2 and 21; 7 and 11; 3 and 21; 7 and 19; 7 and 25; 1 and 21; 3 and 26; 3 and 14; 7 and 21; and 7 and 14 and a second nucleic acid encoding a Staphylococcus aureus Cas9 (SaCas9);
n. a first nucleic acid encoding a pair of guide RNAs comprising a first and second spacer sequence selected from any one of SEQ ID NOs: 4 and 12; 2 and 10; 4 and 18; 4 and 28; 8 and 12; 4 and 13; 3 and 10; 7 and 12; 7 and 13; 4 and 28; and 7 and 18, and a second nucleic acid encoding a Staphylococcus aureus Cas9 (SaCas9);
o. a first nucleic acid encoding a pair of guide RNAs comprising a first and second spacer sequence selected from any one of SEQ ID NOs: 4 and 12; 2 and 12; 3 and 12; 4 and 20; 4 and 18; 2 and 10; 4 and 10; 1 and 12; 8 and 12; 4 and 13; 7 and 12; 7 and 28; 7 and 18 and a second nucleic acid encoding a Staphylococcus aureus Cas9 (SaCas9);
p. a first nucleic acid encoding a pair of guide RNAs comprising a first and second spacer sequence of SEQ ID NOs: 7 and 12, and a second nucleic acid encoding a Staphylococcus aureus Cas9 (SaCas9);
q. a first nucleic acid encoding a pair of guide RNAs comprising a first and second spacer sequence of SEQ ID NOs: 4 and 12, and a second nucleic acid encoding a Staphylococcus aureus Cas9 (SaCas9); or
r. a first nucleic acid encoding a pair of guide RNAs comprising a first and second spacer sequence of SEQ ID NOs: 4 and 18, and a second nucleic acid encoding a Staphylococcus aureus Cas9 (SaCas9); or
s. a first nucleic acid encoding a pair of guide RNAs comprising a first and second spacer sequence of SEQ ID NOs: 2 and 12, and a second nucleic acid encoding a Staphylococcus aureus Cas9 (SaCas9); or
t. a first nucleic acid encoding a pair of guide RNAs comprising a first and second spacer sequence of SEQ ID NOs: 4 and 13, and a second nucleic acid encoding a Staphylococcus aureus Cas9 (SaCas9); or
u. a first nucleic acid encoding a pair of guide RNAs comprising a first and second spacer sequence of SEQ ID NOs: 8 and 12, and a second nucleic acid encoding a Staphylococcus aureus Cas9 (SaCas9); or
v. a first nucleic acid encoding a pair of guide RNAs comprising a first and second spacer sequence of SEQ ID NOs: 7 and 23, and a second nucleic acid encoding a Staphylococcus aureus Cas9 (SaCas9).

2. The composition of claim 1, further comprising a DNA-PK inhibitor.

3. The composition of claim 2, wherein the DNA-PK inhibitor is Compound 6, Compound 1, or Compound 2.

4. (canceled)

5. (canceled)

6. The composition of claim 1, wherein the guide RNA is an sgRNA.

7. The composition of claim 1, wherein the guide RNA is a modified guide RNA.

8.-11. (canceled)

12. The composition of claim 1, wherein the single nucleic acid molecule is associated with a lipid nanoparticle (LNP) or a viral vector.

13. (canceled)

14. The composition of claim 12, wherein the single nucleic acid molecule is associated with a viral vector, and wherein the viral vector is an adeno-associated virus vector, a lentiviral vector, an integrase-deficient lentiviral vector, an adenoviral vector, a vaccinia viral vector, an alphaviral vector, or a herpes simplex viral vector.

15.-22. (canceled)

23. The composition of claim 1, comprising a nucleic acid encoding SaCas9, wherein the SaCas9 comprises the amino acid sequence of any one of SEQ ID NOs: 711 or 715-717.

24.-31. (canceled)

32. A method of treating Myotonic Dystrophy Type 1 (DM1), the method comprising delivering to a cell a single nucleic acid molecule comprising a nucleic acid encoding a Staphylococcus aureus Cas9 (SaCas9); and:
i) a nucleic acid encoding a guide RNA, wherein the guide RNA comprises:
a. one or more spacer sequences selected from any one of SEQ ID NOs: 1-8, 10-28, and 101-154;
b. one or more spacer sequences comprising at least 20, or 21 contiguous nucleotides of a spacer sequence selected from any one of SEQ ID NOs: 1-8, 10-28, and 101-154; or
c. one or more spacer sequences that are at least 90% identical to any one of SEQ ID NOs: 1-8, 10-28, and 101-154; or
ii) a nucleic acid encoding a pair of guide RNAs comprising:
a. a first and second spacer sequence selected from any one of SEQ ID NOs: 1 and 10; land 11; land 12; land 13; 1 and 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28;
b. a first and second spacer sequence comprising at least 20, or 21 contiguous nucleotides of any of the first and second spacer sequences of i) a.; or
c. a first and second spacer sequence that is at least 90% identical to any of the first and second spacer sequences of i) a. or i) b.

33. (canceled)

34. (canceled)

35. A method of excising a CTG repeat in the 3′ UTR of the DMPK gene, the method comprising delivering to a cell a single nucleic acid molecule comprising a nucleic acid encoding a Staphylococcus aureus Cas9 (SaCas9); and:
i) a nucleic acid encoding a guide RNA, wherein the guide RNA comprises:
a. one or more spacer sequences selected from any one of SEQ ID NOs: 1-8, 10-28, and 101-154;
b. one or more spacer sequences comprising at least 20, or 21 contiguous nucleotides of a spacer sequence selected from any one of SEQ ID NOs: 1-8, 10-28, and 101-154; or
c. one or more spacer sequences that are at least 90% identical to any one of SEQ ID NOs: 1-8, 10-28, and 101-154; or
ii) a nucleic acid encoding a pair of guide RNAs comprising:
a. a first and second spacer sequence selected from SEQ ID NOs: 1 and 10; 1 and 11; land 12; land 13; land 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28;
b. a first and second spacer sequence comprising at least 20, or 21 contiguous nucleotides of any of the first and second spacer sequences of i) a.; or
c. a first and second spacer sequence that is at least 90% identical to any of the first and second spacer sequences of i) a. or i) b.

36. The method of claim 35, wherein the one or more spacer sequence is:
a. selected from any one of SEQ ID NOs: 1, 2, 3, 4, 7, 8, 10, 11, 12, 13, 14, 15, 18, 19, 20, 21, 23, 25, 26, 27, and 28;
b. selected from any one of SEQ ID NOs: 1, 2, 3, 4, 7, 8, 12, 18, and 20;
c. selected from any one of SEQ ID NOs: 1, 2, 3, 4, 7, 8, and 20;
d. selected from any one of SEQ ID NOs: 4, 12, and 18; or
e. selected from any one of SEQ ID NOs: 1, 101, and 102.

37. (canceled)

38. The method of claim 35, wherein the first and second spacer sequences are:
a. selected from SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 8 and 10; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 7 and 10; 1 and 13; 1 and 20; 1 and 28; 4 and 27; 7 and 20; 7 and 23; 7 and 13; 7 and 28; 2 and 27; 8 and 27; 4 and 11; 4 and 25; 4 and 28; 4 and 19; 4 and 15; 8 and 11; 3 and 27; 2 and 25; 2 and 11; 7 and 18; 3 and 25; 8 and 15; 8 and 25; 3 and 11; 3 and 19; 1 and 15; 3 and 15; 1 and 27; 2 and 15; 2 and 19; 1 and 11; 1 and 25; 8 and 19; 4 and 21; 8 and 21; 7 and 27; 7 and 15; 1 and 19; 2 and 21; 7 and 11; 3 and 21; 4 and 14; 7 and 19; 4 and 26; 8 and 26; 7 and 25; 1 and 21; 3 and 26; 2 and 26; 8 and 14; 1 and 14; 2 and 14; 3 and 14; 1 and 26; 7 and 21; 7 and 14; and 7 and 26;
b. selected from any one of SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 8 and 10; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 7 and 10; 1 and 13; 1 and 20; 1 and 28; 4 and 27; 7 and 20; 7 and 23; 7 and 13; 7 and 28; 2 and 27; 8 and 27; 4 and 11; and 4 and 25;
c. selected from any one of SEQ ID NOs: 4 and 12; 2 and 12; 3 and 12; 4 and 20; 4 and 18; 2 and 10; 4 and 28; 1 and 12; 8 and 12; 4 and 13; 4 and 23; 3 and 10; 8 and 20; 1 and 10; 2 and 23; 2 and 20; 8 and 23; 1 and 18; 2 and 13; 2 and 18; 3 and 18; 2 and 28; 7 and 12; 8 and 18; 3 and 20; 3 and 23; 2 and 13; 1 and 23; 8 and 13; 3 and 28; 8 and 28; 1 and 13; 1 and 20; 1 and 28; 4 and 27; 7 and 20; 7 and 23; 7 and 13; 7 and 28; 2 and 27; 4 and 11; 4 and 25; 4 and 28; 4 and 19; 4 and 15; 8 and 11; 3 and 27; 2 and 25; 2 and 11; 7 and 18; 3 and 25; 8 and 15; 3 and 11; 3 and 19; 1 and 15; 3 and 15; 1 and 27; 2 and 15; 2 and 19; 1 and 11; 1 and 25; 4 and 21; 8 and 21; 7 and 27; 7 and 15; 1 and 19; 2 and 21; 7 and 11; 3 and 21; 7 and 19; 7 and 25; 1 and 21; 3 and 26; 3 and 14; 7 and 21; and 7 and 14;
d. selected from any one of SEQ ID NOs: 4 and 12; 4 and 18; 2 and 10; 4 and 28; 8 and 12; 4 and 13; 3 and 10; 7 and 12; 7 and 13; 4 and 28; and 7 and 18;
e. selected from any one of SEQ ID NOs: 4 and 12; 2 and 12; 3 and 12; 4 and 20; 4 and 18; 2 and 10; 4 and 10; 1 and 12; 8 and 12; 4 and 13; 7 and 12; 7 and 28; 7 and 18; or
f. selected from any one of SEQ ID NOs: 7 and 12; 4 and 12; 4 and 18; 8 and 12; or 7 and 23; or
g. selected from any one of SEQ ID NOs: 4 and 12; 4 and 18; 8 and 12.

39. (canceled)

40. The method of claim 32, further comprising administering a DNA-PK inhibitor.

41. The method of claim 40, wherein the DNA-PK inhibitor is Compound 6, Compound 1, or Compound 2.

42. (canceled)

43. (canceled)

44. The method of claim 32, wherein the SaCas9 comprises the amino acid sequence of any one of SEQ ID NOs: 711 or 715-717.

45.-53. (canceled)

54. The composition of claim 1, wherein the one or more guide RNAs or pair of guide RNAs are sgRNAs comprising a scaffold sequence selected from SEQ ID NOs: 500, 910, 911, 912, 920, or 921.

55.-66. (canceled)

67. The composition of claim 1, wherein the single nucleic acid molecule is an AAV vector, wherein the vector comprises from 5′ to 3′ with respect to the plus strand: a promoter for expression of a nucleic acid encoding a first sgRNA, a nucleic acid encoding the first sgRNA guide sequence, a first sgRNA scaffold sequence, a promoter for expression of a nucleic acid encoding a SaCas9 (e.g., CK8e), a nucleic acid encoding a SaCas9, a polyadenylation sequence, a promoter for expression of a second sgRNA, the second sgRNA guide sequence, and a second sgRNA scaffold sequence.

68.-72. (canceled)

73. The composition of claim 67, wherein the first sgRNA comprises SaU4 (SEQ ID NO: 4) and the second sgRNA comprises SaD4 (SEQ ID NO: 12), or wherein the first sgRNA comprises SaU7 (SEQ ID NO: 7) and the second sgRNA comprises SaD10 (SEQ ID NO: 18).

74.-90. (canceled)

91. A composition comprising a first nucleic acid molecule and a second nucleic acid molecule, wherein the first nucleic acid molecule encodes a Staphylococcus aureus Cas9 (SaCas9) and the second nucleic acid molecule encodes: one or more guide RNAs comprising:
a. a first and second spacer sequence selected from any one of SEQ ID NOs: 1 and 10; land 11; land 12; land 13; land 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28;
b. a first and second spacer sequence comprising at least 20, or 21 contiguous nucleotides of any of the first and second spacer sequences of a.;
c. a first and second spacer sequence that is at least 90% identical to any of the first and second spacer sequences of a. or b.

92. The composition of claim 91, wherein the first nucleic acid molecule does not encode a guide RNA.

93. The composition of claim 91, wherein the first nucleic acid molecule encodes:
a. a first and second spacer sequence selected from any one of SEQ ID NOs: 1 and 10; land 11; land 12; land 13; land 14; 1 and 15; 1 and 16; 1 and 17; 1 and 18; 1 and 19; 1 and 20; 1 and 21; 1 and 22; 1 and 23; 1 and 24; 1 and 25; 1 and 26; 1 and 27; 1 and 28; 2 and 10; 2 and 11; 2 and 12; 2 and 13; 2 and 14; 2 and 15; 2 and 16; 2 and 17; 2 and 18; 2 and 19; 2 and 20; 2 and 21; 2 and 22; 2 and 23; 2 and 24; 2 and 25; 2 and 26; 2 and 27; 2 and 28; 3 and 10; 3 and 11; 3 and 12; 3 and 13; 3 and 14; 3 and 15; 3 and 16; 3 and 17; 3 and 18; 3 and 19; 3 and 20; 3 and 21; 3 and 22; 3 and 23; 3 and 24; 3 and 25; 3 and 26; 3 and 27; 3 and 28; 4 and 10; 4 and 11; 4 and 12; 4 and 13; 4 and 14; 4 and 15; 4 and 16; 4 and 17; 4 and 18; 4 and 19; 4 and 20; 4 and 21; 4 and 22; 4 and 23; 4 and 24; 4 and 25; 4 and 26; 4 and 27; 4 and 28; 5 and 10; 5 and 11; 5 and 12; 5 and 13; 5 and 14; 5 and 15; 5 and 16; 5 and 17; 5 and 18; 5 and 19; 5 and 20; 5 and 21; 5 and 22; 5 and 23; 5 and 24; 5 and 25; 5 and 26; 5 and 27; 5 and 28; 6 and 10; 6 and 11; 6 and 12; 6 and 13; 6 and 14; 6 and 15; 6 and 16; 6 and 17; 6 and 18; 6 and 19; 6 and 20; 6 and 21; 6 and 22; 6 and 23; 6 and 24; 6 and 25; 6 and 26; 6 and 27; 6 and 28; 7 and 10; 7 and 11; 7 and 12; 7 and 13; 7 and 14; 7 and 15; 7 and 16; 7 and 17; 7 and 18; 7 and 19; 7 and 20; 7 and 21; 7 and 22; 7 and 23; 7 and 24; 7 and 25; 7 and 26; 7 and 27; 7 and 28; 8 and 10; 8 and 11; 8 and 12; 8 and 13; 8 and 14; 8 and 15; 8 and 16; 8 and 17; 8 and 18; 8 and 19; 8 and 20; 8 and 21; 8 and 22; 8 and 23; 8 and 24; 8 and 25; 8 and 26; 8 and 27; and 8 and 28;
b. a first and second spacer sequence comprising at least 20, or 21 contiguous nucleotides of any of the first and second spacer sequences of a.;
c. a first and second spacer sequence that is at least 90% identical to any of the first and second spacer sequences of a. or b.

94.-124. (canceled)


SEQUENCE LISTING (943 sequences)

SEQ ID NO: 1 - DNA, 22 bp, synthetic construct
  misc_feature 1..22: note = Synthetic
  source 1..22: mol_type = other DNA
gccccggagtcgaagacagttc

SEQ ID NO: 2 - DNA, 22 bp, synthetic construct
  misc_feature 1..22: note = Synthetic
  source 1..22: mol_type = other DNA
actcagtcttccaacggggccc

SEQ ID NO: 3 - DNA, 22 bp, synthetic construct
  misc_feature 1..22: note = Synthetic
  source 1..22: mol_type = other DNA
actccggggccccgttggaaga

SEQ ID NO: 4 - DNA, 22 bp, synthetic construct
  misc_feature 1..22: note = Synthetic
  source 1..22: mol_type = other DNA
ccagttcacaaccgctccgagc

SEQ ID NO: 5 - DNA, 22 bp, synthetic construct
  misc_feature 1..22: note = Synthetic
  source 1..22: mol_type = other DNA
ccccggccgctagggggcgggc

SEQ ID NO: 6 - DNA, 22 bp, synthetic construct
  misc_feature 1..22: note = Synthetic
  source 1..22: mol_type = other DNA
ctagcggccggggagggagggg

SEQ ID NO: 7 - DNA, 22 bp, synthetic construct
  misc_feature 1..22: note = Synthetic
  source 1..22: mol_type = other DNA
cgcggccggcgaacggggctcg

SEQ ID NO: 8 - DNA, 22 bp, synthetic construct
  misc_feature 1..22: note = Synthetic
  source 1..22: mol_type = other DNA
ggctcgaagggtccttgtagcc

SEQ ID NO: 9 - DNA, 22 bp, synthetic construct
  misc_feature 1..22: note = Synthetic
  source 1..22: mol_type = other DNA
ctgctgctgctgctgctgctgg

SEQ ID NO: 10 - DNA, 22 bp, synthetic construct
  misc_feature 1..22: note = Synthetic
  source 1..22: mol_type = other DNA
tcggccaggctgaggccctgac

SEQ ID NO: 11 - DNA, 22 bp, synthetic construct
  misc_feature 1..22: note = Synthetic
  source 1..22: mol_type = other DNA
caacgataggtgggggtgcgtg

SEQ ID NO: 12 - DNA, 22 bp, synthetic construct
  misc_feature 1..22: note = Synthetic
  source 1..22: mol_type = other DNA
actttgcgaaccaacgataggt

SEQ ID NO: 13 - DNA, 22 bp, synthetic construct
  misc_feature 1..22: note = Synthetic
  source 1..22: mol_type = other DNA
gggtttggcaaaagcaaatttc

SEQ ID NO: 14 - DNA, 22 bp, synthetic construct
  misc_feature 1..22: note = Synthetic
  source 1..22: mol_type = other DNA
cttttgccaaacccgctttttc

SEQ ID NO: 15 - DNA, 22 bp, synthetic construct
  misc_feature 1..22: note = Synthetic
  source 1..22: mol_type = other DNA
ggggcgcgggatccccgaaaaa

SEQ ID NO: 16 - DNA, 22 bp, synthetic construct
  misc_feature 1..22: note = Synthetic
  source 1..22: mol_type = other DNA
agcgcaagtgaggaggggggcg

SEQ ID NO: 17 - DNA, 22 bp, synthetic construct
  misc_feature 1..22: note = Synthetic
  source 1..22: mol_type = other DNA
cggctccgcccgcttcggcggt

SEQ ID NO: 18 - DNA, 22 bp, synthetic construct
  misc_feature 1..22: note = Synthetic
  source 1..22: mol_type = other DNA
ttgggggtcctgtagcctgtca

SEQ ID NO: 19 - DNA, 22 bp, synthetic construct
  misc_feature 1..22: note = Synthetic
  source 1..22: mol_type = other DNA
caaaacgtggattggggttgtt

SEQ ID NO: 20 - DNA, 22 bp, synthetic construct
  misc_feature 1..22: note = Synthetic
  source 1..22: mol_type = other DNA
tcagtgcatccaaaacgtggat

SEQ ID NO: 21 - DNA, 22 bp, synthetic construct
  misc_feature 1..22: note = Synthetic
  source 1..22: mol_type = other DNA
cccaacaaccccaatccacgtt

SEQ ID NO: 22 - DNA, 22 bp, synthetic construct
  misc_feature 1..22: note = Synthetic
  source 1..22: mol_type = other DNA
ggggtctcagtgcatccaaaac

SEQ ID NO: 23 - DNA, 22 bp, synthetic construct
  misc_feature 1..22: note = Synthetic
  source 1..22: mol_type = other DNA
gacaataaataccgaggaatgt

SEQ ID NO: 24 - DNA, 22 bp, synthetic construct
  misc_feature 1..22: note = Synthetic
  source 1..22: mol_type = other DNA
tggggacagacaataaataccg

SEQ ID NO: 25 - DNA, 22 bp, synthetic construct
  misc_feature 1..22: note = Synthetic
  source 1..22: mol_type = other DNA
tttattcgcgagggtcgggggt

SEQ ID NO: 26 - DNA, 22 bp, synthetic construct
  misc_feature 1..22: note = Synthetic
  source 1..22: mol_type = other DNA
gggccttttattcgcgagggtc

SEQ ID NO: 27 - DNA, 22 bp, synthetic construct
  misc_feature 1..22: note = Synthetic
  source 1..22: mol_type = other DNA
aggacccccacccccgaccctc

SEQ ID NO: 28 - DNA, 22 bp, synthetic construct
  misc_feature 1..22: note = Synthetic
  source 1..22: mol_type = other DNA
agatggagggccttttattcgc

SEQ ID NO: 29-100 - skipped (72 entries)

SEQ ID NO: 101 - DNA, 21 bp, synthetic construct
  misc_feature 1..21: note = Synthetic
  source 1..21: mol_type = other DNA
ccccggagtcgaagacagttc

SEQ ID NO: 102 - DNA, 20 bp, synthetic construct
  misc_feature 1..20: note = Synthetic
  source 1..20: mol_type = other DNA
cccggagtcgaagacagttc

SEQ ID NO: 103 - DNA, 21 bp, synthetic construct
  misc_feature 1..21: note = Synthetic
  source 1..21: mol_type = other DNA
ctcagtcttccaacggggccc

SEQ ID NO: 104 - DNA, 20 bp, synthetic construct
  misc_feature 1..20: note = Synthetic
  source 1..20: mol_type = other DNA
tcagtcttccaacggggccc

SEQ ID NO: 105 - DNA, 21 bp, synthetic construct
  misc_feature 1..21: note = Synthetic
  source 1..21: mol_type = other DNA
actccggggccccgttggaag

SEQ ID NO: 106 - DNA, 20 bp, synthetic construct
  misc_feature 1..20: note = Synthetic
  source 1..20: mol_type = other DNA
actccggggccccgttggaa

SEQ ID NO: 107 - DNA, 21 bp, synthetic construct
  misc_feature 1..21: note = Synthetic
  source 1..21: mol_type = other DNA
ccagttcacaaccgctccgag

SEQ ID NO: 108 - DNA, 20 bp, synthetic construct
  misc_feature 1..20: note = Synthetic
  source 1..20: mol_type = other DNA
ccagttcacaaccgctccga

SEQ ID NO: 109 - DNA, 21 bp, synthetic construct
  misc_feature 1..21: note = Synthetic
  source 1..21: mol_type = other DNA
cccggccgctagggggcgggc

SEQ ID NO: 110 - DNA, 20 bp, synthetic construct
  misc_feature 1..20: note = Synthetic
  source 1..20: mol_type = other DNA
ccggccgctagggggcgggc

SEQ ID NO: 111 - DNA, 21 bp, synthetic construct
  misc_feature 1..21: note = Synthetic
  source 1..21: mol_type = other DNA
ctagcggccggggagggaggg

SEQ ID NO: 112 - DNA, 20 bp, synthetic construct
  misc_feature 1..20: note = Synthetic
  source 1..20: mol_type = other DNA
ctagcggccggggagggagg

SEQ ID NO: 113 - DNA, 21 bp, synthetic construct
  misc_feature 1..21: note = Synthetic
  source 1..21: mol_type = other DNA
cgcggccggcgaacggggctc

SEQ ID NO: 114 - DNA, 20 bp, synthetic construct
  misc_feature 1..20: note = Synthetic
  source 1..20: mol_type = other DNA
cgcggccggcgaacggggct

SEQ ID NO: 115 - DNA, 21 bp, synthetic construct
  misc_feature 1..21: note = Synthetic
  source 1..21: mol_type = other DNA
ggctcgaagggtccttgtagc

SEQ ID NO: 116 - DNA, 20 bp, synthetic construct
  misc_feature 1..20: note = Synthetic
  source 1..20: mol_type = other DNA
ggctcgaagggtccttgtag

SEQ ID NO: 117 - DNA, 21 bp, synthetic construct
  misc_feature 1..21: note = Synthetic
  source 1..21: mol_type = other DNA
tcggccaggctgaggccctga

SEQ ID NO: 118 - DNA, 20 bp, synthetic construct
  misc_feature 1..20: note = Synthetic
  source 1..20: mol_type = other DNA
tcggccaggctgaggccctg

SEQ ID NO: 119 - DNA, 21 bp, synthetic construct
  misc_feature 1..21: note = Synthetic
  source 1..21: mol_type = other DNA
aacgataggtgggggtgcgtg

SEQ ID NO: 120 - DNA, 20 bp, synthetic construct
  misc_feature 1..20: note = Synthetic
  source 1..20: mol_type = other DNA
acgataggtgggggtgcgtg

SEQ ID NO: 121 - DNA, 21 bp, synthetic construct
  misc_feature 1..21: note = Synthetic
  source 1..21: mol_type = other DNA
ctttgcgaaccaacgataggt

SEQ ID NO: 122 - DNA, 20 bp, synthetic construct
  misc_feature 1..20: note = Synthetic
  source 1..20: mol_type = other DNA
tttgcgaaccaacgataggt

SEQ ID NO: 123 - DNA, 21 bp, synthetic construct
  misc_feature 1..21: note = Synthetic
  source 1..21: mol_type = other DNA
ggtttggcaaaagcaaatttc

SEQ ID NO: 124 - DNA, 20 bp, synthetic construct
  misc_feature 1..20: note = Synthetic
  source 1..20: mol_type = other DNA
gtttggcaaaagcaaatttc

SEQ ID NO: 125 - DNA, 21 bp, synthetic construct
  misc_feature 1..21: note = Synthetic
  source 1..21: mol_type = other DNA
cttttgccaaacccgcttttt

SEQ ID NO: 126 - DNA, 20 bp, synthetic construct
  misc_feature 1..20: note = Synthetic
  source 1..20: mol_type = other DNA
cttttgccaaacccgctttt

SEQ ID NO: 127 - DNA, 21 bp, synthetic construct
  misc_feature 1..21: note = Synthetic
  source 1..21: mol_type = other DNA
gggcgcgggatccccgaaaaa

SEQ ID NO: 128 - DNA, 20 bp, synthetic construct
  misc_feature 1..20: note = Synthetic
  source 1..20: mol_type = other DNA
ggcgcgggatccccgaaaaa

SEQ ID NO: 129 - DNA, 21 bp, synthetic construct
  misc_feature 1..21: note = Synthetic
  source 1..21: mol_type = other DNA
gcgcaagtgaggaggggggcg

SEQ ID NO: 130 - DNA, 20 bp, synthetic construct
  misc_feature 1..20: note = Synthetic
  source 1..20: mol_type = other DNA
cgcaagtgaggaggggggcg

SEQ ID NO: 131 - DNA, 21 bp, synthetic construct
  misc_feature 1..21: note = Synthetic
  source 1..21: mol_type = other DNA
cggctccgcccgcttcggcgg

SEQ ID NO: 132 - DNA, 20 bp, synthetic construct
  misc_feature 1..20: note = Synthetic
  source 1..20: mol_type = other DNA
cggctccgcccgcttcggcg

SEQ ID NO: 133 - DNA, 21 bp, synthetic construct
  misc_feature 1..21: note = Synthetic
  source 1..21: mol_type = other DNA
tgggggtcctgtagcctgtca

SEQ ID NO: 134 - DNA, 20 bp, synthetic construct
  misc_feature 1..20: note = Synthetic
  source 1..20: mol_type = other DNA
gggggtcctgtagcctgtca

SEQ ID NO: 135 - DNA, 21 bp, synthetic construct
  misc_feature 1..21: note = Synthetic
  source 1..21: mol_type = other DNA
aaaacgtggattggggttgtt

SEQ ID NO: 136 - DNA, 20 bp, synthetic construct
  misc_feature 1..20: note = Synthetic
  source 1..20: mol_type = other DNA
aaacgtggattggggttgtt

SEQ ID NO: 137 - DNA, 21 bp, synthetic construct
  misc_feature 1..21: note = Synthetic
  source 1..21: mol_type = other DNA
cagtgcatccaaaacgtggat

SEQ ID NO: 138 - DNA, 20 bp, synthetic construct
  misc_feature 1..20: note = Synthetic
  source 1..20: mol_type = other DNA
agtgcatccaaaacgtggat

SEQ ID NO: 139 - DNA, 21 bp, synthetic construct
  misc_feature 1..21: note = Synthetic
  source 1..21: mol_type = other DNA
cccaacaaccccaatccacgt

SEQ ID NO: 140 - DNA, 20 bp, synthetic construct
  misc_feature 1..20: note = Synthetic
  source 1..20: mol_type = other DNA
cccaacaaccccaatccacg

SEQ ID NO: 141 - DNA, 21 bp, synthetic construct
  misc_feature 1..21: note = Synthetic
  source 1..21: mol_type = other DNA
gggtctcagtgcatccaaaac

SEQ ID NO: 142 - DNA, 20 bp, synthetic construct
  misc_feature 1..20: note = Synthetic
  source 1..20: mol_type = other DNA
ggtctcagtgcatccaaaac

SEQ ID NO: 143 - DNA, 21 bp, synthetic construct
  misc_feature 1..21: note = Synthetic
  source 1..21: mol_type = other DNA
acaataaataccgaggaatgt

SEQ ID NO: 144 - DNA, 20 bp, synthetic construct
  misc_feature 1..20: note = Synthetic
  source 1..20: mol_type = other DNA
caataaataccgaggaatgt

SEQ ID NO: 145 - DNA, 21 bp, synthetic construct
  misc_feature 1..21: note = Synthetic
  source 1..21: mol_type = other DNA
ggggacagacaataaataccg

SEQ ID NO: 146 - DNA, 20 bp, synthetic construct
  misc_feature 1..20: note = Synthetic
  source 1..20: mol_type = other DNA
gggacagacaataaataccg

SEQ ID NO: 147 - DNA, 21 bp, synthetic construct
  misc_feature 1..21: note = Synthetic
  source 1..21: mol_type = other DNA
ttattcgcgagggtcgggggt

SEQ ID NO: 148 - DNA, 20 bp, synthetic construct
  misc_feature 1..20: note = Synthetic
  source 1..20: mol_type = other DNA
tattcgcgagggtcgggggt

SEQ ID NO: 149 - DNA, 21 bp, synthetic construct
  misc_feature 1..21: note = Synthetic
  source 1..21: mol_type = other DNA
ggccttttattcgcgagggtc

SEQ ID NO: 150 - DNA, 20 bp, synthetic construct
  misc_feature 1..20: note = Synthetic
  source 1..20: mol_type = other DNA
gccttttattcgcgagggtc

SEQ ID NO: 151 - DNA, 21 bp, synthetic construct
  misc_feature 1..21: note = Synthetic
  source 1..21: mol_type = other DNA
aggacccccacccccgaccct

SEQ ID NO: 152 - DNA, 20 bp, synthetic construct
  misc_feature 1..20: note = Synthetic
  source 1..20: mol_type = other DNA
aggacccccacccccgaccc

SEQ ID NO: 153 - DNA, 21 bp, synthetic construct
  misc_feature 1..21: note = Synthetic
  source 1..21: mol_type = other DNA
gatggagggccttttattcgc

SEQ ID NO: 154 - DNA, 20 bp, synthetic construct
  misc_feature 1..20: note = Synthetic
  source 1..20: mol_type = other DNA
atggagggccttttattcgc

SEQ ID NO: 155-499 - skipped (345 entries)

SEQ ID NO: 500 - DNA, 86 bp, synthetic construct
  misc_feature 1..86: note = Synthetic
  source 1..86: mol_type = other DNA
gtttaagtactctgtgctggaaacagcacagaatctacttaaacaaggcaaaatgccgtgtttatctcgtcaacttgttggcgaga

SEQ ID NO: 501-699 - skipped (199 entries)

SEQ ID NO: 700 - DNA, 450 bp, synthetic construct
  misc_feature 1..450: note = Synthetic
  source 1..450: mol_type = other DNA
ctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 701 - DNA, 436 bp, synthetic construct
  misc_feature 1..436: note = Synthetic
  source 1..436: mol_type = other DNA
tgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 702 - DNA, 472 bp, synthetic construct
  misc_feature 1..472: note = Synthetic
  source 1..472: mol_type = other DNA
cgagtccaacacccgtgggaatcccatgggcaccatggcccctcgctccaaaaatgctttcgcgtcgcgcagacactgctcggtagtttcggggatcagcgtttgagtaagagcccgcgtctgaaccctccgcgccgccccggccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaa

SEQ ID NO: 703 - DNA, 108 bp, synthetic construct
  misc_feature 1..108: note = Synthetic
  source 1..108: mol_type = other DNA
gctcggcgcgcccatatttgcatgtcgctatgtgttctgggaaatcaccataaacgtgaaatgtctttggatttgggaatcttataagttctgtatgagaccacggta

SEQ ID NO: 704 - DNA, 251 bp, synthetic construct
  misc_feature 1..251: note = Synthetic
  source 1..251: mol_type = other DNA
tgacggcgcgccctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggta

SEQ ID NO: 705 - DNA, 249 bp, synthetic construct
  misc_feature 1..249: note = Synthetic
  source 1..249: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 706 - DNA, 243 bp, synthetic construct
  misc_feature 1..243: note = Synthetic
  source 1..243: mol_type = other DNA
ctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctc

SEQ ID NO: 707 - DNA, 100 bp, synthetic construct
  misc_feature 1..100: note = Synthetic
  source 1..100: mol_type = other DNA
aatatttgcatgtcgctatgtgttctgggaaatcaccataaacgtgaaatgtctttggatttgggaatcttataagttctgtatgagaccactctttccc

SEQ ID NO: 708 - protein, 1057 aa, synthetic construct
  REGION 1..1057: note = Synthetic
MNQKFILGLDIGITSVGYGLIDYETKNIIDAGVRLFPEANVENNEGRRSKRGSRRLKRRRIHRLDRVKHLLAEYDLLDLTNIPKSTNPYQTRVKGLNEKLSKDELVIALLHIAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGKGSPFGWEGNIKKWFEQMMGHCTYFPEELRSVKYSYSAELFNALNDLNNLVITRDEDAKLNYGEKFQIIENVFKQKKTPNLKQIAIEIGVHETEIKGYRVNKSGTPEFTEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKRTFIQSINVINKVIEKYGIPEDIIIELARENNSDDRKKFINNLQKKNEATRKRINEIIGQTGNQNAKRIVEKIRLHDQQEGKCLYSLKDIPLEDLLRNPNNYDIDHIIPRSVSFDDSMHNKVLVRREQNAKKNNQTPYQYLTSGYADIKYSVFKQHVLNLAENKDRMTKKKREYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 709 - DNA, 143 bp, synthetic construct
  misc_feature 1..143: note = Synthetic
  source 1..143: mol_type = other DNA
ggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 710 - DNA, 137 bp, synthetic construct
  misc_feature 1..137: note = Synthetic
  source 1..137: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagaggga

SEQ ID NO: 711 - protein, 1052 aa, synthetic construct
  REGION 1..1052: note = Synthetic
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 712 - protein, 1053 aa, synthetic construct
  REGION 1..1053: note = Synthetic
MNQKFILGLDIGITSVGYGLIDYETKNIIDAGVRLFPEANVENNEGRRSKRGSRRLKRRRIHRLERVKSLLSEYKIISGLAPTNNQPYNIRVKGLTEQLTKDELAVALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTEFKLYHDLKSVLFDQSILENEDVLDQIAEILTIYQDKDSIKSKLTELDILLNEEDKENIAQLTGYNGTHRLSLKCIRLVLEEQWYSSRNQMEIFTHLNIKPKKINLTAANKIPKAMIDEFILSPVVKRTFIQSINVINKVIEKYGIPEDIIIELARENNSDDRKKFINNLQKKNEATRKRINEIIGQTGNQNAKRIVEKIRLHDQQEGKCLYSLESIALMDLLNNPQNYEVDHIIPRSVAFDNSIHNKVLVKQIENSKKGNRTPYQYLNSSDAKLSYNQFKQHILNLSKSKDRISKKKKDYLLEERDINKFEVQKEFINRNLVDTRYATRELTSYLKAYFSANNMDVKVKTINGSFTNHLRKVWRFDKYRNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 713 - protein, 7 aa, synthetic construct
  REGION 1..7: note = Synthetic
PKKKRKV

SEQ ID NO: 714 - protein, 16 aa, synthetic construct
  REGION 1..16: note = Synthetic
KRPAATKKAGQAKKKK

SEQ ID NO: 715 - protein, 1052 aa, synthetic construct
  REGION 1..1052: note = Synthetic
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 716 - protein, 1052 aa, synthetic construct
  REGION 1..1052: note = Synthetic
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELASVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDAQIAIFARLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATAGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 717 - protein, 1052 aa, synthetic construct
  REGION 1..1052: note = Synthetic
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELASVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDAQIAIFARLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATAGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 718 - protein, 1057 aa, synthetic construct
  REGION 1..1057: note = Synthetic
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 719 - protein, 1057 aa, synthetic construct
  REGION 1..1057: note = Synthetic
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 720 - protein, 1057 aa, synthetic construct
  REGION 1..1057: note = Synthetic
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 721 - protein, 1057 aa, synthetic construct
  REGION 1..1057: note = Synthetic
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 722 - protein, 1054 aa, synthetic construct
  REGION 1..1054: note = Synthetic
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHNINVSSEDEDASNELSTKEQINRNNKLLKDKYVCEVQLQRLKEGQIRGEKNRFKTTDILKEIDQLLKVQKDYHNLDIDFINQYKEIVETRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 723 - DNA, 20 bp, synthetic construct
  misc_feature 1..20: note = Synthetic
  source 1..20: mol_type = other DNA
cgctaggaagcagccaatga

SEQ ID NO: 724 - DNA, 20 bp, synthetic construct
  misc_feature 1..20: note = Synthetic
  source 1..20: mol_type = other DNA
tagctcctcccagaccttcg

SEQ ID NO: 725 - DNA, 18 bp, synthetic construct
  misc_feature 1..18: note = Synthetic
  source 1..18: mol_type = other DNA
aatgacgagttcggacgg

SEQ ID NO: 726-799 - skipped (74 entries)

SEQ ID NO: 800 - DNA, 20 bp, synthetic construct
  misc_feature 1..20: note = Synthetic
  source 1..20: mol_type = other DNA
ggggatcacagaccatttct

SEQ ID NO: 801 - DNA, 19 bp, synthetic construct
  misc_feature 1..19: note = Synthetic
  source 1..19: mol_type = other DNA
tggaggatggaacacggac

SEQ ID NO: 802 - DNA, 24 bp, synthetic construct
  misc_feature 1..24: note = Synthetic
  source 1..24: mol_type = other DNA
ttctttcggccaggctgaggccct

SEQ ID NO: 803 - DNA, 15 bp, synthetic construct
  misc_feature 1..15: note = Synthetic
  source 1..15: mol_type = other DNA
ctagcggccggggag

SEQ ID NO: 804 - DNA, 17 bp, synthetic construct
  misc_feature 1..17: note = Synthetic
  source 1..17: mol_type = other DNA
agcagcattcccggcta

SEQ ID NO: 805 - DNA, 24 bp, synthetic construct
  misc_feature 1..24: note = Synthetic
  source 1..24: mol_type = other DNA
cgaacggggctcgaagggtccttg

SEQ ID NO: 806 - DNA, 20 bp, synthetic construct
  misc_feature 1..20: note = Synthetic
  source 1..20: mol_type = other DNA
ggatatgtgaccatgctacc

SEQ ID NO: 807 - DNA, 20 bp, synthetic construct
  misc_feature 1..20: note = Synthetic
  source 1..20: mol_type = other DNA
gggttgtatccagtacctct

SEQ ID NO: 808 - DNA, 25 bp, synthetic construct
  misc_feature 1..25: note = Synthetic
  source 1..25: mol_type = other DNA
tgtcctgttccttcccccagcccca

SEQ ID NO: 809-900 - skipped (92 entries)

SEQ ID NO: 901 - DNA, 219 bp, synthetic construct
  misc_feature 1..219: note = Synthetic
  source 1..219: mol_type = other DNA
gagggcctatttcccatgattccttcatatttgcatatacgatacaaggctgttagagagataattggaattaatttgactgtaaacacaaagatataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 902 - DNA, 189 bp, synthetic construct
  misc_feature 1..189: note = Synthetic
  source 1..189: mol_type = other DNA
gagggcctatttcccatgattccttcatatttgcatatacgatacaaggctgttagagagataattggaattaatttgacgtttgcagttttaaaattatgttttaaaatggactatcatatgcttaccgtaacttgaaagtatttcgatttcttggctttatatatcttgtggaaaggacgaaacacc

SEQ ID NO: 903 - DNA, 159 bp, synthetic construct
  misc_feature 1..159: note = Synthetic
  source 1..159: mol_type = other DNA
gagggcctatttcccatgattccttcatatttgcatatacgatacaaggctgttagagagataatattatgttttaaaatggactatcatatgcttaccgtaacttgaaagtatttcgatttcttggctttatatatcttgtggaaaggacgaaacacc

SEQ ID NO: 904 - DNA, 129 bp, synthetic construct
  misc_feature 1..129: note = Synthetic
  source 1..129: mol_type = other DNA
gagggcctatttcccatgattccttcatatttgcatatacgatacaaggcggactatcatatgcttaccgtaacttgaaagtatttcgatttcttggctttatatatcttgtggaaaggacgaaacacc

SEQ ID NO: 905 - skipped

SEQ ID NO: 906 - DNA, 213 bp, synthetic construct
  misc_feature 1..213: note = Synthetic
  source 1..213: mol_type = other DNA
ctgcagtatttagcatgccccacccatctgcaaggcattctggatagtgtcaaaacagccggaaatcaagtccgtttatctcaaactttagcatttaaattagattttagttaaatttcctgctgaagctctagtacgataagcaacttgacctaagtgtaaagttgagacttccttcaggtttatatagcttgtgcgccgcttgggtacctc

SEQ ID NO: 907 - DNA, 183 bp, synthetic construct
  misc_feature 1..183: note = Synthetic
  source 1..183: mol_type = other DNA
ctgcagtatttagcatgccccacccatctgcaaggcattctggatagtgtcaaaacagccggaaatcaagtccgtttatcttaaatttcctgctgaagctctagtacgataagcaacttgacctaagtgtaaagttgagacttccttcaggtttatatagcttgtgcgccgcttgggtacctc

SEQ ID NO: 908 - DNA, 153 bp, synthetic construct
  misc_feature 1..153: note = Synthetic
  source 1..153: mol_type = other DNA
ctgcagtatttagcatgccccacccatctgcaaggcattctggatagtgtcaaaacagccggaaatagctctagtacgataagcaacttgacctaagtgtaaagttgagacttccttcaggtttatatagcttgtgcgccgcttgggtacctc

SEQ ID NO: 909 - DNA, 123 bp, synthetic construct
  misc_feature 1..123: note = Synthetic
  source 1..123: mol_type = other DNA
ctgcagtatttagcatgccccacccatctgcaaggcattctggatagtgtcagcaacttgacctaagtgtaaagttgagacttccttcaggtttatatagcttgtgcgccgcttgggtacctc

SEQ ID NO: 910 - DNA, 77 bp, synthetic construct
  misc_feature 1..77: note = Synthetic
  source 1..77: mol_type = other DNA
gttttagtactctggaaacagaatctactaaaacaaggcaaaatgccgtgtttatctcgtcaacttgttggcgagat

SEQ ID NO: 911 - DNA, 87 bp, synthetic construct
  misc_feature 1..87: note = Synthetic
  source 1..87: mol_type = other DNA
gtttaagtactctgtgctggaaacagcacagaatctacttaaacaaggcaaaatgccgtgtttatctcgtcaacttgttggcgagat

SEQ ID NO: 912 - DNA, 77 bp, synthetic construct
  misc_feature 1..77: note = Synthetic
  source 1..77: mol_type = other DNA
gtttaagtactctggaaacagaatctacttaaacaaggcaaaatgccgtgtttatctcgtcaacttgttggcgagat

SEQ ID NO: 913 - skipped

SEQ ID NO: 914 - DNA, 3156 bp, synthetic construct
  misc_feature 1..3156: note = Synthetic
  source 1..3156: mol_type = other DNA
aagcgcaattacatcctgggcctggatatcggcatcacctccgtgggctacggcatcatcgactatgagacacgggatgtgatcgacgccggcgtgagactgttcaaggaggccaacgtggagaacaatgagggccggcggagcaagaggggagcaaggcgcctgaagcggagaaggcgccacagaatccagagagtgaagaagctgctgttcgattacaacctgctgaccgaccactccgagctgtctggcatcaatccttatgaggcccgggtgaagggcctgtcccagaagctgtctgaggaggagttttctgccgccctgctgcacctggcaaagaggagaggcgtgcacaacgtgaatgaggtggaggaggacaccggcaacgagctgagcacaaaggagcagatcagccgcaattccaaggccctggaggagaagtatgtggccgagctgcagctggagcggctgaagaaggatggcgaggtgaggggctccatcaatcgcttcaagacctctgactacgtgaaggaggccaagcagctgctgaaggtgcagaaggcctaccaccagctggatcagagctttatcgatacatatatcgacctgctggagaccaggcgcacatactatgagggaccaggagagggctcccccttcggctggaaggacatcaaggagtggtacgagatgctgatgggccactgcacctattttccagaggagctgagatccgtgaagtacgcctataacgccgatctgtacaacgccctgaatgacctgaacaacctggtcatcaccagggatgagaacgagaagctggagtactatgagaagttccagatcatcgagaacgtgttcaagcagaagaagaagcctacactgaagcagatcgccaaggagatcctggtgaacgaggaggacatcaagggctaccgcgtgaccagcacaggcaagccagagttcaccaatctgaaggtgtatcacgatatcaaggacatcacagcccggaaggagatcatcgagaacgccgagctgctggatcagatcgccaagatcctgaccatctatcagagctccgaggacatccaggaggagctgaccaacctgaatagcgagctgacacaggaggagatcgagcagatcagcaatctgaagggctacaccggcacacacaacctgtccctgaaggccatcaatctgatcctggatgagctgtggcacacaaacgacaatcagatcgccatctttaacaggctgaagctggtgccaaagaaggtggacctgagccagcagaaggagatcccaaccacactggtggacgatttcatcctgtcccccgtggtgaagcggagcttcatccagagcatcaaagtgatcaacgccatcatcaagaagtacggcctgcccaatgatatcatcatcgagctggccagggagaagaactctaaggacgcccagaagatgatcaatgagatgcagaagaggaaccgccagaccaatgagcggatcgaggagatcatcagaaccacaggcaaggagaacgccaagtacctgatcgagaagatcaagctgcacgatatgcaggagggcaagtgtctgtatagcctggaggccatccctctggaggacctgctgaacaatccattcaactacgaggtggatcacatcatcccccggagcgtgagcttcgacaattcctttaacaataaggtgctggtgaagcaggaggagaactctaagaagggcaataggacccctttccagtacctgtctagctccgattctaagatcagctacgagaccttcaagaagcacatcctgaatctggccaagggcaagggccgcatctctaagaccaagaaggagtacctgctggaggagcgggacatcaacagattcagcgtgcagaaggacttcatcaaccggaatctggtggacaccagatacgccacacgcggcctgatgaatctgctgcggtcctatttcagagtgaacaatctggatgtgaaggtgaagagcatcaacggcggcttcacctcctttctgcggagaaagtggaagtttaagaaggagagaaacaagggctataagcaccacgccgaggatgccctgatcatcgccaatgccgacttcatctttaaggagtggaagaagctggacaaggccaagaaagtgatggagaaccagatgttcgaggagaagcaggccgagagcatgcccgagatcgagaccgagcaggagtacaaggagattttcatcacacctcaccagatcaagcacatcaaggacttcaaggactacaagtattcccacagggtggataagaagcccaaccgcgagctgatcaatgacaccctgtattctacaaggaaggacgataagggcaataccctgatcgtgaacaatctgaacggcctgtacgacaaggataatgacaagctgaagaagctgatcaacaagagccccgagaagctgctgatgtaccaccacgatcctcagacatatcagaagctgaagctgatcatggagcagtacggcgacgagaagaacccactgtataagtactatgaggagaccggcaactacctgacaaagtattccaagaaggataatggccccgtgatcaagaagatcaagtactatggcaacaagctgaatgcccacctggacatcaccgacgattaccccaacagccggaataaggtggtgaagctgagcctgaagccatacaggttcgacgtgtacctggacaacggcgtgtataagtttgtgacagtgaagaatctggatgtgatcaagaaggagaactactatgaagtgaatagcaagtgctacgaggaggccaagaagctgaagaagatcagcaaccaggccgagttcatcgcctctttttacaacaatgacctgatcaagatcaatggcgagctgtatagagtgatcggcgtgaacaatgatctgctgaaccgcatcgaagtgaatatgatcgacatcacctaccgggagtatctggagaacatgaatgataagaggccccctcgcatcatcaagaccatcgcctctaagacacagagcatcaagaagtactctacagacatcctgggcaacctgtatgaggtgaagagcaagaagcaccctcagatcatcaagaagggc

SEQ ID NO: 915 - DNA, 21 bp, synthetic construct
  misc_feature 1..21: note = Synthetic
  source 1..21: mol_type = other DNA
ccaaagaagaagcggaaggtc

SEQ ID NO: 916 - protein, 7 aa, synthetic construct
  REGION 1..7: note = Synthetic
PKKKRKV

SEQ ID NO: 917 - DNA, 48 bp, synthetic construct
  misc_feature 1..48: note = Synthetic
  source 1..48: mol_type = other DNA
aagcgacctgccgccacaaagaaggctggacaggctaagaagaagaaa

SEQ ID NO: 918 - protein, 16 aa, synthetic construct
  REGION 1..16: note = Synthetic
KRPAATKKAGQAKKKK

SEQ ID NO: 919 - DNA, 49 bp, synthetic construct
  misc_feature 1..49: note = Synthetic
  source 1..49: mol_type = other DNA
aataaaatatctttattttcattacatctgtgtgttggttttttgtgtg

SEQ ID NO: 920 - DNA, 87 bp, synthetic construct
  misc_feature 1..87: note = Synthetic
  source 1..87: mol_type = other DNA
gtttcagtactctgtgctggaaacagcacagaatctactgaaacaaggcaaaatgccgtgtttatctcgtcaacttgttggcgagat

SEQ ID NO: 921 - DNA, 77 bp, synthetic construct
  misc_feature 1..77: note = Synthetic
  source 1..77: mol_type = other DNA
gtttcagtactctggaaacagaatctactgaaacaaggcaaaatgccgtgtttatctcgtcaacttgttggcgagat

SEQ ID NO: 922 - DNA, 773 bp, synthetic construct
  misc_feature 1..773: note = Synthetic
  source 1..773: mol_type = other DNA
tgtactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactttgcgaaccaacgataggtgtttcagtactctggaaacagaatctactgaaacaaggcaaaatgccgtgtttatctcgtcaacttgttggcgagatttttttctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcgccagttcacaaccgctccgagcgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 923 - DNA, 773 bp, synthetic construct
  misc_feature 1..773: note = Synthetic
  source 1..773: mol_type = other DNA
tgtactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttgggggtcctgtagcctgtcagtttcagtactctggaaacagaatctactgaaacaaggcaaaatgccgtgtttatctcgtcaacttgttggcgagatttttttctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcgccagttcacaaccgctccgagcgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 924 - DNA, 772 bp, synthetic construct
  misc_feature 1..772: note = Synthetic
  source 1..772: mol_type = other DNA
tgtactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactttgcgaaccaacgataggtgtttcagtactctggaaacagaatctactgaaacaaggcaaaatgccgtgtttatctcgtcaacttgttggcgagatttttttctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcggctcgaagggtccttgtagccgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 925 - DNA, 773 bp, synthetic construct
  misc_feature 1..773: note = Synthetic
  source 1..773: mol_type = other DNA
tgtactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactttgcgaaccaacgataggtgtttcagtactctggaaacagaatctactgaaacaaggcaaaatgccgtgtttatctcgtcaacttgttggcgagatttttttctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcgactcagtcttccaacggggcccgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 926 - DNA, 772 bp, synthetic construct
  misc_feature 1..772: note = Synthetic
  source 1..772: mol_type = other DNA
tgtactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccagttcacaaccgctccgagcgtttcagtactctggaaacagaatctactgaaacaaggcaaaatgccgtgtttatctcgtcaacttgttggcgagatttttttctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcgggtttggcaaaagcaaatttcgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 927 - DNA, 773 bp, synthetic construct
  misc_feature 1..773: note = Synthetic
  source 1..773: mol_type = other DNA
tgtactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccagttcacaaccgctccgagcgtttcagtactctggaaacagaatctactgaaacaaggcaaaatgccgtgtttatctcgtcaacttgttggcgagatttttttctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcgactttgcgaaccaacgataggtgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 928 - DNA, 779 bp, synthetic construct
  misc_feature 1..779: note = Synthetic
  source 1..779: mol_type = other DNA
tgtactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactttgcgaaccaacgataggtgtttcagtactctggaaacagaatctactgaaacaaggcaaaatgccgtgtttatctcgtcaacttgttggcgagatttttttgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccagttcacaaccgctccgagcgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 929 - DNA, 773 bp, synthetic construct
  misc_feature 1..773: note = Synthetic
  source 1..773: mol_type = other DNA
tgtactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcgcggccggcgaacggggctcggtttcagtactctggaaacagaatctactgaaacaaggcaaaatgccgtgtttatctcgtcaacttgttggcgagatttttttctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcgttgggggtcctgtagcctgtcagtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 930 - DNA, 771 bp, synthetic construct
  misc_feature 1..771: note = Synthetic
  source 1..771: mol_type = other DNA
tgtactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccccggagtcgaagacagttcgtttcagtactctggaaacagaatctactgaaacaaggcaaaatgccgtgtttatctcgtcaacttgttggcgagatttttttctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcgccccggagtcgaagacagttcgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 931 - DNA, 771 bp, synthetic construct
  misc_feature 1..771: note = Synthetic
  source 1..771: mol_type = other DNA
tgtactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcaacatcctggggcacaagcgtttcagtactctggaaacagaatctactgaaacaaggcaaaatgccgtgtttatctcgtcaacttgttggcgagatttttttctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcggcaacatcctggggcacaagcgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 932 - DNA, 743 bp, synthetic construct
  misc_feature 1..743: note = Synthetic
  source 1..743: mol_type = other DNA
tgtactagtgagggcctatttcccatgattccttcatatttgcatatacgatacaaggctgttagagagataattggaattaatttgactgtaaacacaaagatataatttcttgggtagtttgcagttttaaaattatgttttaaaatggactatcatatgcttaccgtaacttgaaagtatttcgatttcttggctttatatatcttgtggaaaggacgaaacaccgactttgcgaaccaacgataggtgtttcagtactctggaaacagaatctactgaaacaaggcaaaatgccgtgtttatctcgtcaacttgttggcgagatttttttctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcgccagttcacaaccgctccgagcgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 933 - DNA, 713 bp, synthetic construct
  misc_feature 1..713: note = Synthetic
  source 1..713: mol_type = other DNA
tgtactagtgagggcctatttcccatgattccttcatatttgcatatacgatacaaggctgttagagagataattggaattaatttgacgtttgcagttttaaaattatgttttaaaatggactatcatatgcttaccgtaacttgaaagtatttcgatttcttggctttatatatcttgtggaaaggacgaaacaccgactttgcgaaccaacgataggtgtttcagtactctggaaacagaatctactgaaacaaggcaaaatgccgtgtttatctcgtcaacttgttggcgagatttttttctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcgccagttcacaaccgctccgagcgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 934 - DNA, 683 bp, synthetic construct
  misc_feature 1..683: note = Synthetic
  source 1..683: mol_type = other DNA
tgtactagtgagggcctatttcccatgattccttcatatttgcatatacgatacaaggctgttagagagataatattatgttttaaaatggactatcatatgcttaccgtaacttgaaagtatttcgatttcttggctttatatatcttgtggaaaggacgaaacaccgactttgcgaaccaacgataggtgtttcagtactctggaaacagaatctactgaaacaaggcaaaatgccgtgtttatctcgtcaacttgttggcgagatttttttctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcgccagttcacaaccgctccgagcgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 935 - DNA, 653 bp, synthetic construct
  misc_feature 1..653: note = Synthetic
  source 1..653: mol_type = other DNA
tgtactagtgagggcctatttcccatgattccttcatatttgcatatacgatacaaggcggactatcatatgcttaccgtaacttgaaagtatttcgatttcttggctttatatatcttgtggaaaggacgaaacaccgactttgcgaaccaacgataggtgtttcagtactctggaaacagaatctactgaaacaaggcaaaatgccgtgtttatctcgtcaacttgttggcgagatttttttctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcgccagttcacaaccgctccgagcgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 936 - DNA, 743 bp, synthetic construct
  misc_feature 1..743: note = Synthetic
  source 1..743: mol_type = other DNA
tgtactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactttgcgaaccaacgataggtgtttcagtactctggaaacagaatctactgaaacaaggcaaaatgccgtgtttatctcgtcaacttgttggcgagatttttttctgcagtatttagcatgccccacccatctgcaaggcattctggatagtgtcaaaacagccggaaatcaagtccgtttatctcaaactttagcatttaaattagattttagttaaatttcctgctgaagctctagtacgataagcaacttgacctaagtgtaaagttgagacttccttcaggtttatatagcttgtgcgccgcttgggtacctcgccagttcacaaccgctccgagcgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 937 - DNA, 713 bp, synthetic construct
  misc_feature 1..713: note = Synthetic
  source 1..713: mol_type = other DNA
tgtactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactttgcgaaccaacgataggtgtttcagtactctggaaacagaatctactgaaacaaggcaaaatgccgtgtttatctcgtcaacttgttggcgagatttttttctgcagtatttagcatgccccacccatctgcaaggcattctggatagtgtcaaaacagccggaaatcaagtccgtttatcttaaatttcctgctgaagctctagtacgataagcaacttgacctaagtgtaaagttgagacttccttcaggtttatatagcttgtgcgccgcttgggtacctcgccagttcacaaccgctccgagcgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 938 - DNA, 683 bp, synthetic construct
  misc_feature 1..683: note = Synthetic
  source 1..683: mol_type = other DNA
tgtactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactttgcgaaccaacgataggtgtttcagtactctggaaacagaatctactgaaacaaggcaaaatgccgtgtttatctcgtcaacttgttggcgagatttttttctgcagtatttagcatgccccacccatctgcaaggcattctggatagtgtcaaaacagccggaaatagctctagtacgataagcaacttgacctaagtgtaaagttgagacttccttcaggtttatatagcttgtgcgccgcttgggtacctcgccagttcacaaccgctccgagcgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 939 - DNA, 653 bp, synthetic construct
  misc_feature 1..653: note = Synthetic
  source 1..653: mol_type = other DNA
tgtactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactttgcgaaccaacgataggtgtttcagtactctggaaacagaatctactgaaacaaggcaaaatgccgtgtttatctcgtcaacttgttggcgagatttttttctgcagtatttagcatgccccacccatctgcaaggcattctggatagtgtcagcaacttgacctaagtgtaaagttgagacttccttcaggtttatatagcttgtgcgccgcttgggtacctcgccagttcacaaccgctccgagcgtttcagtactctggaaacagaatctactgaaacaaggcaaaatgccgtgtttatctcgtcaacttgttggcgagatttttttagatctgcccatgtaaggaggcaaggcctggggacacccgagatgcctggttataattaa

SEQ ID NO: 940 - protein, 4 aa, synthetic construct
  REGION 1..4: note = Synthetic
GSVD

SEQ ID NO: 941 - protein, 4 aa, synthetic construct
  REGION 1..4: note = Synthetic
GSGS

SEQ ID NO: 942 - protein, 9 aa, synthetic construct
  REGION 1..9: note = Synthetic
PAAKKKKLD

SEQ ID NO: 943 - DNA, 27 bp, synthetic construct
  misc_feature 1..27: note = Synthetic
  source 1..27: mol_type = other DNA
ccggcagctaagaaaaagaaactggat